SEQUENCE LISTING (9 sequences)

SEQ ID NO: 1 - protein, 270 aa, Pycnoporus coccineus
HAAFWDKSMYGFNVTAQTFPYDNRPQVPLYNMTFDQWWFHGHKDYPPNEGDFFELPAGGEVNSIISCDKGATPFYESSPGGDSGYGSNSPCPGQPMSEYHTTGIDDVKGCCMAIAYKPDVNDVQPDDFVVFSCNSTCVWEMNTKFEIPKLPACPEGGCHCAWFWIHSYDSGAEQIYMNGFKCKVTGDVGTQPLGKPAVPRRCGADPDHGKPDPTPGNCTIGAKTPMYWYQREGNNMFEDTYDAPYYNPLYGFNDGAQNDIFMDGVIASLA

SEQ ID NO: 2 - protein, 269 aa, Pycnoporus coccineus
HIAFWHNSMYGFNVTEQTFPYDNRPVVPLQYMTFQEWWFHNHLDYPPHPGDFFDFPAGKAATAELACNKGATTWFNSSEGGNIQNGNDPCPGSPPSEYHTTGIDDVKGCAMAIAYESDVRKIKPEDFTVFSVNQTCVWYRFTDFQVPERMPPCPPGGCHCAWFWIHSPDSGGEQIYMNGFQCNITGSTSHVPLAKPKVARRCGADPDHGKPDAVPGNCTYGAKQPLYWLQKEGNNEFDDYIAPPFYNDLYNFKDGAQNDIFVDSYPDGI

SEQ ID NO: 3 - protein, 269 aa, Pycnoporus coccineus
HVAAFVKGMYCEGGPDPNNYNPNSNTPVNPLWDLPFEQWWMQADRGCNKAPPPDGASVALPAGGQFTVELAHNQAQTTLSFNGQFAGEWPDGQPHPENWSGPGSPPDCIQDDGAMHTNNQTMAAGTAWAISYNDDISKVTMDNLVVFSVLEHTPWKRIATYDVPKDLPACPAGGCYCAWLWVPNGCGEPNMYMANYRCHVTNTTSTKQLAQAKPPTWCGGDSSKCTKGAKQMIAWNQATGNNVQVPNGASPGYNINMGWAPGAQNDIFA

SEQ ID NO: 4 - DNA, 810 bp, Pycnoporus coccineus
cacgccgcgttctgggacaagtccatgtacggcttcaacgtcaccgcacagaccttcccctacgacaaccggccccaggttccgctctacaacatgactttcgaccaatggtggttccacggtcacaaagactacccacccaacgagggcgatttcttcgaactccctgcgggtggagaggtgaacagcatcatctcctgcgataagggtgcgactccgttctacgagtcgtctccgggcggagattcgggctatggcagcaactctccttgccccgggcagcccatgtccgagtaccacacgacgggcattgatgatgtcaagggctgctgcatggccatcgcgtacaagcccgatgtcaacgatgtgcagccagacgacttcgtggtcttctcctgcaactcgacgtgcgtttgggagatgaacaccaagtttgagatcccgaagctccctgcctgccccgaaggtggttgccattgcgcctggttctggattcactcctacgatagcggtgctgagcagatctatatgaatggtttcaagtgcaaagtaaccggcgacgtaggcactcagcctctcggcaagcctgccgtcccgcgcaggtgcggtgccgaccctgatcacggcaagcctgatcctactcccggtaattgtactatcggggccaagacgcccatgtactggtaccagcgggagggtaacaacatgttcgaagacacttatgacgccccgtactataacccactctacggcttcaacgatggggcgcagaacgacattttcatggatggtgtcatcgcgtccctcgca

SEQ ID NO: 5 - DNA, 807 bp, Pycnoporus coccineus
cacatcgccttctggcataacagcatgtacgggttcaatgtgacggaacagacgttcccttacgacaaccgccccgtcgtcccgcttcagtacatgaccttccaagaatggtggttccacaaccacctcgactacccgccccacccgggcgacttcttcgacttcccggccggcaaggccgcgacggcggagctcgcgtgcaacaagggcgcgaccacctggttcaactcctccgagggcggcaacatccagaacggcaacgacccgtgcccggggagccccccgagcgagtaccacacgacgggcatcgacgacgtgaagggctgcgcgatggcgatcgcgtacgagtccgacgtcaggaagatcaagcccgaggacttcaccgtcttcagcgtgaaccagacgtgcgtctggtaccgcttcacggacttccaggtccccgagcgcatgccgccgtgccctcctggcggctgtcactgcgcgtggttctggatccactcgcccgatagcggcggtgagcagatctacatgaacggcttccagtgcaacatcaccggctccacgtcccacgtcccgctcgcaaagcccaaagtcgctcgccgctgcggtgcggacccggaccacggcaagcccgacgccgtccccggcaactgcacatacggtgcgaagcagcccctctactggctccagaaggagggcaacaacgagttcgacgactacatcgcgccgccgttctacaacgacctgtacaacttcaaggacggcgcgcagaacgacatcttcgtcgactcgtaccccgacggcatc

SEQ ID NO: 6 - DNA, 861 bp, Pycnoporus coccineus
atgttctggacggttcccgttacactggctctcgcctccggggcctctgcccatgttgccgccttcgtgaagggcatgtactgcgagggtggccccgaccctaacaactacaaccccaactccaacactcccgtcaaccctctctgggatcttcctttcgagcagtggtggatgcaggccgaccgtggctgcaacaaggctcctcctcctgacggtgcctccgttgctctgcctgccggcggccagttcactgttgagctcgcccacaaccaggcccagacgaccttatccttcaatggacagttcgctggcgagtggcctgatggccagcctcacccagagaactggagcggccccggtagcccccctgactgcatccaggacgacggtgccatgcataccaacaaccagacaatggccgctggtaccgcttgggccatttcctacaatgacgacatctccaaggtgaccatggacaacctggttgttttctctgtccttgagcacaccccttggaagcgcatcgccacctatgatgtccccaaggatctccctgcttgccctgctggcggctgctactgcgcctggctttgggttcccaacgggtgcggcgagcccaacatgtacatggccaactacaggtgccatgtcaccaacaccacctcgaccaagcagcttgctcaagccaagccccccacctggtgtggcggtgactcttccaagtgcaccaagggcgctaagcagatgattgcttggaaccaggccacaggcaacaatgtccaagttcccaatggcgcttcccccggctacaacatcaacatgggctgggctcccggtgctcagaacgacatcttcgcgtag

SEQ ID NO: 7 - protein, 185 aa, Pycnoporus coccineus
HIAFWHNSMYGFNVTEQTFPYDNRPVVPLQYMTFQEWWFHNHLDYPPHPGDFFDFPAGKAATAELACNKGATTWFNSSEGGNIQNGNDPCPGSPPSEYHTTGIDDVKGCAMAIAYESDVRKIKPEDFTVFSVNQTCVWYRFTDFQVPERMPPCPPGGCHCAWFWIHSPDSGGEQIYMNGFQCNIT

SEQ ID NO: 8 - protein, 17 aa, Artificial Sequence
  Signal peptide
MFWTVPVTLALASGASA

SEQ ID NO: 9 - protein, 286 aa, Pycnoporus coccineus
MFWTVPVTLALASGASAHVAAFVKGMYCEGGPDPNNYNPNSNTPVNPLWDLPFEQWWMQADRGCNKAPPPDGASVALPAGGQFTVELAHNQAQTTLSFNGQFAGEWPDGQPHPENWSGPGSPPDCIQDDGAMHTNNQTMAAGTAWAISYNDDISKVTMDNLVVFSVLEHTPWKRIATYDVPKDLPACPAGGCYCAWLWVPNGCGEPNMYMANYRCHVTNTTSTKQLAQAKPPTWCGGDSSKCTKGAKQMIAWNQATGNNVQVPNGASPGYNINMGWAPGAQNDIFA

(19) United States
(12) Patent Application Publication
(10) Pub. No.: US 2020/0157591 A1
(43) Pub. Date: May 21, 2020

(54) METHODS OF DEFIBRILLATING CELLULOSIC SUBSTRATES AND PRODUCING CELLULOSES USING A NEW FAMILY OF FUNGAL LYTIC POLYSACCHARIDE MONOOXYGENASES (LPMO)

(71) Applicant: INSTITUT NATIONAL DE LA RECHERCHE AGRONOMIQUE (INRA), Paris (FR)

(72) Inventors: Bernard CATHALA, La Chapelle-sur-Erdre (FR); Jean-Guy BERRIN, Roquevaire (FR); Ana VILLARES, Nantes (FR); Céline MOREAU, Carquefou (FR)

(73) Assignee: INSTITUT NATIONAL DE LA RECHERCHE AGRONOMIQUE (INRA), Paris (FR)

(21) Appl. No.: 16/633,316

(22) PCT Filed: Jul. 31, 2018
(86) PCT No.: PCT/EP2018/070754

§ 371 Date: Jan. 23, 2020

(30) Foreign Application Priority Data
Aug. 2, 2017 (FR) 17 57422

(51) Int. Cl. C12P 19/04; D21H 11/18; D21C 5/00

(52) U.S. Cl. CPC C12P 19/04; C12Y 114/14001; D21C 5/005; D21H 11/18

(57) ABSTRACT

Celluloses, notably nanocelluloses, and more particularly processes for manufacturing cellulose fibers and for defibrillating cellulose-based substrates. Most particularly, processes for defibrillating cellulose-based substrates and manufacturing celluloses, in particular nanocelluloses (NC), using a new family of fungal lytic polysaccharide monooxygenases (LPMOs).

### FIELD OF THE INVENTION

The present invention relates generally to the field of celluloses, notably of nanocelluloses, and more particularly to processes for manufacturing cellulose fibers and for defibrillating cellulose-based substrates.

### TECHNOLOGICAL BACKGROUND

Cellulose is one of the most important natural polymers, a virtually inexhaustible raw material, and an important source of durable materials at the industrial scale.

To date, various forms of cellulose have been identified with a size of the order of a nanometer, referred to by the generic term “nanocelluloses”.

The properties of these nanocelluloses, notably their mechanical properties, their capacity to form films and their viscosity, make them of major interest in numerous industrial fields.

Nanocelluloses are thus used, for example, as dispersant or stabilizing additive in the paper, pharmaceutical, cosmetic or agrifood industries. They are also included in the composition of paints and varnishes.

Nanocelluloses are also used in numerous devices requiring nanometric porosity control, on account of their high specific surface area.

Finally, numerous nanocellulose-based nanocomposite materials are currently being developed. Specifically, the noteworthy mechanical properties of nanocelluloses, their dispersion at the nanometric scale and their hydrophilic nature give them excellent gas-barrier properties. These features notably arouse considerable interest for the manufacture of barrier packagings.

On the basis of their sizes, functions and preparation methods, which themselves depend mainly on the source of the cellulose and on the treatment conditions, nanocelluloses may be classified mainly into two families: cellulose fibrils and cellulose nanocrystals.

Cellulose nanocrystals (also known as NCCs, meaning nanocrystalline celluloses) are generally obtained by hydrolysis with a strong acid under strictly controlled temperature, duration and stirring conditions. Such a treatment makes it possible to attack the amorphous regions of the fibers while leaving the crystalline regions, which are more resistant, intact. The suspension obtained is then washed by successive centrifugations and dialyses in distilled water. The NCCs most conventionally obtained have a length from a few tens of nanometers to about 1 m (notably from 40 nm to 1 m and preferably from 40 nm to 500 nm) and a diameter ranging from 5 to 70 nm, preferably less than 15 nm (typically from 5 to 10 nm).

Cellulose fibrils, commonly referred to as cellulose microfibrils (also known as MFC, meaning “microfibrillated cellulose”) or cellulose nanofibrils (NFC, meaning “nanofibrillated cellulose”) are typically isolated from cellulose materials obtained from biomass, via mechanical processes for stripping the cellulose fibers and releasing the cellulose fibrils.

Although they have been known since the 1950s, nanocelluloses have featured considerably in the literature in the last 10 years.

For example, U.S. Pat. No. 4,483,743 describes a process for manufacturing microfibrillated cellulose, which involves passing a liquid suspension of cellulose through a high-pressure Gaulin homogenizer. Repeated passes of the cellulose suspension make it possible to obtain microfibrils with a width typically ranging from 25 to 100 nm and a much longer length.

In general, the mechanical processes for obtaining cellulose fibrils have the drawback of consuming large amounts of energy. By way of example, it was evaluated that the use of a homogenizer entails an energy consumption of the order of 70 000 kWh/t. This high energy consumption, the corollary of which is the high costs of nanocellulose production, thus remain a considerable curb on their industrial development.

Various strategies for pretreating cellulose fibers have thus been developed in order to reduce the energy consumption required for their mechanical stripping.

A first pretreatment strategy, described, for example, in patent application WO 2007/091942, consists in pretreating the cellulose fibers with cellulases so as to destructure the fiber before applying the mechanical treatment by homogenization.

However, this enzymatic pretreatment is extremely versatile depending on the state of the fiber and notably depending on the prior thermochemical history of the fiber.

Furthermore, the quality of the nanocelluloses obtained (in particular the state of dispersion and notably the lateral size of the nanofibrils which conditions the working properties and the energy yields) are very variable.

A second pretreatment strategy is based on a chemical step of oxidation of the cellulose fibers (for example Saito et al., Biomacromolecules, Vol. 8, No. 8, 2007, pages 2485-2491).

Typically, the fibers are oxidized with an oxidant such as sodium hypochlorite catalyzed with the 2,2,6,6-tetramethylpiperidine-1-oxyl (TEMPO) radical before undergoing the abovementioned mechanical treatment.

The oxidative treatment converts the primary alcohol function in the C₆ position of the glucose unit of cellulose into a carboxylate function, which leads to the introduction of charges at the surface of the cellulose fibers. These charges create electrostatic repulsions which facilitate the stripping and which increase its efficiency.

However, the removal of the reaction products leads to large amounts of highly polluted effluents. In addition, reagent residues persist in the final product and continue to react, ultimately impairing the properties of the cellulose fibers produced via these processes.

Thus, despite the new pretreatment strategies developed, the nanocelluloses production costs remain high, the yields remain uncertain and the quality and properties are variable.

More recently, WO 2016/193617 teaches nanocellulose manufacturing processes, comprising a step of enzymatic treatment of said substrates with a cleavage enzyme belonging to the family of lytic polysaccharide monooxygenases (LPMOs), which are capable of ensuring oxidative cleavage of said cellulose fibers.

For obvious reasons, the provision of novel optimized processes for obtaining nanocelluloses is an ongoing concern. The optimization routes are notably directed toward reducing the energy consumption, favoring a simplified and reproducible procedure, and reduced or even zero toxicity.

The present invention is directed, precisely, toward proposing a process which at least partly meets these expectations.

### SUMMARY OF THE INVENTION

According to a first subject, the invention relates to a process for preparing a cellulose-based substrate for the manufacture of cellulose fibers, which process comprises at least the following steps consisting in:

a) having a cellulose-based substrate which is capable of forming cellulose fibers;

b) placing said substrate in contact with an electron donor, with at least one enzyme belonging to the family of lytic polysaccharide monooxygenases (LPMOs) under conditions that are capable of ensuring oxidative cleavage of said cellulose fibers;

characterized in that said enzyme is a polysaccharide which has an amino acid sequence identity of at least 20% with a reference polypeptide of SEQ ID NO. 1, 2 or 3, by means of the BLAST-P comparison method, said BLAST-P comparison method resulting in an E-value of 10 e⁻³ or less.

According to another subject, the invention relates to the use of a cellulose-based substrate obtained according to the preceding process as cellulose-based starting material for preparing cellulose fibers, in particular nanocellulose, notably cellulose fibrils and/or cellulose nanocrystals, via a defibrillation process, notably a mechanical defibrillation process.

More precisely, according to another subject, the invention relates to a process for defibrillating a cellulose-based substrate, which process comprises at least the following steps:

a) having a cellulose-based substrate obtained according to the process as defined previously and

b) mechanically treating said cellulose-based substrate, so as to extract said cellulose fibers from said substrate.

According to yet another of its subjects, the invention relates to a process for manufacturing cellulose fibers, which process comprises at least the following steps:

a) having a cellulose-based substrate which is capable of forming cellulose fibers;

b) placing said cellulose-based substrate in contact with at least one enzyme belonging to the family of lytic polysaccharide monooxygenases (LPMOs) under conditions that are capable of ensuring oxidative cleavage of said cellulose fibers in the presence of an electron donor;

c) mechanically treating said cellulose-based substrate, so as to manufacture said cellulose fibers from said cellulose-based substrate,

characterized in that said enzyme is a polysaccharide which has an amino acid sequence identity of at least 20% with a reference polypeptide of SEQ ID NO. 1, 2 or 3, by means of the BLAST-P comparison method, said BLAST-P comparison method resulting in an E-value of 10 e⁻³ or less.

According to yet another of its subjects, the invention relates to cellulose fibers derived from an extraction process, and/or from a manufacturing process, as defined above.

The cellulose fibers that are most particularly considered by the invention are cellulose nanofibres, or nanocelluloses.

### DESCRIPTION OF THE FIGURES

FIG. 1: Phylogenetic tree of the family AAxx demonstrating that the members of the family AAxx cluster together strongly and are very remote from the sequences relating to the families AA9, AA10, AA11 and AA13, respectively

FIG. 2: online diagram the active site of the LPMOs of PcAAxx type

FIG. 3A-3B: consensus sequence based on an alignment of 283 sequences belonging to the catalytic module of the families AAxx revealing a first histidine conserved in the family

FIG. 4: Morphology of cellulose fibers from birch (A and B) and from coniferous trees (C and D) under the action of polysaccharide monooxygenases. Light microscopy images of control fibers (A and C) and of fibers subjected to the treatment with an LPMO of PcAAxxA type corresponding to a polypeptide of sequence SEQ ID NO. 1 (B and D)

FIG. 5: State of defibrillation of cellulose fibers under the action of a polysaccharide monooxygenase (PcAAxxA/SEQ ID NO. 1). TEM images (AC) and AFM images (B and D)

### DETAILED DESCRIPTION OF THE INVENTION

The object of the invention is to meet these needs.

In order to overcome the abovementioned drawbacks of the prior art, the present invention proposes a process for manufacturing nanocelluloses based on a step of pre-treatment of cellulose fibers with at least one enzyme belonging to the family of lytic polysaccharide monooxygenases, commonly referred to as “LPMOs”.

The inventors are thus of the opinion that the use of LPMOs makes it possible to propose a novel process for the manufacture of cellulose fibers, including nanocelluloses. Specifically, LPMOs are enzymes whose oxidative function has recently been revealed (Vaaje-Kolstad, 2010; Quinlan et al. 2011; Westereng et al. 2011; Horn et al., 2012; Bey et al., 2013). These enzymes, which improve the degradation of lignocellulose (Harris et al., 2010) are found in the latest generation of industrial enzyme cocktails (e.g. Cellic CTec3). They are copper-dependent enzymes which catalyze the oxidative cleavage of cellulose. These enzymes, formerly classified in the family GH61 (Glycoside Hydrolase) of CAZy (www.cazy.org), have been reclassified in the family AA9 (“Auxiliary Activity” enzymes).

The studies conducted during preliminary tests had the objective of providing the proof of concept of the manufacture of nanocellulose following the action of fungal LPMOs.

Surprisingly, it is now been demonstrated on a papermaking fiber that an enzyme belonging to a new family produced in the heterologous system in the yeast Pichia pastoris could be advantageously used in processes for defibrillating cellulose fibers, and also in processes for manufacturing cellulose fibers, notably nanocelluloses.

This new family of enzymes capable of oxidizing polysaccharides is also referenced herein as the protein family AAxx. The inventors structurally characterized the reference protein PcAAxxB (Genbank #KY769370) from P. coccineus, by resolving the crystallographic structure of its catalytic module at a resolution of 3 Å. They thus provided a structural model for the identification of all the pertinent members belonging to this protein family AAxx, in addition to analysis by sequence alignment of more than 300 proteins having significant similarities with PcAAxxB.

The present invention thus exploits the particular enzymatic properties of a new class of enzyme with polysaccharide oxidase activity, characterized in that: said enzyme with polysaccharide oxidase activity has an amino acid sequence identity of at least 30% with a reference polypeptide of SEQ ID NO. 1, 2 or 3, by means of the BLAST-P comparison method, said BLAST-P comparison method resulting in an E-value of 10 e⁻³ or less.

In particular, the inventors have identified a crystallographic structure of the polypeptide of sequence SEQ ID NO 2.

The enzymes with polysaccharide oxidase activity according to the invention are characterized by the presence of a copper-binding conserved active site, also referred to herein as a copper-binding active site of “histidine brace” type, formed by two Histidine residues and one Tyrosine residue, one of the two histidine residues being the N-terminal histidine after cleavage of the signal peptide.

More specifically, the inventors have shown here that the plurality of the proteins belonging to this new family AAxx can be differentiated from other lytic polysaccharide monooxygenases in that this new family AAxx does not mandatorily bear a carbohydrate-binding module (CBM).

More precisely, the inventors have demonstrated that a polysaccharide-oxidizing enzyme such as those identified in the present patent application can act synergistically with other already-known polysaccharide-oxidizing enzymes such as the lytic polysaccharide monooxygenases (generally referred to as LPMOs) in order to improve the hydrolysis of these polysaccharides.

The experimental data described herein show that the polysaccharide-oxidizing enzymes identified by the inventors can target distinct sugar units constituting a polysaccharide (for example cellulose, hemicellulose or lignocellulose) or else can target chemical groups of sugar units different from those targeted by the already-known LPMOs, such as AA9 (also known as GH61), AA10, AA11 and AA13.

Thus, the inventors have discovered, unexpectedly, that the polysaccharide-oxidizing enzymes under consideration according to the invention can act synergistically with cellulases in order to degrade materials containing polysaccharides, such as materials containing cellulose and materials similar to lignocellulose.

It has also been found that the polysaccharide-oxidizing enzymes identified herein can also act synergistically with LPMOs in order to degrade materials containing polysaccharides, such as materials containing cellulose and materials similar to lignocellulose.

Consequently, the polysaccharide-oxidizing enzymes of the present invention may be considered either separately or in combination with other enzymes that are capable of oxidizing or degrading polysaccharides, and also mixtures thereof. Thus, the inventors have identified a new class of polysaccharide-oxidizing enzymes which may be used in a variety of processes for degrading materials containing polysaccharides, and more particularly in a wide variety of processes for degrading lignocellulosic materials.

These properties are thus advantageous for the preparation of cellulose-based supports for manufacturing cellulose fibers.

Without wishing to be limited by any mechanism of action, the inventors are of the opinion that the polysaccharide oxidase properties of these novel LPMOs can facilitate the manufacture of cellulose fibers from a cellulose-based substrate as follows:
cleavage of the (hemi)cellulosic chains can create fragilities in the fibers facilitating the mechanical stripping essential for nanofibrillation, along the same lines as the endoglucanases currently used; and moreover
the formation of oxidation products would make it possible to introduce charged chemical functions on the surface of the fibers, inducing electrostatic repulsions.

Furthermore, without necessarily being bound by the theory, the inventors are of the opinion that the enzymes AAxx of the invention are capable of preferentially acting on cellulose-bound xylans and more specifically on xylans having a rigidity and a conformation similar to those of the underlying cellulose chains.

The consequence of these combined structural modifications is facilitation of the separation of the fibers down to nanometric dispersion and the formation of cellulose fibers, in particular of cellulose nanofibrils, having novel and advantageous features and functionalities.

The invention thus relates to (i) a novel process for preparing a cellulose-based substrate; (ii) the use of the cellulose-based substrate thus obtained as starting material for forming cellulose fibers, notably nanocelluloses; (iii) a process for defibrillating a cellulose-based substrate; and (iv) a process for manufacturing cellulose fibers.

A process for manufacturing cellulose fibers as defined herein may thus consist of the combined use of a process for preparing a cellulose-based substrate and then of a defibrillation process on said prepared substrate.

The fields of application of the cellulose fibers, in particular nanocelluloses, obtained via said processes are extremely varied. Generically, nanocelluloses target applications in three major fields:
hydrated dispersed systems: Nanocrystals are usually stabilized as colloidal suspensions by electrostatic repulsions induced by the presence of surface charges. They may serve as hydrophobic-phase stabilizers and thus be included in the composition of paints, varnishes, cosmetics, etc. They may also make it possible to disperse functional agents such as opacifiers for applications (for example titanium dioxide in papermaking) or reinforcers (clays).
Porous materials (foams, aerogels, membranes, etc.): The very high specific surface area of nanocelluloses affords access to control of the porosity of the materials at the nanometric level after drying. This control gives access to various types of applications in which porosity or a large specific surface area are key features: filtration membrane, catalytic support, thermal superinsulators.
Nanocomposites: Nanocelluloses have entirely noteworthy mechanical properties. Moreover, their nanometric-scale dispersion and their hydrophilic nature give them gas-barrier properties. These features open the way to their use notably in the field of packagings.

### General Definitions

In general, and throughout the present text, terms such as “comprise” or “include”, and also variations thereof, are liable to include elements other than those explicitly mentioned. These terms may, where appropriate, be replaced with “consisting of”. The articles “a” and “an” also include “more than one”, which includes “a plurality”, or else “two or more”.

The present invention relates to a process for manufacturing nanocelluloses, in particular cellulose fibrils and/or cellulose nanocrystals, from a cellulose-based substrate.

The term “cellulose” refers to a linear homopolysaccharide derived from biomass (including organic matter of plant origin, algae included, cellulose of animal origin and also cellulose of bacterial origin) and constituted of glucose units (or rings) (D-anhydroglucopyranose—AGU, meaning “anhydroglucose unit”) connected together via β-(1-4) glycoside bonds. The repeating unit is a glucose dimer, also known as a cellobiose dimer.

AGUs bear three hydroxyl functions: two secondary alcohols (on the carbons in positions 2 and 3 of the glucose ring) and one primary alcohol (on the carbon in position 6 of the glucose ring).

These polymers combine together via intermolecular bonds of hydrogen bonding type, thus giving cellulose a fibrous structure. In particular, association of the chains formed from cellobiose dimers forms an elementary cellulose nanofibril (the diameter of which is about 5 nm). The association of elementary nanofibrils forms a nanofibril (the diameter of which generally ranges from 50 to 500 nm; which includes 50, 60, 70, 80, 90, 100, 150, 200, 250, 300, 350, 400, 450 and 500 nm). Arrangement of several of these nanofibrils then forms what is generally known as a cellulose fiber.

The term “cellulose fiber” denotes all of the forms of cellulose that are liable to be obtained on conclusion of a process of defibrillation, or stripping, of a cellulose-based substrate; which includes the forms of cellulose having a size of the order of a nanometer, and also the forms of cellulose having a larger size.

The term “nanocelluloses” denotes the various forms of cellulose having a size of the order of a nanometer. According to the invention, this term in particular encompasses two families of nanocelluloses: cellulose nanocrystals and cellulose fibrils.

The terms “cellulose fibrils”, “(cellulose) nanofibrils”, “(cellulose) nanofibers”, “nanofibrillated cellulose”, “(cellulose) microfibrils”, “microfibrillated cellulose” and “cellulose nanofibrils” are synonymous.

Each cellulose nanofibril contains crystalline parts stabilized with a solid network of inter-chain and intra-chain hydrogen bonds. These crystalline regions are separated by amorphous regions.

Removal of the amorphous parts of the cellulose nanofibrils makes it possible to obtain cellulose nanocrystals (NCCs).

NCCs advantageously include at least 50% of crystalline part, more preferably at least 55% of crystalline part. They generally have a diameter ranging from 5 to 70 nm (preferably less than 15 nm) and a length ranging from 40 nm to about 1 am, preferably ranging from 40 nm to 500 nm; which includes 50, 60, 70, 80, 90, 100, 150, 200, 250, 300, 350, 400, 450 and 500 nm.

The terms “cellulose nanocrystals”, “nanocrystalline cellulose”, “cellulose whiskers” and “microcrystals” are synonymous. In the rest of the present patent application, the term “cellulose nanocrystals” (NCCs) will be used generically.

As used herein, a material containing polysaccharides covers a substance or a composition comprising polysaccharides.

The term “polysaccharide” is used in its conventional sense and denotes polymeric carbohydrates composed of long chains of monosaccharides held together with glycoside bonds. During their hydrolysis, the polysaccharides release the monosaccharides or oligosaccharides of which they are constituted.

The term “material containing lignocellulose” used herein refers to a material initially consisting of cellulose, hemicellulose and lignin. This term is synonymous with “lignocellulosic material”. Such a material is often referred to as “biomass”.

According to this definition, a material containing lignocellulose is an example of a cellulose-based substrate that is suitable for forming cellulose fibers, within the meaning of the invention.

As used in the present patent application, a “polysaccharide-oxidizing enzyme” encompasses polypeptides having the following properties:
said polypeptide produces hydrogen peroxide in the presence of oxygen and of an electron-donating compound, such as ascorbates,
said polypeptide increases in a dose-dependent manner, in the absence or presence of an electron-donating compound, the degradation of a material containing polysaccharides such as lignocellulose, mediated by cellulases and/or xylanases,
said polypeptide increases in a dose-dependent manner, in the absence or presence of an electron-donating compound, the degradation of a material containing polysaccharides such as lignocellulose, mediated by cellulases, in the presence of one or more LPMOs, such as the LPMOs selected from the group comprising AA9, AA10, AA11 and AA13.

A “polysaccharide-oxidizing enzyme” according to the invention has been demonstrated to be particularly efficient for oxidizing xylans, and notably xylans absorbed on cellulose. The term “electron-donating compound” is used herein in its usual sense for a person skilled in the art. Thus, an electron-donating compound is a chemical species that is capable of donating electrons to another compound. An electron-donating compound is a reducing agent by means of its capacity to donate electrons, and is itself oxidized when it donates electrons to another chemical species. An electron-donating compound as specified above for the polysaccharide-oxidizing properties encompasses, in a nonexhaustive manner, ascorbates and cellobiose dehydrogenases (CDHs).

In the absence of a reducing agent, such as ascorbate, the reducing agent may advantageously be provided by the biomass (lignin) which may act as electron donor.

As used in the patent application, a “BLAST-P method” (also known as Protein Basic Local Alignment Search Tool method) is an analytical method that is well known to those skilled in the art. The BLAST-P method has notably been described in Altschul et al. (1990, J. Mol. Biol., Vol. 215 (No. 3): 403-410), Altschul et al. (1997, Nucleic Acids Res. Vol. 25: 3389-3402) and Altschul et al. (2005, FEBS J. Vol. 272: 5101-5109). The BLAST-P method may be performed using the NCBI online tool (Internet address: http://blast.ncbi.nlm.nih.gov/Blast.cgi?PAGE=Proteins).

When the BLAST-P method is used in the present patent application, it is preferably used with the following parameters: (i) Expected threshold: 10; (ii) Word Size: 6; (iii) Max Matches in a Query range: 0; (iv) Matrix: BLOSSUM62; (v) Gap costs: Existence 11, Extension 1; (vi) Compositional Adjustments: Conditional compositional score matrix adjustment, (vii) No filter; (viii) No mask.

As is well known, the alignment score, S, is calculated as the sum of the substitution and gap scores. The substitution scores are given in a table (see PAM, BLOSUM below). The gap scores are generally calculated as the sum of the Gs, the gap opening penalty and L, the gap extension penalty. For a gap of a length n, the gap cost would be G+Ln. The choice of the gap costs, G and L are empirical, but it is common practice to choose a high value for G (10-15) and a low value for L (1-2).

An optimum alignment means the alignment of two sequences with the highest possible score.

The amino acid identity represents the extent to which two amino acid sequences have the same residues in the same positions in an alignment, and is often expressed as a percentage.

The BLOSUM (Block Substitution Matrix) matrices are substitution score matrices in which the scores for each position are derived from observation of the frequency of substitution of blocks in local alignments for related proteins. Each matrix is drawn for a particular evolution distance. In the BLOSUM62 matrix, for example, the alignment from which the scores were derived was created from sequences sharing not more than 62% identity. The sequences which have more than 62% identity are represented by a single sequence in the alignment so as not to overrepresent close members of a same family.

As used herein, a E-value (also known as the Expect Value) is a parameter calculated when the BLAST-P method is used, said parameter representing the number of different alignments with equivalent scores or with a score better than S, which are expected to appear by chance in a database search. Thus, the lower the E value, the more the score and the alignment will be significant.

In the context of the present invention, the “percentage of identity” between two polypeptides means the percentage of identical amino acids between the two polypeptide sequences to be compared, obtained after an optimum alignment, this percentage being entirely statistical and the differences between the two polypeptide sequences being randomly distributed over their length. The comparison of two polypeptide sequences is conventionally performed by comparing the sequences after they have been optimally aligned; said comparison must be able to be performed in segments or using an “alignment window”. The optimum alignment of the sequences for their comparison is performed using the BLAST-P comparison software.

In its principle, the percentage of identity between two amino acid sequences is determined by comparing the two optimally aligned sequences within which the nucleic acid sequences to be compared may contain additions or deletions relative to the reference sequence of the optimum alignment between the two polypeptide sequences. The percentage of identity is calculated by determining the number of positions in which an amino acid is identical between the two sequences, preferably between two whole sequences, and then by dividing this number of identical positions by the total number of positions in the alignment window and by multiplying the result by 100 in order to obtain the percentage of identity between the two sequences.

As it is understood in the present patent application, polypeptide sequences having at least 20% amino acid identity with a reference sequence comprise those which have at least 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 28%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with said reference sequence.

Similarly, the “percentage of identity” between two nucleic acid sequences represents the percentage of identical nucleotide residues between the two nucleic acid sequences to be compared, obtained after an optimum alignment, this percentage being purely statistical and the differences between the two sequences being randomly distributed along the length of the sequences. The comparison of two nucleic acid sequences is conventionally performed by comparing the sequences after they have been optimally aligned, said comparison possibly being performed in segments or using an “alignment window”. The optimum alignment of the sequences for the purpose of their comparison is performed using the BLAST-N comparison software.

In principle, the percentage of identity between two nucleic acid sequences is determined by comparing the two optimally aligned sequences in which the nucleic acid sequences to be compared may contain additions or deletions relative to the reference sequence of the optimum alignment between the two sequences. The percentage of identity is calculated according to the number of positions in which the nucleotide residues are identical between the two sequences, preferably between two whole sequences, and then by dividing this number of identical positions by the total number of positions in the alignment window and by multiplying the result by 100 in order to obtain the percentage of identity between the two sequences.

As it is understood herein, the nucleotide sequences having at least 20% identity with the reference sequence include those which have at least 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 28%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with said reference sequence.

As indicated herein, an E value of 10 e⁻³ or less encompasses the E-values of 1 e⁻³ or less, 1 e⁻⁴ or less, 1 e⁻⁵ or less, 1 e⁻⁶ or less, 1 e⁻⁷ or less, 1 e⁻⁸ or less, 1 e⁻⁹ or less, 1 e⁻¹⁰ or less, 1 e⁻²⁰ or less, 1 e⁻³⁰ or less, 1 e⁻⁴⁰ or less, 1 e⁻⁵⁰ or less, 1 e⁻⁶⁰ or less, 1 e⁻⁷⁰ or less, 1 e⁻⁸⁰ or less, 1 e⁻⁹⁰ or less and 1 e⁻¹⁰⁰ or less.

The term “chemical treatment” refers to all the chemical pretreatments which allow the separation and/or release of cellulose, hemicellulose and/or lignin. Examples of these suitable chemical pretreatments include, for example, dilute acids, lime, bases, organic solvents, aqueous ammonia, sulfur dioxide and carbon dioxide. Furthermore, wet oxidation and hydrothermolysis at controlled pH are also considered as chemical pretreatments. The pretreatment methods using aqueous ammonia are notably described in patent applications PCT WO 2006/110891, WO 2006/110899, WO 2006/110900 and WO 2006/110901.

Other examples of suitable pretreatments are described by Schell et al., 2003, Appl. Biochem and Biotechn. Vol. 105-108: 69-85, and Mosier et al., 2005, Bioresource Technology 96: 673-686, and U.S. Publication No. 2002/0164730.

The term “mechanical pretreatment” refers to all the mechanical (or physical) treatments which allow the separation and/or release of cellulose, hemicellulose and/or lignin from a material containing lignocellulose. For example, the mechanical pretreatments include various types of milling, irradiation, steam explosion and hydrothermolysis.

Mechanical pretreatment includes the fragmentation of a solid (mechanical comminution or reduction of the size). The fragmentation of a solid includes the techniques of dry milling, wet milling and vibrating-ball milling. The mechanical pretreatment may also include high pressures and/or high temperatures (steam explosion). In certain representations of the pretreatment step, said step may combine mechanical and chemical pretreatment.

As has been used in the present invention, the term “biological pretreatment” refers to all the biological treatments which allow the separation and/or release of cellulose, hemicellulose and/or lignin from a material containing lignocellulose. Biological pretreatments may involve the application of microorganisms that are capable of dissolving lignin (see, for example, Hsu, 1996, Pretreatment of biomass, in the Handbook on Bioethanol: Production and Utilization, Wyman, ed., Taylor & Francis, Washington, D.C., 179-212; Ghosh and Singh, 1993, Physicochemical and biological treatments for enzymatic/microbial conversion of lignocellulosic biomass, Adv. Appl. Microbiol. 39: 295-333; McMillan, 1994, Pretreating lignocellulosic biomass: a review, in Enzymatic Conversion of Biomass for Fuel Production, Himmel, Baker, and Overend, eds., ACS Symposium Series 566, American Chemical Society, Washington, D.C., chapter 15; Gong, Cao, Du, and Tsao, 1999, Ethanol production from renewable resources, in Advances in Biochemical Engineering/Biotechnology, Scheper, ed., Springer-Verlag Berlin Heidelberg, Germany, 65: 207-241; Olsson and Hahn-Hagerdal, 1996, Fermentation of lignocellulosic hydrolysates for ethanol production, Enz. Microb. Tech. 18: 312-331; and Vallander and Eriksson, 1990, Production of ethanol from lignocellulosic materials: State of the art, Adv. Biochem. Eng. J. Biotechnol. 42: 63-95).

Processes According to the Invention

According to a first subject, the invention relates to a process for preparing a cellulose-based substrate for the manufacture of cellulose fibers, which process comprises at least the following steps:

a) having a cellulose-based substrate which is capable of forming cellulose fibers;

b) placing said substrate in contact, in the presence of an electron donor, with at least one enzyme belonging to the family of lytic polysaccharide monooxygenases (LPMOs) under conditions that are capable of ensuring oxidative cleavage of said cellulose fibers, in order to defibrillate said cellulose-based substrate;

characterized in that said enzyme has an amino acid sequence identity of at least 20% with a reference polypeptide of SEQ ID NO. 1, 2 or 3, by means of the BLAST-P comparison method, said BLAST-P comparison method resulting in an E-value of 10 e⁻³ or less.

According to another subject, the invention relates to the use of a cellulose-based substrate prepared according to the preceding process as cellulose-based starting material for preparing cellulose fibers, in particular nanocellulose, notably cellulose fibrils and/or cellulose nanocrystals, via a defibrillation process, notably a mechanical defibrillation process.

According to a second subject, the invention relates to a process for extracting cellulose fibers, which process comprises at least the following steps:

a) having a cellulose-based substrate which is capable of forming cellulose fibers placed in contact with an electron donor and an enzyme with polysaccharide oxidase activity, under conditions suitable for ensuring oxidative cleavage of said cellulose fibers in the presence of an electron donor, characterized in that said enzyme with polysaccharide oxidase activity is characterized in that it has an amino acid sequence identity of at least 20% with a reference polypeptide of SEQ ID NO. 1, 2 or 3, by means of the BLAST-P comparison method, said BLAST-P comparison method resulting in an E-value of 10 e⁻³ or less; and

b) mechanically treating said cellulose-based substrate, so as to extract said cellulose fibers from said substrate.

According to a third subject, the invention relates to a process for manufacturing cellulose fibers, which process comprises at least the following steps:

a) having a cellulose-based substrate which is capable of forming cellulose fibers;

b) placing said cellulose-based substrate in contact with at least one enzyme belonging to the family of lytic polysaccharide monooxygenases (LPMOs) under conditions that are capable of ensuring oxidative cleavage of said cellulose fibers in the presence of an electron donor;

c) mechanically treating said cellulose-based substrate, so as to manufacture said cellulose fibers from said cellulose-based substrate,

characterized in that said enzyme with polysaccharide oxidase activity has an amino acid sequence identity of at least 20% with a reference polypeptide of SEQ ID NO. 1, 2 or 3, by means of the BLAST-P comparison method, said BLAST-P comparison method resulting in an E-value of 10 e⁻³ or less.

Said enzyme with polysaccharide oxidase activity is mixed with the cellulose-based substrate so as to allow contact between said at least one enzyme and the cellulose fibers.

The enzymatic treatment step is preferably performed with gentle stirring, so as to ensure good dispersion of the enzymes within the fibers. This enzymatic treatment step is performed, for example, for a time ranging from 24 hours to 72 hours (preferably 48 hours).

Preferably, the enzymatic treatment step is performed at a temperature ranging from 30 to 50° C., notably from 30 to 45° C.

The pH of the reaction conditions of the enzyme in contact with the cellulose-based substrate is generally between 3 and 7, which includes between 4 and 7, and notably between 4 and 6.

According to the invention, said at least one LPMO enzyme may be added to the cellulose-based substrate in an enzyme/cellulose ratio ranging from 1/1000 to 1/50, notably from 1/500 to 1/50 or from 1/100 to 1/50 or else from 1/1000 to 1/500, from 1/500 to 1/100.

Preferably, said at least one LPMO enzyme is used at a concentration ranging from 0.001 to 10 g/L, notably from 0.1 to 5 g/L and more preferably from 0.5 to 5 g/L.

According to a particular embodiment, the cellulose-based substrate is subjected to at least two (or even only to two) successive enzymatic treatment steps (in series, advantageously separated by a rinsing step).

The LPMO(s) used in the course of each of these enzymatic treatment steps are identical or different; the conditions (notably the enzyme/substrate ratio) are identical or different between these successive steps.

In this case, the Examples demonstrate that the fibers are entirely destructured.

In a nonexclusive manner, tests of cellulose cleavage with an LPMO enzyme according to the invention may be conducted according to the following protocol: For example, a cleavage test may be performed in a volume of 300 μl of liquid containing 4.4 μM of LPMO enzyme and 1 mM of ascorbate and 0.1% (weight/volume) of cellulose powder swollen with phosphoric acid (PASC: phosphoric acid-swollen cellulose—prepared as described in Wood T M, Methods Enzym. 1988, 160: 19-25) in 50 mM of a sodium acetate buffer at pH 4.8 or 5 μM of cello-oligosaccharides (Megazyme, Wicklow, Ireland) in 10 mM of sodium acetate buffer at pH 4.8.

The enzymatic reaction may be performed in an incubated 2 ml tube in a thermomixer (Eppendorf, Montesson, France) at 50° C. and 580 rpm (revolutions per minute).

The fibers are placed in contact with the enzymes (at a concentration of between 1 and 5 g/L and in enzyme/cellulose ratios of 1/50, 1/100, 1/500 and 1/1000) and with ascorbate (2 mM) and then subjected to gentle stirring for 48 hours at 40° C.

After 16 hours of incubation, the sample is maintained at 100° C. for 10 minutes in order to stop the enzymatic reaction, and then centrifuged at 16 000 revolutions per minute (rpm) for 15 minutes at 4° C. in order to separate the solution fraction from the remaining insoluble fraction.

The treated fibers are then subjected to a mechanical action with a homogenizer-disperser (Ultra-Turrax, power 500 W, maximum speed for 3 minutes), followed by an ultrasonication treatment for 3 minutes.

According to one embodiment, said processes are characterized in that the cellulose fibers obtained on conclusion of the process are cellulose nanofibrils.

According to one embodiment, said processes are characterized in that the electron donor is chosen from ascorbate, gallate, catechol, reduced glutathione, lignin fragments and fungal carbohydrate dehydrogenases; preferably ascorbate.

According to one embodiment, said processes are characterized in that the cellulose-based substrate is obtained from wood, a cellulose-rich fibrous plant, beetroot, citrus plants, annual straw plants, marine animals, algae, fungi or bacteria.

According to one embodiment, said processes are characterized in that the cellulose-based substrate is chosen from chemical paper pulps, preferably chemical wood paper pulps, more preferably at least one from among the following paper pulps: bleached pulps, semi-bleached pulps, unbleached pulps, bisulfite pulps, sulfate pulps, soda pulps, kraft pulps.

According to one embodiment, said processes are characterized in that the cellulose-based substrate is a paper pulp derived from wood, from annual plants or from fibrous plants.

According to one embodiment, said processes are characterized in that said at least one mechanical treatment step comprises at least one of the following mechanical treatments:
a homogenization treatment,
a microfluidization treatment,
an abrasion treatment, and/or
a cryomilling treatment.

According to one embodiment, said processes are characterized in that, following said mechanical treatment step, said process comprises a post-treatment step chosen from: an acid treatment, an enzymatic treatment, an oxidation, an acetylation, a silylation, or alternatively a derivatization of chemical groups borne by said cellulose fibers.

According to another subject, the invention relates to cellulose fibers derived from a defibrillation process and/or from a cellulose fiber manufacturing process as defined above.

Said cellulose fibers may be characterized in that said cellulose fibers, preferably of nanocellulose, include glucose rings, of which at least one carbon atom is oxidized in position(s) C₁ and/or C₄, or even also C₆.

According to a preferred embodiment, the cellulose fibers are cellulose nanofibrils.

Mechanical Treatment Step(s)

The cellulose-based substrate placed in contact with said enzyme is then subjected to at least one mechanical treatment step which is intended to strip the cellulose fibers to obtain nanocelluloses.

Stripping (also known as “fibrillation” or “defibrillation”) consists in separating, via a mechanical phenomenon, the cellulose fibers within the cellulose-based substrate, notably for the manufacture of nanocelluloses.

As demonstrated, the oxidative cleavage of the cellulose fibers, catalyzed with said at least one LPMO, facilitates the stripping of these cellulose fibers during the mechanical treatment step.

This step of mechanical stripping of the cellulose fibers may then be performed under less drastic and thus less energy-intensive conditions. Moreover, the use of LPMOs according to the invention makes it possible to introduce into the cellulose fibers charged groups which induce electrostatic repulsions, without contamination with treatment reagents, as during the use of TEMPO reagents.

Mechanical treatments directed toward stripping cellulose fibers are known to those skilled in the art and may be performed in the process(es) of the invention.

In general, mention may be made of mild mechanical treatments with a homogenizer-disperser (for example an Ultra-Turrax machine) and/or ultrasonication treatments.

Reference may also be made, for example, to the document from N. Lavoine et al. (Carbohydrate Polymers, 2012, (92): 735-64) which notably describes (pages 740 to 744) mechanical treatments for the preparation of microfibrillated cellulose (for example cellulose nanofibrils).

Typically, a mechanical treatment may be chosen from homogenization, microfluidization, abrasion or cryomilling mechanical treatments.

The homogenization treatment involves passing the pretreated cellulose-based substrate, typically a cellulose pulp or a liquid cellulose suspension, through a narrow space under high pressure (as described, for example, in patent U.S. Pat. No. 4,486,743).

This homogenization treatment is preferably performed using a homogenizer such as a Gaulin homogenizer. In such a device, the pretreated cellulose-based substrate, typically in the form of a cellulose suspension, is pumped at high pressure and distributed through small-orificed automatic valve. A rapid succession of opening and closing of the valve subjects the fibers to a substantial drop in pressure (generally of at least 20 MPa) and to a high-speed shear action followed by a high-speed deceleration impact. Passage of the substrate through the orifice is repeated (generally from 8 to 10 times) until the cellulose suspension becomes stable. In order to maintain a temperature of the product within a range extending from 70 to 80° C. during the homogenization treatment, cooling water is generally used.

This homogenization treatment may also be performed using a device such as a microfluidizer (see, for example, Sisqueira et al. Polymer 2010 2(4): 728-65). In such a device, the cellulose suspension passes through a thin chamber which is typically Z-shaped (the dimensions of the channel of which are generally between 200 and 400 μm) under high pressure (about 2070 bar). The high shear rate that is applied (generally greater than 10⁷ s⁻¹) makes it possible to obtain very fine nanofibrils. A variable number of passes (for example from 2 to 30, notably from 10 to 30 or from 5 to 25, and in particular from 5 to 20) with chambers of different sizes may be used, to increase the degree of fibrillation.

The abrasion or milling treatment (see, for example, S. Iwamoto et al., 2007, Applied Physics A89(2): 461-66) is based on the use of a milling device that is capable of exerting shear forces provided by millstones.

The pretreated cellulose-based substrate, generally in the form of a cellulose pulp, is passed between a static millstone and a millstone in rotation, typically at a speed of about 1500 revolutions per minute (rpm). Several passes (generally between 2 and 5) may be necessary to obtain nanometric-sized fibrils.

A device such as a blender (as described, for example, in Unetani K et al., Biomacromolecules, 2011, 12(2), pages 348-53) may also be used to produce microfibrils from the pretreated cellulose-based substrate, for example from a wood fiber suspension.

The cryomilling (or cryocrushing) treatment (Dufresne et al., 1997, Journal of Applied Polymer Science, 64(6): 1185-94) consists in milling a suspension of pretreated cellulose-based substrate which has been frozen beforehand with liquid nitrogen. The ice crystals formed inside the cells explode the cell membranes and release wall fragments. These processes are generally used for the production of cellulose microfibrils from agricultural products or residues.

Step(s) of Post-Treatment of the Cellulose-Based Substrate

In certain embodiments, the process of defibrillation and/or of manufacture of cellulose fibers comprises at least one step of post-treatment of the cellulose-based substrate, performed after said substrate has been subjected to the mechanical treatment.

Generally, said at least one post-treatment step is directed toward increasing the degree of fibrillation of the celluloses (notably of the nanocelluloses) obtained and/or to give said nanocelluloses novel mechanical properties, as a function of the intended applications.

Said at least one post-treatment step may notably be chosen from an acid treatment, an enzymatic treatment, an oxidation, an acetylation, a silylation, or else a derivatization of certain chemical groups borne by the microfibrils. Reference may also be made, for example, to the document from N. Lavoine et al. (Carbohydrate Polymers, 2012, (92): 735-64) which notably describes (point 2.3, pages 747 to 748) post-treatments that may be combined with various mechanical pretreatments and treatments of the cellulose fibers.

Cellulose-Based Substrate

The cellulose-based substrate may be obtained according to the invention from any material of biomass (encompassing organic matter of plant origin, algae included, animal or fungal origin) comprising cellulose-based fibers (i.e. cellulose fibers).

The cellulose-based substrate is advantageously obtained from wood (of which cellulose is the main component), but also from any cellulose-rich fibrous plant, for instance cotton, flax, hemp, bamboo, kapok, coconut fiber (coir), ramie, jute, sisal, raffia, papyrus and certain reeds, sugarcane bagasse, beetroot (and notably beetroot pulp), citrus plants, corn or sorghum stalks, or alternatively annual straw plants.

The cellulose-based substrates may also be obtained from marine animals (for instance tunicates), from algae (for instance Valonia or Cladophora) or from bacteria for bacterial cellulose (for instance bacterial strains of the Gluconacetobacter type).

Depending on the application, cellulose derived from primary walls such as the parenchyma of fruit (for example beetroot, citrus plants, etc.) or from secondary walls, such as wood, will be chosen.

The cellulose-based substrate advantageously consists of a cellulose-based material prepared via chemical or mechanical means, from any cellulose source mentioned above (and notably from wood).

The cellulose-based substrate is advantageously in the form of a suspension of cellulose fibers in a liquid medium (preferably an aqueous medium), or of a cellulose pulp.

The cellulose pulps may be conditioned in the “dry” state, i.e. typically in a state of dryness of greater than or equal to 80%, notably greater than or equal to 90%. The cellulose pulp may then be redispersed in an aqueous medium via a mechanical treatment.

Preferably, the cellulose-based substrate contains at least 90%, notably at least 95% and preferably 100% of cellulose fibers.

Preferably, the cellulose-based substrate is suitable for manufacturing paper or a cellulose-based product. The cellulose-based substrate is thus preferably chosen from papermaking pulps (or paper pulp), and in particular chemical paper pulps.

In general, cellulose pulp and notably paper pulp may contain, in combination with cellulose fibers, hemicellulose and lignin. Preferably, cellulose pulp contains less than 10% and notably less than 5% of lignin and/or of hemicellulose.

Preferably, chemical paper pulps almost exclusively, or even exclusively, contain cellulose fibers.

The paper pulp may be chosen from at least one of the following paper pulps: bleached pulps, semi-bleached pulps, unbleached pulps, bisulfite pulps (bleached or unbleached), sulfate pulps (bleached or unbleached), soda pulps (bleached or unbleached) and Kraft pulps.

It is also possible to use pulps to be dissolved having a low proportion of hemicellulose, preferably less than 10% and notably less than or equal to 5%.

Preferably, the paper pulps used in a process of the invention are wood pulps, notably wood chemical paper pulps.

According to one embodiment, the cellulose-based substrate is a paper pulp derived from wood, from annual plants or from fibrous plants.

A material containing lignocellulose, as defined below, is an example of a cellulose-based substrate that is particularly considered according to the invention.

In a preferred embodiment, the material containing lignocellulose contains at least 30 wt %, preferably at least 50 wt %, even more preferentially at least 70 wt % and even more preferentially at least 90 wt % of lignocellulose. It is noted that the material containing lignocellulose may also contain other components such as protein material, starch, sugars, such as sugars which can ferment and/or sugars which cannot ferment.

A material containing lignocellulose may be any material containing lignocellulose. In a preferred representation, the material containing lignocellulose contains at least 30 wt %, preferably at least 50 wt %, even more preferentially at least 70 wt % and even more preferentially at least 90 wt % of lignocellulose. It is important to understand that the material containing lignocellulose may also contain other components such as protein material, starch, sugars, such as sugars which can ferment and/or sugars which cannot ferment.

The material containing lignocellulose is generally present, for example, in the stalks, leaves, bran, envelopes and rachis of plants or tree leaves, branches, and wood. In a nonlimiting manner, the material containing lignocellulose may also be herbaceous material, agricultural and silvicultural residues, solid municipal waste, paper waste, and pulp and paper milling residues. It should be understood here that the material containing lignocellulose may be in the form of cell wall material of the plant containing lignin, cellulose and hemicellulose in a mixed matrix.

According to certain particular embodiments, the material containing lignocellulose is a lignocellulosic biomass chosen from the group consisting of: grass, switchgrass, spartina, rye grass, reed canarygrass, miscanthus, sugar transformation residues, sugarcane bagasse, agricultural waste, rice straw, rice ball, barley straw, corn ear, cereal straw, wheat straw, canola straw, oat straw, oat husk, corn cane, soybean meal, corn meal, forestry waste, recycled wood pulp fiber, paper slurry, sawdust, hardwood, coniferous wood, agave, and combinations thereof. In a preferred embodiment, the material containing lignocellulose is chosen from the group comprising: corn meal, corn fiber, rice straw, pine wood, wood chips, poplar, bagasse, paper and pulp treatment waste.

Preferably, the material containing lignocellulose is a wood-based lignocellulosic biomass. Other examples of material containing lignocellulose include hardwood, such as popular and birch, softwood, cereal straw such as wheat straw, switchgrass, solid municipal waste, organic industrial waste, office paper and a mixture thereof.

According to illustrated embodiments, the material containing lignocellulose is selected from pine, poplar and wheat straw.

Enzyme of LPMO Type According to the Invention (AAxx Family)

An enzyme according to the invention is defined as an enzyme with polysaccharide oxidase activity, said enzyme having an amino acid sequence identity of at least 20% with a reference polypeptide of SEQ ID NO. 1, 2 or 3, by means of the BLAST-P comparison method, said BLAST-P comparison method resulting in an E-value of 10 e⁻³ or less.

According to certain embodiments, said enzyme with polysaccharide oxidase activity has an amino acid sequence identity of at least 30% with a reference polypeptide of SEQ ID NO. 1, 2 or 3, by means of the BLAST-P comparison method, said BLAST-P comparison method resulting in an E-value of 10 e⁻³ or less.

According to certain embodiments, said enzyme with polysaccharide oxidase activity has an amino acid sequence identity of at least 60% with a reference polypeptide of SEQ ID NO. 1, 2 or 3, by means of the BLAST-P comparison method, said BLAST-P comparison method resulting in an E-value of 10 e⁻³ or less.

According to certain embodiments, said enzyme with polysaccharide oxidase activity has an amino acid sequence identity of at least 90% with a reference polypeptide of SEQ ID NO. 1, 2 or 3, by means of the BLAST-P comparison method, said BLAST-P comparison method resulting in an E-value of 10 e⁻³ or less.

According to certain embodiments, said enzyme with polysaccharide oxidase activity has an amino acid sequence identity of at least 90% with a reference polypeptide of SEQ ID NO. 1, by means of the BLAST-P comparison method, said BLAST-P comparison method resulting in an E-value of 10 e⁻³ or less.

According to certain embodiments, said enzyme with polysaccharide oxidase activity has an amino acid sequence identity of at least 90% with a reference polypeptide of SEQ ID NO. 2, by means of the BLAST-P comparison method, said BLAST-P comparison method resulting in an E-value of 10 e⁻³ or less.

According to certain embodiments, said enzyme with polysaccharide oxidase activity has an amino acid sequence identity of at least 90% with a reference polypeptide of SEQ ID NO. 3, by means of the BLAST-P comparison method, said BLAST-P comparison method resulting in an E-value of 10 e⁻³ or less.

According to certain embodiments, said enzyme with polysaccharide oxidase activity is coded for by a nucleic acid having at least 90% sequence identity with a nucleic acid chosen from the group consisting of SEQ ID NO. 4, SEQ ID NO. 5 and SEQ ID NO. 6.

For illustrative purposes, when an enzyme with polysaccharide oxidase activity of sequence SEQ ID NO. 2 is compared with the reference polypeptide SEQ ID NO. 1 according to the BLAST-P comparison method, (i) said enzyme with polysaccharide oxidase activity of SEQ ID NO. 2 has an amino acid sequence identity of 66% relative to the polypeptide of sequence SEQ ID NO. 1 and (ii) the BLAST-P comparison method results in an E-value of 4 e⁻¹³³.

Also for illustrative purposes, when an enzyme with polysaccharide oxidase activity of sequence SEQ ID NO. 3 is compared with the reference polypeptide SEQ ID NO. 1 according to the BLAST-P comparison method, (i) said enzyme with polysaccharide oxidase activity of SEQ ID NO. 2 has an amino acid sequence identity of 34% relative to the polypeptide of sequence SEQ ID NO. 1 and (ii) the BLAST-P comparison method results in an E-value of 2 e⁻⁴⁰.

According to preferred embodiments, said enzyme with polysaccharide oxidase activity is chosen from the group comprising polypeptides having one of the following GenBank references: AL060293.1; CCA68158.1; CCA68159.1; CCA68161.1; CCA71530.1; CCA72554.1; CCA72555.1; CC030796.1; CCT73728.1; CDM26384.1; CD076981.1; CD076983.1; CD076990.1; CDR41535.1; CDZ98469.1; CDZ98532.1; CDZ98792.1; CDZ98793.1; CEJ62913.1; CEJ80690.1; CEL55274.1; CEL55761.1; CEN61973.1; CEQ41736.1; CRL20539.1; CUA74138.1; CUA75968.1; EEB87294.1; EEB88604.1; EEB93106.1; EGU12035.1; EGU79270.1; EJU02917.1; EJU04796.1; EJU04797.1; EKC98083.1; EKD01731.1; EKD04876.1; EKG10038.1; ELU37011.1; ELU44209.1; EMD31282.1; EMD34047.1; EMT65805.1; ENH74989.1; EPT05587.1; EPT05590.1; EPT05591.1; EUC56978.1; EUC64931.1; EWG51104.1; EWY85510.1; EXK29887.1; EXU99300.1; GA089447.1; GAQ10202.1; GAT49547.1; GAT49548.1; GAT52486.1; GAT61130.1; GAT61131.1; KDE05902.1; KDE09071.1; KDN48575.1; KDN50638.1; KDQ07356.1; KDQ08649.1; KDQ08700.1; KDQ08703.1; KDQ11515.1; KDQ12702.1; KDQ15932.1; KDQ19064.1; KDQ25667.1; KDQ34148.1; KDQ59091.1; KDQ59092.1; KDR69809.1; KDR78641.1; KDR82083.1; KEP48245.1; KEY82804.1; KFG85718.1; KFH41721.1; KFY94807.1; KFZ00858.1; KFZ20368.1; KGB74552.1; KID86720.1; KII89650.1; KII89670.1; KIJ14235.1; KIJ14422.1; KIJ36788.1; KIJ36789.1; KIJ36910.1; KIJ36911.1; KIJ59037.1; KIJ62866.1; KIJ66712.1; KIJ93961.1; KIK01335.1; KIK01364.1; KIK03019.1; KIK24220.1; KIK24223.1; KIK45012.1; KIK47453.1; KIK58046.1; KIK60325.1; KIK64405.1; KIK64418.1; KIK64426.1; KIK64427.1; KIK64461.1; KIK94802.1; KIL59842.1; KIL67972.1; KIL68458.1; KIL88744.1; KIM29500.1; KIM34148.1; KIM35038.1; KIM39331.1; KIM49751.1; KIM57407.1; KIM60439.1; KIM60441.1; KIM60443.1; KIM60444.1; KIM84967.1; KIM93034.1; KIM95301.1; KIM95307.1; KIN08100.1; KIN97734.1; KIN97736.1; KIN97737.1; KIO31600.1; KIP08019.1; KIP08026.1; KIP10435.1; KIR25380.1; KIR50229.1; KIR55806.1; KIR67208.1; KIW62805.1; KIY36322.1; KIY46248.1; KIY46262.1; KIY46497.1; KIY46927.1; KIY47293.1; KIY51548.1; KIY64670.1; KIY68736.1; KIY71843.1; KJA14486.1; KJA19114.1; KJA20550.1; KJA20613.1; KJK82496.1; KKO98459.1; KKP01653.1; KLT38889.1; KLT39034.1; KLT43002.1; KLT43602.1; KLT43893.1; KLT46239.1; KMK54965.1; KNZ77897.1; KNZ78922.1; KPA38710.1; KPI34779.1; KPM37038.1; KPV71930.1; KPV77521.1; KPV77742.1; KTB29212.1; KTB33212.1; KUE98996.1; KUE99426.1; KWU44348.1; KWU44477.1; KXH42132.1; KXH43636.1; KXH51881.1; KXN82218.1; KXN84873.1; KXN89494.1; KXN90938.1; KXN93349.1; KYQ38716.1; KYQ40395.1; KYQ41811.1; KZL64940.1; KZ090689.1; KZ090691.1; KZP14545.1; KZP23879.1; KZS91941.1; KZT07581.1; KZT07590.1; KZT20429.1; KZT29895.1; KZT40257.1; KZT57664.1; KZT57666.1; KZT73200.1; KZT73202.1; KZV68182.1; KZV68185.1; KZV69208.1; KZV72373.1; KZV79310.1; KZV79844.1; KZV82398.1; KZV83782.1; KZV85461.1; KZV85472.1; KZV86197.1; KZV88440.1; KZV88442.1; KZV88448.1; KZV96582.1; KZV97371.1; KZV97738.1; KZV97742.1; KZV98356.1; KZV99282.1; KZW00468.1; KZW00469.1; OAA59408.1; OAA71978.1; OAG11613.1; OAG40496.1; OAL02191.1; OAL28870.1; OAL45637.1; OAP54840.1; OAQ60454.1; OAQ77899.1; OAQ86421.1; OAQ94383.1; OAQ97907.1; OAX34821.1; XP_001263997.1; XP_001796117.1; XP_001829371.2; XP_001835502.2; XP_001835509.1; XP_001836582.1; XP_001840021.2; XP_001877230.1; XP_001878077.1; XP_001885228.1; XP_001905249.1; XP_003035108.1; XP_003035505.1; XP_003036605.1; XP_003042172.1; XP_003191958.1; XP_006458724.1; XP_006459911.1; XP_006963793.1; XP_007001773.1; XP_007003269.1; XP_007262604.1; XP_007299807.1; XP_007301417.1; XP_007306950.1; XP_007318869.1; XP_007318871.1; XP_007319142.1; XP_007327029.1; XP_007329615.1; XP_007337360.1; XP_007343208.1; XP_007346002.1; XP_007349200.1; XP_007349275.1; XP_007351346.1; XP_007351348.1; XP_007351349.1; XP_007351518.1; XP_007352398.1; XP_007353707.1; XP_007359130.1; XP_007362490.1; XP_007362492.1; XP_007362499.1; XP_007362779.1; XP_007388801.1; XP_007388810.1; XP_007393138.1; XP_007393767.1; XP_007581903.1; XP_007600909.1; XP_007746185.1; XP_007765609.1; XP_007768205.1; XP_007792087.1; XP_007792157.1; XP_007826256.1; XP_007849383.1; XP_007867180.1; XP_007867564.1; XP_008039133.1; XP_008039347.1; XP_008039803.1; XP_008039807.1; XP_008718658.1; XP_008731148.1; XP_009256644.1; XP_009545121.1; XP_009545122.1; XP_011321625.1; XP_012046198.1; XP_012181508.1; XP_012183613.1; XP_013257070.1; XP_013271081.1; XP_013277879.1; XP_013332110.1; XP_013943298.1; XP_013944931.1; XP_013954691.1; XP_013960458.1; XP_014176455.1; XP_014180074.1; XP_014180075.1; XP_014181917.1; XP_014543483.1; XP_014573268.1; XP_016242373.1; XP_016271225.1; XP_016275235.1; XP_016275300.1; XP_016610141.1; XP_016620042.1; XP_016630521.1; XP_567250.1; XP_753127.1 and XP_778151.1.

According to preferred embodiments, said enzyme with polysaccharide oxidase activity consists of a recombinant protein. According to some of these preferred embodiments, said recombinant protein is produced in a yeast which has been genetically transformed to produce said recombinant enzyme with polysaccharide oxidase activity.

According to certain preferred embodiments, said enzyme with polysaccharide oxidase activity has an amino acid sequence comprising, or even consisting of, SEQ ID NO. 1, SEQ ID NO. 2, SEQ ID NO. 3, and/or at least one of the enzymes identified by their GenBank number above.

According to certain embodiments, said enzyme with polysaccharide oxidase activity is coded for by a nucleic acid having a nucleic acid sequence chosen from a group comprising SEQ ID NO. 4, SEQ ID NO. 5 and SEQ ID NO. 6.

According to certain preferred embodiments, several enzymes with polysaccharide oxidase activity may be used in the same process. In particular, the cellulose-based support under consideration may be placed (or may have been placed) in contact with a plurality of enzymes with polysaccharide oxidase activity according to the invention, and most particularly a plurality of enzymes with polysaccharide oxidase activity comprising an amino acid sequence comprising, or even consisting of, SEQ ID NO. 1, SEQ ID NO. 2, SEQ ID NO. 3, and/or at least one of the enzymes identified by their GenBank number above.

According to certain preferred embodiments, several enzymes with polysaccharide oxidase activity may be used in the same process. In particular, the cellulose-based support under consideration may be placed (or may have been placed) in contact with a plurality of enzymes with polysaccharide oxidase activity according to the invention, and most particularly a plurality of enzymes with polysaccharide oxidase activity coded for by a nucleic acid chosen from a group from among SEQ ID NO. 4, SEQ ID NO. 5 and SEQ ID NO. 6, and/or at least one of the enzymes identified by their GenBank number above.

According to some of these embodiments, said enzyme with polysaccharide oxidase activity, or said plurality of enzymes, is used in a composition with polysaccharide oxidase activity.

Thus, according to these embodiments, said plurality of enzymes with polysaccharide oxidase activity comprises between 2 and 10 different enzymes with polysaccharide oxidase activity, which includes 2, 3, 4, 5, 6, 7, 8, 9, 10 different enzymes with polysaccharide oxidase activity.

According to certain even more particular embodiments, a composition with polysaccharide oxidase activity is capable of comprising one or more other enzymes with polysaccharide oxidase activity chosen from LPMOs, which includes the enzymes chosen from a group comprising: AA9, AA10, AA11 and AA13 LPMOs.

Methods for Identifying the Polypeptides Belonging to the AAxx Family

The inventors describe here the crystallographic structure of the catalytic module PcAAxxB (JGI ID 1372210; GenBank ID #KY769370) belonging to an enzyme with polysaccharide oxidase activity. Close to 300 enzymes with polysaccharide oxidase activity according to the invention, which belong to the same enzymatic family, including the polypeptides of sequence SEQ ID NO. 1, SEQ ID NO. 2 and SEQ ID NO. 3, are also disclosed.

For reference, the sequence SEQ ID NO. 3 corresponds to the polypeptide SEQ ID NO. 9 after cleavage of the signal peptide; and the sequence SEQ ID NO. 8 corresponds to the cleaved signal peptide.

Thus, by combining these structural data and the consensus sequences derivable from this selection of enzymes with polysaccharide oxidase activity, it is possible to determine any variant with polysaccharide oxidase activity under consideration.

In a nonlimiting manner, bioinformatic methods exist for identifying novel variants forming part of said novel family of LPMOs, using, on the one hand, in-silico modeling based on said crystallographic structure, and, on the other hand, said sequence alignments. Examples of such programs that are capable of producing such three-dimensional models (homology & comparative modeling) include:
MODELLER© V. 9.18 (see B. Webb, A. Sali. Comparative Protein Structure Modeling Using Modeller. Current Protocols in Bioinformatics, John Wiley & Sons, Inc., 5.6.1-5.6.32, 2014);
I-TASSER V. 5.1 (see J Yang, R Yan, A Roy, D Xu, J Poisson, Y Zhang. The I-TASSER Suite: Protein structure and function prediction. Nature Methods, 12: 7-8 (2015)) and
SWISS-MODEL homology-modelling server (see Biasini M, Bienert S, Waterhouse A, Arnold K, Studer G, Schmidt T, Kiefer F, Cassarino T G, Bertoni M, Bordoli L, Schwede T (2014). SWISS-MODEL: modelling protein tertiary and quaternary structure using evolutionary information (Nucleic Acids Research 2014 (1 Jul. 2014) 42 (W1): W252-W258).

Thus, according to one embodiment, it is possible to identify enzymes with polysaccharide oxidase activity according to a method comprising the following steps:

a1) identifying one or more polypeptides liable to have polysaccharide oxidase activity;

a2) identifying experimental coordinates of a main chain of a reference polypeptide, including or not including the positions of the side chains, said main chain and/or said side chains being included in a polypeptide of sequence SEQ ID NO. 2 or SEQ ID NO. 7, or a fragment thereof with polysaccharide oxidase activity;

a3) identifying a sequence alignment of one or more of said candidate polypeptides with a reference polypeptide having a sequence identity of 20% or more with SEQ ID NO. 1, SEQ ID NO. 2, SEQ ID NO 3 or SEQ ID NO. 7 according to the BLAST-P comparison method, characterized in that said one or more candidate polypeptides have an E-value of 10 e⁻³ or less;

b) determining from a1), a2) and a3) the theoretical coordinates of said one or more candidate polypeptides;

c) determining the RMSD (root-mean-square deviation) of said theoretical coordinates with the experimental coordinates of said reference polypeptide;

it being understood that an increased RMSD of said theoretical coordinates relative to the experimental coordinates beyond a reference threshold indicates a lower probability of having polysaccharide oxidase activity;

it being understood that a decreased RMSD of said theoretical coordinates relative to the experimental coordinates below a reference threshold indicates a higher probability of having polysaccharide oxidase activity;

d) optionally, choosing the one or more candidate polypeptides whose theoretical coordinates define a site of interaction with copper of “histidine brace active site” type, formed by two histidine residues and a tyrosine, one of the two histidine residues being and N-terminal histidine.

### EXAMPLES

Materials and Methods

A.1. Cloning and Production of the Genes

The nucleotide sequence was synthesized by codon optimization using P. pastoris (GenScript, Piscataway, USA), and then inserted with the native signal sequence into a vector pPICZαA (Invitrogen, Cergy-Pontoise, France) using the restriction sites BstBI and XbaI, in phase with the C-terminal (His)₆ tag sequence. The strain P. pastoris X33 and the vector pPICZαA are components of the Easy Select Expression System (Invitrogen), and all the media and protocols are described in the manufacturer's manual (Invitrogen).

Transformation of the competent P. pastoris X33 colonies was performed by electroporation using a recombinant plasmid pPICZαA linearized with PmeI as described in Bennati-Granier et al. (Biotechnol. Biofuels 8, 90 (2015)).

The zeocin-resistant transformants were then tested (screened) as a function of their protein production. The best transformants were cultured in 2 liters of BMGY containing 1 ml·l⁻¹ of Pichia mineral salts (PTM4) as follows: (2 g·l⁻¹ CuSO₄.5H₂O, 3 g·l⁻¹ MnSO₄.H₂O, 0.2 g·l⁻¹ NazMoO₄.2H₂O, 0.02 g·l⁻¹ H₃BO₃, 0.5 g·l⁻¹ CaSO₄.2H₂O, 0.5 g·l⁻¹ CaCl₂, 12.5 g·l⁻¹ ZnSO₄.7H₂O, 22 g·l⁻¹ FeSO₄.7H₂O, biotin 0.2 g·l⁻¹, H₂SO₄ 1 ml·l⁻¹); with stirring at 30° C. (200 rpm) up to an OD₆₀₀ of between 2 and 6.

The cells are then transferred into 400 mL of BMMY containing 1 ml·l⁻¹ of PTM4 salts at 20° C. with stirring (200 rpm) for 3 days, supplemented with 3% (v/v) of methanol each day.

Purification was performed as already described in Bennati-Granier et al. (2015) and the concentrated protein was dialyzed in a sodium acetate buffer, pH 5.2 and stored at 4° C.

A.2. Production of LPMOs of AAxx Type by P. coccineus

The transformant producing the largest amount of protein was selected for culturing in a bioreactor. This is performed in 1.3 L New Brunswick BioFlo® 115 fermenters (Eppendorf, Hamburg, Germany) according to the protocol for the fermentation of P. pastoris (Invitrogen) with a few modifications. P. pastoris is cultivated in solid YPD agar medium (20 g·L⁻¹ peptone, 10 g·L⁻¹ yeast extract, 20 g·L⁻¹ glucose, 20 g·L⁻¹ agar). 100 mL of BMGY medium contained in a 500 mL flask are inoculated from an isolated P. pastoris colony and incubated at 30° C. in a rotary incubator with stirring (200 rpm) for 16 to 18 hours until an OD 600 of between 4 and 6 is obtained. A 10% (v/v) inoculum originating from this preculture serves to seed the bioreactor. The first step of the batch culture is performed in 400 mL of minimum medium containing 40 g·L⁻¹ glycerol; 26.7 mL·L⁻¹ H₃PO₄; 14.9 g·L⁻¹ MgSO₄.7H₂O; 0.93 g·L⁻¹ CaSO₄.2H₂O; 7.7 g·L⁻¹ KCl; 4.13 g·L⁻¹ KOH; 4.35 mL·L⁻¹ PTM 1 salt solution (6 g·L⁻¹ CuSO₄.5H₂O, 0.08 g·L⁻¹ NaI, 3 g·L⁻¹ MnSO₄.H₂O, 0.2 g·L⁻¹ NazMoO₄.2H₂O, 0.02 g·L⁻¹ H₃BO₃, 0.5 g·L⁻¹ CoCl₂, 20 g·L⁻¹ ZnCl₂.7H₂O, 0.2 g·L⁻¹ biotin, 5 mL·L⁻¹ H₂SO₄, 65 g·L⁻¹ FeSO₄-7H₂O). This step is performed at 30° C., with stirring at 400 rpm and at a pH maintained at 5 by adding ammonium hydroxide (28% v/v). The oxygenation of the medium is controlled at 20% by cascade oxygen enrichment (0-50%) at a gas flow rate of 0.5 v.v.m. 200 mL of Pluriol 8100 (BASF, Ludwigshafen, Germany) are added to the culture as antifoaming agent. After culturing for 20 to 24 hours, the second phase starts by simultaneous addition of 50 g of sorbitol and 0.5% of methanol (v/v) to the bioreactor so that the yeasts switch over to metabolism of the methanol (about 5 hours). During this phase, the stirring is increased to 500 rpm and the pH is gradually raised to pH 6 by adding ammonium hydroxide (28% v/v). The induction phase (phase 3) starts by adding a solution of methanol containing 12 mL·L⁻¹ of PTM1 salts (containing copper) in fed-batch mode. The initial flow rate is 1.47 mL·h⁻¹ and is increased, after about 14 hours of incubation, to 2.94 mL·h⁻¹. The induction phase is performed at 20° C. and the dissolved oxygen concentration is maintained at 20% by stirring (800 rpm), gas flow rate (0.2-1 v.v.m.) and the oxygen cascade (0-50%). The induction phase is continued for 144 hours.

A.3. Purification of the LPMOs of AAxx Type from P. pastoris

The culture supernatants are recovered after centrifugation at 2700×g for 5 minutes at 4° C. and are passed through 0.45 m filters (Millipore, Molsheim, France) in order to remove the remaining cells. For the enzymes bearing a 6×his tag, the pH is adjusted to 7.8 and the supernatants are filtered a second time through 0.2 m filters before being deposited in a 5 mL His Trap HP column (GE Healthcare, Buc, France) connected to the Akta Xpress system (GE Healthcare). The column is equilibrated in a buffer containing 50 mM of Tris HCl pH 7.8 and 150 mM of NaCl (buffer A) before loading. After depositing the supernatants, the column is washed with 5 column volumes (CV) of buffer A containing 10 mM of imidazole. Elution is then performed by passing 5 CV of buffer A containing 150 mM of imidazole. The fractions containing the purified protein are pooled and concentrated using a 3 kDa Vivaspin concentrator (Sartorius, Palaiseau, France) before being loaded onto a HiLoad 16/600 Superdex 75 Prep Grade column (GE Healthcare) for separation in 50 mM of acetate buffer at pH 5.2. Gel filtration analysis showed that the PcAAxx proteins are monomeric in solution, even after addition of copper. For the enzymes not containing the 6×his tag, the salts contained in the culture medium are diluted tenfold in 20 mM of Tris-HCl pH 8 and the proteins are then concentrated using a Pellicon-2 cassette with a 10 kDa cut-off (Millipore) until a volume of about 200 mL is obtained, which is then loaded into a 20 mL High Prep DEAE column (GE Healthcare). The proteins are then eluted from the column in a linear gradient of 1 M NaCl (0 to 700 mM in 200 mL). The fractions are then analyzed by SDS-PAGE to check for the presence of the recombinant protein, and the fractions containing the protein are pooled in the same sample and concentrated. The concentrated proteins are finally incubated overnight in a solution containing their molar equivalent of copper, before another separation step using a HiLoad 16/600 Superdex 75 Prep Grade column in 50 mM of acetate buffer at pH 5.2. Finally, the fractions containing the purified proteins are pooled in the same sample and concentrated with a 3 kDa Vivaspin concentration column (Sartorius).

A.4. Crystallization of the Purified PcAAxxB Proteins

The purified PcAAxxB proteins (JGI ID 1372210; GenBank ID #KY769370) are concentrated with a 10 kDa polyether sulfone Vivaspin concentrator (Sartorius). The protein concentration is determined by measuring the A280 nm of the solution with a Nanodrop ND-2000 (Wilmington, Del., USA). All the crystallization experiments were performed at 20° C. by the sitting drop technique (vapor diffusion) on a 96-well crystallization plate (Swissci) with a Mosquito® Crystal crystallization robot (TTP Labtech). The reservoirs contain 40 μL of commercial screening solution and the crystallization drops are prepared by mixing 100 nL of reservoir solution with 100, 200 and 300 nL of solution containing the protein to be crystallized. A first result was obtained after a week using one of the conditions prepared with the AmSO 4 screen solution (Qiagen) containing 2.4 M (NH₄)₂SO₄ and 0.1 M of citric acid at pH 4. In order to obtain crystals capable of generating a diffraction signal, this crystallization condition was optimized by mixing the protein solution at 28 mg·mL⁻¹ with a precipitation solution composed of 2.4 M (NH₄)₂SO₄ and 0.1M of citric acid at pH 4.4 in a volume ratio of 3/1. The PcAAxxB crystals are generated in a week at a size of 0.15×0.15×0.05 mm. The crystals belong to the P41212 space group and have axes of 204×204×110 Å and two molecules per asymmetric unit.

A.5. Data Collection, Structure Determination and Refinement

For the purposes of cryoprotection, the PcAAxx crystals are incubated for 5 minutes in 2.4M Li₂SO₄ solution to replace the 2.4M of (NH₄)₂SO₄ of the stock solution, before instantaneous cooling in liquid nitrogen. Since the first X-ray fluorescence scan attempts on the native crystals did not reveal the significant presence of copper atoms in the crystals, a derived crystal is obtained by introducing a heavy atom by incubating the crystals in the reservoir solution supplemented with 55 mM of the gadoteridol-gadolinium (Gd) complex before cooling. The initial diffraction data were collected with an ID23-1 beamline, whereas the MAD data were collected with an ID30B beamline in the European Synchrotron Radiation Facility (ESRF) laboratory, Grenoble, France. The data were then indexed and integrated in the P41212 space group using XDS. The data processing required was performed with the CCP4 software suite. Determination of the substructure of GD3+ and the subsequent phasing combined with solvent flattening was performed with SHELXC/D/E, and using the SAD data collected on the edge of the Gd, which made it possible to obtain a correlation coefficient of 71.8%. From the experimental phases, an initial model containing 526 residues (out of 584) was automatically constructed with Buccaneer and then manually completed with Coot (44). This initial model was used for the rigid body refinement, followed by the restricted refinement against the native data with the Refmac software. A random set of 5% of reflections was used to allow validation of the model. The quality of the model was determined with the internal Coot modules and using the Molprobity server. The collected data and the refinement statistics are collated below:

TABLE 1
Refinement statistics

| Properties | Value |
|---|---|
| Space group | P41212 |
| Unit cell parameters | 203.75 203.75 110.59 |
| a, b, c, α, β, γ | 90.00 90.00 90.00 |
| Wavelength (Å) | 0.97625 |
| Resolution range (Å) | 44.47-3.01 |
| Completude (%) (outer shell) | 98.5 (99.4) |
| Multiplicity (outer shell) | 8.3 (8.6) |
| <I/σ (I)> (outer shell) | 15.9 (2.0) |
| Rwork/Rfree | 0.23/0.24 |
| Ramachandran statistics (%) | 100% |
| (favorable positions) | |

A. 6. Bioinformatic Analysis of the AAxx LPMOs

The sequences of the AAxx from P. coccineus (Genbank ID KY769369 and KY769370) were compared with the NCBI non-redundant sequence database with BlastP (29) in February 2016. The analyses by Blast using AAxx did not make it possible to find AA9s, AA10s, AA11s, or AA13s with significant scores, and inversely. MUSCLE was used to perform the multiple alignments. In order to avoid interferences due to the presence or absence of additional residues, the signal peptides and the C-terminal extensions were removed. The bioinformatic analyses were performed using 286 fungal genomes sequenced and shared beforehand by JGI collaborators. The protein clusters are available by means of the JGI (https://goo.gl/ZAa2NX) for each of the varieties of fungi. 100 protein alignments originating from selected protein clusters were cleaned beforehand and fused in order to generate a phylogenetic tree. These clusters are present in one copy, as far as is possible, in one copy in all the varieties of fungi to improve the score Σ1/n (n represents the number of copies in the genome). The sequences originating from the clusters were aligned with Mafft and cleaned with Gblocks, and the phylogenetic tree was generated by concatenation of the alignments with Fasttree. The tree is visualized with Dendroscope and Bio::phylo.

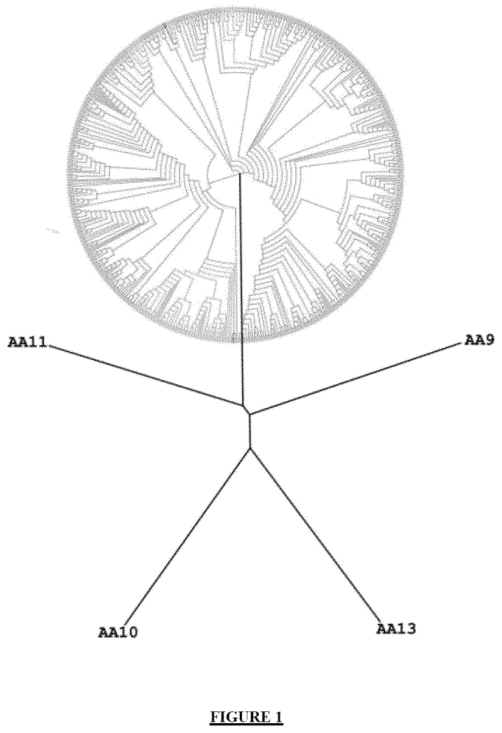

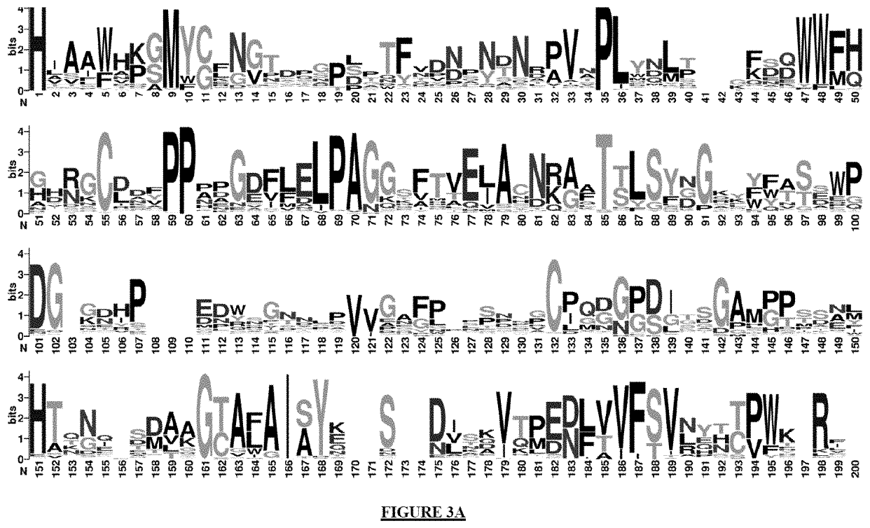

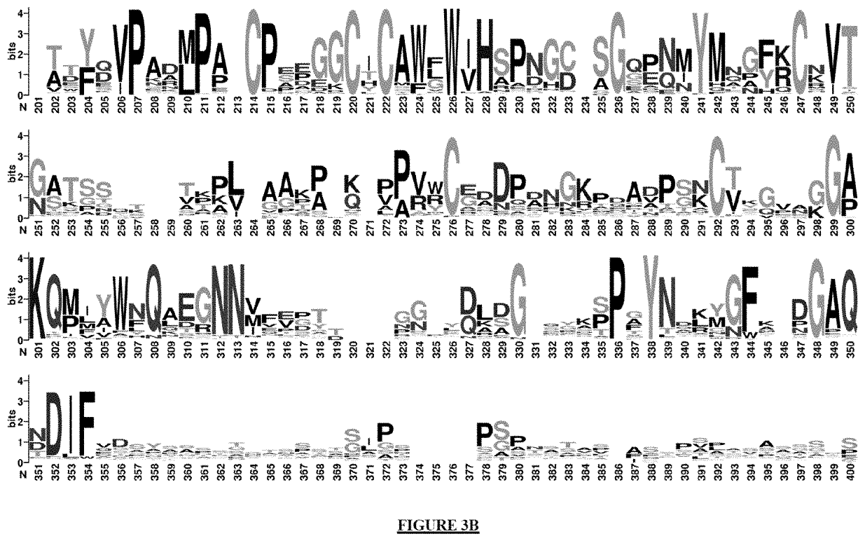

See FIG. 1 for the phylogenetic tree and FIGS. 3A and 3B corresponding to the consensus sequence of the catalytic module.

A. 7. Copper Loading Protocol

The AAxx enzymes were loaded with copper using copper salts (sulfate or acetate) during or after purification. The proteins were incubated with 10 molar equivalents of copper salts between 2 hours and overnight at 4° C. The excess copper was then removed by diafiltration with 3 kDa centricons or by gel filtration chromatography. The presence of copper may be determined by inductively-coupled plasma mass spectrometry (ICP-MS), as described below.

A. 8. ICP-MS Analysis

Before the analysis, the samples are mineralized in a mixture containing 2/3 of nitric acid (Sigma-Aldrich, 65% Purissime) and 1/3 of hydrochloric acid (Fluka, 37%, Trace Select) at 120° C. The residues are diluted in ultrapure water (2 mL) before the ICP-MS analysis. The instrument for performing the ICP-MS analysis is an ICAP Q machine (ThermoElectron, Les Ullis, France), equipped with a collision chamber. The calibration curve is obtained by dilution of a certified multi-element solution (Sigma-Aldrich). The copper concentrations are determined with the Plasmalab software (Thermo-Electron) at a mass of interest of m/z=63.

A.9. Defibrillation Tests

Aqueous dispersions of Kraft cellulose fibers are adjusted to pH 5.2 in acetate buffer (50 mM) in a final reaction volume of 5 mL. The polypeptide of sequence SEQ ID NO. 1 (LPMO AAxx) is added to the fibers to a final concentration of 20 mg·g⁻¹ in the presence of 1 mM ascorbic acid. The enzymatic incubation is performed at 40° C. with gentle stirring for 48 hours. The samples are then dispersed with a Polytron PT2100 homogenizer (Kinematica AG, Germany) for 3 minutes, and then subjected to ultrasonication with a QSonica Q700 sonicator (20 kHz, QSonica LLC., Newtown, USA) at an ultrasonication power of 350 W for 3 minutes as described previously (Villares et al, Sci Rep. 2017 Jan. 10; 7: 40262. doi: 10.1038/srep40262). The reference sample is subjected to the same treatment, in the absence of enzyme. The cellulose fibers (reference fibers and fibers treated with PcAAxx) are deposited on a glass slide and observed with a BX51 polarizing microscope (Olympus France S.A.S.) with a 4× objective. The images are taken with a U-CMAD3 camera (Olympus Japan).

The light microscopy images and the atomic force microscopy (AFM) images and the transmission electron microscopy (TEM) images were taken after depositing the fibers at a concentration of 0.1 g/L onto solid substrates, after drying with a stream of nitrogen.

Results

### Example 1: Crystallographic Structure of the Catalytic Module of PcAAxxB (JGI ID 1372210; GenBank ID #KY769370) at 3.0 Å

The crystallographic structure of the catalytic module of PcAAxxB (JGI ID 1372210; GenBank ID #KY769370), refined to a resolution of 3.0 Å, reveals a structured core and a catalytic site formed according to a “histidine brace” canonical coordination mode, exposed at the surface.

PcAAxxB (#KY769370) was produced in large amounts in Pichia pastoris and purified until homogeneous.

The structure of PcAAxxB was resolved by the MAD (multiple-wavelength anomalous dispersion) technique from the gadolinium signal, and then refined to a resolution of 3.0 Å. The protein core is structured as a large antiparallel β sandwich, this folding being globally similar to that already identified for other families.

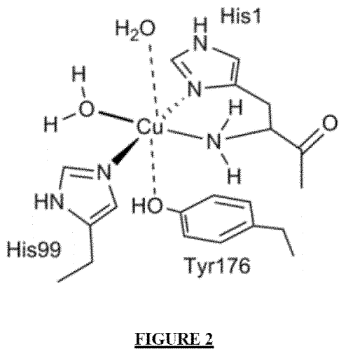

The active site of PcAAxxB consists of His 1, His99 and Tyr176, which form a “histidine brace” canonical coordination mode (see FIG. 2).

In contrast with the flat surfaces of interaction with the ligand that are ordinarily observable in LPMOs of AA9 type, the surface of PcAAxxB has a rippled shape presenting a clamp formed by two protruding loops, which is visible using the pdb (Protein Data Bank) format. Five N-glycans are attached to the crystallographic structure of PcAAxxB, via the following asparagine residues: Asn13, Asn76, Asn133, Asn183 and Asn217.

The crystallographic structure also indicates the presence of 10 cysteine residues involved in five disulfide bridges, at the following positions: Cys67 & Cys90; Cys109 & Cys136; Cys153 & Cys158; Cys160 & Cys182; Cys202 & Cys218.

The crystal structure includes two molecules per asymmetric unit. The coordinates of the A chain are presented here in the pdb format. The B chain, which also forms part of the asymmetric unit, is not shown here.

When it is visualized using the PyMOL© Viewer 1.7.4.5 Edu software (Schrodinger, LLC), the structure is characterized by six β leaflets forming an antiparallel β-sandwich core, the limits of which (with reference to the sequence SEQ ID NO. 2) are defined as follows:
from residue Phe53 to Glu64;
from residue Cys109 to Ala114;
from residue Thr128 to Asn133;
from residue Phe141 to Val146;
from residue Cys158 to Trp164;
from residue Met177 to Thr185.

The sequence SEQ ID NO. 7 corresponds to the minimum fragment of sequence SEQ ID NO. 2 comprising the three amino acids involved in the copper-binding catalytic triad, including the histidine residue in the N-terminal position, and also comprising the antiparallel β-sandwich, up to residue Thr185.

The following residues included in the sequence SEQ ID NO. 2 may also be positioned relative to a consensus sequence derived from FIG. 3, as follows:

Table 1A: Consensus sequence and position on SEQ ID NO. 2

| Consensus sequence | 1 | 20-35 | 42-60 | 78-151 | 136-226 | 146-241 | 193-338 |
|---|---|---|---|---|---|---|---|
| Amino acid | H | P | P | H | W | Y | Y |
| Position SEQ ID 2 | 1 | 28 | 47 | 99 | 164 | 176 | 246 |

Table 1B: Consensus sequence and position on SEQ ID NO. 2 (cysteine residues)

| Consensus sequence | 1 | 20-35 | 42-60 | 78-151 | 136-226 | 146-241 |
|---|---|---|---|---|---|---|
| Amino Acid | C | C | C | C | C | C |
| Position SEQ ID 2 | 153 | 158 | 160 | 182 | 202 | 218 |

### Example 2: Defibrillation of Cellulose-Based Supports Derived from Birch and from Coniferous Trees Under the Action of the Lytic Polysaccharide Monooxygenases (LPMOs)

The fibers are placed in contact with an enzyme with polysaccharide oxidase activity according to the invention, of sequence SEQ ID NO. 1, and ascorbate, and then subjected to gentle stirring for 48 hours. In the absence of enzyme, the fibers remain intact and no modification is observed.

After 48 hours of treatment in the presence of the enzyme, a start of defibrillation is observed after mechanical dispersion of the fibers.

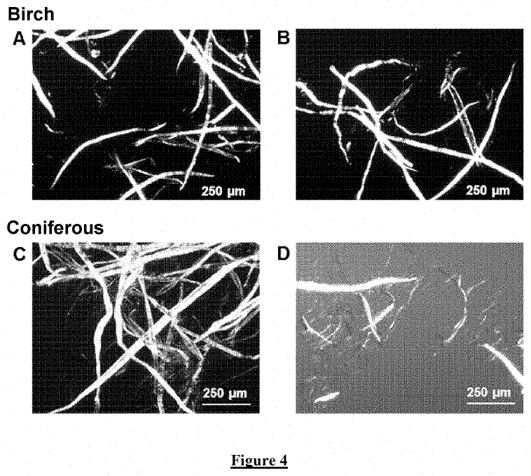

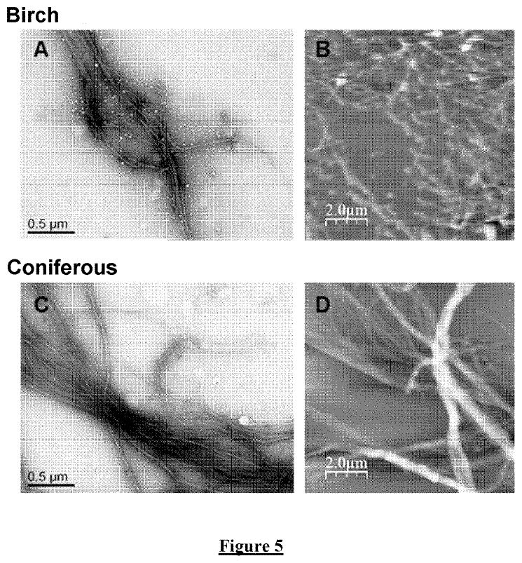

The dispersions were then analyzed by light microscopy, transmission electron microscopy (TEM) and atomic force microscopy (AFM). The images show structures of nanometric sizes. The fibers are entirely destructured, revealing the crystalline zones of the fiber (FIGS. 4 and 5).

### Example 3: Production of Oxygen Peroxide by the Polypeptides of Sequences SEQ ID NO. 1, SEQ ID NO. 2 and SEQ ID NO. 3

In order to determine the function of the polypeptides of sequence SEQ ID NO. 1, SEQ ID NO. 2 and SEQ ID NO. 3, their capacity to produce hydrogen peroxide (H₂O₂) was evaluated, in the presence of oxygen and of an electron donor (ascorbate). Significant production of H₂O₂ was detected. This production is similar in amplitude to that observed for the LPMOs of other families described in the literature, such as the LPMOs AA9 of P. anserina (Bennati-Granier et al., 2015, Biotechnol. Biofuels 8, 90).

### Example 4: Polysaccharide Degradation Activity of Polypeptides of Sequences SEQ ID NO. 1, SEQ ID NO. 2 and SEQ ID NO. 3, in a Test of Sequential Degradation of Lignocellulose

The degradation of lignocellulosic biomass by polypeptides of sequences SEQ ID NO. 1, SEQ ID NO. 2 and of a cellulase cocktail from T. reesei was tested by sequential reactions. Cellulose-based supports of “poplar” type were first incubated with 2.2 μM (equivalent to 70 μg) of polypeptide of sequence SEQ ID NO. 1 or 2.2 μM (equivalent to 70 μg) of polypeptide of sequence SEQ ID NO. 2 for 48 hours; after this, 10 μg of T. reesei TR3012 cellulase cocktail were added. The reactions were incubated for 24 hours.

Analysis of the soluble sugars released was performed according to several methodologies (DNS assay, RTU assay and HPAEC), which showed an improvement in the release of glucose and of cello-oligosaccharides. Addition of supplementary amounts of polypeptides of sequences SEQ ID NO. 1 or SEQ ID NO. 2 resulted in a proportional increase in glucose release.

### Example 5: Polysaccharide Degradation Activity of Polypeptides of Sequences SEQ ID NO. 1 and SEQ ID NO. 2 in a Test of Sequential Degradation of Lignocellulose, in the Presence of LPMO of AA9 Type

The degradation of lignocellulosic biomass by polypeptides of sequences SEQ ID NO. 1, in combination with LPMOs of AA9 type was tested in sequential reactions.

Cellulose-based supports of “poplar” type were first incubated with (i) a control medium, (ii) 2.2 μM (equivalent to 35 μg) of polypeptide SEQ ID NO. 1, (iii) 2.2. μM of LPMO of AA9 type and (iv) 1.1 μM of polypeptide of SEQ ID NO. 1 and 1.1 μM of LPMO of AA9 type, for 48 hours; after this, 10 μg of T. reesei TR3012 cellulase cocktail were added. The reactions were incubated for 24 hours.

Analysis of the soluble sugars released was performed according to several methodologies (DNS assay, RTU assay and HPAEC), which showed an improvement in the release of glucose and of cello-oligosaccharides.

SEQUENCE LISTING
SEQ ID NO. 1
HAAFWDKSMYGFNVTAQTFPYDNRPQVPLYNMTFDQWWFHGHKDYPPNEGD
FFELPAGGEVNSIISCDKGATPFYESSPGGDSGYGSNSPCPGQPMSEYHTT
GIDDVKGCCMAIAYKPDVNDVQPDDFVVFSCNSTCVWEMNTKFEIPKLPAC
PEGGCHCAWFWIHSYDSGAEQIYMNGFKCKVTGDVGTQPLGKPAVPRRCGA
DPDHGKPDPTPGNCTIGAKTPMYWYQREGNNMFEDTYDAPYYNPLYGFNDG
AQNDIFMDGVIASLA
SEQ ID NO. 2
HIAFWHNSMYGFNVTEQTFPYDNRPVVPLQYMTFQEWWFHNHLDYPPHPGD
FFDFPAGKAATAELACNKGATTWFNSSEGGNIQNGNDPCPGSPPSEYHTTG
IDDVKGCAMAIAYESDVRKIKPEDFTVFSVNQTCVWYRFTDFQVPERMPPC
PPGGCHCAWFWIHSPDSGGEQIYMNGFQCNITGSTSHVPLAKPKVARRCGA
DPDHGKPDAVPGNCTYGAKQPLYWLQKEGNNEFDDYIAPPFYNDLYNFKDG
AQNDIFVDSYPDGI
SEQ ID NO. 3
HVAAFVKGMYCEGGPDPNNYNPNSNTPVNPLWDLPFEQWWMQADRGCNKAP
PPDGASVALPAGGQFTVELAHNQAQTTLSFNGQFAGEWPDGQPHPENWSGP
GSPPDCIQDDGAMHTNNQTMAAGTAWAISYNDDISKVTMDNLVVFSVLEHT
PWKRIATYDVPKDLPACPAGGCYCAWLWVPNGCGEPNMYMANYRCHVTNTT
STKQLAQAKPPTWCGGDSSKCTKGAKQMIAWNQATGNNVQVPNGASPGYNI
NMGWAPGAQNDIFA
SEQ ID NO. 4
CACGCCGCGTTCTGGGACAAGTCCATGTACGGCTTCAACGTCACCGCACAG
ACCTTCCCCTACGACAACCGGCCCCAGGTTCCGCTCTACAACATGACTTTC
GACCAATGGTGGTTCCACGGTCACAAAGACTACCCACCCAACGAGGGCGAT
TTCTTCGAACTCCCTGCGGGTGGAGAGGTGAACAGCATCATCTCCTGCGAT
AAGGGTGCGACTCCGTTCTACGAGTCGTCTCCGGGCGGAGATTCGGGCTAT
GGCAGCAACTCTCCTTGCCCCGGGCAGCCCATGTCCGAGTACCACACGACG
GGCATTGATGATGTCAAGGGCTGCTGCATGGCCATCGCGTACAAGCCCGAT
GTCAACGATGTGCAGCCAGACGACTTCGTGGTCTTCTCCTGCAACTCGACG
TGCGTTTGGGAGATGAACACCAAGTTTGAGATCCCGAAGCTCCCTGCCTGC
CCCGAAGGTGGTTGCCATTGCGCCTGGTTCTGGATTCACTCCTACGATAGC
GGTGCTGAGCAGATCTATATGAATGGTTTCAAGTGCAAAGTAACCGGCGAC
GTAGGCACTCAGCCTCTCGGCAAGCCTGCCGTCCCGCGCAGGTGCGGTGCC
GACCCTGATCACGGCAAGCCTGATCCTACTCCCGGTAATTGTACTATCGGG
GCCAAGACGCCCATGTACTGGTACCAGCGGGAGGGTAACAACATGTTCGAA
GACACTTATGACGCCCCGTACTATAACCCACTCTACGGCTTCAACGATGGG
GCGCAGAACGACATTTTCATGGATGGTGTCATCGCGTCCCTCGCA
SEQ ID NO. 5
CACATCGCCTTCTGGCATAACAGCATGTACGGGTTCAATGTGACGGAACAG
ACGTTCCCTTACGACAACCGCCCCGTCGTCCCGCTTCAGTACATGACCTTC
CAAGAATGGTGGTTCCACAACCACCTCGACTACCCGCCCCACCCGGGCGAC
TTCTTCGACTTCCCGGCCGGCAAGGCCGCGACGGCGGAGCTCGCGTGCAAC
AAGGGCGCGACCACCTGGTTCAACTCCTCCGAGGGCGGCAACATCCAGAAC
GGCAACGACCCGTGCCCGGGGAGCCCCCCGAGCGAGTACCACACGACGGGC
ATCGACGACGTGAAGGGCTGCGCGATGGCGATCGCGTACGAGTCCGACGTC
AGGAAGATCAAGCCCGAGGACTTCACCGTCTTCAGCGTGAACCAGACGTGC
GTCTGGTACCGCTTCACGGACTTCCAGGTCCCCGAGCGCATGCCGCCGTGC
CCTCCTGGCGGCTGTCACTGCGCGTGGTTCTGGATCCACTCGCCCGATAGC
GGCGGTGAGCAGATCTACATGAACGGCTTCCAGTGCAACATCACCGGCTCC
ACGTCCCACGTCCCGCTCGCAAAGCCCAAAGTCGCTCGCCGCTGCGGTGCG
GACCCGGACCACGGCAAGCCCGACGCCGTCCCCGGCAACTGCACATACGGT
GCGAAGCAGCCCCTCTACTGGCTCCAGAAGGAGGGCAACAACGAGTTCGAC
GACTACATCGCGCCGCCGTTCTACAACGACCTGTACAACTTCAAGGACGGC
GCGCAGAACGACATCTTCGTCGACTCGTACCCCGACGGCATC
SEQ ID NO. 6
ATGTTCTGGACGGTTCCCGTTACACTGGCTCTCGCCTCCGGGGCCTCTGCC
CATGTTGCCGCCTTCGTGAAGGGCATGTACTGCGAGGGTGGCCCCGACCCT
AACAACTACAACCCCAACTCCAACACTCCCGTCAACCCTCTCTGGGATCTT
CCTTTCGAGCAGTGGTGGATGCAGGCCGACCGTGGCTGCAACAAGGCTCCT
CCTCCTGACGGTGCCTCCGTTGCTCTGCCTGCCGGCGGCCAGTTCACTGTT
GAGCTCGCCCACAACCAGGCCCAGACGACCTTATCCTTCAATGGACAGTTC
GCTGGCGAGTGGCCTGATGGCCAGCCTCACCCAGAGAACTGGAGCGGCCCC
GGTAGCCCCCCTGACTGCATCCAGGACGACGGTGCCATGCATACCAACAAC
CAGACAATGGCCGCTGGTACCGCTTGGGCCATTTCCTACAATGACGACATC
TCCAAGGTGACCATGGACAACCTGGTTGTTTTCTCTGTCCTTGAGCACACC
CCTTGGAAGCGCATCGCCACCTATGATGTCCCCAAGGATCTCCCTGCTTGC
CCTGCTGGCGGCTGCTACTGCGCCTGGCTTTGGGTTCCCAACGGGTGCGGC
GAGCCCAACATGTACATGGCCAACTACAGGTGCCATGTCACCAACACCACC
TCGACCAAGCAGCTTGCTCAAGCCAAGCCCCCCACCTGGTGTGGCGGTGAC
TCTTCCAAGTGCACCAAGGGCGCTAAGCAGATGATTGCTTGGAACCAGGCC
ACAGGCAACAATGTCCAAGTTCCCAATGGCGCTTCCCCCGGCTACAACATC
AACATGGGCTGGGCTCCCGGTGCTCAGAACGACATCTTCGCGTAG
SEQ ID NO. 7
HIAFWHNSMYGFNVTEQTFPYDNRPVVPLQYMTFQEWWFHNHLDYPPHPGD
FFDFPAGKAATAELACNKGATTWFNSSEGGNIQNGNDPCPGSPPSEYHTTG
IDDVKGCAMATAYESDVRKIKPEDFTVFSVNQTCVWYRFTDFQVPERMPPC
PPGGCHCAWFWIHSPDSGGEQIYMNGFQCNIT
SEQ ID No 8
MFWTVPVTLALASGASA
SEQ ID NO. 9
MFWTVPVTLALASGASAHVAAFVKGMYCEGGPDPNNYNPNSNTPVNPLWDL
PFEQWWMQADRGCNKAPPPDGASVALPAGGQFTVELAHNQAQTTLSFNGQF
AGEWPDGQPHPENWSGPGSPPDCIQDDGAMHTNNQTMAAGTAWAISYNDDI
SKVTMDNLVVFSVLEHTPWKRIATYDVPKDLPACPAGGCYCAWLWVPNGCG
EPNMYMANYRCHVTNTTSTKQLAQAKPPTWCGGDSSKCTKGAKQMIAWNQA
TGNNVQVPNGASPGYNINMGWAPGAQNDIFA

CRYSTALLOGRAPHIC STRUCTURE OF A POLYPEPTIDE OF SEQUENCE SEQ ID NO. 2

| | | | | | |
|---|---|---|---|---|---|
| MODRES | NAG | A | 300 | NAG-b-D | RENAME |
| MODRES | NAG | A | 301 | NAG-b-D | RENAME |
| MODRES | NAG | A | 310 | NAG-b-D | RENAME |
| MODRES | NAG | A | 311 | NAG-b-D | RENAME |
| MODRES | MAN | A | 313 | MAN-a-D | RENAME |
| MODRES | MAN | A | 314 | MAN-a-D | RENAME |
| MODRES | MAN | A | 315 | MAN-a-D | RENAME |
| MODRES | NAG | A | 320 | NAG-b-D | RENAME |
| MODRES | NAG | A | 321 | NAG-b-D | RENAME |
| MODRES | MAN | A | 323 | MAN-a-D | RENAME |
| MODRES | MAN | A | 324 | MAN-a-D | RENAME |
| MODRES | NAG | A | 330 | NAG-b-D | RENAME |
| MODRES | NAG | A | 331 | NAG-b-D | RENAME |
| MODRES | MAN | A | 333 | MAN-a-D | RENAME |
| MODRES | NAG | A | 340 | NAG-b-D | RENAME |
| MODRES | NAG | A | 341 | NAG-b-D | RENAME |
| MODRES | MAN | A | 343 | MAN-a-D | RENAME |
| MODRES | MAN | A | 344 | MAN-a-D | RENAME |
| MODRES | MAN | A | 345 | MAN-a-D | RENAME |
| MODRES | MAN | A | 346 | MAN-a-D | RENAME |

| | | | | | | | |
|---|---|---|---|---|---|---|---|
| SSBOND | 1 | CYS | A | 67 | CYS | A | 90 |
| SSBOND | 2 | CYS | A | 109 | CYS | A | 136 |
| SSBOND | 3 | CYS | A | 153 | CYS | A | 158 |
| SSBOND | 4 | CYS | A | 160 | CYS | A | 182 |
| SSBOND | 5 | CYS | A | 202 | CYS | A | 218 |

| | | | | | | | | | |
|---|---|---|---|---|---|---|---|---|---|
| LINKR | C1 | NAG | A | 300 | ND2 | ASN | A | 13 | NAG-ASN |
| LINKR | C1 | NAG | A | 310 | ND2 | ASN | A | 76 | NAG-ASN |
| LINKR | C1 | NAG | A | 320 | ND2 | ASN | A | 133 | NAG-ASN |
| LINKR | C1 | NAG | A | 330 | ND2 | ASN | A | 183 | NAG-ASN |
| LINKR | C1 | NAG | A | 340 | ND2 | ASN | A | 217 | NAG-ASN |
| LINKR | | NAG | A | 300 | | NAG | A | 301 | BETA1-4 |
| LINKR | | NAG | A | 301 | | BMA | A | 302 | BETA1-4 |
| LINKR | | NAG | A | 310 | | NAG | A | 311 | BETA1-4 |
| LINKR | | NAG | A | 311 | | BMA | A | 312 | BETA1-4 |
| LINKR | | BMA | A | 312 | | MAN | A | 313 | ALPHA1-6 |
| LINKR | | MAN | A | 313 | | MAN | A | 314 | ALPHA1-3 |
| LINKR | | BMA | A | 312 | | MAN | A | 315 | ALPHA1-3 |
| LINKR | | NAG | A | 320 | | NAG | A | 321 | BETA1-4 |
| LINKR | | NAG | A | 321 | | BMA | A | 322 | BETA1-4 |
| LINKR | | BMA | A | 322 | | MAN | A | 323 | ALPHA1-3 |
| LINKR | | BMA | A | 322 | | MAN | A | 324 | ALPHA1-6 |
| LINKR | | NAG | A | 330 | | NAG | A | 331 | BETA1-4 |
| LINKR | | NAG | A | 331 | | BMA | A | 332 | BETA1-4 |
| LINKR | | BMA | A | 332 | | MAN | A | 333 | ALPHA1-3 |
| LINKR | | NAG | A | 340 | | NAG | A | 341 | BETA1-4 |
| LINKR | | NAG | A | 341 | | BMA | A | 342 | BETA1-4 |
| LINKR | | BMA | A | 342 | | MAN | A | 343 | ALPHA1-3 |
| LINKR | | MAN | A | 343 | | MAN | A | 345 | ALPHA1-2 |
| LINKR | | BMA | A | 342 | | MAN | A | 344 | ALPHA1-6 |
| LINKR | | MAN | A | 343 | | MAN | A | 346 | ALPHA1-6 |

| | | | | | | | |
|---|---|---|---|---|---|---|---|
| CRYST1 | 203.754 | 203.754 | 110.586 | 90.00 | 90.00 | 90.00 | P 41 21 2 |

| | | | | |
|---|---|---|---|---|
| SCALE1 | 0.004908 | 0.000000 | 0.000000 | 0.00000 |
| SCALE2 | −0.000000 | 0.004908 | 0.000000 | 0.00000 |
| SCALE3 | 0.000000 | −0.000000 | 0.009043 | 0.00000 |

| | | | | | | | | | | |
|---|---|---|---|---|---|---|---|---|---|---|
| ATOM | 1 | N | HIS | A | 1 | 84.207 | 23.023 | 6.334 | 1.00 91.64 | N |
| ATOM | 2 | CA | HIS | A | 1 | 84.466 | 23.579 | 7.697 | 1.00 92.54 | C |
| ATOM | 3 | CB | HIS | A | 1 | 83.152 | 23.732 | 8.477 | 1.00 96.32 | C |
| ATOM | 4 | CG | HIS | A | 1 | 82.286 | 24.858 | 7.994 | 1.00103.06 | C |
| ATOM | 5 | ND1 | HIS | A | 1 | 81.800 | 24.929 | 6.705 | 1.00107.25 | N |
| ATOM | 6 | CE1 | HIS | A | 1 | 81.067 | 26.021 | 6.570 | 1.00107.65 | C |
| ATOM | 7 | NE2 | HIS | A | 1 | 81.051 | 26.657 | 7.728 | 1.00107.39 | N |
| ATOM | 8 | CD2 | HIS | A | 1 | 81.802 | 25.948 | 8.636 | 1.00107.70 | C |
| ATOM | 9 | C | HIS | A | 1 | 85.475 | 22.714 | 8.468 | 1.00 89.46 | C |
| ATOM | 10 | O | HIS | A | 1 | 85.099 | 21.813 | 9.220 | 1.00 87.30 | O |
| ATOM | 11 | N | ILE | A | 2 | 86.758 | 22.994 | 8.247 | 1.00 85.02 | N |
| ATOM | 12 | CA | ILE | A | 2 | 87.864 | 22.287 | 8.897 | 1.00 82.47 | C |
| ATOM | 13 | CB | ILE | A | 2 | 88.504 | 21.243 | 7.951 | 1.00 82.74 | C |
| ATOM | 14 | CG1 | ILE | A | 2 | 87.504 | 20.143 | 7.573 | 1.00 84.37 | C |
| ATOM | 15 | CD1 | ILE | A | 2 | 87.087 | 19.238 | 8.714 | 1.00 84.11 | C |
| ATOM | 16 | CG2 | ILE | A | 2 | 89.744 | 20.610 | 8.573 | 1.00 82.26 | C |
| ATOM | 17 | C | ILE | A | 2 | 88.930 | 23.304 | 9.288 | 1.00 82.66 | C |
| ATOM | 18 | O | ILE | A | 2 | 89.091 | 24.331 | 8.623 | 1.00 81.32 | O |
| ATOM | 19 | N | ALA | A | 3 | 89.659 | 23.015 | 10.364 | 1.00 83.36 | N |
| ATOM | 20 | CA | ALA | A | 3 | 90.744 | 23.883 | 10.810 | 1.00 83.72 | C |
| ATOM | 21 | CB | ALA | A | 3 | 90.229 | 24.884 | 11.825 | 1.00 85.46 | C |
| ATOM | 22 | C | ALA | A | 3 | 91.897 | 23.099 | 11.410 | 1.00 82.57 | C |
| ATOM | 23 | O | ALA | A | 3 | 91.711 | 22.006 | 11.943 | 1.00 82.43 | O |
| ATOM | 24 | N | PHE | A | 4 | 93.091 | 23.676 | 11.307 | 1.00 82.50 | N |
| ATOM | 25 | CA | PHE | A | 4 | 94.289 | 23.131 | 11.939 | 1.00 82.14 | C |
| ATOM | 26 | CB | PHE | A | 4 | 95.544 | 23.679 | 11.248 | 1.00 81.58 | C |
| ATOM | 27 | CG | PHE | A | 4 | 96.801 | 23.534 | 12.055 | 1.00 82.24 | C |
| ATOM | 28 | CD1 | PHE | A | 4 | 97.531 | 22.356 | 12.025 | 1.00 84.74 | C |
| ATOM | 29 | CE1 | PHE | A | 4 | 98.696 | 22.226 | 12.763 | 1.00 85.69 | C |
| ATOM | 30 | CZ | PHE | A | 4 | 99.145 | 23.281 | 13.536 | 1.00 86.18 | C |
| ATOM | 31 | CE2 | PHE | A | 4 | 98.430 | 24.463 | 13.568 | 1.00 85.11 | C |
| ATOM | 32 | CD2 | PHE | A | 4 | 97.267 | 24.587 | 12.829 | 1.00 82.90 | C |
| ATOM | 33 | C | PHE | A | 4 | 94.243 | 23.540 | 13.408 | 1.00 79.27 | C |
| ATOM | 34 | O | PHE | A | 4 | 94.223 | 24.733 | 13.713 | 1.00 78.92 | O |
| ATOM | 35 | N | TRP | A | 5 | 94.214 | 22.556 | 14.308 | 1.00 76.19 | N |
| ATOM | 36 | CA | TRP | A | 5 | 94.078 | 22.815 | 15.748 | 1.00 73.59 | C |
| ATOM | 37 | CB | TRP | A | 5 | 92.972 | 21.947 | 16.342 | 1.00 71.23 | C |
| ATOM | 38 | CG | TRP | A | 5 | 91.616 | 22.342 | 15.888 | 1.00 71.66 | C |
| ATOM | 39 | CD1 | TRP | A | 5 | 90.794 | 21.643 | 15.057 | 1.00 71.88 | C |
| ATOM | 40 | NE1 | TRP | A | 5 | 89.622 | 22.333 | 14.860 | 1.00 73.14 | N |
| ATOM | 41 | CE2 | TRP | A | 5 | 89.674 | 23.506 | 15.565 | 1.00 73.24 | C |
| ATOM | 42 | CD2 | TRP | A | 5 | 90.919 | 23.547 | 16.223 | 1.00 72.48 | C |
| ATOM | 43 | CE3 | TRP | A | 5 | 91.226 | 24.656 | 17.018 | 1.00 72.79 | C |
| ATOM | 44 | CZ3 | TRP | A | 5 | 90.291 | 25.675 | 17.132 | 1.00 73.08 | C |
| ATOM | 45 | CH2 | TRP | A | 5 | 89.059 | 25.606 | 16.466 | 1.00 74.22 | C |
| ATOM | 46 | CZ2 | TRP | A | 5 | 88.733 | 24.533 | 15.679 | 1.00 74.18 | C |
| ATOM | 47 | C | TRP | A | 5 | 95.360 | 22.586 | 16.533 | 1.00 75.33 | C |
| ATOM | 48 | O | TRP | A | 5 | 95.922 | 21.491 | 16.518 | 1.00 74.09 | O |
| ATOM | 49 | N | HIS | A | 6 | 95.804 | 23.632 | 17.227 | 1.00 80.24 | N |
| ATOM | 50 | CA | HIS | A | 6 | 96.984 | 23.570 | 18.089 | 1.00 80.86 | C |
| ATOM | 51 | CB | HIS | A | 6 | 98.257 | 23.467 | 17.251 | 1.00 78.51 | C |
| ATOM | 52 | CG | HIS | A | 6 | 99.432 | 22.941 | 18.008 | 1.00 76.30 | C |
| ATOM | 53 | ND1 | HIS | A | 6 | 100.512 | 23.725 | 18.351 | 1.00 76.28 | N |
| ATOM | 54 | CE1 | HIS | A | 6 | 101.395 | 22.993 | 19.006 | 1.00 76.89 | C |
| ATOM | 55 | NE2 | HIS | A | 6 | 100.923 | 21.763 | 19.104 | 1.00 76.64 | N |
| ATOM | 56 | CD2 | HIS | A | 6 | 99.699 | 21.704 | 18.485 | 1.00 74.93 | C |
| ATOM | 57 | C | HIS | A | 6 | 97.027 | 24.823 | 18.964 | 1.00 83.70 | C |
| ATOM | 58 | O | HIS | A | 6 | 96.542 | 25.872 | 18.553 | 1.00 87.03 | O |
| ATOM | 59 | N | ASN | A | 7 | 97.613 | 24.720 | 20.156 | 1.00 84.93 | N |
| ATOM | 60 | CA | ASN | A | 7 | 97.683 | 25.864 | 21.083 | 1.00 82.64 | C |
| ATOM | 61 | CB | ASN | A | 7 | 98.139 | 25.415 | 22.476 | 1.00 82.43 | C |
| ATOM | 62 | CG | ASN | A | 7 | 99.559 | 24.883 | 22.488 | 1.00 85.12 | C |
| ATOM | 63 | OD1 | ASN | A | 7 | 100.174 | 24.686 | 21.440 | 1.00 88.15 | O |
| ATOM | 64 | ND2 | ASN | A | 7 | 100.089 | 24.647 | 23.682 | 1.00 87.40 | N |
| ATOM | 65 | C | ASN | A | 7 | 98.571 | 27.017 | 20.592 | 1.00 80.24 | C |
| ATOM | 66 | O | ASN | A | 7 | 98.520 | 28.120 | 21.138 | 1.00 81.89 | O |
| ATOM | 67 | N | SER | A | 8 | 99.379 | 26.753 | 19.568 | 1.00 76.92 | N |
| ATOM | 68 | CA | SER | A | 8 | 100.251 | 27.760 | 18.971 | 1.00 76.31 | C |
| ATOM | 69 | CB | SER | A | 8 | 101.521 | 27.099 | 18.439 | 1.00 78.16 | C |
| ATOM | 70 | OG | SER | A | 8 | 101.219 | 26.179 | 17.403 | 1.00 76.69 | O |
| ATOM | 71 | C | SER | A | 8 | 99.578 | 28.518 | 17.828 | 1.00 74.65 | C |
| ATOM | 72 | O | SER | A | 8 | 100.198 | 29.385 | 17.213 | 1.00 74.69 | O |
| ATOM | 73 | N | MET | A | 9 | 98.324 | 28.191 | 17.536 | 1.00 73.77 | N |
| ATOM | 74 | CA | MET | A | 9 | 97.611 | 28.834 | 16.441 | 1.00 74.84 | C |
| ATOM | 75 | CB | MET | A | 9 | 96.458 | 27.949 | 15.954 | 1.00 74.96 | C |
| ATOM | 76 | CG | MET | A | 9 | 95.228 | 27.964 | 16.847 | 1.00 74.10 | C |
| ATOM | 77 | SD | MET | A | 9 | 94.042 | 26.680 | 16.421 | 1.00 74.41 | S |
| ATOM | 78 | CE | MET | A | 9 | 93.364 | 27.372 | 14.914 | 1.00 77.74 | C |
| ATOM | 79 | C | MET | A | 9 | 97.063 | 30.188 | 16.849 | 1.00 75.28 | C |
| ATOM | 80 | O | MET | A | 9 | 96.799 | 30.440 | 18.024 | 1.00 83.04 | O |
| ATOM | 81 | N | TYR | A | 10 | 96.888 | 31.053 | 15.859 | 1.00 74.31 | N |
| ATOM | 82 | CA | TYR | A | 10 | 96.248 | 32.339 | 16.074 | 1.00 74.32 | C |
| ATOM | 83 | CB | TYR | A | 10 | 96.636 | 33.330 | 14.973 | 1.00 72.98 | C |
| ATOM | 84 | CG | TYR | A | 10 | 98.026 | 33.895 | 15.150 | 1.00 71.66 | C |
| ATOM | 85 | CD1 | TYR | A | 10 | 99.150 | 33.108 | 14.941 | 1.00 72.01 | C |
| ATOM | 86 | CE1 | TYR | A | 10 | 100.426 | 33.619 | 15.115 | 1.00 72.90 | C |
| ATOM | 87 | CZ | TYR | A | 10 | 100.586 | 34.935 | 15.497 | 1.00 73.32 | C |
| ATOM | 88 | OH | TYR | A | 10 | 101.845 | 35.457 | 15.668 | 1.00 75.71 | O |
| ATOM | 89 | CE2 | TYR | A | 10 | 99.486 | 35.738 | 15.707 | 1.00 73.41 | C |
| ATOM | 90 | CD2 | TYR | A | 10 | 98.216 | 35.216 | 15.535 | 1.00 73.15 | C |
| ATOM | 91 | C | TYR | A | 10 | 94.740 | 32.106 | 16.111 | 1.00 75.52 | C |
| ATOM | 92 | O | TYR | A | 10 | 94.209 | 31.290 | 15.354 | 1.00 77.48 | O |
| ATOM | 93 | N | GLY | A | 11 | 94.059 | 32.813 | 17.004 | 1.00 76.98 | N |
| ATOM | 94 | CA | GLY | A | 11 | 92.618 | 32.658 | 17.176 | 1.00 77.07 | C |
| ATOM | 95 | C | GLY | A | 11 | 92.251 | 31.299 | 17.739 | 1.00 77.32 | C |
| ATOM | 96 | O | GLY | A | 11 | 91.217 | 30.735 | 17.388 | 1.00 78.34 | O |
| ATOM | 97 | N | PHE | A | 12 | 93.100 | 30.779 | 18.620 | 1.00 76.88 | N |
| ATOM | 98 | CA | PHE | A | 12 | 92.893 | 29.466 | 19.228 | 1.00 77.53 | C |
| ATOM | 99 | CB | PHE | A | 12 | 94.060 | 29.150 | 20.169 | 1.00 79.12 | C |
| ATOM | 100 | CG | PHE | A | 12 | 93.945 | 27.823 | 20.868 | 1.00 82.77 | C |
| ATOM | 101 | CD1 | PHE | A | 12 | 93.956 | 26.638 | 20.146 | 1.00 83.25 | C |
| ATOM | 102 | CE1 | PHE | A | 12 | 93.863 | 25.415 | 20.785 | 1.00 83.58 | C |
| ATOM | 103 | CZ | PHE | A | 12 | 93.771 | 25.363 | 22.166 | 1.00 84.84 | C |
| ATOM | 104 | CE2 | PHE | A | 12 | 93.768 | 26.534 | 22.900 | 1.00 85.08 | C |
| ATOM | 105 | CD2 | PHE | A | 12 | 93.861 | 27.755 | 22.253 | 1.00 85.04 | C |
| ATOM | 106 | C | PHE | A | 12 | 91.560 | 29.370 | 19.972 | 1.00 78.05 | C |
| ATOM | 107 | O | PHE | A | 12 | 90.881 | 28.343 | 19.906 | 1.00 79.95 | O |
| ATOM | 108 | N | ASN | A | 13 | 91.187 | 30.445 | 20.664 | 1.00 79.85 | N |
| ATOM | 109 | CA | ASN | A | 13 | 89.955 | 30.483 | 21.455 | 1.00 80.66 | C |
| ATOM | 110 | CB | ASN | A | 13 | 90.259 | 31.013 | 22.860 | 1.00 82.92 | C |
| ATOM | 111 | CG | ASN | A | 13 | 91.025 | 30.020 | 23.719 | 1.00 85.75 | C |
| ATOM | 112 | OD1 | ASN | A | 13 | 91.179 | 28.849 | 23.368 | 1.00 86.69 | O |
| ATOM | 113 | ND2 | ASN | A | 13 | 91.509 | 30.497 | 24.869 | 1.00 88.38 | N |
| ATOM | 114 | C | ASN | A | 13 | 88.835 | 31.321 | 20.839 | 1.00 80.10 | C |
| ATOM | 115 | O | ASN | A | 13 | 87.829 | 31.582 | 21.499 | 1.00 79.99 | O |
| ATOM | 116 | N | VAL | A | 14 | 88.996 | 31.732 | 19.582 | 1.00 80.60 | N |
| ATOM | 117 | CA | VAL | A | 14 | 87.972 | 32.528 | 18.898 | 1.00 79.17 | C |
| ATOM | 118 | CB | VAL | A | 14 | 88.473 | 33.066 | 17.539 | 1.00 78.29 | C |
| ATOM | 119 | CG1 | VAL | A | 14 | 87.331 | 33.669 | 16.731 | 1.00 78.86 | C |
| ATOM | 120 | CG2 | VAL | A | 14 | 89.567 | 34.101 | 17.751 | 1.00 79.23 | C |
| ATOM | 121 | C | VAL | A | 14 | 86.715 | 31.690 | 18.685 | 1.00 79.96 | C |
| ATOM | 122 | O | VAL | A | 14 | 86.795 | 30.543 | 18.244 | 1.00 82.35 | O |
| ATOM | 123 | N | THR | A | 15 | 85.562 | 32.271 | 19.007 | 1.00 81.16 | N |
| ATOM | 124 | CA | THR | A | 15 | 84.278 | 31.594 | 18.853 | 1.00 83.25 | C |
| ATOM | 125 | CB | THR | A | 15 | 83.560 | 31.430 | 20.208 | 1.00 82.34 | C |
| ATOM | 126 | OG1 | THR | A | 15 | 83.185 | 32.715 | 20.719 | 1.00 83.75 | O |
| ATOM | 127 | CG2 | THR | A | 15 | 84.461 | 30.721 | 21.209 | 1.00 80.67 | C |
| ATOM | 128 | C | THR | A | 15 | 83.385 | 32.364 | 17.880 | 1.00 86.18 | C |
| ATOM | 129 | O | THR | A | 15 | 83.780 | 33.402 | 17.344 | 1.00 86.80 | O |
| ATOM | 130 | N | GLU | A | 16 | 82.185 | 31.839 | 17.656 | 1.00 90.27 | N |
| ATOM | 131 | CA | GLU | A | 16 | 81.237 | 32.423 | 16.714 | 1.00 95.92 | C |
| ATOM | 132 | CB | GLU | A | 16 | 79.978 | 31.557 | 16.663 | 1.00 99.78 | C |
| ATOM | 133 | CG | GLU | A | 16 | 78.944 | 31.990 | 15.636 | 1.00101.53 | C |
| ATOM | 134 | CD | GLU | A | 16 | 77.828 | 30.973 | 15.465 | 1.00104.50 | C |
| ATOM | 135 | OE1 | GLU | A | 16 | 78.104 | 29.755 | 15.519 | 1.00106.60 | O |
| ATOM | 136 | OE2 | GLU | A | 16 | 76.670 | 31.390 | 15.262 | 1.00104.82 | O |
| ATOM | 137 | C | GLU | A | 16 | 80.870 | 33.868 | 17.052 | 1.00 98.70 | C |
| ATOM | 138 | O | GLU | A | 16 | 80.799 | 34.715 | 16.159 | 1.00100.81 | O |
| ATOM | 139 | N | GLN | A | 17 | 80.660 | 34.144 | 18.337 | 1.00101.15 | N |
| ATOM | 140 | CA | GLN | A | 17 | 80.240 | 35.477 | 18.794 | 1.00101.61 | C |
| ATOM | 141 | CB | GLN | A | 17 | 79.328 | 35.375 | 20.035 | 1.00106.37 | C |
| ATOM | 142 | CG | GLN | A | 17 | 79.957 | 34.784 | 21.301 | 1.00110.91 | C |
| ATOM | 143 | CD | GLN | A | 17 | 79.913 | 33.260 | 21.369 | 1.00112.06 | C |
| ATOM | 144 | OE1 | GLN | A | 17 | 79.731 | 32.579 | 20.358 | 1.00112.42 | O |
| ATOM | 145 | NE2 | GLN | A | 17 | 80.090 | 32.721 | 22.571 | 1.00111.28 | N |
| ATOM | 146 | C | GLN | A | 17 | 81.395 | 36.460 | 19.063 | 1.00 95.82 | C |
| ATOM | 147 | O | GLN | A | 17 | 81.150 | 37.635 | 19.333 | 1.00 91.71 | O |
| ATOM | 148 | N | THR | A | 18 | 82.639 | 35.990 | 18.977 | 1.00 93.69 | N |
| ATOM | 149 | CA | THR | A | 18 | 83.815 | 36.837 | 19.228 | 1.00 91.59 | C |
| ATOM | 150 | CB | THR | A | 18 | 85.127 | 36.039 | 19.082 | 1.00 89.62 | C |
| ATOM | 151 | OG1 | THR | A | 18 | 85.113 | 34.926 | 19.982 | 1.00 91.84 | O |
| ATOM | 152 | CG2 | THR | A | 18 | 86.335 | 36.912 | 19.388 | 1.00 88.68 | C |
| ATOM | 153 | C | THR | A | 18 | 83.873 | 38.056 | 18.303 | 1.00 91.59 | C |
| ATOM | 154 | O | THR | A | 18 | 84.147 | 39.165 | 18.756 | 1.00 93.09 | O |
| ATOM | 155 | N | PHE | A | 19 | 83.631 | 37.838 | 17.013 | 1.00 92.07 | N |
| ATOM | 156 | CA | PHE | A | 19 | 83.607 | 38.916 | 16.022 | 1.00 91.00 | C |
| ATOM | 157 | CB | PHE | A | 19 | 84.372 | 38.500 | 14.758 | 1.00 89.62 | C |
| ATOM | 158 | CG | PHE | A | 19 | 85.861 | 38.393 | 14.941 | 1.00 86.57 | C |
| ATOM | 159 | CD1 | PHE | A | 19 | 86.713 | 39.338 | 14.388 | 1.00 84.95 | C |
| ATOM | 160 | CE1 | PHE | A | 19 | 88.085 | 39.234 | 14.543 | 1.00 84.49 | C |
| ATOM | 161 | CZ | PHE | A | 19 | 88.621 | 38.176 | 15.253 | 1.00 84.63 | C |
| ATOM | 162 | CE2 | PHE | A | 19 | 87.785 | 37.223 | 15.802 | 1.00 85.95 | C |
| ATOM | 163 | CD2 | PHE | A | 19 | 86.413 | 37.332 | 15.643 | 1.00 86.68 | C |
| ATOM | 164 | C | PHE | A | 19 | 82.154 | 39.213 | 15.652 | 1.00 91.80 | C |
| ATOM | 165 | O | PHE | A | 19 | 81.265 | 38.417 | 15.966 | 1.00 92.30 | O |
| ATOM | 166 | N | PRO | A | 20 | 81.900 | 40.359 | 14.985 | 1.00 92.36 | N |
| ATOM | 167 | CA | PRO | A | 20 | 80.544 | 40.650 | 14.497 | 1.00 93.18 | C |
| ATOM | 168 | CB | PRO | A | 20 | 80.708 | 41.983 | 13.761 | 1.00 93.72 | C |
| ATOM | 169 | CG | PRO | A | 20 | 81.914 | 42.610 | 14.364 | 1.00 93.17 | C |
| ATOM | 170 | CD | PRO | A | 20 | 82.827 | 41.476 | 14.724 | 1.00 92.10 | C |
| ATOM | 171 | C | PRO | A | 20 | 80.048 | 39.579 | 13.529 | 1.00 92.47 | C |
| ATOM | 172 | O | PRO | A | 20 | 78.861 | 39.253 | 13.516 | 1.00 94.83 | O |
| ATOM | 173 | N | TYR | A | 21 | 80.973 | 39.050 | 12.731 | 1.00 89.94 | N |
| ATOM | 174 | CA | TYR | A | 21 | 80.694 | 37.973 | 11.791 | 1.00 87.48 | C |
| ATOM | 175 | CB | TYR | A | 21 | 81.251 | 38.310 | 10.402 | 1.00 86.87 | C |
| ATOM | 176 | CG | TYR | A | 21 | 82.767 | 38.336 | 10.269 | 1.00 85.77 | C |
| ATOM | 177 | CD1 | TYR | A | 21 | 83.520 | 39.408 | 10.744 | 1.00 85.72 | C |
| ATOM | 178 | CE1 | TYR | A | 21 | 84.900 | 39.438 | 10.599 | 1.00 86.03 | C |
| ATOM | 179 | CZ | TYR | A | 21 | 85.545 | 38.393 | 9.961 | 1.00 85.88 | C |
| ATOM | 180 | OH | TYR | A | 21 | 86.915 | 38.409 | 9.808 | 1.00 85.79 | O |
| ATOM | 181 | CE2 | TYR | A | 21 | 84.817 | 37.327 | 9.472 | 1.00 86.31 | C |
| ATOM | 182 | CD2 | TYR | A | 21 | 83.440 | 37.305 | 9.622 | 1.00 85.97 | C |
| ATOM | 183 | C | TYR | A | 21 | 81.291 | 36.676 | 12.326 | 1.00 85.29 | C |
| ATOM | 184 | O | TYR | A | 21 | 82.034 | 36.690 | 13.307 | 1.00 85.15 | O |
| ATOM | 185 | N | ASP | A | 22 | 80.952 | 35.556 | 11.695 | 1.00 84.23 | N |
| ATOM | 186 | CA | ASP | A | 22 | 81.479 | 34.259 | 12.112 | 1.00 81.39 | C |
| ATOM | 187 | CB | ASP | A | 22 | 80.601 | 33.118 | 11.595 | 1.00 79.88 | C |
| ATOM | 188 | CG | ASP | A | 22 | 80.934 | 31.781 | 12.235 | 1.00 80.48 | C |
| ATOM | 189 | OD1 | ASP | A | 22 | 81.957 | 31.686 | 12.950 | 1.00 80.95 | O |
| ATOM | 190 | OD2 | ASP | A | 22 | 80.167 | 30.817 | 12.025 | 1.00 77.89 | O |
| ATOM | 191 | C | ASP | A | 22 | 82.899 | 34.119 | 11.584 | 1.00 80.14 | C |
| ATOM | 192 | O | ASP | A | 22 | 83.105 | 33.974 | 10.377 | 1.00 83.56 | O |
| ATOM | 193 | N | ASN | A | 23 | 83.871 | 34.176 | 12.490 | 1.00 78.77 | N |
| ATOM | 194 | CA | ASN | A | 23 | 85.278 | 34.094 | 12.118 | 1.00 78.72 | C |
| ATOM | 195 | CB | ASN | A | 23 | 85.956 | 35.449 | 12.324 | 1.00 79.93 | C |
| ATOM | 196 | CG | ASN | A | 23 | 87.323 | 35.515 | 11.656 | 1.00 82.32 | C |
| ATOM | 197 | OD1 | ASN | A | 23 | 88.345 | 35.469 | 12.332 | 1.00 87.78 | O |
| ATOM | 198 | ND2 | ASN | A | 23 | 87.349 | 35.564 | 10.327 | 1.00 82.73 | N |
| ATOM | 199 | C | ASN | A | 23 | 86.021 | 33.010 | 12.901 | 1.00 77.85 | C |
| ATOM | 200 | O | ASN | A | 23 | 87.188 | 33.174 | 13.256 | 1.00 76.33 | O |
| ATOM | 201 | N | ARG | A | 24 | 85.346 | 31.896 | 13.167 | 1.00 77.43 | N |
| ATOM | 202 | CA | ARG | A | 24 | 85.985 | 30.779 | 13.851 | 1.00 78.84 | C |
| ATOM | 203 | CB | ARG | A | 24 | 84.961 | 29.708 | 14.215 | 1.00 82.46 | C |
| ATOM | 204 | CG | ARG | A | 24 | 83.948 | 30.142 | 15.261 | 1.00 87.00 | C |
| ATOM | 205 | CD | ARG | A | 24 | 82.841 | 29.113 | 15.404 | 1.00 90.70 | C |
| ATOM | 206 | NE | ARG | A | 24 | 81.997 | 29.063 | 14.213 | 1.00 94.41 | N |
| ATOM | 207 | CZ | ARG | A | 24 | 81.062 | 28.146 | 13.978 | 1.00100.00 | C |
| ATOM | 208 | NH1 | ARG | A | 24 | 80.832 | 27.168 | 14.850 | 1.00102.61 | N |
| ATOM | 209 | NH2 | ARG | A | 24 | 80.355 | 28.203 | 12.855 | 1.00102.93 | N |
| ATOM | 210 | C | ARG | A | 24 | 87.049 | 30.181 | 12.936 | 1.00 77.88 | C |
| ATOM | 211 | O | ARG | A | 24 | 86.931 | 30.270 | 11.715 | 1.00 79.51 | O |
| ATOM | 212 | N | PRO | A | 25 | 88.092 | 29.562 | 13.517 | 1.00 76.89 | N |
| ATOM | 213 | CA | PRO | A | 25 | 89.160 | 28.944 | 12.722 | 1.00 76.11 | C |
| ATOM | 214 | CB | PRO | A | 25 | 89.920 | 28.115 | 13.758 | 1.00 75.95 | C |
| ATOM | 215 | CG | PRO | A | 25 | 89.723 | 28.849 | 15.034 | 1.00 75.64 | C |
| ATOM | 216 | CD | PRO | A | 25 | 88.354 | 29.457 | 14.964 | 1.00 75.85 | C |
| ATOM | 217 | C | PRO | A | 25 | 88.683 | 28.040 | 11.575 | 1.00 76.14 | C |
| ATOM | 218 | O | PRO | A | 25 | 89.365 | 27.944 | 10.553 | 1.00 78.16 | O |
| ATOM | 219 | N | VAL | A | 26 | 87.534 | 27.386 | 11.750 | 1.00 74.03 | N |
| ATOM | 220 | CA | VAL | A | 26 | 87.001 | 26.463 | 10.742 | 1.00 74.16 | C |
| ATOM | 221 | CB | VAL | A | 26 | 86.097 | 25.379 | 11.375 | 1.00 75.02 | C |
| ATOM | 222 | CG1 | VAL | A | 26 | 86.876 | 24.575 | 12.405 | 1.00 76.87 | C |
| ATOM | 223 | CG2 | VAL | A | 26 | 84.841 | 25.980 | 11.996 | 1.00 75.97 | C |
| ATOM | 224 | C | VAL | A | 26 | 86.235 | 27.141 | 9.608 | 1.00 76.22 | C |
| ATOM | 225 | O | VAL | A | 26 | 85.985 | 26.517 | 8.578 | 1.00 80.23 | O |
| ATOM | 226 | N | VAL | A | 27 | 85.862 | 28.405 | 9.788 | 1.00 77.19 | N |
| ATOM | 227 | CA | VAL | A | 27 | 85.106 | 29.132 | 8.766 | 1.00 77.16 | C |
| ATOM | 228 | CB | VAL | A | 27 | 84.644 | 30.512 | 9.286 | 1.00 78.69 | C |
| ATOM | 229 | CG1 | VAL | A | 27 | 84.097 | 31.382 | 8.159 | 1.00 81.69 | C |
| ATOM | 230 | CG2 | VAL | A | 27 | 83.600 | 30.338 | 10.378 | 1.00 78.01 | C |
| ATOM | 231 | C | VAL | A | 27 | 85.940 | 29.305 | 7.493 | 1.00 75.90 | C |
| ATOM | 232 | O | VAL | A | 27 | 87.110 | 29.679 | 7.569 | 1.00 76.15 | O |
| ATOM | 233 | N | PRO | A | 28 | 85.340 | 29.026 | 6.321 | 1.00 76.46 | N |
| ATOM | 234 | CA | PRO | A | 28 | 86.056 | 29.172 | 5.053 | 1.00 77.60 | C |
| ATOM | 235 | CB | PRO | A | 28 | 85.147 | 28.453 | 4.056 | 1.00 76.68 | C |
| ATOM | 236 | CG | PRO | A | 28 | 83.790 | 28.562 | 4.637 | 1.00 77.52 | C |
| ATOM | 237 | CD | PRO | A | 28 | 83.994 | 28.461 | 6.119 | 1.00 78.17 | C |
| ATOM | 238 | C | PRO | A | 28 | 86.248 | 30.626 | 4.631 | 1.00 78.58 | C |
| ATOM | 239 | O | PRO | A | 28 | 85.462 | 31.496 | 5.015 | 1.00 81.59 | O |
| ATOM | 240 | N | LEU | A | 29 | 87.292 | 30.865 | 3.841 | 1.00 77.97 | N |
| ATOM | 241 | CA | LEU | A | 29 | 87.631 | 32.196 | 3.347 | 1.00 79.27 | C |
| ATOM | 242 | CB | LEU | A | 29 | 89.133 | 32.447 | 3.503 | 1.00 77.54 | C |
| ATOM | 243 | CG | LEU | A | 29 | 89.675 | 32.775 | 4.904 | 1.00 76.54 | C |
| ATOM | 244 | CD1 | LEU | A | 29 | 91.176 | 32.541 | 4.937 | 1.00 75.32 | C |
| ATOM | 245 | CD2 | LEU | A | 29 | 89.013 | 31.971 | 6.011 | 1.00 76.40 | C |
| ATOM | 246 | C | LEU | A | 29 | 87.228 | 32.296 | 1.880 | 1.00 82.98 | C |
| ATOM | 247 | O | LEU | A | 29 | 87.705 | 31.521 | 1.050 | 1.00 85.82 | O |
| ATOM | 248 | N | GLN | A | 30 | 86.360 | 33.256 | 1.563 | 1.00 85.08 | N |
| ATOM | 249 | CA | GLN | A | 30 | 85.833 | 33.409 | 0.207 | 1.00 84.07 | C |
| ATOM | 250 | CB | GLN | A | 30 | 84.644 | 32.461 | 0.010 | 1.00 83.65 | C |
| ATOM | 251 | CG | GLN | A | 30 | 83.505 | 32.673 | 1.004 | 1.00 85.40 | C |
| ATOM | 252 | CD | GLN | A | 30 | 82.633 | 31.442 | 1.217 | 1.00 85.90 | C |
| ATOM | 253 | OE1 | GLN | A | 30 | 83.118 | 30.311 | 1.215 | 1.00 83.42 | O |
| ATOM | 254 | NE2 | GLN | A | 30 | 81.341 | 31.665 | 1.432 | 1.00 86.12 | N |
| ATOM | 255 | C | GLN | A | 30 | 85.427 | 34.856 | −0.081 | 1.00 85.69 | C |
| ATOM | 256 | O | GLN | A | 30 | 84.653 | 35.455 | 0.666 | 1.00 86.01 | O |
| ATOM | 257 | N | TYR | A | 31 | 85.965 | 35.405 | −1.167 | 1.00 89.33 | N |
| ATOM | 258 | CA | TYR | A | 31 | 85.709 | 36.788 | −1.591 | 1.00 91.39 | C |
| ATOM | 259 | CB | TYR | A | 31 | 84.343 | 36.903 | −2.281 | 1.00 91.41 | C |
| ATOM | 260 | CG | TYR | A | 31 | 84.183 | 38.164 | −3.108 | 1.00 92.21 | C |
| ATOM | 261 | CD1 | TYR | A | 31 | 85.077 | 38.463 | −4.132 | 1.00 93.65 | C |
| ATOM | 262 | CE1 | TYR | A | 31 | 84.937 | 39.607 | −4.898 | 1.00 94.01 | C |
| ATOM | 263 | CZ | TYR | A | 31 | 83.888 | 40.466 | −4.653 | 1.00 94.14 | C |
| ATOM | 264 | OH | TYR | A | 31 | 83.753 | 41.599 | −5.418 | 1.00 94.67 | O |
| ATOM | 265 | CE2 | TYR | A | 31 | 82.982 | 40.191 | −3.647 | 1.00 94.39 | C |
| ATOM | 266 | CD2 | TYR | A | 31 | 83.131 | 39.043 | −2.884 | 1.00 93.28 | C |
| ATOM | 267 | C | TYR | A | 31 | 85.835 | 37.782 | −0.434 | 1.00 89.84 | C |
| ATOM | 268 | O | TYR | A | 31 | 84.935 | 38.585 | −0.175 | 1.00 91.00 | O |
| ATOM | 269 | N | MET | A | 32 | 86.975 | 37.710 | 0.248 | 1.00 87.23 | N |
| ATOM | 270 | CA | MET | A | 32 | 87.272 | 38.560 | 1.394 | 1.00 84.81 | C |
| ATOM | 271 | CB | MET | A | 32 | 87.742 | 37.702 | 2.566 | 1.00 83.80 | C |
| ATOM | 272 | CG | MET | A | 32 | 86.653 | 36.922 | 3.268 | 1.00 84.29 | C |
| ATOM | 273 | SD | MET | A | 32 | 87.372 | 35.892 | 4.557 | 1.00 88.43 | S |
| ATOM | 274 | CE | MET | A | 32 | 85.911 | 35.425 | 5.482 | 1.00 88.00 | C |
| ATOM | 275 | C | MET | A | 32 | 88.370 | 39.561 | 1.072 | 1.00 83.30 | C |
| ATOM | 276 | O | MET | A | 32 | 89.194 | 39.332 | 0.187 | 1.00 82.99 | O |
| ATOM | 277 | N | THR | A | 33 | 88.379 | 40.665 | 1.812 | 1.00 84.00 | N |
| ATOM | 278 | CA | THR | A | 33 | 89.443 | 41.655 | 1.708 | 1.00 85.10 | C |
| ATOM | 279 | CB | THR | A | 33 | 89.066 | 42.968 | 2.408 | 1.00 86.62 | C |
| ATOM | 280 | OG1 | THR | A | 33 | 88.682 | 42.694 | 3.761 | 1.00 85.39 | O |
| ATOM | 281 | CG2 | THR | A | 33 | 87.911 | 43.640 | 1.687 | 1.00 88.78 | C |
| ATOM | 282 | C | THR | A | 33 | 90.679 | 41.072 | 2.374 | 1.00 84.97 | C |
| ATOM | 283 | O | THR | A | 33 | 90.570 | 40.134 | 3.167 | 1.00 84.64 | O |
| ATOM | 284 | N | PHE | A | 34 | 91.847 | 41.626 | 2.068 | 1.00 84.71 | N |
| ATOM | 285 | CA | PHE | A | 34 | 93.096 | 41.070 | 2.584 | 1.00 84.09 | C |
| ATOM | 286 | CB | PHE | A | 34 | 94.300 | 41.925 | 2.201 | 1.00 82.95 | C |
| ATOM | 287 | CG | PHE | A | 34 | 95.605 | 41.323 | 2.623 | 1.00 81.81 | C |
| ATOM | 288 | CD1 | PHE | A | 34 | 96.087 | 40.193 | 1.984 | 1.00 81.66 | C |
| ATOM | 289 | CE1 | PHE | A | 34 | 97.283 | 39.618 | 2.369 | 1.00 81.94 | C |
| ATOM | 290 | CZ | PHE | A | 34 | 98.014 | 40.170 | 3.406 | 1.00 80.79 | C |
| ATOM | 291 | CE2 | PHE | A | 34 | 97.540 | 41.294 | 4.057 | 1.00 81.75 | C |
| ATOM | 292 | CD2 | PHE | A | 34 | 96.339 | 41.863 | 3.668 | 1.00 82.13 | C |
| ATOM | 293 | C | PHE | A | 34 | 93.078 | 40.861 | 4.094 | 1.00 85.55 | C |
| ATOM | 294 | O | PHE | A | 34 | 93.405 | 39.779 | 4.571 | 1.00 83.63 | O |
| ATOM | 295 | N | GLN | A | 35 | 92.682 | 41.892 | 4.835 | 1.00 89.59 | N |
| ATOM | 296 | CA | GLN | A | 35 | 92.652 | 41.828 | 6.298 | 1.00 92.31 | C |
| ATOM | 297 | CB | GLN | A | 35 | 92.176 | 43.159 | 6.884 | 1.00 97.58 | C |
| ATOM | 298 | CG | GLN | A | 35 | 92.242 | 43.228 | 8.401 | 1.00100.31 | C |
| ATOM | 299 | CD | GLN | A | 35 | 91.806 | 44.578 | 8.923 | 1.00104.73 | C |
| ATOM | 300 | OE1 | GLN | A | 35 | 90.700 | 45.025 | 8.633 | 1.00108.24 | O |
| ATOM | 301 | NE2 | GLN | A | 35 | 92.663 | 45.233 | 9.703 | 1.00108.47 | N |
| ATOM | 302 | C | GLN | A | 35 | 91.768 | 40.698 | 6.819 | 1.00 87.97 | C |
| ATOM | 303 | O | GLN | A | 35 | 92.081 | 40.082 | 7.840 | 1.00 88.49 | O |
| ATOM | 304 | N | GLU | A | 36 | 90.672 | 40.432 | 6.115 | 1.00 83.87 | N |
| ATOM | 305 | CA | GLU | A | 36 | 89.734 | 39.383 | 6.508 | 1.00 81.96 | C |
| ATOM | 306 | CB | GLU | A | 36 | 88.388 | 39.590 | 5.809 | 1.00 82.61 | C |
| ATOM | 307 | CG | GLU | A | 36 | 87.670 | 40.854 | 6.258 | 1.00 84.88 | C |
| ATOM | 308 | CD | GLU | A | 36 | 86.424 | 41.172 | 5.449 | 1.00 85.92 | C |
| ATOM | 309 | OE1 | GLU | A | 36 | 86.237 | 40.599 | 4.356 | 1.00 88.20 | O |
| ATOM | 310 | OE2 | GLU | A | 36 | 85.633 | 42.019 | 5.908 | 1.00 87.08 | O |
| ATOM | 311 | C | GLU | A | 36 | 90.264 | 37.969 | 6.244 | 1.00 78.74 | C |
| ATOM | 312 | O | GLU | A | 36 | 90.151 | 37.099 | 7.110 | 1.00 79.13 | O |
| ATOM | 313 | N | TRP | A | 37 | 90.850 | 37.739 | 5.068 | 1.00 74.66 | N |
| ATOM | 314 | CA | TRP | A | 37 | 91.333 | 36.397 | 4.715 | 1.00 71.51 | C |
| ATOM | 315 | CB | TRP | A | 37 | 91.230 | 36.128 | 3.192 | 1.00 70.80 | C |
| ATOM | 316 | CG | TRP | A | 37 | 92.341 | 36.670 | 2.332 | 1.00 67.87 | C |
| ATOM | 317 | CD1 | TRP | A | 37 | 92.305 | 37.789 | 1.559 | 1.00 68.80 | C |
| ATOM | 318 | NE1 | TRP | A | 37 | 93.506 | 37.958 | 0.912 | 1.00 69.34 | N |
| ATOM | 319 | CE2 | TRP | A | 37 | 94.342 | 36.932 | 1.257 | 1.00 66.64 | C |
| ATOM | 320 | CD2 | TRP | A | 37 | 93.638 | 36.096 | 2.143 | 1.00 65.97 | C |
| ATOM | 321 | CE3 | TRP | A | 37 | 94.277 | 34.962 | 2.649 | 1.00 67.44 | C |
| ATOM | 322 | CZ3 | TRP | A | 37 | 95.578 | 34.702 | 2.256 | 1.00 68.15 | C |
| ATOM | 323 | CH2 | TRP | A | 37 | 96.251 | 35.555 | 1.371 | 1.00 68.14 | C |
| ATOM | 324 | CZ2 | TRP | A | 37 | 95.650 | 36.671 | 0.862 | 1.00 67.60 | C |
| ATOM | 325 | C | TRP | A | 37 | 92.741 | 36.106 | 5.246 | 1.00 69.80 | C |
| ATOM | 326 | O | TRP | A | 37 | 93.022 | 34.984 | 5.657 | 1.00 68.47 | O |
| ATOM | 327 | N | TRP | A | 38 | 93.619 | 37.105 | 5.240 | 1.00 72.07 | N |
| ATOM | 328 | CA | TRP | A | 38 | 95.006 | 36.918 | 5.697 | 1.00 75.67 | C |
| ATOM | 329 | CB | TRP | A | 38 | 95.836 | 38.173 | 5.417 | 1.00 77.42 | C |
| ATOM | 330 | CG | TRP | A | 38 | 97.241 | 38.121 | 5.933 | 1.00 78.07 | C |
| ATOM | 331 | CD1 | TRP | A | 38 | 97.750 | 38.830 | 6.979 | 1.00 79.20 | C |
| ATOM | 332 | NE1 | TRP | A | 38 | 99.078 | 38.532 | 7.153 | 1.00 79.67 | N |
| ATOM | 333 | CE2 | TRP | A | 38 | 99.453 | 37.612 | 6.211 | 1.00 78.00 | C |
| ATOM | 334 | CD2 | TRP | A | 38 | 98.318 | 37.326 | 5.426 | 1.00 78.69 | C |
| ATOM | 335 | CE3 | TRP | A | 38 | 98.435 | 36.401 | 4.383 | 1.00 81.00 | C |
| ATOM | 336 | CZ3 | TRP | A | 38 | 99.666 | 35.795 | 4.164 | 1.00 80.23 | C |
| ATOM | 337 | CH2 | TRP | A | 38 | 100.773 | 36.102 | 4.966 | 1.00 78.28 | C |
| ATOM | 338 | CZ2 | TRP | A | 38 | 100.686 | 37.002 | 5.992 | 1.00 77.56 | C |
| ATOM | 339 | C | TRP | A | 38 | 95.084 | 36.554 | 7.184 | 1.00 76.24 | C |
| ATOM | 340 | O | TRP | A | 38 | 94.643 | 37.316 | 8.046 | 1.00 72.52 | O |
| ATOM | 341 | N | PHE | A | 39 | 95.648 | 35.378 | 7.461 | 1.00 77.18 | N |
| ATOM | 342 | CA | PHE | A | 39 | 95.744 | 34.828 | 8.819 | 1.00 76.64 | C |
| ATOM | 343 | CB | PHE | A | 39 | 96.652 | 35.693 | 9.705 | 1.00 77.48 | C |
| ATOM | 344 | CG | PHE | A | 39 | 98.059 | 35.181 | 9.814 | 1.00 78.53 | C |
| ATOM | 345 | CD1 | PHE | A | 39 | 98.464 | 34.469 | 10.936 | 1.00 79.16 | C |
| ATOM | 346 | CE1 | PHE | A | 39 | 99.762 | 33.996 | 11.045 | 1.00 79.01 | C |
| ATOM | 347 | CZ | PHE | A | 39 | 100.670 | 34.227 | 10.025 | 1.00 79.14 | C |
| ATOM | 348 | CE2 | PHE | A | 39 | 100.278 | 34.935 | 8.902 | 1.00 78.45 | C |
| ATOM | 349 | CD2 | PHE | A | 39 | 98.978 | 35.407 | 8.801 | 1.00 78.90 | C |
| ATOM | 350 | C | PHE | A | 39 | 94.382 | 34.604 | 9.482 | 1.00 76.52 | C |
| ATOM | 351 | O | PHE | A | 39 | 94.301 | 34.448 | 10.702 | 1.00 81.02 | O |
| ATOM | 352 | N | HIS | A | 40 | 93.327 | 34.553 | 8.669 | 1.00 74.90 | N |
| ATOM | 353 | CA | HIS | A | 40 | 91.956 | 34.369 | 9.146 | 1.00 74.06 | C |
| ATOM | 354 | CB | HIS | A | 40 | 91.817 | 33.016 | 9.857 | 1.00 69.89 | C |
| ATOM | 355 | CG | HIS | A | 40 | 90.460 | 32.401 | 9.730 | 1.00 66.79 | C |
| ATOM | 356 | ND1 | HIS | A | 40 | 89.299 | 33.085 | 10.019 | 1.00 66.58 | N |
| ATOM | 357 | CE1 | HIS | A | 40 | 88.262 | 32.290 | 9.821 | 1.00 65.80 | C |
| ATOM | 358 | NE2 | HIS | A | 40 | 88.709 | 31.111 | 9.428 | 1.00 65.20 | N |
| ATOM | 359 | CD2 | HIS | A | 40 | 90.081 | 31.153 | 9.365 | 1.00 65.32 | C |
| ATOM | 360 | C | HIS | A | 40 | 91.552 | 35.513 | 10.078 | 1.00 78.21 | C |
| ATOM | 361 | O | HIS | A | 40 | 90.708 | 35.339 | 10.959 | 1.00 79.42 | O |
| ATOM | 362 | N | ASN | A | 41 | 92.157 | 36.683 | 9.867 | 1.00 82.14 | N |
| ATOM | 363 | CA | ASN | A | 41 | 91.928 | 37.861 | 10.703 | 1.00 82.77 | C |
| ATOM | 364 | CB | ASN | A | 41 | 90.579 | 38.500 | 10.345 | 1.00 80.83 | C |
| ATOM | 365 | CG | ASN | A | 41 | 90.473 | 39.946 | 10.794 | 1.00 79.26 | C |
| ATOM | 366 | OD1 | ASN | A | 41 | 91.480 | 40.641 | 10.938 | 1.00 78.80 | O |
| ATOM | 367 | ND2 | ASN | A | 41 | 89.246 | 40.413 | 10.996 | 1.00 79.09 | N |
| ATOM | 368 | C | ASN | A | 41 | 92.010 | 37.528 | 12.198 | 1.00 84.66 | C |
| ATOM | 369 | O | ASN | A | 41 | 91.164 | 37.952 | 12.989 | 1.00 88.85 | O |
| ATOM | 370 | N | HIS | A | 42 | 93.030 | 36.753 | 12.568 | 1.00 84.35 | N |
| ATOM | 371 | CA | HIS | A | 42 | 93.252 | 36.351 | 13.959 | 1.00 84.97 | C |
| ATOM | 372 | CB | HIS | A | 42 | 93.240 | 34.823 | 14.093 | 1.00 83.97 | C |
| ATOM | 373 | CG | HIS | A | 42 | 91.890 | 34.204 | 13.916 | 1.00 83.92 | C |
| ATOM | 374 | ND1 | HIS | A | 42 | 91.717 | 32.943 | 13.387 | 1.00 86.21 | N |
| ATOM | 375 | CE1 | HIS | A | 42 | 90.428 | 32.654 | 13.355 | 1.00 84.60 | C |
| ATOM | 376 | NE2 | HIS | A | 42 | 89.759 | 33.686 | 13.838 | 1.00 82.81 | N |
| ATOM | 377 | CD2 | HIS | A | 42 | 90.650 | 34.669 | 14.195 | 1.00 82.87 | C |
| ATOM | 378 | C | HIS | A | 42 | 94.569 | 36.885 | 14.530 | 1.00 86.81 | C |
| ATOM | 379 | O | HIS | A | 42 | 95.054 | 36.371 | 15.539 | 1.00 88.94 | O |
| ATOM | 380 | N | LEU | A | 43 | 95.141 | 37.915 | 13.906 | 1.00 87.62 | N |
| ATOM | 381 | CA | LEU | A | 43 | 96.409 | 38.480 | 14.379 | 1.00 87.31 | C |
| ATOM | 382 | CB | LEU | A | 43 | 96.950 | 39.518 | 13.390 | 1.00 85.45 | C |
| ATOM | 383 | CG | LEU | A | 43 | 97.366 | 38.975 | 12.018 | 1.00 84.56 | C |
| ATOM | 384 | CD1 | LEU | A | 43 | 97.778 | 40.109 | 11.092 | 1.00 84.45 | C |
| ATOM | 385 | CD2 | LEU | A | 43 | 98.489 | 37.957 | 12.145 | 1.00 84.05 | C |
| ATOM | 386 | C | LEU | A | 43 | 96.285 | 39.086 | 15.781 | 1.00 90.04 | C |
| ATOM | 387 | O | LEU | A | 43 | 97.224 | 39.010 | 16.576 | 1.00 91.88 | O |
| ATOM | 388 | N | ASP | A | 44 | 95.122 | 39.658 | 16.089 | 1.00 90.94 | N |
| ATOM | 389 | CA | ASP | A | 44 | 94.872 | 40.239 | 17.413 | 1.00 90.81 | C |
| ATOM | 390 | CB | ASP | A | 44 | 93.751 | 41.285 | 17.342 | 1.00 91.78 | C |
| ATOM | 391 | CG | ASP | A | 44 | 94.126 | 42.494 | 16.497 | 1.00 93.05 | C |
| ATOM | 392 | OD1 | ASP | A | 44 | 95.329 | 42.704 | 16.227 | 1.00 93.42 | O |
| ATOM | 393 | OD2 | ASP | A | 44 | 93.210 | 43.246 | 16.105 | 1.00 95.74 | O |
| ATOM | 394 | C | ASP | A | 44 | 94.545 | 39.189 | 18.481 | 1.00 88.30 | C |
| ATOM | 395 | O | ASP | A | 44 | 94.294 | 39.538 | 19.634 | 1.00 92.18 | O |
| ATOM | 396 | N | TYR | A | 45 | 94.553 | 37.912 | 18.102 | 1.00 86.07 | N |
| ATOM | 397 | CA | TYR | A | 45 | 94.333 | 36.810 | 19.038 | 1.00 86.13 | C |
| ATOM | 398 | CB | TYR | A | 45 | 93.001 | 36.116 | 18.743 | 1.00 83.98 | C |
| ATOM | 399 | CG | TYR | A | 45 | 91.804 | 37.000 | 18.991 | 1.00 83.67 | C |
| ATOM | 400 | CD1 | TYR | A | 45 | 91.391 | 37.924 | 18.038 | 1.00 83.99 | C |
| ATOM | 401 | CE1 | TYR | A | 45 | 90.297 | 38.743 | 18.261 | 1.00 83.73 | C |
| ATOM | 402 | CZ | TYR | A | 45 | 89.601 | 38.644 | 19.450 | 1.00 83.17 | C |
| ATOM | 403 | OH | TYR | A | 45 | 88.513 | 39.457 | 19.672 | 1.00 82.14 | O |
| ATOM | 404 | CE2 | TYR | A | 45 | 89.992 | 37.733 | 20.414 | 1.00 83.17 | C |
| ATOM | 405 | CD2 | TYR | A | 45 | 91.089 | 36.920 | 20.182 | 1.00 82.66 | C |
| ATOM | 406 | C | TYR | A | 45 | 95.491 | 35.818 | 18.937 | 1.00 86.55 | C |
| ATOM | 407 | O | TYR | A | 45 | 95.294 | 34.660 | 18.566 | 1.00 86.50 | O |
| ATOM | 408 | N | PRO | A | 46 | 96.709 | 36.270 | 19.281 | 1.00 88.41 | N |
| ATOM | 409 | CA | PRO | A | 46 | 97.863 | 35.396 | 19.162 | 1.00 88.47 | C |
| ATOM | 410 | CB | PRO | A | 46 | 99.037 | 36.370 | 19.243 | 1.00 88.57 | C |
| ATOM | 411 | CG | PRO | A | 46 | 98.546 | 37.474 | 20.115 | 1.00 88.79 | C |
| ATOM | 412 | CD | PRO | A | 46 | 97.045 | 37.515 | 19.997 | 1.00 89.40 | C |
| ATOM | 413 | C | PRO | A | 46 | 97.936 | 34.393 | 20.304 | 1.00 89.24 | C |
| ATOM | 414 | O | PRO | A | 46 | 97.334 | 34.616 | 21.353 | 1.00 91.06 | O |
| ATOM | 415 | N | PRO | A | 47 | 98.682 | 33.295 | 20.108 | 1.00 89.74 | N |
| ATOM | 416 | CA | PRO | A | 47 | 98.854 | 32.294 | 21.153 | 1.00 89.39 | C |
| ATOM | 417 | CB | PRO | A | 47 | 99.518 | 31.142 | 20.405 | 1.00 89.86 | C |
| ATOM | 418 | CG | PRO | A | 47 | 100.352 | 31.832 | 19.379 | 1.00 89.43 | C |
| ATOM | 419 | CD | PRO | A | 47 | 99.525 | 33.004 | 18.932 | 1.00 89.34 | C |
| ATOM | 420 | C | PRO | A | 47 | 99.786 | 32.818 | 22.235 | 1.00 90.42 | C |
| ATOM | 421 | O | PRO | A | 47 | 100.293 | 33.936 | 22.120 | 1.00 90.12 | O |
| ATOM | 422 | N | HIS | A | 48 | 100.019 | 32.023 | 23.273 | 1.00 93.02 | N |
| ATOM | 423 | CA | HIS | A | 48 | 100.965 | 32.424 | 24.307 | 1.00 94.38 | C |
| ATOM | 424 | CB | HIS | A | 48 | 100.828 | 31.565 | 25.564 | 1.00 95.13 | C |
| ATOM | 425 | CG | HIS | A | 48 | 99.486 | 31.671 | 26.221 | 1.00 95.66 | C |
| ATOM | 426 | ND1 | HIS | A | 48 | 98.803 | 32.864 | 26.331 | 1.00 97.19 | N |
| ATOM | 427 | CE1 | HIS | A | 48 | 97.659 | 32.658 | 26.959 | 1.00 97.52 | C |
| ATOM | 428 | NE2 | HIS | A | 48 | 97.581 | 31.377 | 27.272 | 1.00 97.13 | N |
| ATOM | 429 | CD2 | HIS | A | 48 | 98.713 | 30.738 | 26.826 | 1.00 95.64 | C |
| ATOM | 430 | C | HIS | A | 48 | 102.376 | 32.351 | 23.718 | 1.00 96.28 | C |
| ATOM | 431 | O | HIS | A | 48 | 102.644 | 31.505 | 22.862 | 1.00 96.29 | O |
| ATOM | 432 | N | PRO | A | 49 | 103.281 | 33.240 | 24.166 | 1.00 97.64 | N |
| ATOM | 433 | CA | PRO | A | 49 | 104.614 | 33.331 | 23.557 | 1.00 98.49 | C |
| ATOM | 434 | CB | PRO | A | 49 | 105.323 | 34.391 | 24.414 | 1.00 99.83 | C |
| ATOM | 435 | CG | PRO | A | 49 | 104.229 | 35.170 | 25.054 | 1.00 98.41 | C |
| ATOM | 436 | CD | PRO | A | 49 | 103.136 | 34.177 | 25.295 | 1.00 97.68 | C |
| ATOM | 437 | C | PRO | A | 49 | 105.431 | 32.036 | 23.539 | 1.00 97.48 | C |
| ATOM | 438 | O | PRO | A | 49 | 106.244 | 31.849 | 22.636 | 1.00 96.07 | O |
| ATOM | 439 | N | GLY | A | 50 | 105.216 | 31.159 | 24.519 | 1.00 97.60 | N |
| ATOM | 440 | CA | GLY | A | 50 | 105.978 | 29.912 | 24.628 | 1.00 96.66 | C |
| ATOM | 441 | C | GLY | A | 50 | 105.441 | 28.715 | 23.858 | 1.00 94.24 | C |
| ATOM | 442 | O | GLY | A | 50 | 106.114 | 27.688 | 23.777 | 1.00 96.57 | O |
| ATOM | 443 | N | ASP | A | 51 | 104.238 | 28.835 | 23.296 | 1.00 91.17 | N |
| ATOM | 444 | CA | ASP | A | 51 | 103.607 | 27.733 | 22.562 | 1.00 88.43 | C |
| ATOM | 445 | CB | ASP | A | 51 | 102.084 | 27.821 | 22.685 | 1.00 88.07 | C |
| ATOM | 446 | CG | ASP | A | 51 | 101.602 | 27.681 | 24.121 | 1.00 88.98 | C |
| ATOM | 447 | OD1 | ASP | A | 51 | 102.338 | 27.107 | 24.952 | 1.00 87.99 | O |
| ATOM | 448 | OD2 | ASP | A | 51 | 100.477 | 28.141 | 24.416 | 1.00 88.52 | O |
| ATOM | 449 | C | ASP | A | 51 | 104.007 | 27.717 | 21.081 | 1.00 87.45 | C |
| ATOM | 450 | O | ASP | A | 51 | 103.668 | 28.632 | 20.328 | 1.00 82.48 | O |
| ATOM | 451 | N | PHE | A | 52 | 104.729 | 26.669 | 20.681 | 1.00 86.67 | N |
| ATOM | 452 | CA | PHE | A | 52 | 105.177 | 26.489 | 19.299 | 1.00 86.78 | C |
| ATOM | 453 | CB | PHE | A | 52 | 106.681 | 26.747 | 19.178 | 1.00 88.12 | C |
| ATOM | 454 | CG | PHE | A | 52 | 107.071 | 28.187 | 19.330 | 1.00 89.61 | C |
| ATOM | 455 | CD1 | PHE | A | 52 | 107.381 | 28.709 | 20.577 | 1.00 90.55 | C |
| ATOM | 456 | CE1 | PHE | A | 52 | 107.750 | 30.034 | 20.716 | 1.00 91.55 | C |
| ATOM | 457 | CZ | PHE | A | 52 | 107.815 | 30.856 | 19.604 | 1.00 92.08 | C |
| ATOM | 458 | CE2 | PHE | A | 52 | 107.513 | 30.347 | 18.354 | 1.00 90.65 | C |
| ATOM | 459 | CD2 | PHE | A | 52 | 107.148 | 29.019 | 18.222 | 1.00 90.14 | C |
| ATOM | 460 | C | PHE | A | 52 | 104.900 | 25.073 | 18.806 | 1.00 86.43 | C |
| ATOM | 461 | O | PHE | A | 52 | 104.956 | 24.116 | 19.580 | 1.00 89.33 | O |
| ATOM | 462 | N | PHE | A | 53 | 104.608 | 24.955 | 17.514 | 1.00 84.53 | N |
| ATOM | 463 | CA | PHE | A | 53 | 104.450 | 23.658 | 16.861 | 1.00 82.72 | C |
| ATOM | 464 | CB | PHE | A | 53 | 103.408 | 23.744 | 15.744 | 1.00 79.11 | C |
| ATOM | 465 | CG | PHE | A | 53 | 103.052 | 22.415 | 15.135 | 1.00 74.50 | C |
| ATOM | 466 | CD1 | PHE | A | 53 | 101.884 | 21.762 | 15.500 | 1.00 72.03 | C |
| ATOM | 467 | CE1 | PHE | A | 53 | 101.551 | 20.539 | 14.944 | 1.00 70.56 | C |
| ATOM | 468 | CZ | PHE | A | 53 | 102.389 | 19.954 | 14.012 | 1.00 71.23 | C |
| ATOM | 469 | CE2 | PHE | A | 53 | 103.556 | 20.594 | 13.636 | 1.00 71.89 | C |
| ATOM | 470 | CD2 | PHE | A | 53 | 103.883 | 21.817 | 14.194 | 1.00 73.25 | C |
| ATOM | 471 | C | PHE | A | 53 | 105.820 | 23.292 | 16.290 | 1.00 83.28 | C |
| ATOM | 472 | O | PHE | A | 53 | 106.380 | 24.038 | 15.488 | 1.00 82.27 | O |
| ATOM | 473 | N | ASP | A | 54 | 106.356 | 22.147 | 16.700 | 1.00 84.85 | N |
| ATOM | 474 | CA | ASP | A | 54 | 107.699 | 21.737 | 16.288 | 1.00 85.30 | C |
| ATOM | 475 | CB | ASP | A | 54 | 108.404 | 21.003 | 17.436 | 1.00 87.15 | C |
| ATOM | 476 | CG | ASP | A | 54 | 108.726 | 21.918 | 18.607 | 1.00 88.60 | C |
| ATOM | 477 | OD1 | ASP | A | 54 | 109.024 | 23.113 | 18.382 | 1.00 87.90 | O |
| ATOM | 478 | OD2 | ASP | A | 54 | 108.698 | 21.435 | 19.758 | 1.00 93.39 | O |
| ATOM | 479 | C | ASP | A | 54 | 107.708 | 20.870 | 15.029 | 1.00 83.18 | C |
| ATOM | 480 | O | ASP | A | 54 | 106.981 | 19.880 | 14.941 | 1.00 83.58 | O |
| ATOM | 481 | N | PHE | A | 55 | 108.541 | 21.258 | 14.065 | 1.00 80.53 | N |
| ATOM | 482 | CA | PHE | A | 55 | 108.739 | 20.499 | 12.832 | 1.00 78.93 | C |
| ATOM | 483 | CB | PHE | A | 55 | 108.573 | 21.403 | 11.604 | 1.00 79.62 | C |
| ATOM | 484 | CG | PHE | A | 55 | 107.140 | 21.707 | 11.263 | 1.00 78.94 | C |
| ATOM | 485 | CD1 | PHE | A | 55 | 106.328 | 20.729 | 10.701 | 1.00 78.40 | C |
| ATOM | 486 | CE1 | PHE | A | 55 | 105.009 | 20.997 | 10.388 | 1.00 76.98 | C |
| ATOM | 487 | CZ | PHE | A | 55 | 104.486 | 22.254 | 10.624 | 1.00 77.19 | C |
| ATOM | 488 | CE2 | PHE | A | 55 | 105.283 | 23.242 | 11.175 | 1.00 77.28 | C |
| ATOM | 489 | CD2 | PHE | A | 55 | 106.603 | 22.967 | 11.492 | 1.00 77.85 | C |
| ATOM | 490 | C | PHE | A | 55 | 110.137 | 19.882 | 12.844 | 1.00 77.06 | C |
| ATOM | 491 | O | PHE | A | 55 | 111.126 | 20.594 | 12.670 | 1.00 76.91 | O |
| ATOM | 492 | N | PRO | A | 56 | 110.228 | 18.556 | 13.051 | 1.00 75.75 | N |
| ATOM | 493 | CA | PRO | A | 56 | 111.536 | 17.916 | 13.097 | 1.00 77.64 | C |
| ATOM | 494 | CB | PRO | A | 56 | 111.263 | 16.636 | 13.886 | 1.00 77.83 | C |
| ATOM | 495 | CG | PRO | A | 56 | 109.837 | 16.308 | 13.603 | 1.00 76.68 | C |
| ATOM | 496 | CD | PRO | A | 56 | 109.136 | 17.579 | 13.209 | 1.00 76.19 | C |
| ATOM | 497 | C | PRO | A | 56 | 112.080 | 17.594 | 11.707 | 1.00 80.24 | C |
| ATOM | 498 | O | PRO | A | 56 | 111.481 | 16.802 | 10.979 | 1.00 83.61 | O |
| ATOM | 499 | N | ALA | A | 57 | 113.214 | 18.200 | 11.359 | 1.00 80.81 | N |
| ATOM | 500 | CA | ALA | A | 57 | 113.845 | 17.992 | 10.058 | 1.00 79.94 | C |
| ATOM | 501 | CB | ALA | A | 57 | 115.173 | 18.730 | 9.993 | 1.00 79.03 | C |
| ATOM | 502 | C | ALA | A | 57 | 114.053 | 16.510 | 9.766 | 1.00 81.02 | C |
| ATOM | 503 | O | ALA | A | 57 | 114.479 | 15.753 | 10.636 | 1.00 82.40 | O |
| ATOM | 504 | N | GLY | A | 58 | 113.730 | 16.103 | 8.541 | 1.00 83.86 | N |
| ATOM | 505 | CA | GLY | A | 58 | 113.888 | 14.716 | 8.109 | 1.00 84.69 | C |
| ATOM | 506 | C | GLY | A | 58 | 112.937 | 13.736 | 8.771 | 1.00 84.18 | C |
| ATOM | 507 | O | GLY | A | 58 | 113.135 | 12.524 | 8.674 | 1.00 85.98 | O |
| ATOM | 508 | N | LYS | A | 59 | 111.899 | 14.255 | 9.427 | 1.00 82.88 | N |
| ATOM | 509 | CA | LYS | A | 59 | 110.931 | 13.430 | 10.147 | 1.00 83.70 | C |
| ATOM | 510 | CB | LYS | A | 59 | 111.279 | 13.390 | 11.639 | 1.00 87.21 | C |
| ATOM | 511 | CG | LYS | A | 59 | 112.457 | 12.494 | 11.985 | 1.00 90.93 | C |
| ATOM | 512 | CD | LYS | A | 59 | 112.880 | 12.671 | 13.433 | 1.00 95.35 | C |
| ATOM | 513 | CE | LYS | A | 59 | 113.889 | 11.613 | 13.850 | 1.00100.46 | C |
| ATOM | 514 | NZ | LYS | A | 59 | 114.320 | 11.783 | 15.267 | 1.00103.42 | N |
| ATOM | 515 | C | LYS | A | 59 | 109.507 | 13.947 | 9.962 | 1.00 81.27 | C |
| ATOM | 516 | O | LYS | A | 59 | 109.292 | 15.090 | 9.554 | 1.00 79.60 | O |
| ATOM | 517 | N | ALA | A | 60 | 108.540 | 13.091 | 10.271 | 1.00 79.91 | N |
| ATOM | 518 | CA | ALA | A | 60 | 107.128 | 13.437 | 10.156 | 1.00 79.46 | C |
| ATOM | 519 | CB | ALA | A | 60 | 106.282 | 12.173 | 10.104 | 1.00 81.71 | C |
| ATOM | 520 | C | ALA | A | 60 | 106.678 | 14.321 | 11.315 | 1.00 77.96 | C |
| ATOM | 521 | O | ALA | A | 60 | 107.222 | 14.246 | 12.418 | 1.00 81.06 | O |
| ATOM | 522 | N | ALA | A | 61 | 105.681 | 15.158 | 11.049 | 1.00 74.15 | N |
| ATOM | 523 | CA | ALA | A | 61 | 105.096 | 16.029 | 12.056 | 1.00 72.68 | C |
| ATOM | 524 | CB | ALA | A | 61 | 105.499 | 17.470 | 11.806 | 1.00 71.45 | C |
| ATOM | 525 | C | ALA | A | 61 | 103.584 | 15.879 | 11.991 | 1.00 73.76 | C |
| ATOM | 526 | O | ALA | A | 61 | 102.958 | 16.323 | 11.031 | 1.00 76.05 | O |
| ATOM | 527 | N | THR | A | 62 | 102.998 | 15.252 | 13.007 | 1.00 74.78 | N |
| ATOM | 528 | CA | THR | A | 62 | 101.556 | 15.021 | 13.029 | 1.00 74.22 | C |
| ATOM | 529 | CB | THR | A | 62 | 101.182 | 13.889 | 13.999 | 1.00 74.44 | C |
| ATOM | 530 | OG1 | THR | A | 62 | 101.914 | 12.706 | 13.655 | 1.00 77.29 | O |
| ATOM | 531 | CG2 | THR | A | 62 | 99.690 | 13.590 | 13.929 | 1.00 74.77 | C |
| ATOM | 532 | C | THR | A | 62 | 100.789 | 16.284 | 13.415 | 1.00 73.32 | C |
| ATOM | 533 | O | THR | A | 62 | 101.075 | 16.906 | 14.437 | 1.00 73.85 | O |
| ATOM | 534 | N | ALA | A | 63 | 99.818 | 16.648 | 12.581 | 1.00 71.65 | N |
| ATOM | 535 | CA | ALA | A | 63 | 98.959 | 17.799 | 12.812 | 1.00 71.44 | C |
| ATOM | 536 | CB | ALA | A | 63 | 99.055 | 18.763 | 11.646 | 1.00 72.36 | C |
| ATOM | 537 | C | ALA | A | 63 | 97.529 | 17.313 | 12.972 | 1.00 72.07 | C |
| ATOM | 538 | O | ALA | A | 63 | 97.175 | 16.254 | 12.454 | 1.00 72.86 | O |
| ATOM | 539 | N | GLU | A | 64 | 96.713 | 18.080 | 13.691 | 1.00 73.76 | N |
| ATOM | 540 | CA | GLU | A | 64 | 95.314 | 17.714 | 13.916 | 1.00 74.96 | C |
| ATOM | 541 | CB | GLU | A | 64 | 94.972 | 17.740 | 15.404 | 1.00 76.45 | C |
| ATOM | 542 | CG | GLU | A | 64 | 95.780 | 16.747 | 16.224 | 1.00 79.88 | C |
| ATOM | 543 | CD | GLU | A | 64 | 95.271 | 16.604 | 17.645 | 1.00 83.26 | C |
| ATOM | 544 | OE1 | GLU | A | 64 | 94.084 | 16.260 | 17.822 | 1.00 84.32 | O |
| ATOM | 545 | OE2 | GLU | A | 64 | 96.063 | 16.818 | 18.587 | 1.00 85.29 | O |
| ATOM | 546 | C | GLU | A | 64 | 94.378 | 18.630 | 13.139 | 1.00 74.74 | C |
| ATOM | 547 | O | GLU | A | 64 | 94.387 | 19.850 | 13.323 | 1.00 74.92 | O |
| ATOM | 548 | N | LEU | A | 65 | 93.577 | 18.024 | 12.268 | 1.00 73.51 | N |
| ATOM | 549 | CA | LEU | A | 65 | 92.629 | 18.744 | 11.431 | 1.00 71.37 | C |
| ATOM | 550 | CB | LEU | A | 65 | 92.931 | 18.473 | 9.956 | 1.00 71.01 | C |
| ATOM | 551 | CG | LEU | A | 65 | 94.395 | 18.637 | 9.526 | 1.00 71.64 | C |
| ATOM | 552 | CD1 | LEU | A | 65 | 94.593 | 18.148 | 8.102 | 1.00 73.33 | C |
| ATOM | 553 | CD2 | LEU | A | 65 | 94.859 | 20.078 | 9.664 | 1.00 71.74 | C |
| ATOM | 554 | C | LEU | A | 65 | 91.232 | 18.257 | 11.782 | 1.00 69.60 | C |
| ATOM | 555 | O | LEU | A | 65 | 90.983 | 17.052 | 11.773 | 1.00 68.72 | O |
| ATOM | 556 | N | ALA | A | 66 | 90.328 | 19.181 | 12.104 | 1.00 69.80 | N |
| ATOM | 557 | CA | ALA | A | 66 | 88.965 | 18.808 | 12.481 | 1.00 71.86 | C |
| ATOM | 558 | CB | ALA | A | 66 | 88.952 | 18.233 | 13.893 | 1.00 72.50 | C |
| ATOM | 559 | C | ALA | A | 66 | 87.972 | 19.962 | 12.377 | 1.00 75.05 | C |
| ATOM | 560 | O | ALA | A | 66 | 88.357 | 21.130 | 12.313 | 1.00 76.83 | O |
| ATOM | 561 | N | CYS | A | 67 | 86.689 | 19.611 | 12.367 | 1.00 79.66 | N |
| ATOM | 562 | CA | CYS | A | 67 | 85.606 | 20.587 | 12.285 | 1.00 84.59 | C |
| ATOM | 563 | CB | CYS | A | 67 | 84.324 | 19.913 | 11.791 | 1.00 89.06 | C |
| ATOM | 564 | SG | CYS | A | 67 | 83.623 | 18.703 | 12.944 | 1.00 99.46 | S |
| ATOM | 565 | C | CYS | A | 67 | 85.349 | 21.253 | 13.632 | 1.00 85.01 | C |
| ATOM | 566 | O | CYS | A | 67 | 84.738 | 22.321 | 13.691 | 1.00 85.07 | O |
| ATOM | 567 | N | ASN | A | 68 | 85.799 | 20.609 | 14.707 | 1.00 89.37 | N |
| ATOM | 568 | CA | ASN | A | 68 | 85.644 | 21.151 | 16.053 | 1.00 93.19 | C |
| ATOM | 569 | CB | ASN | A | 68 | 84.401 | 20.587 | 16.750 | 1.00 98.47 | C |
| ATOM | 570 | CG | ASN | A | 68 | 83.562 | 21.678 | 17.382 | 1.00106.32 | C |
| ATOM | 571 | OD1 | ASN | A | 68 | 84.040 | 22.438 | 18.231 | 1.00111.29 | O |
| ATOM | 572 | ND2 | ASN | A | 68 | 82.315 | 21.785 | 16.947 | 1.00110.61 | N |
| ATOM | 573 | C | ASN | A | 68 | 86.866 | 20.854 | 16.898 | 1.00 89.54 | C |
| ATOM | 574 | O | ASN | A | 68 | 87.495 | 19.803 | 16.752 | 1.00 91.68 | O |
| ATOM | 575 | N | LYS | A | 69 | 87.186 | 21.778 | 17.795 | 1.00 86.26 | N |
| ATOM | 576 | CA | LYS | A | 69 | 88.306 | 21.598 | 18.706 | 1.00 84.37 | C |
| ATOM | 577 | CB | LYS | A | 69 | 88.599 | 22.902 | 19.446 | 1.00 84.78 | C |
| ATOM | 578 | CG | LYS | A | 69 | 89.835 | 22.845 | 20.325 | 1.00 85.39 | C |
| ATOM | 579 | CD | LYS | A | 69 | 90.389 | 24.228 | 20.637 | 1.00 86.09 | C |
| ATOM | 580 | CE | LYS | A | 69 | 89.394 | 25.097 | 21.390 | 1.00 86.95 | C |
| ATOM | 581 | NZ | LYS | A | 69 | 89.991 | 26.410 | 21.760 | 1.00 87.92 | N |
| ATOM | 582 | C | LYS | A | 69 | 88.029 | 20.447 | 19.683 | 1.00 82.18 | C |
| ATOM | 583 | O | LYS | A | 69 | 88.958 | 19.835 | 20.203 | 1.00 82.98 | O |
| ATOM | 584 | N | GLY | A | 70 | 86.750 | 20.150 | 19.913 | 1.00 80.13 | N |
| ATOM | 585 | CA | GLY | A | 70 | 86.350 | 19.014 | 20.742 | 1.00 78.14 | C |
| ATOM | 586 | C | GLY | A | 70 | 86.766 | 17.677 | 20.153 | 1.00 77.52 | C |
| ATOM | 587 | O | GLY | A | 70 | 86.987 | 16.712 | 20.885 | 1.00 76.75 | O |
| ATOM | 588 | N | ALA | A | 71 | 86.878 | 17.622 | 18.826 | 1.00 78.12 | N |
| ATOM | 589 | CA | ALA | A | 71 | 87.305 | 16.410 | 18.126 | 1.00 75.95 | C |
| ATOM | 590 | CB | ALA | A | 71 | 86.765 | 16.412 | 16.704 | 1.00 77.32 | C |
| ATOM | 591 | C | ALA | A | 71 | 88.826 | 16.241 | 18.109 | 1.00 75.03 | C |
| ATOM | 592 | O | ALA | A | 71 | 89.329 | 15.288 | 17.521 | 1.00 77.75 | O |
| ATOM | 593 | N | THR | A | 72 | 89.552 | 17.157 | 18.748 | 1.00 75.74 | N |
| ATOM | 594 | CA | THR | A | 72 | 91.009 | 17.092 | 18.825 | 1.00 77.19 | C |
| ATOM | 595 | CB | THR | A | 72 | 91.642 | 18.360 | 18.225 | 1.00 77.47 | C |
| ATOM | 596 | OG1 | THR | A | 72 | 91.168 | 19.515 | 18.927 | 1.00 74.39 | O |
| ATOM | 597 | CG2 | THR | A | 72 | 91.290 | 18.485 | 16.752 | 1.00 80.25 | C |
| ATOM | 598 | C | THR | A | 72 | 91.464 | 16.948 | 20.276 | 1.00 80.88 | C |
| ATOM | 599 | O | THR | A | 72 | 90.646 | 16.952 | 21.196 | 1.00 82.69 | O |
| ATOM | 600 | N | THR | A | 73 | 92.776 | 16.827 | 20.471 | 1.00 83.90 | N |
| ATOM | 601 | CA | THR | A | 73 | 93.373 | 16.708 | 21.805 | 1.00 84.61 | C |
| ATOM | 602 | CB | THR | A | 73 | 94.884 | 16.416 | 21.707 | 1.00 85.56 | C |
| ATOM | 603 | OG1 | THR | A | 73 | 95.130 | 15.511 | 20.625 | 1.00 85.62 | O |
| ATOM | 604 | CG2 | THR | A | 73 | 95.410 | 15.811 | 23.004 | 1.00 88.69 | C |
| ATOM | 605 | C | THR | A | 73 | 93.189 | 17.985 | 22.634 | 1.00 84.43 | C |
| ATOM | 606 | O | THR | A | 73 | 93.260 | 17.951 | 23.863 | 1.00 87.77 | O |
| ATOM | 607 | N | TRP | A | 74 | 92.934 | 19.102 | 21.958 | 1.00 82.29 | N |
| ATOM | 608 | CA | TRP | A | 74 | 92.762 | 20.392 | 22.617 | 1.00 80.19 | C |
| ATOM | 609 | CB | TRP | A | 74 | 93.267 | 21.503 | 21.692 | 1.00 81.10 | C |
| ATOM | 610 | CG | TRP | A | 74 | 94.650 | 21.231 | 21.197 | 1.00 81.68 | C |
| ATOM | 611 | CD1 | TRP | A | 74 | 95.000 | 20.674 | 20.003 | 1.00 80.85 | C |
| ATOM | 612 | NE1 | TRP | A | 74 | 96.365 | 20.556 | 19.917 | 1.00 82.91 | N |
| ATOM | 613 | CE2 | TRP | A | 74 | 96.923 | 21.033 | 21.073 | 1.00 83.06 | C |
| ATOM | 614 | CD2 | TRP | A | 74 | 95.870 | 21.458 | 21.907 | 1.00 82.46 | C |
| ATOM | 615 | CE3 | TRP | A | 74 | 96.175 | 21.991 | 23.163 | 1.00 81.89 | C |
| ATOM | 616 | CZ3 | TRP | A | 74 | 97.504 | 22.080 | 23.541 | 1.00 83.00 | C |
| ATOM | 617 | CH2 | TRP | A | 74 | 98.530 | 21.649 | 22.689 | 1.00 83.09 | C |
| ATOM | 618 | CZ2 | TRP | A | 74 | 98.262 | 21.124 | 21.456 | 1.00 82.68 | C |
| ATOM | 619 | C | TRP | A | 74 | 91.308 | 20.640 | 23.015 | 1.00 78.66 | C |
| ATOM | 620 | O | TRP | A | 74 | 90.870 | 21.787 | 23.103 | 1.00 77.18 | O |
| ATOM | 621 | N | PHE | A | 75 | 90.576 | 19.562 | 23.290 | 1.00 79.48 | N |
| ATOM | 622 | CA | PHE | A | 75 | 89.155 | 19.645 | 23.638 | 1.00 81.86 | C |
| ATOM | 623 | CB | PHE | A | 75 | 88.525 | 18.248 | 23.675 | 1.00 81.03 | C |
| ATOM | 624 | CG | PHE | A | 75 | 89.025 | 17.382 | 24.800 | 1.00 81.49 | C |
| ATOM | 625 | CD1 | PHE | A | 75 | 88.464 | 17.474 | 26.067 | 1.00 82.81 | C |
| ATOM | 626 | CE1 | PHE | A | 75 | 88.920 | 16.679 | 27.106 | 1.00 82.06 | C |
| ATOM | 627 | CZ | PHE | A | 75 | 89.945 | 15.776 | 26.886 | 1.00 81.50 | C |
| ATOM | 628 | CE2 | PHE | A | 75 | 90.513 | 15.675 | 25.629 | 1.00 82.26 | C |
| ATOM | 629 | CD2 | PHE | A | 75 | 90.052 | 16.473 | 24.593 | 1.00 81.87 | C |
| ATOM | 630 | C | PHE | A | 75 | 88.883 | 20.350 | 24.966 | 1.00 84.27 | C |
| ATOM | 631 | O | PHE | A | 75 | 87.794 | 20.879 | 25.167 | 1.00 83.13 | O |
| ATOM | 632 | N | ASN | A | 76 | 89.857 | 20.344 | 25.874 | 1.00 88.11 | N |
| ATOM | 633 | CA | ASN | A | 76 | 89.682 | 20.985 | 27.186 | 1.00 89.63 | C |
| ATOM | 634 | CB | ASN | A | 76 | 90.845 | 20.651 | 28.129 | 1.00 92.20 | C |
| ATOM | 635 | CG | ASN | A | 76 | 92.193 | 21.063 | 27.577 | 1.00 94.54 | C |
| ATOM | 636 | OD1 | ASN | A | 76 | 92.288 | 21.819 | 26.609 | 1.00 96.72 | O |
| ATOM | 637 | ND2 | ASN | A | 76 | 93.249 | 20.564 | 28.198 | 1.00 98.59 | N |
| ATOM | 638 | C | ASN | A | 76 | 89.463 | 22.498 | 27.114 | 1.00 88.24 | C |
| ATOM | 639 | O | ASN | A | 76 | 88.887 | 23.084 | 28.029 | 1.00 90.63 | O |
| ATOM | 640 | N | SER | A | 77 | 89.933 | 23.123 | 26.036 | 1.00 87.91 | N |
| ATOM | 641 | CA | SER | A | 77 | 89.700 | 24.548 | 25.801 | 1.00 88.76 | C |
| ATOM | 642 | CB | SER | A | 77 | 90.924 | 25.195 | 25.151 | 1.00 87.82 | C |
| ATOM | 643 | OG | SER | A | 77 | 91.255 | 24.559 | 23.934 | 1.00 88.97 | O |
| ATOM | 644 | C | SER | A | 77 | 88.456 | 24.775 | 24.934 | 1.00 89.89 | C |
| ATOM | 645 | O | SER | A | 77 | 88.060 | 25.916 | 24.702 | 1.00 90.07 | O |
| ATOM | 646 | N | SER | A | 78 | 87.850 | 23.689 | 24.453 | 1.00 94.92 | N |
| ATOM | 647 | CA | SER | A | 78 | 86.629 | 23.761 | 23.649 | 1.00 99.03 | C |
| ATOM | 648 | CB | SER | A | 78 | 86.590 | 22.628 | 22.623 | 1.00 99.62 | C |
| ATOM | 649 | OG | SER | A | 78 | 86.188 | 21.405 | 23.222 | 1.00 97.38 | O |
| ATOM | 650 | C | SER | A | 78 | 85.422 | 23.637 | 24.574 | 1.00102.60 | C |
| ATOM | 651 | O | SER | A | 78 | 85.491 | 22.969 | 25.600 | 1.00103.92 | O |
| ATOM | 652 | N | GLU | A | 79 | 84.313 | 24.267 | 24.207 | 1.00108.55 | N |
| ATOM | 653 | CA | GLU | A | 79 | 83.107 | 24.256 | 25.060 | 1.00114.49 | C |
| ATOM | 654 | CB | GLU | A | 79 | 82.200 | 25.440 | 24.691 | 1.00123.71 | C |
| ATOM | 655 | CG | GLU | A | 79 | 80.904 | 25.532 | 25.489 | 1.00132.76 | C |
| ATOM | 656 | CD | GLU | A | 79 | 80.009 | 26.677 | 25.043 | 1.00140.84 | C |
| ATOM | 657 | OE1 | GLU | A | 79 | 80.450 | 27.508 | 24.217 | 1.00147.37 | O |
| ATOM | 658 | OE2 | GLU | A | 79 | 78.857 | 26.748 | 25.519 | 1.00143.38 | O |
| ATOM | 659 | C | GLU | A | 79 | 82.293 | 22.953 | 25.091 | 1.00109.97 | C |
| ATOM | 660 | O | GLU | A | 79 | 81.727 | 22.609 | 26.129 | 1.00106.29 | O |
| ATOM | 661 | N | GLY | A | 80 | 82.220 | 22.242 | 23.971 | 1.00110.25 | N |
| ATOM | 662 | CA | GLY | A | 80 | 81.446 | 20.997 | 23.891 | 1.00107.47 | C |
| ATOM | 663 | C | GLY | A | 80 | 81.995 | 19.802 | 24.660 | 1.00104.12 | C |
| ATOM | 664 | O | GLY | A | 80 | 81.264 | 18.857 | 24.953 | 1.00104.62 | O |
| ATOM | 665 | N | GLY | A | 81 | 83.275 | 19.851 | 25.012 | 1.00100.72 | N |
| ATOM | 666 | CA | GLY | A | 81 | 83.952 | 18.716 | 25.629 | 1.00 97.65 | C |
| ATOM | 667 | C | GLY | A | 81 | 84.530 | 17.772 | 24.592 | 1.00 93.95 | C |
| ATOM | 668 | O | GLY | A | 81 | 84.514 | 18.060 | 23.394 | 1.00 92.80 | O |
| ATOM | 669 | N | ASN | A | 82 | 85.027 | 16.633 | 25.063 | 1.00 92.19 | N |
| ATOM | 670 | CA | ASN | A | 82 | 85.684 | 15.651 | 24.207 | 1.00 90.58 | C |
| ATOM | 671 | CB | ASN | A | 82 | 86.543 | 14.713 | 25.058 | 1.00 90.22 | C |
| ATOM | 672 | CG | ASN | A | 82 | 87.370 | 13.761 | 24.221 | 1.00 90.16 | C |
| ATOM | 673 | OD1 | ASN | A | 82 | 87.340 | 13.811 | 22.994 | 1.00 93.93 | O |
| ATOM | 674 | ND2 | ASN | A | 82 | 88.116 | 12.889 | 24.882 | 1.00 90.44 | N |
| ATOM | 675 | C | ASN | A | 82 | 84.721 | 14.806 | 23.376 | 1.00 91.56 | C |
| ATOM | 676 | O | ASN | A | 82 | 83.892 | 14.076 | 23.922 | 1.00 92.50 | O |
| ATOM | 677 | N | ILE | A | 83 | 84.850 | 14.902 | 22.054 | 1.00 91.59 | N |
| ATOM | 678 | CA | ILE | A | 83 | 84.067 | 14.083 | 21.128 | 1.00 92.28 | C |
| ATOM | 679 | CB | ILE | A | 83 | 83.002 | 14.924 | 20.383 | 1.00 92.95 | C |
| ATOM | 680 | CG1 | ILE | A | 83 | 83.655 | 15.994 | 19.494 | 1.00 93.81 | C |
| ATOM | 681 | CD1 | ILE | A | 83 | 82.671 | 16.825 | 18.698 | 1.00 94.08 | C |
| ATOM | 682 | CG2 | ILE | A | 83 | 82.048 | 15.565 | 21.378 | 1.00 93.58 | C |
| ATOM | 683 | C | ILE | A | 83 | 84.953 | 13.371 | 20.103 | 1.00 92.93 | C |
| ATOM | 684 | O | ILE | A | 83 | 84.443 | 12.795 | 19.141 | 1.00 95.28 | O |
| ATOM | 685 | N | GLN | A | 84 | 86.269 | 13.390 | 20.316 | 1.00 91.26 | N |
| ATOM | 686 | CA | GLN | A | 84 | 87.202 | 12.832 | 19.337 | 1.00 91.95 | C |
| ATOM | 687 | CB | GLN | A | 84 | 88.656 | 13.183 | 19.686 | 1.00 93.42 | C |
| ATOM | 688 | CG | GLN | A | 84 | 89.228 | 12.543 | 20.940 | 1.00 92.49 | C |
| ATOM | 689 | CD | GLN | A | 84 | 90.513 | 13.214 | 21.412 | 1.00 91.92 | C |
| ATOM | 690 | OE1 | GLN | A | 84 | 90.641 | 13.518 | 22.588 | 1.00 93.06 | O |
| ATOM | 691 | NE2 | GLN | A | 84 | 91.453 | 13.469 | 20.497 | 1.00 89.11 | N |
| ATOM | 692 | C | GLN | A | 84 | 87.021 | 11.331 | 19.159 | 1.00 88.78 | C |
| ATOM | 693 | O | GLN | A | 84 | 86.706 | 10.617 | 20.110 | 1.00 87.28 | O |
| ATOM | 694 | N | ASN | A | 85 | 87.207 | 10.875 | 17.923 | 1.00 88.53 | N |
| ATOM | 695 | CA | ASN | A | 85 | 86.994 | 9.478 | 17.560 | 1.00 86.85 | C |
| ATOM | 696 | CB | ASN | A | 85 | 85.559 | 9.296 | 17.050 | 1.00 88.39 | C |
| ATOM | 697 | CG | ASN | A | 85 | 85.231 | 7.852 | 16.728 | 1.00 90.44 | C |
| ATOM | 698 | OD1 | ASN | A | 85 | 85.629 | 6.938 | 17.448 | 1.00 91.39 | O |
| ATOM | 699 | ND2 | ASN | A | 85 | 84.495 | 7.640 | 15.644 | 1.00 95.33 | N |
| ATOM | 700 | C | ASN | A | 85 | 88.000 | 9.008 | 16.507 | 1.00 82.78 | C |
| ATOM | 701 | O | ASN | A | 85 | 87.668 | 8.860 | 15.331 | 1.00 82.04 | O |
| ATOM | 702 | N | GLY | A | 86 | 89.235 | 8.784 | 16.946 | 1.00 80.60 | N |
| ATOM | 703 | CA | GLY | A | 86 | 90.294 | 8.289 | 16.072 | 1.00 78.14 | C |
| ATOM | 704 | C | GLY | A | 86 | 90.655 | 9.265 | 14.972 | 1.00 76.72 | C |
| ATOM | 705 | O | GLY | A | 86 | 90.790 | 10.462 | 15.217 | 1.00 78.98 | O |
| ATOM | 706 | N | ASN | A | 87 | 90.792 | 8.751 | 13.753 | 1.00 76.52 | N |
| ATOM | 707 | CA | ASN | A | 87 | 91.156 | 9.571 | 12.591 | 1.00 76.73 | C |
| ATOM | 708 | CB | ASN | A | 87 | 91.836 | 8.710 | 11.516 | 1.00 76.38 | C |
| ATOM | 709 | CG | ASN | A | 87 | 93.330 | 8.566 | 11.734 | 1.00 75.91 | C |
| ATOM | 710 | OD1 | ASN | A | 87 | 94.020 | 9.537 | 12.034 | 1.00 72.57 | O |
| ATOM | 711 | ND2 | ASN | A | 87 | 93.842 | 7.354 | 11.552 | 1.00 78.45 | N |
| ATOM | 712 | C | ASN | A | 87 | 90.006 | 10.355 | 11.953 | 1.00 77.29 | C |
| ATOM | 713 | O | ASN | A | 87 | 90.212 | 11.019 | 10.940 | 1.00 78.45 | O |
| ATOM | 714 | N | ASP | A | 88 | 88.811 | 10.293 | 12.535 | 1.00 77.80 | N |
| ATOM | 715 | CA | ASP | A | 88 | 87.659 | 11.015 | 11.994 | 1.00 79.67 | C |
| ATOM | 716 | CB | ASP | A | 88 | 86.369 | 10.517 | 12.654 | 1.00 79.19 | C |
| ATOM | 717 | CG | ASP | A | 88 | 85.114 | 11.001 | 11.945 | 1.00 81.31 | C |
| ATOM | 718 | OD1 | ASP | A | 88 | 85.179 | 11.989 | 11.182 | 1.00 84.32 | O |
| ATOM | 719 | OD2 | ASP | A | 88 | 84.050 | 10.382 | 12.154 | 1.00 84.65 | O |
| ATOM | 720 | C | ASP | A | 88 | 87.826 | 12.530 | 12.209 | 1.00 81.10 | C |
| ATOM | 721 | O | ASP | A | 88 | 87.862 | 12.985 | 13.354 | 1.00 85.52 | O |
| ATOM | 722 | N | PRO | A | 89 | 87.937 | 13.311 | 11.112 | 1.00 80.28 | N |
| ATOM | 723 | CA | PRO | A | 89 | 88.069 | 14.765 | 11.244 | 1.00 80.13 | C |
| ATOM | 724 | CB | PRO | A | 89 | 88.398 | 15.231 | 9.816 | 1.00 81.36 | C |
| ATOM | 725 | CG | PRO | A | 89 | 88.773 | 14.005 | 9.063 | 1.00 81.57 | C |
| ATOM | 726 | CD | PRO | A | 89 | 87.996 | 12.898 | 9.700 | 1.00 81.53 | C |
| ATOM | 727 | C | PRO | A | 89 | 86.802 | 15.462 | 11.738 | 1.00 81.61 | C |
| ATOM | 728 | O | PRO | A | 89 | 86.882 | 16.565 | 12.271 | 1.00 82.89 | O |
| ATOM | 729 | N | CYS | A | 90 | 85.643 | 14.842 | 11.545 | 1.00 83.76 | N |
| ATOM | 730 | CA | CYS | A | 90 | 84.397 | 15.408 | 12.052 | 1.00 87.12 | C |
| ATOM | 731 | CB | CYS | A | 90 | 83.756 | 16.336 | 11.019 | 1.00 89.21 | C |
| ATOM | 732 | SG | CYS | A | 90 | 82.536 | 17.452 | 11.749 | 1.00 97.73 | S |
| ATOM | 733 | C | CYS | A | 90 | 83.441 | 14.291 | 12.475 | 1.00 87.04 | C |
| ATOM | 734 | O | CYS | A | 90 | 82.627 | 13.831 | 11.676 | 1.00 88.42 | O |
| ATOM | 735 | N | PRO | A | 91 | 83.560 | 13.834 | 13.736 | 1.00 88.68 | N |
| ATOM | 736 | CA | PRO | A | 91 | 82.711 | 12.766 | 14.265 | 1.00 87.95 | C |
| ATOM | 737 | CB | PRO | A | 91 | 83.210 | 12.595 | 15.706 | 1.00 88.48 | C |
| ATOM | 738 | CG | PRO | A | 91 | 84.600 | 13.125 | 15.697 | 1.00 89.41 | C |
| ATOM | 739 | CD | PRO | A | 91 | 84.582 | 14.252 | 14.712 | 1.00 89.53 | C |
| ATOM | 740 | C | PRO | A | 91 | 81.231 | 13.133 | 14.254 | 1.00 87.89 | C |
| ATOM | 741 | O | PRO | A | 91 | 80.864 | 14.231 | 14.670 | 1.00 88.66 | O |
| ATOM | 742 | N | GLY | A | 92 | 80.400 | 12.216 | 13.768 | 1.00 88.83 | N |
| ATOM | 743 | CA | GLY | A | 92 | 78.956 | 12.427 | 13.711 | 1.00 88.73 | C |
| ATOM | 744 | C | GLY | A | 92 | 78.455 | 13.014 | 12.404 | 1.00 87.94 | C |
| ATOM | 745 | O | GLY | A | 92 | 77.247 | 13.115 | 12.200 | 1.00 92.01 | O |
| ATOM | 746 | N | SER | A | 93 | 79.372 | 13.401 | 11.519 | 1.00 85.11 | N |
| ATOM | 747 | CA | SER | A | 93 | 79.009 | 13.950 | 10.211 | 1.00 83.68 | C |
| ATOM | 748 | CB | SER | A | 93 | 79.288 | 15.455 | 10.156 | 1.00 83.96 | C |
| ATOM | 749 | OG | SER | A | 93 | 80.668 | 15.729 | 10.309 | 1.00 86.87 | O |
| ATOM | 750 | C | SER | A | 93 | 79.765 | 13.203 | 9.102 | 1.00 81.71 | C |
| ATOM | 751 | O | SER | A | 93 | 80.841 | 12.653 | 9.346 | 1.00 79.60 | O |
| ATOM | 752 | N | PRO | A | 94 | 79.195 | 13.171 | 7.881 | 1.00 81.27 | N |
| ATOM | 753 | CA | PRO | A | 94 | 79.796 | 12.420 | 6.781 | 1.00 79.29 | C |
| ATOM | 754 | CB | PRO | A | 94 | 78.625 | 12.248 | 5.816 | 1.00 81.13 | C |
| ATOM | 755 | CG | PRO | A | 94 | 77.800 | 13.467 | 6.022 | 1.00 80.76 | C |
| ATOM | 756 | CD | PRO | A | 94 | 77.957 | 13.858 | 7.462 | 1.00 80.51 | C |
| ATOM | 757 | C | PRO | A | 94 | 80.948 | 13.170 | 6.107 | 1.00 78.51 | C |
| ATOM | 758 | O | PRO | A | 94 | 81.217 | 14.320 | 6.464 | 1.00 77.25 | O |
| ATOM | 759 | N | PRO | A | 95 | 81.628 | 12.527 | 5.134 | 1.00 76.99 | N |
| ATOM | 760 | CA | PRO | A | 95 | 82.752 | 13.168 | 4.438 | 1.00 75.51 | C |
| ATOM | 761 | CB | PRO | A | 95 | 83.280 | 12.063 | 3.515 | 1.00 74.85 | C |
| ATOM | 762 | CG | PRO | A | 95 | 82.794 | 10.793 | 4.113 | 1.00 76.11 | C |
| ATOM | 763 | CD | PRO | A | 95 | 81.460 | 11.127 | 4.709 | 1.00 76.90 | C |
| ATOM | 764 | C | PRO | A | 95 | 82.383 | 14.411 | 3.622 | 1.00 76.85 | C |
| ATOM | 765 | O | PRO | A | 95 | 83.273 | 15.177 | 3.255 | 1.00 73.77 | O |
| ATOM | 766 | N | SER | A | 96 | 81.094 | 14.606 | 3.337 | 1.00 79.96 | N |
| ATOM | 767 | CA | SER | A | 96 | 80.635 | 15.806 | 2.632 | 1.00 81.01 | C |
| ATOM | 768 | CB | SER | A | 96 | 79.130 | 15.742 | 2.356 | 1.00 82.03 | C |
| ATOM | 769 | OG | SER | A | 96 | 78.380 | 15.812 | 3.556 | 1.00 81.00 | O |
| ATOM | 770 | C | SER | A | 96 | 80.958 | 17.063 | 3.436 | 1.00 81.78 | C |
| ATOM | 771 | O | SER | A | 96 | 81.075 | 18.153 | 2.876 | 1.00 82.50 | O |
| ATOM | 772 | N | GLU | A | 97 | 81.095 | 16.898 | 4.751 | 1.00 84.85 | N |
| ATOM | 773 | CA | GLU | A | 97 | 81.482 | 17.987 | 5.638 | 1.00 85.15 | C |
| ATOM | 774 | CB | GLU | A | 97 | 81.290 | 17.571 | 7.102 | 1.00 87.89 | C |
| ATOM | 775 | CG | GLU | A | 97 | 81.230 | 18.728 | 8.093 | 1.00 89.90 | C |
| ATOM | 776 | CD | GLU | A | 97 | 82.591 | 19.298 | 8.462 | 1.00 90.87 | C |
| ATOM | 777 | OE1 | GLU | A | 97 | 82.633 | 20.470 | 8.886 | 1.00 90.84 | O |
| ATOM | 778 | OE2 | GLU | A | 97 | 83.615 | 18.586 | 8.340 | 1.00 90.58 | O |
| ATOM | 779 | C | GLU | A | 97 | 82.932 | 18.407 | 5.363 | 1.00 82.63 | C |
| ATOM | 780 | O | GLU | A | 97 | 83.256 | 19.591 | 5.449 | 1.00 80.59 | O |
| ATOM | 781 | N | TYR | A | 98 | 83.795 | 17.451 | 5.010 | 1.00 81.53 | N |
| ATOM | 782 | CA | TYR | A | 98 | 85.179 | 17.784 | 4.653 | 1.00 79.61 | C |
| ATOM | 783 | CB | TYR | A | 98 | 86.205 | 16.673 | 4.983 | 1.00 79.95 | C |
| ATOM | 784 | CG | TYR | A | 98 | 85.804 | 15.496 | 5.847 | 1.00 80.21 | C |
| ATOM | 785 | CD1 | TYR | A | 98 | 85.165 | 15.671 | 7.065 | 1.00 83.17 | C |
| ATOM | 786 | CE1 | TYR | A | 98 | 84.821 | 14.580 | 7.851 | 1.00 86.71 | C |
| ATOM | 787 | CZ | TYR | A | 98 | 85.150 | 13.294 | 7.436 | 1.00 85.74 | C |
| ATOM | 788 | OH | TYR | A | 98 | 84.815 | 12.204 | 8.217 | 1.00 82.99 | O |
| ATOM | 789 | CE2 | TYR | A | 98 | 85.816 | 13.102 | 6.243 | 1.00 81.75 | C |
| ATOM | 790 | CD2 | TYR | A | 98 | 86.147 | 14.197 | 5.464 | 1.00 79.31 | C |
| ATOM | 791 | C | TYR | A | 98 | 85.281 | 18.111 | 3.148 | 1.00 78.58 | C |
| ATOM | 792 | O | TYR | A | 98 | 86.383 | 18.165 | 2.602 | 1.00 75.58 | O |
| ATOM | 793 | N | HIS | A | 99 | 84.138 | 18.308 | 2.488 | 1.00 78.17 | N |
| ATOM | 794 | CA | HIS | A | 99 | 84.070 | 18.625 | 1.056 | 1.00 78.49 | C |
| ATOM | 795 | CB | HIS | A | 99 | 84.615 | 20.029 | 0.775 | 1.00 80.65 | C |
| ATOM | 796 | CG | HIS | A | 99 | 83.839 | 21.122 | 1.438 | 1.00 84.17 | C |
| ATOM | 797 | ND1 | HIS | A | 99 | 82.534 | 21.414 | 1.104 | 1.00 86.58 | N |
| ATOM | 798 | CE1 | HIS | A | 99 | 82.108 | 22.423 | 1.841 | 1.00 87.71 | C |
| ATOM | 799 | NE2 | HIS | A | 99 | 83.091 | 22.802 | 2.637 | 1.00 87.96 | N |
| ATOM | 800 | CD2 | HIS | A | 99 | 84.187 | 22.006 | 2.402 | 1.00 85.49 | C |
| ATOM | 801 | C | HIS | A | 99 | 84.760 | 17.591 | 0.157 | 1.00 77.06 | C |
| ATOM | 802 | O | HIS | A | 99 | 85.602 | 17.933 | −0.678 | 1.00 75.08 | O |
| ATOM | 803 | N | THR | A | 100 | 84.385 | 16.328 | 0.333 | 1.00 76.26 | N |
| ATOM | 804 | CA | THR | A | 100 | 84.905 | 15.243 | −0.494 | 1.00 75.33 | C |
| ATOM | 805 | CB | THR | A | 100 | 86.259 | 14.719 | 0.026 | 1.00 75.69 | C |
| ATOM | 806 | OG1 | THR | A | 100 | 86.691 | 13.618 | −0.781 | 1.00 73.34 | O |
| ATOM | 807 | CG2 | THR | A | 100 | 86.161 | 14.268 | 1.487 | 1.00 76.67 | C |
| ATOM | 808 | C | THR | A | 100 | 83.904 | 14.094 | −0.565 | 1.00 77.28 | C |
| ATOM | 809 | O | THR | A | 100 | 83.156 | 13.852 | 0.381 | 1.00 78.28 | O |
| ATOM | 810 | N | THR | A | 101 | 83.893 | 13.401 | −1.700 | 1.00 79.81 | N |
| ATOM | 811 | CA | THR | A | 101 | 83.006 | 12.257 | −1.908 | 1.00 78.80 | C |
| ATOM | 812 | CB | THR | A | 101 | 82.726 | 12.026 | −3.405 | 1.00 78.80 | C |
| ATOM | 813 | OG1 | THR | A | 101 | 83.949 | 11.727 | −4.091 | 1.00 80.62 | O |
| ATOM | 814 | CG2 | THR | A | 101 | 82.095 | 13.259 | −4.025 | 1.00 78.50 | C |
| ATOM | 815 | C | THR | A | 101 | 83.630 | 10.999 | −1.314 | 1.00 77.63 | C |
| ATOM | 816 | O | THR | A | 101 | 82.924 | 10.127 | −0.809 | 1.00 79.35 | O |
| ATOM | 817 | N | GLY | A | 102 | 84.957 | 10.912 | −1.389 | 1.00 74.75 | N |
| ATOM | 818 | CA | GLY | A | 102 | 85.700 | 9.780 | −0.843 | 1.00 73.71 | C |
| ATOM | 819 | C | GLY | A | 102 | 87.185 | 10.075 | −0.760 | 1.00 72.34 | C |
| ATOM | 820 | O | GLY | A | 102 | 87.616 | 11.198 | −1.013 | 1.00 71.11 | O |
| ATOM | 821 | N | ILE | A | 103 | 87.971 | 9.062 | −0.411 | 1.00 73.72 | N |
| ATOM | 822 | CA | ILE | A | 103 | 89.422 | 9.218 | −0.295 | 1.00 77.88 | C |
| ATOM | 823 | CB | ILE | A | 103 | 90.077 | 7.954 | 0.313 | 1.00 78.60 | C |
| ATOM | 824 | CG1 | ILE | A | 103 | 91.526 | 8.238 | 0.723 | 1.00 79.37 | C |
| ATOM | 825 | CD1 | ILE | A | 103 | 92.155 | 7.125 | 1.531 | 1.00 81.18 | C |
| ATOM | 826 | CG2 | ILE | A | 103 | 90.005 | 6.774 | −0.650 | 1.00 79.39 | C |
| ATOM | 827 | C | ILE | A | 103 | 90.080 | 9.560 | −1.638 | 1.00 81.78 | C |
| ATOM | 828 | O | ILE | A | 103 | 91.135 | 10.193 | −1.677 | 1.00 83.03 | O |
| ATOM | 829 | N | ASP | A | 104 | 89.454 | 9.131 | −2.730 | 1.00 88.15 | N |
| ATOM | 830 | CA | ASP | A | 104 | 89.956 | 9.401 | −4.083 | 1.00 91.83 | C |
| ATOM | 831 | CB | ASP | A | 104 | 89.357 | 8.399 | −5.089 | 1.00 98.41 | C |
| ATOM | 832 | CG | ASP | A | 104 | 87.828 | 8.292 | −4.998 | 1.00106.90 | C |
| ATOM | 833 | OD1 | ASP | A | 104 | 87.191 | 9.093 | −4.276 | 1.00108.74 | O |
| ATOM | 834 | OD2 | ASP | A | 104 | 87.263 | 7.390 | −5.653 | 1.00115.22 | O |
| ATOM | 835 | C | ASP | A | 104 | 89.704 | 10.844 | −4.547 | 1.00 87.09 | C |
| ATOM | 836 | O | ASP | A | 104 | 90.380 | 11.332 | −5.454 | 1.00 85.40 | O |
| ATOM | 837 | N | ASP | A | 105 | 88.747 | 11.520 | −3.913 | 1.00 84.52 | N |
| ATOM | 838 | CA | ASP | A | 105 | 88.347 | 12.878 | −4.300 | 1.00 83.78 | C |
| ATOM | 839 | CB | ASP | A | 105 | 86.818 | 12.984 | −4.220 | 1.00 83.39 | C |
| ATOM | 840 | CG | ASP | A | 105 | 86.278 | 14.287 | −4.782 | 1.00 83.83 | C |
| ATOM | 841 | OD1 | ASP | A | 105 | 86.840 | 14.809 | −5.772 | 1.00 84.71 | O |
| ATOM | 842 | OD2 | ASP | A | 105 | 85.271 | 14.779 | −4.231 | 1.00 83.05 | O |
| ATOM | 843 | C | ASP | A | 105 | 89.006 | 13.990 | −3.462 | 1.00 82.38 | C |
| ATOM | 844 | O | ASP | A | 105 | 88.697 | 15.169 | −3.637 | 1.00 84.48 | O |
| ATOM | 845 | N | VAL | A | 106 | 89.915 | 13.623 | −2.562 | 1.00 80.21 | N |
| ATOM | 846 | CA | VAL | A | 106 | 90.636 | 14.616 | −1.758 | 1.00 76.79 | C |
| ATOM | 847 | CB | VAL | A | 106 | 91.240 | 14.007 | −0.473 | 1.00 76.30 | C |
| ATOM | 848 | CG1 | VAL | A | 106 | 90.152 | 13.342 | 0.361 | 1.00 75.86 | C |
| ATOM | 849 | CG2 | VAL | A | 106 | 92.364 | 13.028 | −0.792 | 1.00 75.64 | C |
| ATOM | 850 | C | VAL | A | 106 | 91.732 | 15.252 | −2.613 | 1.00 77.16 | C |
| ATOM | 851 | O | VAL | A | 106 | 92.360 | 14.571 | −3.425 | 1.00 78.52 | O |
| ATOM | 852 | N | LYS | A | 107 | 91.964 | 16.550 | −2.423 | 1.00 76.96 | N |
| ATOM | 853 | CA | LYS | A | 107 | 92.907 | 17.302 | −3.262 | 1.00 76.64 | C |
| ATOM | 854 | CB | LYS | A | 107 | 92.202 | 18.528 | −3.844 | 1.00 78.36 | C |
| ATOM | 855 | CG | LYS | A | 107 | 90.971 | 18.201 | −4.675 | 1.00 80.98 | C |
| ATOM | 856 | CD | LYS | A | 107 | 91.330 | 17.445 | −5.943 | 1.00 83.14 | C |
| ATOM | 857 | CE | LYS | A | 107 | 90.093 | 17.097 | −6.748 | 1.00 85.25 | C |
| ATOM | 858 | NZ | LYS | A | 107 | 90.459 | 16.404 | −8.012 | 1.00 89.06 | N |
| ATOM | 859 | C | LYS | A | 107 | 94.197 | 17.739 | −2.568 | 1.00 75.69 | C |
| ATOM | 860 | O | LYS | A | 107 | 95.007 | 18.443 | −3.166 | 1.00 76.63 | O |
| ATOM | 861 | N | GLY | A | 108 | 94.386 | 17.345 | −1.313 | 1.00 75.76 | N |
| ATOM | 862 | CA | GLY | A | 108 | 95.621 | 17.655 | −0.597 | 1.00 74.81 | C |
| ATOM | 863 | C | GLY | A | 108 | 95.705 | 19.048 | 0.000 | 1.00 74.07 | C |
| ATOM | 864 | O | GLY | A | 108 | 95.023 | 19.975 | −0.442 | 1.00 74.11 | O |
| ATOM | 865 | N | CYS | A | 109 | 96.561 | 19.179 | 1.012 | 1.00 73.65 | N |
| ATOM | 866 | CA | CYS | A | 109 | 96.774 | 20.438 | 1.719 | 1.00 74.38 | C |
| ATOM | 867 | CB | CYS | A | 109 | 95.952 | 20.472 | 3.003 | 1.00 77.23 | C |
| ATOM | 868 | SG | CYS | A | 109 | 96.511 | 19.304 | 4.250 | 1.00 86.71 | S |
| ATOM | 869 | C | CYS | A | 109 | 98.258 | 20.590 | 2.034 | 1.00 70.37 | C |
| ATOM | 870 | O | CYS | A | 109 | 99.021 | 19.636 | 1.884 | 1.00 70.18 | O |
| ATOM | 871 | N | ALA | A | 110 | 98.661 | 21.774 | 2.492 | 1.00 68.05 | N |
| ATOM | 872 | CA | ALA | A | 110 | 100.080 | 22.073 | 2.693 | 1.00 67.71 | C |
| ATOM | 873 | CB | ALA | A | 110 | 100.606 | 22.832 | 1.484 | 1.00 66.87 | C |
| ATOM | 874 | C | ALA | A | 110 | 100.396 | 22.864 | 3.960 | 1.00 67.08 | C |
| ATOM | 875 | O | ALA | A | 110 | 99.507 | 23.404 | 4.615 | 1.00 65.06 | O |
| ATOM | 876 | N | MET | A | 111 | 101.687 | 22.911 | 4.283 | 1.00 66.62 | N |
| ATOM | 877 | CA | MET | A | 111 | 102.214 | 23.716 | 5.376 | 1.00 68.59 | C |
| ATOM | 878 | CB | MET | A | 111 | 102.953 | 22.855 | 6.408 | 1.00 69.75 | C |
| ATOM | 879 | CG | MET | A | 111 | 102.147 | 21.746 | 7.071 | 1.00 71.01 | C |
| ATOM | 880 | SD | MET | A | 111 | 100.703 | 22.240 | 8.040 | 1.00 71.63 | S |
| ATOM | 881 | CE | MET | A | 111 | 101.331 | 23.608 | 8.969 | 1.00 71.26 | C |
| ATOM | 882 | C | MET | A | 111 | 103.216 | 24.694 | 4.768 | 1.00 70.65 | C |
| ATOM | 883 | O | MET | A | 111 | 104.177 | 24.273 | 4.118 | 1.00 69.63 | O |
| ATOM | 884 | N | ALA | A | 112 | 102.993 | 25.989 | 4.972 | 1.00 72.73 | N |
| ATOM | 885 | CA | ALA | A | 112 | 103.926 | 27.013 | 4.511 | 1.00 73.22 | C |
| ATOM | 886 | CB | ALA | A | 112 | 103.182 | 28.138 | 3.816 | 1.00 74.60 | C |
| ATOM | 887 | C | ALA | A | 112 | 104.715 | 27.550 | 5.699 | 1.00 75.00 | C |
| ATOM | 888 | O | ALA | A | 112 | 104.319 | 27.354 | 6.849 | 1.00 74.54 | O |
| ATOM | 889 | N | ILE | A | 113 | 105.829 | 28.223 | 5.417 | 1.00 75.76 | N |
| ATOM | 890 | CA | ILE | A | 113 | 106.684 | 28.781 | 6.467 | 1.00 75.57 | C |
| ATOM | 891 | CB | ILE | A | 113 | 107.799 | 27.787 | 6.871 | 1.00 74.30 | C |
| ATOM | 892 | CG1 | ILE | A | 113 | 108.525 | 28.264 | 8.132 | 1.00 72.52 | C |
| ATOM | 893 | CD1 | ILE | A | 113 | 109.418 | 27.209 | 8.747 | 1.00 71.73 | C |
| ATOM | 894 | CG2 | ILE | A | 113 | 108.787 | 27.567 | 5.729 | 1.00 74.42 | C |
| ATOM | 895 | C | ILE | A | 113 | 107.318 | 30.108 | 6.057 | 1.00 78.06 | C |
| ATOM | 896 | O | ILE | A | 113 | 107.704 | 30.299 | 4.903 | 1.00 78.43 | O |
| ATOM | 897 | N | ALA | A | 114 | 107.418 | 31.017 | 7.023 | 1.00 81.36 | N |
| ATOM | 898 | CA | ALA | A | 114 | 108.054 | 32.312 | 6.827 | 1.00 82.37 | C |
| ATOM | 899 | CB | ALA | A | 114 | 107.010 | 33.411 | 6.755 | 1.00 82.04 | C |
| ATOM | 900 | C | ALA | A | 114 | 109.006 | 32.554 | 7.992 | 1.00 84.16 | C |
| ATOM | 901 | O | ALA | A | 114 | 108.574 | 32.630 | 9.144 | 1.00 85.37 | O |
| ATOM | 902 | N | TYR | A | 115 | 110.297 | 32.668 | 7.688 | 1.00 84.64 | N |
| ATOM | 903 | CA | TYR | A | 115 | 111.326 | 32.873 | 8.709 | 1.00 84.66 | C |
| ATOM | 904 | CB | TYR | A | 115 | 112.710 | 32.503 | 8.165 | 1.00 82.76 | C |
| ATOM | 905 | CG | TYR | A | 115 | 112.967 | 31.012 | 8.120 | 1.00 81.09 | C |
| ATOM | 906 | CD1 | TYR | A | 115 | 114.000 | 30.445 | 8.856 | 1.00 80.03 | C |
| ATOM | 907 | CE1 | TYR | A | 115 | 114.239 | 29.082 | 8.820 | 1.00 79.47 | C |
| ATOM | 908 | CZ | TYR | A | 115 | 113.434 | 28.265 | 8.053 | 1.00 77.13 | C |
| ATOM | 909 | OH | TYR | A | 115 | 113.668 | 26.915 | 8.019 | 1.00 73.14 | O |
| ATOM | 910 | CE2 | TYR | A | 115 | 112.398 | 28.800 | 7.319 | 1.00 79.12 | C |
| ATOM | 911 | CD2 | TYR | A | 115 | 112.167 | 30.165 | 7.358 | 1.00 81.10 | C |
| ATOM | 912 | C | TYR | A | 115 | 111.304 | 34.313 | 9.219 | 1.00 86.07 | C |
| ATOM | 913 | O | TYR | A | 115 | 112.121 | 35.147 | 8.821 | 1.00 85.81 | O |
| ATOM | 914 | N | GLU | A | 116 | 110.346 | 34.586 | 10.100 | 1.00 87.27 | N |
| ATOM | 915 | CA | GLU | A | 116 | 110.166 | 35.905 | 10.685 | 1.00 91.13 | C |
| ATOM | 916 | CB | GLU | A | 116 | 109.296 | 36.769 | 9.766 | 1.00 93.22 | C |
| ATOM | 917 | CG | GLU | A | 116 | 108.943 | 38.140 | 10.326 | 1.00 97.30 | C |
| ATOM | 918 | CD | GLU | A | 116 | 110.159 | 39.000 | 10.616 | 1.00101.54 | C |
| ATOM | 919 | OE1 | GLU | A | 116 | 111.055 | 39.082 | 9.748 | 1.00107.29 | O |
| ATOM | 920 | OE2 | GLU | A | 116 | 110.211 | 39.609 | 11.706 | 1.00102.79 | O |
| ATOM | 921 | C | GLU | A | 116 | 109.524 | 35.765 | 12.065 | 1.00 93.80 | C |
| ATOM | 922 | O | GLU | A | 116 | 108.479 | 35.132 | 12.210 | 1.00 92.53 | O |
| ATOM | 923 | N | SER | A | 117 | 110.159 | 36.358 | 13.071 | 1.00 97.53 | N |
| ATOM | 924 | CA | SER | A | 117 | 109.664 | 36.303 | 14.448 | 1.00 97.24 | C |
| ATOM | 925 | CB | SER | A | 117 | 110.806 | 36.585 | 15.435 | 1.00100.11 | C |
| ATOM | 926 | OG | SER | A | 117 | 111.643 | 37.633 | 14.972 | 1.00105.40 | O |
| ATOM | 927 | C | SER | A | 117 | 108.489 | 37.255 | 14.694 | 1.00 95.90 | C |
| ATOM | 928 | O | SER | A | 117 | 107.653 | 36.990 | 15.558 | 1.00 94.55 | O |
| ATOM | 929 | N | ASP | A | 118 | 108.431 | 38.358 | 13.946 | 1.00 96.01 | N |
| ATOM | 930 | CA | ASP | A | 118 | 107.349 | 39.340 | 14.078 | 1.00 97.66 | C |
| ATOM | 931 | CB | ASP | A | 118 | 107.911 | 40.765 | 13.984 | 1.00 99.77 | C |
| ATOM | 932 | CG | ASP | A | 118 | 106.853 | 41.842 | 14.212 | 1.00101.82 | C |
| ATOM | 933 | OD1 | ASP | A | 118 | 105.746 | 41.528 | 14.704 | 1.00103.70 | O |
| ATOM | 934 | OD2 | ASP | A | 118 | 107.141 | 43.018 | 13.904 | 1.00104.07 | O |
| ATOM | 935 | C | ASP | A | 118 | 106.288 | 39.116 | 12.999 | 1.00 97.06 | C |
| ATOM | 936 | O | ASP | A | 118 | 106.510 | 39.421 | 11.829 | 1.00 98.49 | O |
| ATOM | 937 | N | VAL | A | 119 | 105.130 | 38.604 | 13.408 | 1.00 96.94 | N |
| ATOM | 938 | CA | VAL | A | 119 | 104.030 | 38.294 | 12.483 | 1.00 97.43 | C |
| ATOM | 939 | CB | VAL | A | 119 | 102.807 | 37.719 | 13.242 | 1.00 99.06 | C |
| ATOM | 940 | CG1 | VAL | A | 119 | 102.134 | 38.786 | 14.098 | 1.00 99.23 | C |
| ATOM | 941 | CG2 | VAL | A | 119 | 101.810 | 37.093 | 12.276 | 1.00100.11 | C |
| ATOM | 942 | C | VAL | A | 119 | 103.581 | 39.492 | 11.637 | 1.00 96.97 | C |
| ATOM | 943 | O | VAL | A | 119 | 103.140 | 39.322 | 10.500 | 1.00 96.72 | O |
| ATOM | 944 | N | ARG | A | 120 | 103.705 | 40.695 | 12.193 | 1.00 97.92 | N |
| ATOM | 945 | CA | ARG | A | 120 | 103.284 | 41.919 | 11.508 | 1.00 99.51 | C |
| ATOM | 946 | CB | ARG | A | 120 | 103.353 | 43.115 | 12.466 | 1.00103.11 | C |
| ATOM | 947 | CG | ARG | A | 120 | 102.446 | 43.008 | 13.686 | 1.00105.30 | C |
| ATOM | 948 | CD | ARG | A | 120 | 100.973 | 43.100 | 13.315 | 1.00105.70 | C |
| ATOM | 949 | NE | ARG | A | 120 | 100.099 | 42.829 | 14.454 | 1.00105.98 | N |
| ATOM | 950 | CZ | ARG | A | 120 | 98.769 | 42.875 | 14.415 | 1.00106.94 | C |
| ATOM | 951 | NH1 | ARG | A | 120 | 98.134 | 43.189 | 13.290 | 1.00107.36 | N |
| ATOM | 952 | NH2 | ARG | A | 120 | 98.066 | 42.610 | 15.511 | 1.00108.34 | N |
| ATOM | 953 | C | ARG | A | 120 | 104.108 | 42.222 | 10.255 | 1.00 98.14 | C |
| ATOM | 954 | O | ARG | A | 120 | 103.621 | 42.887 | 9.341 | 1.00 98.42 | O |
| ATOM | 955 | N | LYS | A | 121 | 105.348 | 41.739 | 10.217 | 1.00 96.66 | N |
| ATOM | 956 | CA | LYS | A | 121 | 106.230 | 41.955 | 9.069 | 1.00 97.00 | C |
| ATOM | 957 | CB | LYS | A | 121 | 107.695 | 41.967 | 9.517 | 1.00100.76 | C |
| ATOM | 958 | CG | LYS | A | 121 | 108.074 | 43.177 | 10.355 | 1.00105.33 | C |
| ATOM | 959 | CD | LYS | A | 121 | 109.582 | 43.288 | 10.515 | 1.00108.74 | C |
| ATOM | 960 | CE | LYS | A | 121 | 109.983 | 44.630 | 11.105 | 1.00111.26 | C |
| ATOM | 961 | NZ | LYS | A | 121 | 111.458 | 44.828 | 11.062 | 1.00114.12 | N |
| ATOM | 962 | C | LYS | A | 121 | 106.052 | 40.929 | 7.948 | 1.00 95.26 | C |
| ATOM | 963 | O | LYS | A | 121 | 106.583 | 41.120 | 6.854 | 1.00 97.97 | O |
| ATOM | 964 | N | ILE | A | 122 | 105.310 | 39.853 | 8.207 | 1.00 93.34 | N |
| ATOM | 965 | CA | ILE | A | 122 | 105.126 | 38.788 | 7.214 | 1.00 91.06 | C |
| ATOM | 966 | CB | ILE | A | 122 | 104.603 | 37.482 | 7.850 | 1.00 89.31 | C |
| ATOM | 967 | CG1 | ILE | A | 122 | 105.591 | 36.969 | 8.900 | 1.00 90.30 | C |
| ATOM | 968 | CD1 | ILE | A | 122 | 105.068 | 35.813 | 9.722 | 1.00 90.61 | C |
| ATOM | 969 | CG2 | ILE | A | 122 | 104.398 | 36.411 | 6.784 | 1.00 87.46 | C |
| ATOM | 970 | C | ILE | A | 122 | 104.167 | 39.215 | 6.107 | 1.00 91.89 | C |
| ATOM | 971 | O | ILE | A | 122 | 103.072 | 39.711 | 6.378 | 1.00 92.60 | O |
| ATOM | 972 | N | LYS | A | 123 | 104.593 | 39.000 | 4.864 | 1.00 93.11 | N |
| ATOM | 973 | CA | LYS | A | 123 | 103.795 | 39.318 | 3.684 | 1.00 95.03 | C |
| ATOM | 974 | CB | LYS | A | 123 | 104.570 | 40.271 | 2.776 | 1.00 98.86 | C |
| ATOM | 975 | CG | LYS | A | 123 | 104.764 | 41.651 | 3.385 | 1.00102.64 | C |
| ATOM | 976 | CD | LYS | A | 123 | 105.857 | 42.439 | 2.684 | 1.00105.78 | C |
| ATOM | 977 | CE | LYS | A | 123 | 106.091 | 43.771 | 3.377 | 1.00108.87 | C |
| ATOM | 978 | NZ | LYS | A | 123 | 107.317 | 44.453 | 2.879 | 1.00111.78 | N |
| ATOM | 979 | C | LYS | A | 123 | 103.439 | 38.027 | 2.945 | 1.00 92.62 | C |
| ATOM | 980 | O | LYS | A | 123 | 104.125 | 37.017 | 3.103 | 1.00 91.55 | O |
| ATOM | 981 | N | PRO | A | 124 | 102.368 | 38.053 | 2.129 | 1.00 89.62 | N |
| ATOM | 982 | CA | PRO | A | 124 | 101.920 | 36.841 | 1.431 | 1.00 89.50 | C |
| ATOM | 983 | CB | PRO | A | 124 | 100.746 | 37.338 | 0.581 | 1.00 91.29 | C |
| ATOM | 984 | CG | PRO | A | 124 | 100.301 | 38.592 | 1.241 | 1.00 90.38 | C |
| ATOM | 985 | CD | PRO | A | 124 | 101.553 | 39.220 | 1.756 | 1.00 89.45 | C |
| ATOM | 986 | C | PRO | A | 124 | 102.984 | 36.211 | 0.536 | 1.00 89.46 | C |
| ATOM | 987 | O | PRO | A | 124 | 102.998 | 34.994 | 0.358 | 1.00 89.19 | O |
| ATOM | 988 | N | GLU | A | 125 | 103.867 | 37.041 | −0.011 | 1.00 89.63 | N |
| ATOM | 989 | CA | GLU | A | 125 | 104.938 | 36.569 | −0.881 | 1.00 89.62 | C |
| ATOM | 990 | CB | GLU | A | 125 | 105.574 | 37.735 | −1.655 | 1.00 95.09 | C |
| ATOM | 991 | CG | GLU | A | 125 | 104.612 | 38.588 | −2.484 | 1.00 99.97 | C |
| ATOM | 992 | CD | GLU | A | 125 | 103.940 | 39.715 | −1.704 | 1.00102.20 | C |
| ATOM | 993 | OE1 | GLU | A | 125 | 104.465 | 40.128 | −0.647 | 1.00102.51 | O |
| ATOM | 994 | OE2 | GLU | A | 125 | 102.882 | 40.202 | −2.161 | 1.00103.21 | O |
| ATOM | 995 | C | GLU | A | 125 | 106.033 | 35.846 | −0.096 | 1.00 86.13 | C |
| ATOM | 996 | O | GLU | A | 125 | 106.828 | 35.119 | −0.683 | 1.00 82.74 | O |
| ATOM | 997 | N | ASP | A | 126 | 106.077 | 36.053 | 1.221 | 1.00 86.04 | N |
| ATOM | 998 | CA | ASP | A | 126 | 107.130 | 35.479 | 2.067 | 1.00 83.80 | C |
| ATOM | 999 | CB | ASP | A | 126 | 107.259 | 36.269 | 3.375 | 1.00 86.37 | C |
| ATOM | 1000 | CG | ASP | A | 126 | 107.607 | 37.735 | 3.148 | 1.00 89.43 | C |
| ATOM | 1001 | OD1 | ASP | A | 126 | 108.014 | 38.095 | 2.023 | 1.00 90.53 | O |
| ATOM | 1002 | OD2 | ASP | A | 126 | 107.479 | 38.529 | 4.105 | 1.00 91.56 | O |
| ATOM | 1003 | C | ASP | A | 126 | 106.937 | 33.997 | 2.385 | 1.00 79.71 | C |
| ATOM | 1004 | O | ASP | A | 126 | 107.919 | 33.277 | 2.575 | 1.00 76.75 | O |
| ATOM | 1005 | N | PHE | A | 127 | 105.687 | 33.539 | 2.440 | 1.00 77.69 | N |
| ATOM | 1006 | CA | PHE | A | 127 | 105.406 | 32.127 | 2.739 | 1.00 76.03 | C |
| ATOM | 1007 | CB | PHE | A | 127 | 103.907 | 31.876 | 2.959 | 1.00 74.67 | C |
| ATOM | 1008 | CG | PHE | A | 127 | 103.461 | 32.070 | 4.377 | 1.00 74.73 | C |
| ATOM | 1009 | CD1 | PHE | A | 127 | 103.999 | 31.292 | 5.392 | 1.00 75.69 | C |
| ATOM | 1010 | CE1 | PHE | A | 127 | 103.590 | 31.452 | 6.703 | 1.00 75.83 | C |
| ATOM | 1011 | CZ | PHE | A | 127 | 102.619 | 32.386 | 7.014 | 1.00 75.24 | C |
| ATOM | 1012 | CE2 | PHE | A | 127 | 102.064 | 33.159 | 6.012 | 1.00 75.56 | C |
| ATOM | 1013 | CD2 | PHE | A | 127 | 102.481 | 32.998 | 4.700 | 1.00 74.80 | C |
| ATOM | 1014 | C | PHE | A | 127 | 105.929 | 31.164 | 1.670 | 1.00 76.28 | C |
| ATOM | 1015 | O | PHE | A | 127 | 105.753 | 31.386 | 0.471 | 1.00 74.14 | O |
| ATOM | 1016 | N | THR | A | 128 | 106.567 | 30.093 | 2.139 | 1.00 75.69 | N |
| ATOM | 1017 | CA | THR | A | 128 | 107.117 | 29.044 | 1.291 | 1.00 74.38 | C |
| ATOM | 1018 | CB | THR | A | 128 | 108.643 | 28.953 | 1.456 | 1.00 74.05 | C |
| ATOM | 1019 | OG1 | THR | A | 128 | 109.244 | 30.200 | 1.090 | 1.00 74.56 | O |
| ATOM | 1020 | CG2 | THR | A | 128 | 109.220 | 27.834 | 0.598 | 1.00 74.60 | C |
| ATOM | 1021 | C | THR | A | 128 | 106.526 | 27.704 | 1.708 | 1.00 74.11 | C |
| ATOM | 1022 | O | THR | A | 128 | 106.618 | 27.328 | 2.875 | 1.00 74.98 | O |
| ATOM | 1023 | N | VAL | A | 129 | 105.926 | 26.987 | 0.761 | 1.00 72.30 | N |
| ATOM | 1024 | CA | VAL | A | 129 | 105.366 | 25.665 | 1.040 | 1.00 70.81 | C |
| ATOM | 1025 | CB | VAL | A | 129 | 104.408 | 25.200 | −0.075 | 1.00 69.00 | C |
| ATOM | 1026 | CG1 | VAL | A | 129 | 103.927 | 23.778 | 0.179 | 1.00 68.01 | C |
| ATOM | 1027 | CG2 | VAL | A | 129 | 103.226 | 26.152 | −0.189 | 1.00 69.56 | C |
| ATOM | 1028 | C | VAL | A | 129 | 106.525 | 24.682 | 1.177 | 1.00 72.26 | C |
| ATOM | 1029 | O | VAL | A | 129 | 107.254 | 24.448 | 0.211 | 1.00 74.48 | O |
| ATOM | 1030 | N | PHE | A | 130 | 106.701 | 24.129 | 2.378 | 1.00 71.94 | N |
| ATOM | 1031 | CA | PHE | A | 130 | 107.815 | 23.214 | 2.656 | 1.00 71.73 | C |
| ATOM | 1032 | CB | PHE | A | 130 | 108.617 | 23.687 | 3.876 | 1.00 73.22 | C |
| ATOM | 1033 | CG | PHE | A | 130 | 107.899 | 23.525 | 5.185 | 1.00 73.63 | C |
| ATOM | 1034 | CD1 | PHE | A | 130 | 107.111 | 24.548 | 5.692 | 1.00 74.85 | C |
| ATOM | 1035 | CE1 | PHE | A | 130 | 106.451 | 24.402 | 6.902 | 1.00 76.17 | C |
| ATOM | 1036 | CZ | PHE | A | 130 | 106.577 | 23.226 | 7.620 | 1.00 75.83 | C |
| ATOM | 1037 | CE2 | PHE | A | 130 | 107.363 | 22.200 | 7.125 | 1.00 75.44 | C |
| ATOM | 1038 | CD2 | PHE | A | 130 | 108.022 | 22.354 | 5.918 | 1.00 74.02 | C |
| ATOM | 1039 | C | PHE | A | 130 | 107.387 | 21.760 | 2.844 | 1.00 71.33 | C |
| ATOM | 1040 | O | PHE | A | 130 | 108.229 | 20.862 | 2.806 | 1.00 70.69 | O |
| ATOM | 1041 | N | SER | A | 131 | 106.094 | 21.525 | 3.053 | 1.00 70.85 | N |
| ATOM | 1042 | CA | SER | A | 131 | 105.587 | 20.165 | 3.209 | 1.00 71.30 | C |
| ATOM | 1043 | CB | SER | A | 131 | 105.675 | 19.720 | 4.669 | 1.00 73.51 | C |
| ATOM | 1044 | OG | SER | A | 131 | 105.301 | 18.360 | 4.805 | 1.00 74.55 | O |
| ATOM | 1045 | C | SER | A | 131 | 104.149 | 20.060 | 2.719 | 1.00 69.79 | C |
| ATOM | 1046 | O | SER | A | 131 | 103.352 | 20.980 | 2.901 | 1.00 67.79 | O |
| ATOM | 1047 | N | VAL | A | 132 | 103.834 | 18.924 | 2.103 | 1.00 70.57 | N |
| ATOM | 1048 | CA | VAL | A | 132 | 102.519 | 18.674 | 1.518 | 1.00 70.58 | C |
| ATOM | 1049 | CB | VAL | A | 132 | 102.561 | 18.867 | −0.020 | 1.00 69.36 | C |
| ATOM | 1050 | CG1 | VAL | A | 132 | 101.234 | 18.487 | −0.664 | 1.00 69.12 | C |
| ATOM | 1051 | CG2 | VAL | A | 132 | 102.931 | 20.302 | −0.365 | 1.00 69.14 | C |
| ATOM | 1052 | C | VAL | A | 132 | 102.070 | 17.248 | 1.819 | 1.00 70.20 | C |
| ATOM | 1053 | O | VAL | A | 132 | 102.897 | 16.343 | 1.946 | 1.00 72.95 | O |
| ATOM | 1054 | N | ASN | A | 133 | 100.760 | 17.067 | 1.957 | 1.00 68.16 | N |
| ATOM | 1055 | CA | ASN | A | 133 | 100.165 | 15.743 | 2.071 | 1.00 70.49 | C |
| ATOM | 1056 | CB | ASN | A | 133 | 99.778 | 15.417 | 3.514 | 1.00 72.09 | C |
| ATOM | 1057 | CG | ASN | A | 133 | 99.396 | 13.956 | 3.699 | 1.00 72.52 | C |
| ATOM | 1058 | OD1 | ASN | A | 133 | 98.600 | 13.412 | 2.932 | 1.00 72.44 | O |
| ATOM | 1059 | ND2 | ASN | A | 133 | 99.962 | 13.316 | 4.724 | 1.00 74.04 | N |
| ATOM | 1060 | C | ASN | A | 133 | 98.955 | 15.720 | 1.148 | 1.00 72.68 | C |
| ATOM | 1061 | O | ASN | A | 133 | 97.956 | 16.379 | 1.414 | 1.00 73.03 | O |
| ATOM | 1062 | N | GLN | A | 134 | 99.057 | 14.969 | 0.056 | 1.00 74.63 | N |
| ATOM | 1063 | CA | GLN | A | 134 | 98.015 | 14.954 | −0.969 | 1.00 74.97 | C |
| ATOM | 1064 | CB | GLN | A | 134 | 98.548 | 14.351 | −2.269 | 1.00 76.21 | C |
| ATOM | 1065 | CG | GLN | A | 134 | 99.637 | 15.191 | −2.909 | 1.00 77.57 | C |
| ATOM | 1066 | CD | GLN | A | 134 | 100.043 | 14.681 | −4.275 | 1.00 80.24 | C |
| ATOM | 1067 | OE1 | GLN | A | 134 | 99.592 | 13.623 | −4.718 | 1.00 82.97 | O |
| ATOM | 1068 | NE2 | GLN | A | 134 | 100.902 | 15.434 | −4.952 | 1.00 81.02 | N |
| ATOM | 1069 | C | GLN | A | 134 | 96.732 | 14.247 | −0.554 | 1.00 74.40 | C |
| ATOM | 1070 | O | GLN | A | 134 | 95.684 | 14.473 | −1.157 | 1.00 80.04 | O |
| ATOM | 1071 | N | THR | A | 135 | 96.812 | 13.384 | 0.452 | 1.00 74.31 | N |
| ATOM | 1072 | CA | THR | A | 135 | 95.631 | 12.701 | 0.965 | 1.00 77.03 | C |
| ATOM | 1073 | CB | THR | A | 135 | 95.939 | 11.235 | 1.327 | 1.00 75.26 | C |
| ATOM | 1074 | OG1 | THR | A | 135 | 96.708 | 10.634 | 0.280 | 1.00 74.35 | O |
| ATOM | 1075 | CG2 | THR | A | 135 | 94.652 | 10.444 | 1.522 | 1.00 75.12 | C |
| ATOM | 1076 | C | THR | A | 135 | 95.165 | 13.486 | 2.190 | 1.00 82.39 | C |
| ATOM | 1077 | O | THR | A | 135 | 95.116 | 12.959 | 3.307 | 1.00 82.05 | O |
| ATOM | 1078 | N | CYS | A | 136 | 94.824 | 14.756 | 1.972 | 1.00 84.83 | N |
| ATOM | 1079 | CA | CYS | A | 136 | 94.499 | 15.645 | 3.077 | 1.00 86.52 | C |
| ATOM | 1080 | CB | CYS | A | 136 | 95.175 | 16.990 | 2.920 | 1.00 86.38 | C |
| ATOM | 1081 | SG | CYS | A | 136 | 94.984 | 17.997 | 4.401 | 1.00 83.29 | S |
| ATOM | 1082 | C | CYS | A | 136 | 93.022 | 15.895 | 3.277 | 1.00 90.46 | C |
| ATOM | 1083 | O | CYS | A | 136 | 92.241 | 15.963 | 2.326 | 1.00 85.99 | O |
| ATOM | 1084 | N | VAL | A | 137 | 92.688 | 16.096 | 4.548 | 1.00 94.32 | N |
| ATOM | 1085 | CA | VAL | A | 137 | 91.324 | 16.244 | 5.012 | 1.00 87.90 | C |
| ATOM | 1086 | CB | VAL | A | 137 | 90.656 | 17.503 | 4.443 | 1.00 88.76 | C |
| ATOM | 1087 | CG1 | VAL | A | 137 | 89.238 | 17.622 | 4.957 | 1.00 94.38 | C |
| ATOM | 1088 | CG2 | VAL | A | 137 | 91.460 | 18.733 | 4.840 | 1.00 86.92 | C |
| ATOM | 1089 | C | VAL | A | 137 | 90.590 | 14.932 | 4.727 | 1.00 85.62 | C |
| ATOM | 1090 | O | VAL | A | 137 | 89.553 | 14.884 | 4.065 | 1.00 85.99 | O |
| ATOM | 1091 | N | TRP | A | 138 | 91.219 | 13.862 | 5.207 | 1.00 81.26 | N |
| ATOM | 1092 | CA | TRP | A | 138 | 90.633 | 12.533 | 5.250 | 1.00 80.06 | C |
| ATOM | 1093 | CB | TRP | A | 138 | 91.206 | 11.622 | 4.161 | 1.00 78.20 | C |
| ATOM | 1094 | CG | TRP | A | 138 | 90.416 | 10.367 | 4.023 | 1.00 74.91 | C |
| ATOM | 1095 | CD1 | TRP | A | 138 | 90.803 | 9.111 | 4.369 | 1.00 74.39 | C |
| ATOM | 1096 | NE1 | TRP | A | 138 | 89.790 | 8.218 | 4.115 | 1.00 76.74 | N |
| ATOM | 1097 | CE2 | TRP | A | 138 | 88.714 | 8.900 | 3.612 | 1.00 76.01 | C |
| ATOM | 1098 | CD2 | TRP | A | 138 | 89.073 | 10.259 | 3.545 | 1.00 74.70 | C |
| ATOM | 1099 | CE3 | TRP | A | 138 | 88.142 | 11.181 | 3.055 | 1.00 74.89 | C |
| ATOM | 1100 | CZ3 | TRP | A | 138 | 86.896 | 10.722 | 2.655 | 1.00 76.10 | C |
| ATOM | 1101 | CH2 | TRP | A | 138 | 86.567 | 9.361 | 2.733 | 1.00 77.44 | C |
| ATOM | 1102 | CZ2 | TRP | A | 138 | 87.460 | 8.436 | 3.207 | 1.00 77.43 | C |
| ATOM | 1103 | C | TRP | A | 138 | 90.904 | 11.958 | 6.643 | 1.00 80.76 | C |
| ATOM | 1104 | O | TRP | A | 138 | 89.991 | 11.459 | 7.303 | 1.00 79.36 | O |
| ATOM | 1105 | N | TYR | A | 139 | 92.166 | 12.034 | 7.071 | 1.00 82.51 | N |
| ATOM | 1106 | CA | TYR | A | 139 | 92.581 | 11.601 | 8.401 | 1.00 81.56 | C |
| ATOM | 1107 | CB | TYR | A | 139 | 93.916 | 10.861 | 8.339 | 1.00 80.89 | C |
| ATOM | 1108 | CG | TYR | A | 139 | 94.018 | 9.811 | 7.257 | 1.00 80.17 | C |
| ATOM | 1109 | CD1 | TYR | A | 139 | 93.330 | 8.606 | 7.359 | 1.00 80.27 | C |
| ATOM | 1110 | CE1 | TYR | A | 139 | 93.437 | 7.637 | 6.374 | 1.00 82.65 | C |
| ATOM | 1111 | CZ | TYR | A | 139 | 94.248 | 7.865 | 5.272 | 1.00 81.80 | C |
| ATOM | 1112 | OH | TYR | A | 139 | 94.359 | 6.909 | 4.287 | 1.00 81.17 | O |
| ATOM | 1113 | CE2 | TYR | A | 139 | 94.946 | 9.050 | 5.155 | 1.00 80.06 | C |
| ATOM | 1114 | CD2 | TYR | A | 139 | 94.832 | 10.011 | 6.144 | 1.00 79.78 | C |
| ATOM | 1115 | C | TYR | A | 139 | 92.750 | 12.826 | 9.296 | 1.00 81.52 | C |
| ATOM | 1116 | O | TYR | A | 139 | 93.288 | 13.844 | 8.860 | 1.00 82.26 | O |
| ATOM | 1117 | N | ARG | A | 140 | 92.303 | 12.717 | 10.546 | 1.00 81.61 | N |
| ATOM | 1118 | CA | ARG | A | 140 | 92.458 | 13.798 | 11.522 | 1.00 80.81 | C |
| ATOM | 1119 | CB | ARG | A | 140 | 91.682 | 13.503 | 12.802 | 1.00 80.92 | C |
| ATOM | 1120 | CG | ARG | A | 140 | 91.965 | 14.496 | 13.916 | 1.00 81.79 | C |
| ATOM | 1121 | CD | ARG | A | 140 | 90.903 | 14.448 | 14.997 | 1.00 85.18 | C |
| ATOM | 1122 | NE | ARG | A | 140 | 90.964 | 13.227 | 15.803 | 1.00 86.47 | N |
| ATOM | 1123 | CZ | ARG | A | 140 | 91.715 | 13.062 | 16.892 | 1.00 84.38 | C |
| ATOM | 1124 | NH1 | ARG | A | 140 | 91.672 | 11.905 | 17.545 | 1.00 85.39 | N |
| ATOM | 1125 | NH2 | ARG | A | 140 | 92.506 | 14.034 | 17.339 | 1.00 83.04 | N |
| ATOM | 1126 | C | ARG | A | 140 | 93.927 | 14.003 | 11.857 | 1.00 79.50 | C |
| ATOM | 1127 | O | ARG | A | 140 | 94.402 | 15.133 | 11.904 | 1.00 79.49 | O |
| ATOM | 1128 | N | PHE | A | 141 | 94.631 | 12.903 | 12.110 | 1.00 78.69 | N |
| ATOM | 1129 | CA | PHE | A | 141 | 96.063 | 12.951 | 12.362 | 1.00 78.46 | C |
| ATOM | 1130 | CB | PHE | A | 141 | 96.504 | 11.793 | 13.256 | 1.00 79.59 | C |
| ATOM | 1131 | CG | PHE | A | 141 | 95.953 | 11.862 | 14.650 | 1.00 80.07 | C |
| ATOM | 1132 | CD1 | PHE | A | 141 | 96.507 | 12.721 | 15.588 | 1.00 80.90 | C |
| ATOM | 1133 | CE1 | PHE | A | 141 | 96.005 | 12.785 | 16.875 | 1.00 83.01 | C |
| ATOM | 1134 | CZ | PHE | A | 141 | 94.941 | 11.980 | 17.240 | 1.00 82.71 | C |
| ATOM | 1135 | CE2 | PHE | A | 141 | 94.382 | 11.119 | 16.314 | 1.00 82.15 | C |
| ATOM | 1136 | CD2 | PHE | A | 141 | 94.888 | 11.062 | 15.029 | 1.00 81.39 | C |
| ATOM | 1137 | C | PHE | A | 141 | 96.782 | 12.898 | 11.022 | 1.00 77.36 | C |
| ATOM | 1138 | O | PHE | A | 141 | 97.094 | 11.821 | 10.518 | 1.00 77.71 | O |
| ATOM | 1139 | N | THR | A | 142 | 97.023 | 14.072 | 10.447 | 1.00 76.07 | N |
| ATOM | 1140 | CA | THR | A | 142 | 97.678 | 14.188 | 9.150 | 1.00 76.31 | C |
| ATOM | 1141 | CB | THR | A | 142 | 96.990 | 15.256 | 8.278 | 1.00 76.58 | C |
| ATOM | 1142 | OG1 | THR | A | 142 | 95.643 | 14.850 | 7.996 | 1.00 77.62 | O |
| ATOM | 1143 | CG2 | THR | A | 142 | 97.737 | 15.450 | 6.964 | 1.00 77.49 | C |
| ATOM | 1144 | C | THR | A | 142 | 99.147 | 14.545 | 9.334 | 1.00 76.47 | C |
| ATOM | 1145 | O | THR | A | 142 | 99.472 | 15.623 | 9.829 | 1.00 76.20 | O |
| ATOM | 1146 | N | ASP | A | 143 | 100.027 | 13.633 | 8.927 | 1.00 77.81 | N |
| ATOM | 1147 | CA | ASP | A | 143 | 101.468 | 13.842 | 9.038 | 1.00 78.46 | C |
| ATOM | 1148 | CB | ASP | A | 143 | 102.212 | 12.500 | 9.032 | 1.00 81.65 | C |
| ATOM | 1149 | CG | ASP | A | 143 | 101.879 | 11.633 | 10.239 | 1.00 83.58 | C |
| ATOM | 1150 | OD1 | ASP | A | 143 | 101.038 | 12.040 | 11.070 | 1.00 85.51 | O |
| ATOM | 1151 | OD2 | ASP | A | 143 | 102.467 | 10.539 | 10.354 | 1.00 84.80 | O |
| ATOM | 1152 | C | ASP | A | 143 | 101.993 | 14.713 | 7.903 | 1.00 74.97 | C |
| ATOM | 1153 | O | ASP | A | 143 | 101.479 | 14.670 | 6.785 | 1.00 77.14 | O |
| ATOM | 1154 | N | PHE | A | 144 | 103.021 | 15.498 | 8.210 | 1.00 71.79 | N |
| ATOM | 1155 | CA | PHE | A | 144 | 103.688 | 16.348 | 7.237 | 1.00 71.00 | C |
| ATOM | 1156 | CB | PHE | A | 144 | 103.379 | 17.820 | 7.507 | 1.00 69.59 | C |
| ATOM | 1157 | CG | PHE | A | 144 | 101.965 | 18.210 | 7.182 | 1.00 69.05 | C |
| ATOM | 1158 | CD1 | PHE | A | 144 | 100.983 | 18.185 | 8.158 | 1.00 69.25 | C |
| ATOM | 1159 | CE1 | PHE | A | 144 | 99.678 | 18.543 | 7.860 | 1.00 69.31 | C |
| ATOM | 1160 | CZ | PHE | A | 144 | 99.341 | 18.931 | 6.577 | 1.00 67.47 | C |
| ATOM | 1161 | CE2 | PHE | A | 144 | 100.311 | 18.962 | 5.593 | 1.00 67.42 | C |
| ATOM | 1162 | CD2 | PHE | A | 144 | 101.614 | 18.600 | 5.896 | 1.00 68.29 | C |
| ATOM | 1163 | C | PHE | A | 144 | 105.190 | 16.100 | 7.311 | 1.00 73.12 | C |
| ATOM | 1164 | O | PHE | A | 144 | 105.820 | 16.379 | 8.328 | 1.00 71.85 | O |
| ATOM | 1165 | N | GLN | A | 145 | 105.755 | 15.570 | 6.227 | 1.00 78.05 | N |
| ATOM | 1166 | CA | GLN | A | 145 | 107.182 | 15.262 | 6.170 | 1.00 77.96 | C |
| ATOM | 1167 | CB | GLN | A | 145 | 107.504 | 14.360 | 4.975 | 1.00 80.55 | C |
| ATOM | 1168 | CG | GLN | A | 145 | 106.704 | 13.064 | 4.915 | 1.00 82.81 | C |
| ATOM | 1169 | CD | GLN | A | 145 | 106.892 | 12.190 | 6.142 | 1.00 85.81 | C |
| ATOM | 1170 | OE1 | GLN | A | 145 | 107.994 | 12.091 | 6.688 | 1.00 87.14 | O |
| ATOM | 1171 | NE2 | GLN | A | 145 | 105.816 | 11.537 | 6.575 | 1.00 87.99 | N |
| ATOM | 1172 | C | GLN | A | 145 | 107.965 | 16.559 | 6.056 | 1.00 76.90 | C |
| ATOM | 1173 | O | GLN | A | 145 | 107.702 | 17.368 | 5.168 | 1.00 77.08 | O |
| ATOM | 1174 | N | VAL | A | 146 | 108.920 | 16.754 | 6.961 | 1.00 75.97 | N |
| ATOM | 1175 | CA | VAL | A | 146 | 109.732 | 17.964 | 6.982 | 1.00 75.59 | C |
| ATOM | 1176 | CB | VAL | A | 146 | 110.064 | 18.389 | 8.425 | 1.00 75.15 | C |
| ATOM | 1177 | CG1 | VAL | A | 146 | 110.804 | 19.720 | 8.442 | 1.00 75.39 | C |
| ATOM | 1178 | CG2 | VAL | A | 146 | 108.795 | 18.483 | 9.259 | 1.00 74.27 | C |
| ATOM | 1179 | C | VAL | A | 146 | 111.029 | 17.690 | 6.231 | 1.00 76.30 | C |
| ATOM | 1180 | O | VAL | A | 146 | 111.608 | 16.613 | 6.385 | 1.00 76.96 | O |
| ATOM | 1181 | N | PRO | A | 147 | 111.487 | 18.649 | 5.404 | 1.00 78.68 | N |
| ATOM | 1182 | CA | PRO | A | 147 | 112.755 | 18.431 | 4.701 | 1.00 81.84 | C |
| ATOM | 1183 | CB | PRO | A | 147 | 112.881 | 19.659 | 3.787 | 1.00 80.60 | C |
| ATOM | 1184 | CG | PRO | A | 147 | 111.513 | 20.234 | 3.693 | 1.00 79.84 | C |
| ATOM | 1185 | CD | PRO | A | 147 | 110.823 | 19.894 | 4.977 | 1.00 79.51 | C |
| ATOM | 1186 | C | PRO | A | 147 | 113.941 | 18.347 | 5.661 | 1.00 86.08 | C |
| ATOM | 1187 | O | PRO | A | 147 | 113.938 | 19.000 | 6.706 | 1.00 86.09 | O |
| ATOM | 1188 | N | GLU | A | 148 | 114.939 | 17.545 | 5.307 | 1.00 91.96 | N |
| ATOM | 1189 | CA | GLU | A | 148 | 116.120 | 17.368 | 6.150 | 1.00 97.64 | C |
| ATOM | 1190 | CB | GLU | A | 148 | 116.968 | 16.200 | 5.636 | 1.00101.74 | C |
| ATOM | 1191 | CG | GLU | A | 148 | 118.213 | 15.911 | 6.463 | 1.00107.11 | C |
| ATOM | 1192 | CD | GLU | A | 148 | 119.079 | 14.827 | 5.853 | 1.00113.41 | C |
| ATOM | 1193 | OE1 | GLU | A | 148 | 118.525 | 13.806 | 5.393 | 1.00120.31 | O |
| ATOM | 1194 | OE2 | GLU | A | 148 | 120.318 | 14.993 | 5.842 | 1.00118.08 | O |
| ATOM | 1195 | C | GLU | A | 148 | 116.971 | 18.637 | 6.223 | 1.00 98.80 | C |
| ATOM | 1196 | O | GLU | A | 148 | 117.497 | 18.973 | 7.284 | 1.00 99.60 | O |
| ATOM | 1197 | N | ARG | A | 149 | 117.092 | 19.345 | 5.102 | 1.00100.31 | N |
| ATOM | 1198 | CA | ARG | A | 149 | 117.944 | 20.535 | 5.026 | 1.00102.05 | C |
| ATOM | 1199 | CB | ARG | A | 149 | 118.496 | 20.691 | 3.602 | 1.00109.61 | C |
| ATOM | 1200 | CG | ARG | A | 149 | 119.367 | 19.534 | 3.140 | 1.00116.76 | C |
| ATOM | 1201 | CD | ARG | A | 149 | 119.858 | 19.745 | 1.716 | 1.00125.71 | C |
| ATOM | 1202 | NE | ARG | A | 149 | 120.677 | 18.626 | 1.239 | 1.00133.21 | N |
| ATOM | 1203 | CZ | ARG | A | 149 | 121.985 | 18.479 | 1.460 | 1.00133.30 | C |
| ATOM | 1204 | NH1 | ARG | A | 149 | 122.666 | 19.375 | 2.173 | 1.00135.95 | N |
| ATOM | 1205 | NH2 | ARG | A | 149 | 122.619 | 17.417 | 0.970 | 1.00129.76 | N |
| ATOM | 1206 | C | ARG | A | 149 | 117.261 | 21.839 | 5.471 | 1.00 96.88 | C |
| ATOM | 1207 | O | ARG | A | 149 | 117.676 | 22.925 | 5.055 | 1.00 95.81 | O |
| ATOM | 1208 | N | MET | A | 150 | 116.233 | 21.746 | 6.316 | 1.00 90.98 | N |
| ATOM | 1209 | CA | MET | A | 150 | 115.560 | 22.943 | 6.824 | 1.00 89.04 | C |
| ATOM | 1210 | CB | MET | A | 150 | 114.235 | 22.594 | 7.512 | 1.00 88.98 | C |
| ATOM | 1211 | CG | MET | A | 150 | 113.137 | 22.104 | 6.582 | 1.00 89.61 | C |
| ATOM | 1212 | SD | MET | A | 150 | 112.456 | 23.348 | 5.461 | 1.00 90.13 | S |
| ATOM | 1213 | CE | MET | A | 150 | 111.604 | 24.446 | 6.585 | 1.00 88.70 | C |
| ATOM | 1214 | C | MET | A | 150 | 116.472 | 23.659 | 7.818 | 1.00 88.28 | C |
| ATOM | 1215 | O | MET | A | 150 | 116.906 | 23.054 | 8.797 | 1.00 89.76 | O |
| ATOM | 1216 | N | PRO | A | 151 | 116.768 | 24.949 | 7.570 | 1.00 87.50 | N |
| ATOM | 1217 | CA | PRO | A | 151 | 117.634 | 25.717 | 8.464 | 1.00 88.75 | C |
| ATOM | 1218 | CB | PRO | A | 151 | 117.986 | 26.949 | 7.630 | 1.00 87.34 | C |
| ATOM | 1219 | CG | PRO | A | 151 | 116.809 | 27.138 | 6.747 | 1.00 87.38 | C |
| ATOM | 1220 | CD | PRO | A | 151 | 116.305 | 25.760 | 6.430 | 1.00 87.98 | C |
| ATOM | 1221 | C | PRO | A | 151 | 116.922 | 26.131 | 9.753 | 1.00 90.96 | C |
| ATOM | 1222 | O | PRO | A | 151 | 115.692 | 26.071 | 9.816 | 1.00 91.16 | O |
| ATOM | 1223 | N | PRO | A | 152 | 117.688 | 26.558 | 10.775 | 1.00 91.63 | N |
| ATOM | 1224 | CA | PRO | A | 152 | 117.083 | 26.959 | 12.038 | 1.00 91.53 | C |
| ATOM | 1225 | CB | PRO | A | 152 | 118.278 | 27.020 | 12.986 | 1.00 92.45 | C |
| ATOM | 1226 | CG | PRO | A | 152 | 119.412 | 27.417 | 12.110 | 1.00 92.47 | C |
| ATOM | 1227 | CD | PRO | A | 152 | 119.150 | 26.757 | 10.786 | 1.00 92.22 | C |
| ATOM | 1228 | C | PRO | A | 152 | 116.417 | 28.324 | 11.934 | 1.00 91.29 | C |
| ATOM | 1229 | O | PRO | A | 152 | 116.912 | 29.203 | 11.227 | 1.00 87.61 | O |
| ATOM | 1230 | N | CYS | A | 153 | 115.303 | 28.490 | 12.638 | 1.00 95.22 | N |
| ATOM | 1231 | CA | CYS | A | 153 | 114.562 | 29.747 | 12.624 | 1.00100.58 | C |
| ATOM | 1232 | CB | CYS | A | 153 | 113.097 | 29.506 | 13.001 | 1.00105.48 | C |
| ATOM | 1233 | SG | CYS | A | 153 | 112.131 | 28.865 | 11.616 | 1.00110.80 | S |
| ATOM | 1234 | C | CYS | A | 153 | 115.197 | 30.783 | 13.546 | 1.00 99.11 | C |
| ATOM | 1235 | O | CYS | A | 153 | 115.962 | 30.426 | 14.440 | 1.00100.03 | O |
| ATOM | 1236 | N | PRO | A | 154 | 114.883 | 32.075 | 13.328 | 1.00 99.89 | N |
| ATOM | 1237 | CA | PRO | A | 154 | 115.453 | 33.146 | 14.152 | 1.00103.15 | C |
| ATOM | 1238 | CB | PRO | A | 154 | 114.838 | 34.426 | 13.556 | 1.00102.45 | C |
| ATOM | 1239 | CG | PRO | A | 154 | 113.722 | 33.979 | 12.679 | 1.00101.24 | C |
| ATOM | 1240 | CD | PRO | A | 154 | 114.096 | 32.614 | 12.206 | 1.00101.08 | C |
| ATOM | 1241 | C | PRO | A | 154 | 115.136 | 33.030 | 15.655 | 1.00106.30 | C |
| ATOM | 1242 | O | PRO | A | 154 | 114.291 | 32.223 | 16.043 | 1.00103.59 | O |
| ATOM | 1243 | N | PRO | A | 155 | 115.820 | 33.835 | 16.494 | 1.00109.83 | N |
| ATOM | 1244 | CA | PRO | A | 155 | 115.691 | 33.817 | 17.956 | 1.00112.03 | C |
| ATOM | 1245 | CB | PRO | A | 155 | 116.332 | 35.143 | 18.369 | 1.00114.04 | C |
| ATOM | 1246 | CG | PRO | A | 155 | 117.382 | 35.371 | 17.341 | 1.00113.75 | C |
| ATOM | 1247 | CD | PRO | A | 155 | 116.841 | 34.809 | 16.056 | 1.00111.64 | C |
| ATOM | 1248 | C | PRO | A | 155 | 114.265 | 33.733 | 18.509 | 1.00111.44 | C |
| ATOM | 1249 | O | PRO | A | 155 | 114.016 | 32.954 | 19.434 | 1.00112.99 | O |
| ATOM | 1250 | N | GLY | A | 156 | 113.345 | 34.518 | 17.951 | 1.00108.40 | N |
| ATOM | 1251 | CA | GLY | A | 156 | 111.954 | 34.533 | 18.417 | 1.00107.86 | C |
| ATOM | 1252 | C | GLY | A | 156 | 111.032 | 33.536 | 17.729 | 1.00106.15 | C |
| ATOM | 1253 | O | GLY | A | 156 | 109.811 | 33.688 | 17.773 | 1.00101.23 | O |
| ATOM | 1254 | N | GLY | A | 157 | 111.609 | 32.511 | 17.105 | 1.00106.69 | N |
| ATOM | 1255 | CA | GLY | A | 157 | 110.837 | 31.524 | 16.355 | 1.00104.57 | C |
| ATOM | 1256 | C | GLY | A | 157 | 110.410 | 32.056 | 14.998 | 1.00101.90 | C |
| ATOM | 1257 | O | GLY | A | 157 | 110.762 | 33.174 | 14.618 | 1.00104.11 | O |
| ATOM | 1258 | N | CYS | A | 158 | 109.652 | 31.247 | 14.266 | 1.00 98.35 | N |
| ATOM | 1259 | CA | CYS | A | 158 | 109.128 | 31.629 | 12.955 | 1.00 98.02 | C |
| ATOM | 1260 | CB | CYS | A | 158 | 109.898 | 30.915 | 11.840 | 1.00 99.92 | C |
| ATOM | 1261 | SG | CYS | A | 158 | 110.210 | 29.171 | 12.180 | 1.00111.88 | S |
| ATOM | 1262 | C | CYS | A | 158 | 107.637 | 31.313 | 12.888 | 1.00 93.44 | C |
| ATOM | 1263 | O | CYS | A | 158 | 107.072 | 30.765 | 13.837 | 1.00 94.72 | O |
| ATOM | 1264 | N | HIS | A | 159 | 107.001 | 31.678 | 11.779 | 1.00 89.00 | N |
| ATOM | 1265 | CA | HIS | A | 159 | 105.567 | 31.461 | 11.600 | 1.00 86.06 | C |
| ATOM | 1266 | CB | HIS | A | 159 | 104.862 | 32.797 | 11.384 | 1.00 86.11 | C |
| ATOM | 1267 | CG | HIS | A | 159 | 104.714 | 33.602 | 12.634 | 1.00 85.86 | C |
| ATOM | 1268 | ND1 | HIS | A | 159 | 105.771 | 34.260 | 13.224 | 1.00 85.50 | N |
| ATOM | 1269 | CE1 | HIS | A | 159 | 105.349 | 34.875 | 14.314 | 1.00 87.12 | C |
| ATOM | 1270 | NE2 | HIS | A | 159 | 104.056 | 34.642 | 14.451 | 1.00 85.96 | N |
| ATOM | 1271 | CD2 | HIS | A | 159 | 103.634 | 33.850 | 13.412 | 1.00 86.34 | C |
| ATOM | 1272 | C | HIS | A | 159 | 105.258 | 30.514 | 10.447 | 1.00 83.23 | C |
| ATOM | 1273 | O | HIS | A | 159 | 105.976 | 30.474 | 9.448 | 1.00 81.54 | O |
| ATOM | 1274 | N | CYS | A | 160 | 104.185 | 29.746 | 10.617 | 1.00 80.95 | N |
| ATOM | 1275 | CA | CYS | A | 160 | 103.723 | 28.792 | 9.620 | 1.00 80.06 | C |
| ATOM | 1276 | CB | CYS | A | 160 | 103.953 | 27.364 | 10.107 | 1.00 82.18 | C |
| ATOM | 1277 | SG | CYS | A | 160 | 105.680 | 26.861 | 10.200 | 1.00 88.40 | S |
| ATOM | 1278 | C | CYS | A | 160 | 102.240 | 28.985 | 9.361 | 1.00 78.26 | C |
| ATOM | 1279 | O | CYS | A | 160 | 101.560 | 29.715 | 10.086 | 1.00 76.85 | O |
| ATOM | 1280 | N | ALA | A | 161 | 101.743 | 28.316 | 8.325 | 1.00 76.08 | N |
| ATOM | 1281 | CA | ALA | A | 161 | 100.328 | 28.373 | 7.982 | 1.00 73.79 | C |
| ATOM | 1282 | CB | ALA | A | 161 | 100.049 | 29.573 | 7.094 | 1.00 73.37 | C |
| ATOM | 1283 | C | ALA | A | 161 | 99.888 | 27.088 | 7.295 | 1.00 71.21 | C |
| ATOM | 1284 | O | ALA | A | 161 | 100.631 | 26.515 | 6.500 | 1.00 72.15 | O |
| ATOM | 1285 | N | TRP | A | 162 | 98.683 | 26.637 | 7.625 | 1.00 69.66 | N |
| ATOM | 1286 | CA | TRP | A | 162 | 98.103 | 25.446 | 7.022 | 1.00 68.18 | C |
| ATOM | 1287 | CB | TRP | A | 162 | 97.434 | 24.575 | 8.083 | 1.00 66.71 | C |
| ATOM | 1288 | CG | TRP | A | 162 | 96.645 | 23.429 | 7.522 | 1.00 64.24 | C |
| ATOM | 1289 | CD1 | TRP | A | 162 | 97.124 | 22.215 | 7.134 | 1.00 63.87 | C |
| ATOM | 1290 | NE1 | TRP | A | 162 | 96.099 | 21.421 | 6.686 | 1.00 62.98 | N |
| ATOM | 1291 | CE2 | TRP | A | 162 | 94.926 | 22.119 | 6.779 | 1.00 62.55 | C |
| ATOM | 1292 | CD2 | TRP | A | 162 | 95.232 | 23.390 | 7.304 | 1.00 62.40 | C |
| ATOM | 1293 | CE3 | TRP | A | 162 | 94.194 | 24.305 | 7.505 | 1.00 63.45 | C |
| ATOM | 1294 | CZ3 | TRP | A | 162 | 92.898 | 23.927 | 7.176 | 1.00 64.28 | C |
| ATOM | 1295 | CH2 | TRP | A | 162 | 92.626 | 22.655 | 6.651 | 1.00 64.06 | C |
| ATOM | 1296 | CZ2 | TRP | A | 162 | 93.625 | 21.739 | 6.448 | 1.00 63.81 | C |
| ATOM | 1297 | C | TRP | A | 162 | 97.083 | 25.886 | 5.988 | 1.00 67.79 | C |
| ATOM | 1298 | O | TRP | A | 162 | 96.149 | 26.617 | 6.309 | 1.00 67.02 | O |
| ATOM | 1299 | N | PHE | A | 163 | 97.266 | 25.439 | 4.750 | 1.00 67.99 | N |
| ATOM | 1300 | CA | PHE | A | 163 | 96.366 | 25.793 | 3.657 | 1.00 65.65 | C |
| ATOM | 1301 | CB | PHE | A | 163 | 97.140 | 26.477 | 2.538 | 1.00 62.67 | C |
| ATOM | 1302 | CG | PHE | A | 163 | 97.753 | 27.784 | 2.935 | 1.00 59.96 | C |
| ATOM | 1303 | CD1 | PHE | A | 163 | 99.060 | 27.843 | 3.393 | 1.00 57.85 | C |
| ATOM | 1304 | CE1 | PHE | A | 163 | 99.628 | 29.055 | 3.745 | 1.00 58.02 | C |
| ATOM | 1305 | CZ | PHE | A | 163 | 98.889 | 30.224 | 3.644 | 1.00 58.37 | C |
| ATOM | 1306 | CE2 | PHE | A | 163 | 97.584 | 30.177 | 3.188 | 1.00 58.49 | C |
| ATOM | 1307 | CD2 | PHE | A | 163 | 97.023 | 28.961 | 2.836 | 1.00 59.66 | C |
| ATOM | 1308 | C | PHE | A | 163 | 95.669 | 24.561 | 3.102 | 1.00 65.65 | C |
| ATOM | 1309 | O | PHE | A | 163 | 96.204 | 23.462 | 3.175 | 1.00 64.46 | O |
| ATOM | 1310 | N | TRP | A | 164 | 94.484 | 24.762 | 2.530 | 1.00 66.84 | N |
| ATOM | 1311 | CA | TRP | A | 164 | 93.709 | 23.672 | 1.948 | 1.00 66.24 | C |
| ATOM | 1312 | CB | TRP | A | 164 | 93.004 | 22.883 | 3.057 | 1.00 66.14 | C |
| ATOM | 1313 | CG | TRP | A | 164 | 92.024 | 21.853 | 2.559 | 1.00 64.96 | C |
| ATOM | 1314 | CD1 | TRP | A | 164 | 92.256 | 20.891 | 1.626 | 1.00 64.45 | C |
| ATOM | 1315 | NE1 | TRP | A | 164 | 91.129 | 20.124 | 1.444 | 1.00 64.11 | N |
| ATOM | 1316 | CE2 | TRP | A | 164 | 90.145 | 20.573 | 2.282 | 1.00 62.02 | C |
| ATOM | 1317 | CD2 | TRP | A | 164 | 90.674 | 21.661 | 3.006 | 1.00 64.79 | C |
| ATOM | 1318 | CE3 | TRP | A | 164 | 89.861 | 22.304 | 3.948 | 1.00 66.51 | C |
| ATOM | 1319 | CZ3 | TRP | A | 164 | 88.559 | 21.847 | 4.128 | 1.00 64.78 | C |
| ATOM | 1320 | CH2 | TRP | A | 164 | 88.065 | 20.761 | 3.389 | 1.00 61.88 | C |
| ATOM | 1321 | CZ2 | TRP | A | 164 | 88.842 | 20.112 | 2.467 | 1.00 60.20 | C |
| ATOM | 1322 | C | TRP | A | 164 | 92.684 | 24.174 | 0.937 | 1.00 65.82 | C |
| ATOM | 1323 | O | TRP | A | 164 | 91.957 | 25.128 | 1.202 | 1.00 65.09 | O |
| ATOM | 1324 | N | ILE | A | 165 | 92.654 | 23.531 | −0.227 | 1.00 68.37 | N |
| ATOM | 1325 | CA | ILE | A | 165 | 91.652 | 23.797 | −1.261 | 1.00 68.83 | C |
| ATOM | 1326 | CB | ILE | A | 165 | 92.247 | 24.510 | −2.490 | 1.00 67.90 | C |
| ATOM | 1327 | CG1 | ILE | A | 165 | 92.757 | 25.897 | −2.091 | 1.00 67.98 | C |
| ATOM | 1328 | CD1 | ILE | A | 165 | 93.456 | 26.654 | −3.199 | 1.00 69.23 | C |
| ATOM | 1329 | CG2 | ILE | A | 165 | 91.195 | 24.624 | −3.588 | 1.00 69.24 | C |
| ATOM | 1330 | C | ILE | A | 165 | 91.067 | 22.451 | −1.664 | 1.00 68.08 | C |
| ATOM | 1331 | O | ILE | A | 165 | 91.808 | 21.515 | −1.962 | 1.00 69.04 | O |
| ATOM | 1332 | N | HIS | A | 166 | 89.742 | 22.366 | −1.682 | 1.00 68.83 | N |
| ATOM | 1333 | CA | HIS | A | 166 | 89.053 | 21.102 | −1.927 | 1.00 71.85 | C |
| ATOM | 1334 | CB | HIS | A | 166 | 87.972 | 20.887 | −0.864 | 1.00 71.51 | C |
| ATOM | 1335 | CG | HIS | A | 166 | 86.856 | 21.878 | −0.925 | 1.00 72.05 | C |
| ATOM | 1336 | ND1 | HIS | A | 166 | 85.731 | 21.680 | −1.693 | 1.00 75.81 | N |
| ATOM | 1337 | CE1 | HIS | A | 166 | 84.918 | 22.711 | −1.555 | 1.00 76.33 | C |
| ATOM | 1338 | NE2 | HIS | A | 166 | 85.478 | 23.574 | −0.727 | 1.00 75.99 | N |
| ATOM | 1339 | CD2 | HIS | A | 166 | 86.693 | 23.077 | −0.320 | 1.00 74.28 | C |
| ATOM | 1340 | C | HIS | A | 166 | 88.446 | 21.000 | −3.331 | 1.00 73.81 | C |
| ATOM | 1341 | O | HIS | A | 166 | 88.442 | 21.962 | −4.096 | 1.00 72.40 | O |
| ATOM | 1342 | N | SER | A | 167 | 87.926 | 19.817 | −3.644 | 1.00 75.65 | N |
| ATOM | 1343 | CA | SER | A | 167 | 87.361 | 19.532 | −4.953 | 1.00 78.55 | C |
| ATOM | 1344 | CB | SER | A | 167 | 87.006 | 18.047 | −5.042 | 1.00 82.08 | C |
| ATOM | 1345 | OG | SER | A | 167 | 86.547 | 17.699 | −6.338 | 1.00 86.01 | O |
| ATOM | 1346 | C | SER | A | 167 | 86.119 | 20.376 | −5.238 | 1.00 78.52 | C |
| ATOM | 1347 | O | SER | A | 167 | 85.413 | 20.761 | −4.307 | 1.00 79.61 | O |
| ATOM | 1348 | N | PRO | A | 168 | 85.852 | 20.677 | −6.526 | 1.00 78.75 | N |
| ATOM | 1349 | CA | PRO | A | 168 | 84.641 | 21.425 | −6.881 | 1.00 78.88 | C |
| ATOM | 1350 | CB | PRO | A | 168 | 84.899 | 21.874 | −8.326 | 1.00 78.02 | C |
| ATOM | 1351 | CG | PRO | A | 168 | 86.326 | 21.574 | −8.603 | 1.00 78.49 | C |
| ATOM | 1352 | CD | PRO | A | 168 | 86.703 | 20.447 | −7.704 | 1.00 78.14 | C |
| ATOM | 1353 | C | PRO | A | 168 | 83.367 | 20.588 | −6.835 | 1.00 80.35 | C |
| ATOM | 1354 | O | PRO | A | 168 | 82.273 | 21.146 | −6.742 | 1.00 81.61 | O |
| ATOM | 1355 | N | ASP | A | 169 | 83.510 | 19.266 | −6.896 | 1.00 80.49 | N |
| ATOM | 1356 | CA | ASP | A | 169 | 82.354 | 18.370 | −6.936 | 1.00 82.01 | C |
| ATOM | 1357 | CB | ASP | A | 169 | 82.756 | 17.006 | −7.512 | 1.00 84.94 | C |
| ATOM | 1358 | CG | ASP | A | 169 | 83.697 | 16.240 | −6.606 | 1.00 87.76 | C |
| ATOM | 1359 | OD1 | ASP | A | 169 | 84.189 | 16.833 | −5.623 | 1.00 92.11 | O |
| ATOM | 1360 | OD2 | ASP | A | 169 | 83.948 | 15.046 | −6.877 | 1.00 90.58 | O |
| ATOM | 1361 | C | ASP | A | 169 | 81.649 | 18.172 | −5.591 | 1.00 80.62 | C |
| ATOM | 1362 | O | ASP | A | 169 | 80.693 | 17.404 | −5.515 | 1.00 80.75 | O |
| ATOM | 1363 | N | SER | A | 170 | 82.113 | 18.839 | −4.536 | 1.00 81.20 | N |
| ATOM | 1364 | CA | SER | A | 170 | 81.455 | 18.742 | −3.232 | 1.00 83.20 | C |
| ATOM | 1365 | CB | SER | A | 170 | 82.153 | 17.712 | −2.346 | 1.00 82.93 | C |
| ATOM | 1366 | OG | SER | A | 170 | 81.443 | 17.534 | −1.132 | 1.00 81.13 | O |
| ATOM | 1367 | C | SER | A | 170 | 81.408 | 20.090 | −2.522 | 1.00 83.08 | C |
| ATOM | 1368 | O | SER | A | 170 | 82.436 | 20.610 | −2.093 | 1.00 82.18 | O |
| ATOM | 1369 | N | GLY | A | 171 | 80.206 | 20.652 | −2.424 | 1.00 84.35 | N |
| ATOM | 1370 | CA | GLY | A | 171 | 79.982 | 21.907 | −1.718 | 1.00 86.21 | C |
| ATOM | 1371 | C | GLY | A | 171 | 80.536 | 23.145 | −2.399 | 1.00 85.87 | C |
| ATOM | 1372 | O | GLY | A | 171 | 81.128 | 23.072 | −3.480 | 1.00 85.11 | O |
| ATOM | 1373 | N | GLY | A | 172 | 80.336 | 24.287 | −1.745 | 1.00 86.38 | N |
| ATOM | 1374 | CA | GLY | A | 172 | 80.805 | 25.574 | −2.247 | 1.00 84.86 | C |
| ATOM | 1375 | C | GLY | A | 172 | 82.315 | 25.662 | −2.207 | 1.00 81.45 | C |
| ATOM | 1376 | O | GLY | A | 172 | 82.956 | 25.046 | −1.357 | 1.00 81.76 | O |
| ATOM | 1377 | N | GLU | A | 173 | 82.880 | 26.442 | −3.120 | 1.00 79.46 | N |
| ATOM | 1378 | CA | GLU | A | 173 | 84.325 | 26.534 | −3.252 | 1.00 80.95 | C |
| ATOM | 1379 | CB | GLU | A | 173 | 84.712 | 26.590 | −4.732 | 1.00 82.70 | C |
| ATOM | 1380 | CG | GLU | A | 173 | 86.160 | 26.234 | −5.000 | 1.00 83.57 | C |
| ATOM | 1381 | CD | GLU | A | 173 | 86.508 | 24.803 | −4.637 | 1.00 86.05 | C |
| ATOM | 1382 | OE1 | GLU | A | 173 | 85.904 | 23.875 | −5.209 | 1.00 88.87 | O |
| ATOM | 1383 | OE2 | GLU | A | 173 | 87.404 | 24.608 | −3.790 | 1.00 89.20 | O |
| ATOM | 1384 | C | GLU | A | 173 | 84.884 | 27.736 | −2.490 | 1.00 80.15 | C |
| ATOM | 1385 | O | GLU | A | 173 | 84.255 | 28.792 | −2.417 | 1.00 77.51 | O |
| ATOM | 1386 | N | GLN | A | 174 | 86.078 | 27.551 | −1.931 | 1.00 81.16 | N |
| ATOM | 1387 | CA | GLN | A | 174 | 86.731 | 28.546 | −1.085 | 1.00 78.07 | C |
| ATOM | 1388 | CB | GLN | A | 174 | 85.999 | 28.623 | 0.256 | 1.00 76.04 | C |
| ATOM | 1389 | CG | GLN | A | 174 | 85.976 | 27.296 | 1.025 | 1.00 75.95 | C |
| ATOM | 1390 | CD | GLN | A | 174 | 84.636 | 26.569 | 1.067 | 1.00 75.19 | C |
| ATOM | 1391 | OE1 | GLN | A | 174 | 84.600 | 25.370 | 1.346 | 1.00 78.72 | O |
| ATOM | 1392 | NE2 | GLN | A | 174 | 83.537 | 27.274 | 0.816 | 1.00 75.03 | N |
| ATOM | 1393 | C | GLN | A | 174 | 88.182 | 28.127 | −0.826 | 1.00 78.53 | C |
| ATOM | 1394 | O | GLN | A | 174 | 88.661 | 27.152 | −1.412 | 1.00 83.04 | O |
| ATOM | 1395 | N | ILE | A | 175 | 88.873 | 28.855 | 0.051 | 1.00 76.75 | N |
| ATOM | 1396 | CA | ILE | A | 175 | 90.186 | 28.425 | 0.545 | 1.00 76.57 | C |
| ATOM | 1397 | CB | ILE | A | 175 | 91.345 | 29.321 | 0.057 | 1.00 76.05 | C |
| ATOM | 1398 | CG1 | ILE | A | 175 | 91.363 | 30.668 | 0.788 | 1.00 75.36 | C |
| ATOM | 1399 | CD1 | ILE | A | 175 | 92.640 | 31.458 | 0.585 | 1.00 74.21 | C |
| ATOM | 1400 | CG2 | ILE | A | 175 | 91.255 | 29.508 | −1.451 | 1.00 77.02 | C |
| ATOM | 1401 | C | ILE | A | 175 | 90.161 | 28.378 | 2.070 | 1.00 76.43 | C |
| ATOM | 1402 | O | ILE | A | 175 | 89.246 | 28.910 | 2.700 | 1.00 77.44 | O |
| ATOM | 1403 | N | TYR | A | 176 | 91.169 | 27.731 | 2.649 | 1.00 77.61 | N |
| ATOM | 1404 | CA | TYR | A | 176 | 91.292 | 27.601 | 4.101 | 1.00 77.05 | C |
| ATOM | 1405 | CB | TYR | A | 176 | 90.988 | 26.167 | 4.531 | 1.00 76.53 | C |
| ATOM | 1406 | CG | TYR | A | 176 | 89.539 | 25.780 | 4.364 | 1.00 76.89 | C |
| ATOM | 1407 | CD1 | TYR | A | 176 | 89.042 | 25.385 | 3.130 | 1.00 77.36 | C |
| ATOM | 1408 | CE1 | TYR | A | 176 | 87.714 | 25.029 | 2.976 | 1.00 77.51 | C |
| ATOM | 1409 | CZ | TYR | A | 176 | 86.865 | 25.067 | 4.063 | 1.00 76.94 | C |
| ATOM | 1410 | OH | TYR | A | 176 | 85.547 | 24.719 | 3.911 | 1.00 79.19 | O |
| ATOM | 1411 | CE2 | TYR | A | 176 | 87.332 | 25.456 | 5.299 | 1.00 76.88 | C |
| ATOM | 1412 | CD2 | TYR | A | 176 | 88.663 | 25.809 | 5.445 | 1.00 78.44 | C |
| ATOM | 1413 | C | TYR | A | 176 | 92.696 | 27.988 | 4.546 | 1.00 77.51 | C |
| ATOM | 1414 | O | TYR | A | 176 | 93.678 | 27.575 | 3.932 | 1.00 75.08 | O |
| ATOM | 1415 | N | MET | A | 177 | 92.784 | 28.783 | 5.610 | 1.00 80.01 | N |
| ATOM | 1416 | CA | MET | A | 177 | 94.074 | 29.209 | 6.154 | 1.00 77.80 | C |
| ATOM | 1417 | CB | MET | A | 177 | 94.488 | 30.561 | 5.577 | 1.00 78.47 | C |
| ATOM | 1418 | CG | MET | A | 177 | 95.844 | 31.040 | 6.070 | 1.00 77.88 | C |
| ATOM | 1419 | SD | MET | A | 177 | 96.289 | 32.658 | 5.429 | 1.00 79.52 | S |
| ATOM | 1420 | CE | MET | A | 177 | 97.914 | 32.856 | 6.160 | 1.00 79.52 | C |
| ATOM | 1421 | C | MET | A | 177 | 94.028 | 29.309 | 7.674 | 1.00 74.45 | C |
| ATOM | 1422 | O | MET | A | 177 | 93.098 | 29.879 | 8.239 | 1.00 73.78 | O |
| ATOM | 1423 | N | ASN | A | 178 | 95.050 | 28.750 | 8.317 | 1.00 72.63 | N |
| ATOM | 1424 | CA | ASN | A | 178 | 95.213 | 28.808 | 9.762 | 1.00 68.48 | C |
| ATOM | 1425 | CB | ASN | A | 178 | 94.727 | 27.510 | 10.410 | 1.00 68.50 | C |
| ATOM | 1426 | CG | ASN | A | 178 | 93.215 | 27.425 | 10.498 | 1.00 71.24 | C |
| ATOM | 1427 | OD1 | ASN | A | 178 | 92.615 | 26.424 | 10.105 | 1.00 73.50 | O |
| ATOM | 1428 | ND2 | ASN | A | 178 | 92.588 | 28.473 | 11.026 | 1.00 72.32 | N |
| ATOM | 1429 | C | ASN | A | 178 | 96.680 | 29.037 | 10.096 | 1.00 68.03 | C |
| ATOM | 1430 | O | ASN | A | 178 | 97.489 | 28.114 | 10.006 | 1.00 67.06 | O |
| ATOM | 1431 | N | GLY | A | 179 | 97.022 | 30.268 | 10.467 | 1.00 69.28 | N |
| ATOM | 1432 | CA | GLY | A | 179 | 98.395 | 30.613 | 10.837 | 1.00 72.01 | C |
| ATOM | 1433 | C | GLY | A | 179 | 98.720 | 30.206 | 12.266 | 1.00 75.63 | C |
| ATOM | 1434 | O | GLY | A | 179 | 97.822 | 30.091 | 13.108 | 1.00 76.00 | O |
| ATOM | 1435 | N | PHE | A | 180 | 100.002 | 29.988 | 12.547 | 1.00 76.09 | N |
| ATOM | 1436 | CA | PHE | A | 180 | 100.427 | 29.579 | 13.883 | 1.00 77.15 | C |
| ATOM | 1437 | CB | PHE | A | 180 | 100.021 | 28.126 | 14.143 | 1.00 78.41 | C |
| ATOM | 1438 | CG | PHE | A | 180 | 100.700 | 27.136 | 13.248 | 1.00 79.99 | C |
| ATOM | 1439 | CD1 | PHE | A | 180 | 100.268 | 26.952 | 11.945 | 1.00 79.59 | C |
| ATOM | 1440 | CE1 | PHE | A | 180 | 100.890 | 26.037 | 11.122 | 1.00 77.76 | C |
| ATOM | 1441 | CZ | PHE | A | 180 | 101.948 | 25.286 | 11.600 | 1.00 76.93 | C |
| ATOM | 1442 | CE2 | PHE | A | 180 | 102.382 | 25.456 | 12.892 | 1.00 79.56 | C |
| ATOM | 1443 | CD2 | PHE | A | 180 | 101.759 | 26.375 | 13.714 | 1.00 80.71 | C |
| ATOM | 1444 | C | PHE | A | 180 | 101.930 | 29.735 | 14.095 | 1.00 79.15 | C |
| ATOM | 1445 | O | PHE | A | 180 | 102.698 | 29.831 | 13.136 | 1.00 78.34 | O |
| ATOM | 1446 | N | GLN | A | 181 | 102.337 | 29.759 | 15.363 | 1.00 81.63 | N |
| ATOM | 1447 | CA | GLN | A | 181 | 103.748 | 29.842 | 15.723 | 1.00 82.49 | C |
| ATOM | 1448 | CB | GLN | A | 181 | 103.922 | 30.335 | 17.160 | 1.00 85.63 | C |
| ATOM | 1449 | CG | GLN | A | 181 | 103.581 | 31.800 | 17.376 | 1.00 87.98 | C |
| ATOM | 1450 | CD | GLN | A | 181 | 103.922 | 32.281 | 18.779 | 1.00 87.06 | C |
| ATOM | 1451 | OE1 | GLN | A | 181 | 104.026 | 31.487 | 19.715 | 1.00 84.92 | O |
| ATOM | 1452 | NE2 | GLN | A | 181 | 104.095 | 33.590 | 18.928 | 1.00 86.83 | N |
| ATOM | 1453 | C | GLN | A | 181 | 104.379 | 28.467 | 15.588 | 1.00 82.49 | C |
| ATOM | 1454 | O | GLN | A | 181 | 103.909 | 27.505 | 16.197 | 1.00 81.59 | O |
| ATOM | 1455 | N | CYS | A | 182 | 105.438 | 28.377 | 14.789 | 1.00 82.33 | N |
| ATOM | 1456 | CA | CYS | A | 182 | 106.152 | 27.123 | 14.597 | 1.00 82.80 | C |
| ATOM | 1457 | CB | CYS | A | 182 | 105.926 | 26.586 | 13.189 | 1.00 85.08 | C |
| ATOM | 1458 | SG | CYS | A | 182 | 106.333 | 27.768 | 11.889 | 1.00 88.27 | S |
| ATOM | 1459 | C | CYS | A | 182 | 107.642 | 27.302 | 14.822 | 1.00 82.53 | C |
| ATOM | 1460 | O | CYS | A | 182 | 108.136 | 28.420 | 14.963 | 1.00 80.43 | O |
| ATOM | 1461 | N | ASN | A | 183 | 108.350 | 26.180 | 14.858 | 1.00 83.98 | N |
| ATOM | 1462 | CA | ASN | A | 183 | 109.789 | 26.178 | 15.038 | 1.00 87.53 | C |
| ATOM | 1463 | CB | ASN | A | 183 | 110.127 | 26.372 | 16.515 | 1.00 94.35 | C |
| ATOM | 1464 | CG | ASN | A | 183 | 111.599 | 26.621 | 16.752 | 1.00 99.90 | C |
| ATOM | 1465 | OD1 | ASN | A | 183 | 112.340 | 27.000 | 15.837 | 1.00102.98 | O |
| ATOM | 1466 | ND2 | ASN | A | 183 | 112.035 | 26.415 | 17.987 | 1.00102.09 | N |
| ATOM | 1467 | C | ASN | A | 183 | 110.364 | 24.867 | 14.518 | 1.00 86.07 | C |
| ATOM | 1468 | O | ASN | A | 183 | 109.675 | 23.846 | 14.509 | 1.00 84.50 | O |
| ATOM | 1469 | N | ILE | A | 184 | 111.621 | 24.899 | 14.084 | 1.00 85.52 | N |
| ATOM | 1470 | CA | ILE | A | 184 | 112.261 | 23.724 | 13.500 | 1.00 85.50 | C |
| ATOM | 1471 | CB | ILE | A | 184 | 112.902 | 24.067 | 12.145 | 1.00 86.78 | C |
| ATOM | 1472 | CG1 | ILE | A | 184 | 111.855 | 24.741 | 11.257 | 1.00 88.90 | C |
| ATOM | 1473 | CD1 | ILE | A | 184 | 112.291 | 24.944 | 9.829 | 1.00 91.71 | C |
| ATOM | 1474 | CG2 | ILE | A | 184 | 113.462 | 22.813 | 11.477 | 1.00 86.77 | C |
| ATOM | 1475 | C | ILE | A | 184 | 113.305 | 23.121 | 14.431 | 1.00 85.05 | C |
| ATOM | 1476 | O | ILE | A | 184 | 114.204 | 23.814 | 14.906 | 1.00 87.23 | O |
| ATOM | 1477 | N | THR | A | 185 | 113.172 | 21.819 | 14.673 | 1.00 84.65 | N |
| ATOM | 1478 | CA | THR | A | 185 | 114.090 | 21.070 | 15.519 | 1.00 84.52 | C |
| ATOM | 1479 | CB | THR | A | 185 | 113.324 | 20.290 | 16.607 | 1.00 83.63 | C |
| ATOM | 1480 | OG1 | THR | A | 185 | 112.565 | 19.231 | 16.010 | 1.00 82.31 | O |
| ATOM | 1481 | CG2 | THR | A | 185 | 112.382 | 21.212 | 17.373 | 1.00 82.88 | C |
| ATOM | 1482 | C | THR | A | 185 | 114.884 | 20.089 | 14.660 | 1.00 87.43 | C |
| ATOM | 1483 | O | THR | A | 185 | 114.461 | 19.731 | 13.562 | 1.00 92.86 | O |
| ATOM | 1484 | N | GLY | A | 186 | 116.033 | 19.654 | 15.163 | 1.00 89.40 | N |
| ATOM | 1485 | CA | GLY | A | 186 | 116.885 | 18.702 | 14.449 | 1.00 89.10 | C |
| ATOM | 1486 | C | GLY | A | 186 | 117.567 | 19.281 | 13.222 | 1.00 87.83 | C |
| ATOM | 1487 | O | GLY | A | 186 | 117.929 | 18.544 | 12.304 | 1.00 86.14 | O |
| ATOM | 1488 | N | SER | A | 187 | 117.760 | 20.599 | 13.215 | 1.00 87.16 | N |
| ATOM | 1489 | CA | SER | A | 187 | 118.376 | 21.288 | 12.084 | 1.00 87.75 | C |
| ATOM | 1490 | CB | SER | A | 187 | 118.233 | 22.803 | 12.249 | 1.00 86.93 | C |
| ATOM | 1491 | OG | SER | A | 187 | 118.824 | 23.495 | 11.163 | 1.00 87.00 | O |
| ATOM | 1492 | C | SER | A | 187 | 119.852 | 20.926 | 11.941 | 1.00 88.80 | C |
| ATOM | 1493 | O | SER | A | 187 | 120.620 | 21.057 | 12.892 | 1.00 91.38 | O |
| ATOM | 1494 | N | THR | A | 188 | 120.234 | 20.467 | 10.750 | 1.00 91.89 | N |
| ATOM | 1495 | CA | THR | A | 188 | 121.626 | 20.126 | 10.444 | 1.00 93.80 | C |
| ATOM | 1496 | CB | THR | A | 188 | 121.803 | 18.608 | 10.225 | 1.00 94.36 | C |
| ATOM | 1497 | OG1 | THR | A | 188 | 121.083 | 18.199 | 9.055 | 1.00 97.73 | O |
| ATOM | 1498 | CG2 | THR | A | 188 | 121.299 | 17.827 | 11.429 | 1.00 95.01 | C |
| ATOM | 1499 | C | THR | A | 188 | 122.085 | 20.876 | 9.193 | 1.00 95.01 | C |
| ATOM | 1500 | O | THR | A | 188 | 122.790 | 20.323 | 8.349 | 1.00 94.25 | O |
| ATOM | 1501 | N | SER | A | 189 | 121.677 | 22.139 | 9.084 | 1.00 97.50 | N |
| ATOM | 1502 | CA | SER | A | 189 | 122.010 | 22.974 | 7.930 | 1.00 98.32 | C |
| ATOM | 1503 | CB | SER | A | 189 | 121.191 | 22.547 | 6.711 | 1.00 97.46 | C |
| ATOM | 1504 | OG | SER | A | 189 | 121.468 | 23.375 | 5.596 | 1.00 96.80 | O |
| ATOM | 1505 | C | SER | A | 189 | 121.746 | 24.447 | 8.228 | 1.00100.26 | C |
| ATOM | 1506 | O | SER | A | 189 | 120.976 | 24.773 | 9.128 | 1.00100.80 | O |
| ATOM | 1507 | N | HIS | A | 190 | 122.402 | 25.327 | 7.475 | 1.00100.89 | N |
| ATOM | 1508 | CA | HIS | A | 190 | 122.232 | 26.775 | 7.622 | 1.00101.15 | C |
| ATOM | 1509 | CB | HIS | A | 190 | 123.489 | 27.394 | 8.244 | 1.00104.71 | C |
| ATOM | 1510 | CG | HIS | A | 190 | 123.642 | 27.110 | 9.705 | 1.00109.21 | C |
| ATOM | 1511 | ND1 | HIS | A | 190 | 124.242 | 25.965 | 10.184 | 1.00110.75 | N |
| ATOM | 1512 | CE1 | HIS | A | 190 | 124.233 | 25.986 | 11.505 | 1.00112.14 | C |
| ATOM | 1513 | NE2 | HIS | A | 190 | 123.651 | 27.104 | 11.900 | 1.00113.10 | N |
| ATOM | 1514 | CD2 | HIS | A | 190 | 123.272 | 27.825 | 10.794 | 1.00111.06 | C |
| ATOM | 1515 | C | HIS | A | 190 | 121.908 | 27.475 | 6.301 | 1.00 98.77 | C |
| ATOM | 1516 | O | HIS | A | 190 | 121.777 | 28.700 | 6.269 | 1.00 99.11 | O |
| ATOM | 1517 | N | VAL | A | 191 | 121.765 | 26.708 | 5.222 | 1.00 96.73 | N |
| ATOM | 1518 | CA | VAL | A | 191 | 121.453 | 27.279 | 3.913 | 1.00 92.97 | C |
| ATOM | 1519 | CB | VAL | A | 191 | 121.734 | 26.286 | 2.765 | 1.00 93.24 | C |
| ATOM | 1520 | CG1 | VAL | A | 191 | 121.399 | 26.919 | 1.420 | 1.00 94.47 | C |
| ATOM | 1521 | CG2 | VAL | A | 191 | 123.188 | 25.832 | 2.791 | 1.00 92.73 | C |
| ATOM | 1522 | C | VAL | A | 191 | 119.975 | 27.671 | 3.907 | 1.00 89.35 | C |
| ATOM | 1523 | O | VAL | A | 191 | 119.106 | 26.804 | 4.023 | 1.00 88.41 | O |
| ATOM | 1524 | N | PRO | A | 192 | 119.685 | 28.977 | 3.763 | 1.00 85.73 | N |
| ATOM | 1525 | CA | PRO | A | 192 | 118.304 | 29.454 | 3.837 | 1.00 85.92 | C |
| ATOM | 1526 | CB | PRO | A | 192 | 118.465 | 30.977 | 3.882 | 1.00 85.56 | C |
| ATOM | 1527 | CG | PRO | A | 192 | 119.733 | 31.239 | 3.152 | 1.00 85.62 | C |
| ATOM | 1528 | CD | PRO | A | 192 | 120.625 | 30.062 | 3.424 | 1.00 85.73 | C |
| ATOM | 1529 | C | PRO | A | 192 | 117.431 | 29.038 | 2.650 | 1.00 87.68 | C |
| ATOM | 1530 | O | PRO | A | 192 | 117.916 | 28.426 | 1.694 | 1.00 87.51 | O |
| ATOM | 1531 | N | LEU | A | 193 | 116.145 | 29.368 | 2.734 | 1.00 88.68 | N |
| ATOM | 1532 | CA | LEU | A | 193 | 115.191 | 29.059 | 1.675 | 1.00 88.77 | C |
| ATOM | 1533 | CB | LEU | A | 193 | 113.762 | 28.982 | 2.228 | 1.00 89.66 | C |
| ATOM | 1534 | CG | LEU | A | 193 | 113.369 | 27.730 | 3.019 | 1.00 91.71 | C |
| ATOM | 1535 | CD1 | LEU | A | 193 | 114.294 | 27.487 | 4.203 | 1.00 93.84 | C |
| ATOM | 1536 | CD2 | LEU | A | 193 | 111.925 | 27.843 | 3.485 | 1.00 90.96 | C |
| ATOM | 1537 | C | LEU | A | 193 | 115.258 | 30.117 | 0.582 | 1.00 90.31 | C |
| ATOM | 1538 | O | LEU | A | 193 | 115.552 | 31.282 | 0.852 | 1.00 90.47 | O |
| ATOM | 1539 | N | ALA | A | 194 | 114.984 | 29.701 | −0.651 | 1.00 91.88 | N |
| ATOM | 1540 | CA | ALA | A | 194 | 114.947 | 30.620 | −1.785 | 1.00 92.62 | C |
| ATOM | 1541 | CB | ALA | A | 194 | 115.054 | 29.851 | −3.092 | 1.00 94.08 | C |
| ATOM | 1542 | C | ALA | A | 194 | 113.647 | 31.413 | −1.744 | 1.00 92.46 | C |
| ATOM | 1543 | O | ALA | A | 194 | 112.663 | 30.965 | −1.151 | 1.00 92.11 | O |
| ATOM | 1544 | N | LYS | A | 195 | 113.645 | 32.593 | −2.361 | 1.00 93.79 | N |
| ATOM | 1545 | CA | LYS | A | 195 | 112.437 | 33.415 | −2.412 | 1.00 96.51 | C |
| ATOM | 1546 | CB | LYS | A | 195 | 112.718 | 34.780 | −3.041 | 1.00101.11 | C |
| ATOM | 1547 | CG | LYS | A | 195 | 113.591 | 35.670 | −2.171 | 1.00108.63 | C |
| ATOM | 1548 | CD | LYS | A | 195 | 113.770 | 37.056 | −2.769 | 1.00116.33 | C |
| ATOM | 1549 | CE | LYS | A | 195 | 114.626 | 37.937 | −1.870 | 1.00118.72 | C |
| ATOM | 1550 | NZ | LYS | A | 195 | 114.852 | 39.289 | −2.453 | 1.00120.76 | N |
| ATOM | 1551 | C | LYS | A | 195 | 111.364 | 32.666 | −3.196 | 1.00 94.08 | C |
| ATOM | 1552 | O | LYS | A | 195 | 111.553 | 32.383 | −4.380 | 1.00 94.57 | O |
| ATOM | 1553 | N | PRO | A | 196 | 110.239 | 32.335 | −2.537 | 1.00 89.68 | N |
| ATOM | 1554 | CA | PRO | A | 196 | 109.214 | 31.518 | −3.174 | 1.00 89.92 | C |
| ATOM | 1555 | CB | PRO | A | 196 | 108.264 | 31.195 | −2.021 | 1.00 90.30 | C |
| ATOM | 1556 | CG | PRO | A | 196 | 108.366 | 32.383 | −1.137 | 1.00 89.69 | C |
| ATOM | 1557 | CD | PRO | A | 196 | 109.794 | 32.837 | −1.224 | 1.00 89.48 | C |
| ATOM | 1558 | C | PRO | A | 196 | 108.480 | 32.251 | −4.291 | 1.00 88.68 | C |
| ATOM | 1559 | O | PRO | A | 196 | 108.298 | 33.467 | −4.217 | 1.00 84.56 | O |
| ATOM | 1560 | N | LYS | A | 197 | 108.062 | 31.498 | −5.308 | 1.00 89.24 | N |
| ATOM | 1561 | CA | LYS | A | 197 | 107.406 | 32.056 | −6.486 | 1.00 90.03 | C |
| ATOM | 1562 | CB | LYS | A | 197 | 108.300 | 31.857 | −7.708 | 1.00 94.64 | C |
| ATOM | 1563 | CG | LYS | A | 197 | 109.639 | 32.561 | −7.567 | 1.00102.22 | C |
| ATOM | 1564 | CD | LYS | A | 197 | 110.545 | 32.364 | −8.768 | 1.00108.84 | C |
| ATOM | 1565 | CE | LYS | A | 197 | 111.819 | 33.181 | −8.599 | 1.00114.08 | C |
| ATOM | 1566 | NZ | LYS | A | 197 | 112.776 | 33.008 | −9.726 | 1.00117.94 | N |
| ATOM | 1567 | C | LYS | A | 197 | 106.035 | 31.434 | −6.711 | 1.00 87.01 | C |
| ATOM | 1568 | O | LYS | A | 197 | 105.739 | 30.359 | −6.192 | 1.00 86.28 | O |
| ATOM | 1569 | N | VAL | A | 198 | 105.205 | 32.120 | −7.492 | 1.00 85.50 | N |
| ATOM | 1570 | CA | VAL | A | 198 | 103.827 | 31.689 | −7.739 | 1.00 84.79 | C |
| ATOM | 1571 | CB | VAL | A | 198 | 103.047 | 32.737 | −8.567 | 1.00 84.93 | C |
| ATOM | 1572 | CG1 | VAL | A | 198 | 101.643 | 32.239 | −8.896 | 1.00 84.34 | C |
| ATOM | 1573 | CG2 | VAL | A | 198 | 102.983 | 34.067 | −7.822 | 1.00 84.73 | C |
| ATOM | 1574 | C | VAL | A | 198 | 103.783 | 30.337 | −8.452 | 1.00 83.78 | C |
| ATOM | 1575 | O | VAL | A | 198 | 104.502 | 30.119 | −9.426 | 1.00 82.25 | O |
| ATOM | 1576 | N | ALA | A | 199 | 102.938 | 29.438 | −7.952 | 1.00 85.82 | N |
| ATOM | 1577 | CA | ALA | A | 199 | 102.791 | 28.103 | −8.529 | 1.00 87.01 | C |
| ATOM | 1578 | CB | ALA | A | 199 | 102.153 | 27.159 | −7.521 | 1.00 87.79 | C |
| ATOM | 1579 | C | ALA | A | 199 | 101.950 | 28.165 | −9.799 | 1.00 86.64 | C |
| ATOM | 1580 | O | ALA | A | 199 | 100.884 | 28.778 | −9.811 | 1.00 85.61 | O |
| ATOM | 1581 | N | ARG | A | 200 | 102.434 | 27.526 | −10.862 | 1.00 87.46 | N |
| ATOM | 1582 | CA | ARG | A | 200 | 101.745 | 27.524 | −12.153 | 1.00 85.78 | C |
| ATOM | 1583 | CB | ARG | A | 200 | 102.754 | 27.716 | −13.284 | 1.00 87.93 | C |
| ATOM | 1584 | CG | ARG | A | 200 | 103.507 | 29.037 | −13.218 | 1.00 90.52 | C |
| ATOM | 1585 | CD | ARG | A | 200 | 104.596 | 29.114 | −14.276 | 1.00 92.57 | C |
| ATOM | 1586 | NE | ARG | A | 200 | 104.039 | 29.134 | −15.626 | 1.00 94.90 | N |
| ATOM | 1587 | CZ | ARG | A | 200 | 103.594 | 30.223 | −16.251 | 1.00 97.54 | C |
| ATOM | 1588 | NH1 | ARG | A | 200 | 103.104 | 30.118 | −17.481 | 1.00100.03 | N |
| ATOM | 1589 | NH2 | ARG | A | 200 | 103.630 | 31.416 | −15.663 | 1.00 98.92 | N |
| ATOM | 1590 | C | ARG | A | 200 | 100.978 | 26.224 | −12.355 | 1.00 83.28 | C |
| ATOM | 1591 | O | ARG | A | 200 | 101.387 | 25.176 | −11.855 | 1.00 84.83 | O |
| ATOM | 1592 | N | ARG | A | 201 | 99.866 | 26.299 | −13.087 | 1.00 82.76 | N |
| ATOM | 1593 | CA | ARG | A | 201 | 99.032 | 25.127 | −13.374 | 1.00 84.44 | C |
| ATOM | 1594 | CB | ARG | A | 201 | 97.675 | 25.557 | −13.940 | 1.00 82.09 | C |
| ATOM | 1595 | CG | ARG | A | 201 | 96.632 | 24.456 | −13.958 | 1.00 79.77 | C |
| ATOM | 1596 | CD | ARG | A | 201 | 95.331 | 24.954 | −14.554 | 1.00 79.28 | C |
| ATOM | 1597 | NE | ARG | A | 201 | 94.242 | 23.990 | −14.392 | 1.00 80.17 | N |
| ATOM | 1598 | CZ | ARG | A | 201 | 93.447 | 23.899 | −13.323 | 1.00 79.41 | C |
| ATOM | 1599 | NH1 | ARG | A | 201 | 92.485 | 22.982 | −13.298 | 1.00 79.97 | N |
| ATOM | 1600 | NH2 | ARG | A | 201 | 93.600 | 24.707 | −12.279 | 1.00 79.24 | N |
| ATOM | 1601 | C | ARG | A | 201 | 99.786 | 24.225 | −14.352 | 1.00 87.56 | C |
| ATOM | 1602 | O | ARG | A | 201 | 99.591 | 24.278 | −15.563 | 1.00 85.14 | O |
| ATOM | 1603 | N | CYS | A | 202 | 100.640 | 23.380 | −13.792 | 1.00 94.65 | N |
| ATOM | 1604 | CA | CYS | A | 202 | 101.609 | 22.608 | −14.559 | 1.00 95.70 | C |
| ATOM | 1605 | CB | CYS | A | 202 | 102.867 | 22.449 | −13.708 | 1.00 99.41 | C |
| ATOM | 1606 | SG | CYS | A | 202 | 104.213 | 21.618 | −14.557 | 1.00111.34 | S |
| ATOM | 1607 | C | CYS | A | 202 | 101.142 | 21.228 | −15.009 | 1.00 93.03 | C |
| ATOM | 1608 | O | CYS | A | 202 | 101.151 | 20.921 | −16.198 | 1.00 90.38 | O |
| ATOM | 1609 | N | GLY | A | 203 | 100.750 | 20.400 | −14.046 | 1.00 94.84 | N |
| ATOM | 1610 | CA | GLY | A | 203 | 100.396 | 19.003 | −14.295 | 1.00 95.40 | C |
| ATOM | 1611 | C | GLY | A | 203 | 99.189 | 18.741 | −15.174 | 1.00 94.49 | C |
| ATOM | 1612 | O | GLY | A | 203 | 98.573 | 19.662 | −15.708 | 1.00 95.78 | O |
| ATOM | 1613 | N | ALA | A | 204 | 98.860 | 17.462 | −15.312 | 1.00 93.19 | N |
| ATOM | 1614 | CA | ALA | A | 204 | 97.767 | 17.027 | −16.167 | 1.00 94.27 | C |
| ATOM | 1615 | CB | ALA | A | 204 | 98.058 | 15.637 | −16.707 | 1.00 94.46 | C |
| ATOM | 1616 | C | ALA | A | 204 | 96.443 | 17.021 | −15.425 | 1.00 98.11 | C |
| ATOM | 1617 | O | ALA | A | 204 | 96.405 | 16.857 | −14.206 | 1.00102.18 | O |
| ATOM | 1618 | N | ASP | A | 205 | 95.361 | 17.206 | −16.176 | 1.00103.96 | N |
| ATOM | 1619 | CA | ASP | A | 205 | 94.000 | 17.120 | −15.642 | 1.00110.17 | C |
| ATOM | 1620 | CB | ASP | A | 205 | 93.586 | 18.402 | −14.895 | 1.00112.33 | C |
| ATOM | 1621 | CG | ASP | A | 205 | 93.754 | 19.665 | −15.729 | 1.00113.97 | C |
| ATOM | 1622 | OD1 | ASP | A | 205 | 94.538 | 19.648 | −16.705 | 1.00121.66 | O |
| ATOM | 1623 | OD2 | ASP | A | 205 | 93.104 | 20.682 | −15.395 | 1.00108.73 | O |
| ATOM | 1624 | C | ASP | A | 205 | 93.024 | 16.790 | −16.777 | 1.00110.88 | C |
| ATOM | 1625 | O | ASP | A | 205 | 92.516 | 17.692 | −17.446 | 1.00112.06 | O |
| ATOM | 1626 | N | PRO | A | 206 | 92.781 | 15.487 | −17.013 | 1.00110.76 | N |
| ATOM | 1627 | CA | PRO | A | 206 | 91.870 | 15.035 | −18.068 | 1.00113.60 | C |
| ATOM | 1628 | CB | PRO | A | 206 | 91.710 | 13.543 | −17.767 | 1.00113.91 | C |
| ATOM | 1629 | CG | PRO | A | 206 | 92.992 | 13.157 | −17.117 | 1.00113.31 | C |
| ATOM | 1630 | CD | PRO | A | 206 | 93.432 | 14.354 | −16.327 | 1.00111.03 | C |
| ATOM | 1631 | C | PRO | A | 206 | 90.500 | 15.726 | −18.064 | 1.00115.91 | C |
| ATOM | 1632 | O | PRO | A | 206 | 89.961 | 16.030 | −19.131 | 1.00114.76 | O |
| ATOM | 1633 | N | ASP | A | 207 | 89.960 | 15.974 | −16.872 | 1.00115.90 | N |
| ATOM | 1634 | CA | ASP | A | 207 | 88.638 | 16.587 | −16.709 | 1.00115.10 | C |
| ATOM | 1635 | CB | ASP | A | 207 | 88.322 | 16.779 | −15.220 | 1.00116.51 | C |
| ATOM | 1636 | CG | ASP | A | 207 | 88.249 | 15.466 | −14.456 | 1.00118.65 | C |
| ATOM | 1637 | OD1 | ASP | A | 207 | 88.492 | 14.399 | −15.061 | 1.00120.29 | O |
| ATOM | 1638 | OD2 | ASP | A | 207 | 87.950 | 15.505 | −13.244 | 1.00117.90 | O |
| ATOM | 1639 | C | ASP | A | 207 | 88.484 | 17.927 | −17.428 | 1.00114.57 | C |
| ATOM | 1640 | O | ASP | A | 207 | 87.439 | 18.192 | −18.023 | 1.00120.87 | O |
| ATOM | 1641 | N | HIS | A | 208 | 89.521 | 18.760 | −17.376 | 1.00113.32 | N |
| ATOM | 1642 | CA | HIS | A | 208 | 89.483 | 20.096 | −17.988 | 1.00113.45 | C |
| ATOM | 1643 | CB | HIS | A | 208 | 90.067 | 21.132 | −17.017 | 1.00115.37 | C |
| ATOM | 1644 | CG | HIS | A | 208 | 89.172 | 21.444 | −15.857 | 1.00117.34 | C |
| ATOM | 1645 | ND1 | HIS | A | 208 | 88.893 | 20.531 | −14.863 | 1.00120.15 | N |
| ATOM | 1646 | CE1 | HIS | A | 208 | 88.082 | 21.080 | −13.975 | 1.00119.71 | C |
| ATOM | 1647 | NE2 | HIS | A | 208 | 87.829 | 22.320 | −14.355 | 1.00121.26 | N |
| ATOM | 1648 | CD2 | HIS | A | 208 | 88.499 | 22.573 | −15.528 | 1.00119.91 | C |
| ATOM | 1649 | C | HIS | A | 208 | 90.182 | 20.175 | −19.356 | 1.00112.20 | C |
| ATOM | 1650 | O | HIS | A | 208 | 90.581 | 21.259 | −19.788 | 1.00107.64 | O |
| ATOM | 1651 | N | GLY | A | 209 | 90.323 | 19.037 | −20.036 | 1.00114.07 | N |
| ATOM | 1652 | CA | GLY | A | 209 | 90.876 | 19.007 | −21.392 | 1.00116.91 | C |
| ATOM | 1653 | C | GLY | A | 209 | 92.220 | 18.320 | −21.533 | 1.00121.22 | C |
| ATOM | 1654 | O | GLY | A | 209 | 92.299 | 17.200 | −22.040 | 1.00125.14 | O |
| ATOM | 1655 | N | LYS | A | 210 | 93.276 | 18.995 | −21.081 | 1.00123.83 | N |
| ATOM | 1656 | CA | LYS | A | 210 | 94.650 | 18.501 | −21.232 | 1.00123.76 | C |
| ATOM | 1657 | CB | LYS | A | 210 | 95.645 | 19.588 | −20.823 | 1.00129.30 | C |
| ATOM | 1658 | CG | LYS | A | 210 | 95.595 | 20.797 | −21.742 | 1.00137.32 | C |
| ATOM | 1659 | CD | LYS | A | 210 | 96.602 | 21.857 | −21.349 | 1.00141.35 | C |
| ATOM | 1660 | CE | LYS | A | 210 | 96.470 | 23.076 | −22.249 | 1.00142.74 | C |
| ATOM | 1661 | NZ | LYS | A | 210 | 97.345 | 24.190 | −21.804 | 1.00145.82 | N |
| ATOM | 1662 | C | LYS | A | 210 | 94.936 | 17.227 | −20.440 | 1.00118.22 | C |
| ATOM | 1663 | O | LYS | A | 210 | 94.935 | 17.261 | −19.210 | 1.00115.67 | O |
| ATOM | 1664 | N | PRO | A | 211 | 95.191 | 16.103 | −21.144 | 1.00112.92 | N |
| ATOM | 1665 | CA | PRO | A | 211 | 95.491 | 14.842 | −20.476 | 1.00111.37 | C |
| ATOM | 1666 | CB | PRO | A | 211 | 94.974 | 13.806 | −21.474 | 1.00111.45 | C |
| ATOM | 1667 | CG | PRO | A | 211 | 95.191 | 14.441 | −22.807 | 1.00112.22 | C |
| ATOM | 1668 | CD | PRO | A | 211 | 95.187 | 15.936 | −22.612 | 1.00112.40 | C |
| ATOM | 1669 | C | PRO | A | 211 | 96.986 | 14.615 | −20.204 | 1.00108.39 | C |
| ATOM | 1670 | O | PRO | A | 211 | 97.336 | 13.704 | −19.453 | 1.00106.62 | O |
| ATOM | 1671 | N | ASP | A | 212 | 97.850 | 15.431 | −20.808 | 1.00106.42 | N |
| ATOM | 1672 | CA | ASP | A | 212 | 99.298 | 15.296 | −20.656 | 1.00105.63 | C |
| ATOM | 1673 | CB | ASP | A | 212 | 99.967 | 15.171 | −22.029 | 1.00108.37 | C |
| ATOM | 1674 | CG | ASP | A | 212 | 99.534 | 13.927 | −22.786 | 1.00111.39 | C |
| ATOM | 1675 | OD1 | ASP | A | 212 | 99.239 | 12.896 | −22.141 | 1.00110.82 | O |
| ATOM | 1676 | OD2 | ASP | A | 212 | 99.505 | 13.982 | −24.034 | 1.00113.97 | O |
| ATOM | 1677 | C | ASP | A | 212 | 99.883 | 16.499 | −19.930 | 1.00101.27 | C |
| ATOM | 1678 | O | ASP | A | 212 | 99.555 | 17.644 | −20.248 | 1.00 99.99 | O |
| ATOM | 1679 | N | ALA | A | 213 | 100.766 | 16.234 | −18.970 | 1.00 96.39 | N |
| ATOM | 1680 | CA | ALA | A | 213 | 101.421 | 17.292 | −18.205 | 1.00 92.95 | C |
| ATOM | 1681 | CB | ALA | A | 213 | 102.143 | 16.702 | −17.007 | 1.00 93.67 | C |
| ATOM | 1682 | C | ALA | A | 213 | 102.404 | 18.066 | −19.077 | 1.00 90.86 | C |
| ATOM | 1683 | O | ALA | A | 213 | 102.891 | 17.548 | −20.081 | 1.00 91.44 | O |
| ATOM | 1684 | N | VAL | A | 214 | 102.694 | 19.303 | −18.683 | 1.00 89.07 | N |
| ATOM | 1685 | CA | VAL | A | 214 | 103.615 | 20.160 | −19.426 | 1.00 90.36 | C |
| ATOM | 1686 | CB | VAL | A | 214 | 102.894 | 21.402 | −19.986 | 1.00 90.82 | C |
| ATOM | 1687 | CG1 | VAL | A | 214 | 103.848 | 22.256 | −20.817 | 1.00 92.38 | C |
| ATOM | 1688 | CG2 | VAL | A | 214 | 101.700 | 20.974 | −20.828 | 1.00 92.12 | C |
| ATOM | 1689 | C | VAL | A | 214 | 104.780 | 20.571 | −18.519 | 1.00 92.80 | C |
| ATOM | 1690 | O | VAL | A | 214 | 104.756 | 21.652 | −17.931 | 1.00 94.19 | O |
| ATOM | 1691 | N | PRO | A | 215 | 105.809 | 19.705 | −18.408 | 1.00 93.71 | N |
| ATOM | 1692 | CA | PRO | A | 215 | 106.980 | 19.938 | −17.548 | 1.00 93.49 | C |
| ATOM | 1693 | CB | PRO | A | 215 | 108.004 | 18.906 | −18.037 | 1.00 93.83 | C |
| ATOM | 1694 | CG | PRO | A | 215 | 107.254 | 17.909 | −18.850 | 1.00 94.23 | C |
| ATOM | 1695 | CD | PRO | A | 215 | 105.857 | 18.391 | −19.077 | 1.00 93.70 | C |
| ATOM | 1696 | C | PRO | A | 215 | 107.585 | 21.339 | −17.653 | 1.00 94.44 | C |
| ATOM | 1697 | O | PRO | A | 215 | 108.031 | 21.893 | −16.649 | 1.00 95.00 | O |
| ATOM | 1698 | N | GLY | A | 216 | 107.604 | 21.896 | −18.861 | 1.00 93.59 | N |
| ATOM | 1699 | CA | GLY | A | 216 | 108.176 | 23.218 | −19.100 | 1.00 91.57 | C |
| ATOM | 1700 | C | GLY | A | 216 | 107.431 | 24.382 | −18.468 | 1.00 91.34 | C |
| ATOM | 1701 | O | GLY | A | 216 | 107.971 | 25.484 | −18.381 | 1.00 93.81 | O |
| ATOM | 1702 | N | ASN | A | 217 | 106.196 | 24.145 | −18.030 | 1.00 90.53 | N |
| ATOM | 1703 | CA | ASN | A | 217 | 105.355 | 25.193 | −17.451 | 1.00 89.29 | C |
| ATOM | 1704 | CB | ASN | A | 217 | 103.918 | 24.992 | −17.933 | 1.00 87.44 | C |
| ATOM | 1705 | CG | ASN | A | 217 | 103.098 | 26.271 | −17.916 | 1.00 86.60 | C |
| ATOM | 1706 | OD1 | ASN | A | 217 | 103.606 | 27.361 | −18.182 | 1.00 82.24 | O |
| ATOM | 1707 | ND2 | ASN | A | 217 | 101.812 | 26.133 | −17.613 | 1.00 88.48 | N |
| ATOM | 1708 | C | ASN | A | 217 | 105.377 | 25.209 | −15.921 | 1.00 90.89 | C |
| ATOM | 1709 | O | ASN | A | 217 | 104.729 | 26.049 | −15.301 | 1.00 93.27 | O |
| ATOM | 1710 | N | CYS | A | 218 | 106.122 | 24.285 | −15.321 | 1.00 92.57 | N |
| ATOM | 1711 | CA | CYS | A | 218 | 106.186 | 24.151 | −13.861 | 1.00 92.13 | C |
| ATOM | 1712 | CB | CYS | A | 218 | 106.800 | 22.797 | −13.471 | 1.00 95.46 | C |
| ATOM | 1713 | SG | CYS | A | 218 | 105.647 | 21.446 | −13.139 | 1.00101.99 | S |
| ATOM | 1714 | C | CYS | A | 218 | 106.993 | 25.242 | −13.171 | 1.00 89.73 | C |
| ATOM | 1715 | O | CYS | A | 218 | 107.875 | 25.855 | −13.769 | 1.00 90.36 | O |
| ATOM | 1716 | N | THR | A | 219 | 106.663 | 25.468 | −11.902 | 1.00 89.47 | N |
| ATOM | 1717 | CA | THR | A | 219 | 107.444 | 26.320 | −11.015 | 1.00 89.67 | C |
| ATOM | 1718 | CB | THR | A | 219 | 106.547 | 27.210 | −10.139 | 1.00 89.31 | C |
| ATOM | 1719 | OG1 | THR | A | 219 | 105.719 | 28.023 | −10.977 | 1.00 89.54 | O |
| ATOM | 1720 | CG2 | THR | A | 219 | 107.386 | 28.109 | −9.241 | 1.00 89.94 | C |
| ATOM | 1721 | C | THR | A | 219 | 108.222 | 25.325 | −10.157 | 1.00 89.67 | C |
| ATOM | 1722 | O | THR | A | 219 | 107.624 | 24.568 | −9.388 | 1.00 88.45 | O |
| ATOM | 1723 | N | TYR | A | 220 | 109.546 | 25.319 | −10.295 | 1.00 88.06 | N |
| ATOM | 1724 | CA | TYR | A | 220 | 110.376 | 24.283 | −9.673 | 1.00 87.99 | C |
| ATOM | 1725 | CB | TYR | A | 220 | 111.469 | 23.834 | −10.651 | 1.00 85.51 | C |
| ATOM | 1726 | CG | TYR | A | 220 | 110.940 | 22.896 | −11.707 | 1.00 84.82 | C |
| ATOM | 1727 | CD1 | TYR | A | 220 | 110.797 | 21.540 | −11.438 | 1.00 85.64 | C |
| ATOM | 1728 | CE1 | TYR | A | 220 | 110.303 | 20.669 | −12.390 | 1.00 87.79 | C |
| ATOM | 1729 | CZ | TYR | A | 220 | 109.938 | 21.150 | −13.629 | 1.00 87.64 | C |
| ATOM | 1730 | OH | TYR | A | 220 | 109.447 | 20.273 | −14.565 | 1.00 92.31 | O |
| ATOM | 1731 | CE2 | TYR | A | 220 | 110.066 | 22.495 | −13.923 | 1.00 85.64 | C |
| ATOM | 1732 | CD2 | TYR | A | 220 | 110.560 | 23.361 | −12.963 | 1.00 84.72 | C |
| ATOM | 1733 | C | TYR | A | 220 | 110.982 | 24.613 | −8.309 | 1.00 89.61 | C |
| ATOM | 1734 | O | TYR | A | 220 | 111.157 | 23.715 | −7.484 | 1.00 90.52 | O |
| ATOM | 1735 | N | GLY | A | 221 | 111.296 | 25.879 | −8.060 | 1.00 88.76 | N |
| ATOM | 1736 | CA | GLY | A | 221 | 111.916 | 26.265 | −6.791 | 1.00 88.20 | C |
| ATOM | 1737 | C | GLY | A | 221 | 110.953 | 26.231 | −5.612 | 1.00 84.48 | C |
| ATOM | 1738 | O | GLY | A | 221 | 110.070 | 25.375 | −5.532 | 1.00 80.92 | O |
| ATOM | 1739 | N | ALA | A | 222 | 111.142 | 27.164 | −4.684 | 1.00 81.28 | N |
| ATOM | 1740 | CA | ALA | A | 222 | 110.228 | 27.326 | −3.564 | 1.00 79.28 | C |
| ATOM | 1741 | CB | ALA | A | 222 | 110.833 | 28.242 | −2.510 | 1.00 80.09 | C |
| ATOM | 1742 | C | ALA | A | 222 | 108.935 | 27.919 | −4.105 | 1.00 76.65 | C |
| ATOM | 1743 | O | ALA | A | 222 | 108.970 | 28.817 | −4.947 | 1.00 77.00 | O |
| ATOM | 1744 | N | LYS | A | 223 | 107.799 | 27.420 | −3.628 | 1.00 75.17 | N |
| ATOM | 1745 | CA | LYS | A | 223 | 106.499 | 27.890 | −4.102 | 1.00 73.94 | C |
| ATOM | 1746 | CB | LYS | A | 223 | 105.717 | 26.727 | −4.719 | 1.00 74.06 | C |
| ATOM | 1747 | CG | LYS | A | 223 | 106.187 | 26.356 | −6.117 | 1.00 72.01 | C |
| ATOM | 1748 | CD | LYS | A | 223 | 105.382 | 25.206 | −6.701 | 1.00 70.96 | C |
| ATOM | 1749 | CE | LYS | A | 223 | 105.802 | 23.859 | −6.137 | 1.00 70.20 | C |
| ATOM | 1750 | NZ | LYS | A | 223 | 107.178 | 23.471 | −6.561 | 1.00 69.98 | N |
| ATOM | 1751 | C | LYS | A | 223 | 105.666 | 28.563 | −3.014 | 1.00 72.76 | C |
| ATOM | 1752 | O | LYS | A | 223 | 105.661 | 28.132 | −1.863 | 1.00 77.03 | O |
| ATOM | 1753 | N | GLN | A | 224 | 104.959 | 29.622 | −3.399 | 1.00 71.94 | N |
| ATOM | 1754 | CA | GLN | A | 224 | 104.062 | 30.330 | −2.490 | 1.00 71.49 | C |
| ATOM | 1755 | CB | GLN | A | 224 | 103.700 | 31.707 | −3.047 | 1.00 69.85 | C |
| ATOM | 1756 | CG | GLN | A | 224 | 104.875 | 32.633 | −3.299 | 1.00 67.77 | C |
| ATOM | 1757 | CD | GLN | A | 224 | 104.429 | 34.012 | −3.742 | 1.00 67.40 | C |
| ATOM | 1758 | OE1 | GLN | A | 224 | 103.238 | 34.263 | −3.939 | 1.00 68.00 | O |
| ATOM | 1759 | NE2 | GLN | A | 224 | 105.384 | 34.919 | −3.894 | 1.00 68.81 | N |
| ATOM | 1760 | C | GLN | A | 224 | 102.772 | 29.537 | −2.320 | 1.00 71.05 | C |
| ATOM | 1761 | O | GLN | A | 224 | 102.475 | 28.664 | −3.132 | 1.00 72.16 | O |
| ATOM | 1762 | N | PRO | A | 225 | 101.989 | 29.846 | −1.270 | 1.00 72.77 | N |
| ATOM | 1763 | CA | PRO | A | 225 | 100.696 | 29.177 | −1.136 | 1.00 72.88 | C |
| ATOM | 1764 | CB | PRO | A | 225 | 100.196 | 29.624 | 0.248 | 1.00 72.02 | C |
| ATOM | 1765 | CG | PRO | A | 225 | 101.396 | 30.150 | 0.956 | 1.00 73.36 | C |
| ATOM | 1766 | CD | PRO | A | 225 | 102.278 | 30.714 | −0.116 | 1.00 73.96 | C |
| ATOM | 1767 | C | PRO | A | 225 | 99.735 | 29.633 | −2.230 | 1.00 72.69 | C |
| ATOM | 1768 | O | PRO | A | 225 | 99.995 | 30.629 | −2.903 | 1.00 73.43 | O |
| ATOM | 1769 | N | LEU | A | 226 | 98.637 | 28.904 | −2.397 | 1.00 72.76 | N |
| ATOM | 1770 | CA | LEU | A | 226 | 97.638 | 29.223 | −3.411 | 1.00 71.49 | C |
| ATOM | 1771 | CB | LEU | A | 226 | 96.976 | 27.935 | −3.908 | 1.00 71.67 | C |
| ATOM | 1772 | CG | LEU | A | 226 | 97.921 | 26.901 | −4.523 | 1.00 72.47 | C |
| ATOM | 1773 | CD1 | LEU | A | 226 | 97.232 | 25.551 | −4.637 | 1.00 72.82 | C |
| ATOM | 1774 | CD2 | LEU | A | 226 | 98.436 | 27.365 | −5.879 | 1.00 73.04 | C |
| ATOM | 1775 | C | LEU | A | 226 | 96.579 | 30.175 | −2.857 | 1.00 71.78 | C |
| ATOM | 1776 | O | LEU | A | 226 | 95.613 | 29.740 | −2.222 | 1.00 75.71 | O |
| ATOM | 1777 | N | TYR | A | 227 | 96.773 | 31.474 | −3.083 | 1.00 69.55 | N |
| ATOM | 1778 | CA | TYR | A | 227 | 95.798 | 32.485 | −2.666 | 1.00 70.32 | C |
| ATOM | 1779 | CB | TYR | A | 227 | 96.469 | 33.819 | −2.358 | 1.00 70.40 | C |
| ATOM | 1780 | CG | TYR | A | 227 | 97.631 | 33.712 | −1.404 | 1.00 70.85 | C |
| ATOM | 1781 | CD1 | TYR | A | 227 | 97.499 | 33.054 | −0.183 | 1.00 72.80 | C |
| ATOM | 1782 | CE1 | TYR | A | 227 | 98.562 | 32.964 | 0.702 | 1.00 71.55 | C |
| ATOM | 1783 | CZ | TYR | A | 227 | 99.771 | 33.541 | 0.371 | 1.00 71.70 | C |
| ATOM | 1784 | OH | TYR | A | 227 | 100.833 | 33.460 | 1.236 | 1.00 71.35 | O |
| ATOM | 1785 | CE2 | TYR | A | 227 | 99.925 | 34.200 | −0.833 | 1.00 73.08 | C |
| ATOM | 1786 | CD2 | TYR | A | 227 | 98.858 | 34.285 | −1.710 | 1.00 71.37 | C |
| ATOM | 1787 | C | TYR | A | 227 | 94.801 | 32.649 | −3.800 | 1.00 69.55 | C |
| ATOM | 1788 | O | TYR | A | 227 | 95.157 | 33.101 | −4.890 | 1.00 66.57 | O |
| ATOM | 1789 | N | TRP | A | 228 | 93.548 | 32.300 | −3.531 | 1.00 70.33 | N |
| ATOM | 1790 | CA | TRP | A | 228 | 92.557 | 32.191 | −4.585 | 1.00 70.72 | C |
| ATOM | 1791 | CB | TRP | A | 228 | 92.648 | 30.765 | −5.144 | 1.00 70.69 | C |
| ATOM | 1792 | CG | TRP | A | 228 | 91.719 | 30.467 | −6.247 | 1.00 70.07 | C |
| ATOM | 1793 | CD1 | TRP | A | 228 | 91.780 | 30.943 | −7.518 | 1.00 71.56 | C |
| ATOM | 1794 | NE1 | TRP | A | 228 | 90.750 | 30.431 | −8.263 | 1.00 71.58 | N |
| ATOM | 1795 | CE2 | TRP | A | 228 | 90.004 | 29.601 | −7.472 | 1.00 68.67 | C |
| ATOM | 1796 | CD2 | TRP | A | 228 | 90.592 | 29.598 | −6.194 | 1.00 68.31 | C |
| ATOM | 1797 | CE3 | TRP | A | 228 | 90.020 | 28.817 | −5.188 | 1.00 68.07 | C |
| ATOM | 1798 | CZ3 | TRP | A | 228 | 88.899 | 28.087 | −5.481 | 1.00 68.67 | C |
| ATOM | 1799 | CH2 | TRP | A | 228 | 88.329 | 28.114 | −6.767 | 1.00 69.73 | C |
| ATOM | 1800 | CZ2 | TRP | A | 228 | 88.871 | 28.862 | −7.770 | 1.00 69.10 | C |
| ATOM | 1801 | C | TRP | A | 228 | 91.139 | 32.504 | −4.106 | 1.00 71.61 | C |
| ATOM | 1802 | O | TRP | A | 228 | 90.819 | 32.346 | −2.928 | 1.00 70.99 | O |
| ATOM | 1803 | N | LEU | A | 229 | 90.312 | 32.980 | −5.035 | 1.00 74.68 | N |
| ATOM | 1804 | CA | LEU | A | 229 | 88.887 | 33.246 | −4.803 | 1.00 75.33 | C |
| ATOM | 1805 | CB | LEU | A | 229 | 88.181 | 31.963 | −4.351 | 1.00 74.13 | C |
| ATOM | 1806 | CG | LEU | A | 229 | 86.713 | 31.815 | −4.751 | 1.00 75.62 | C |
| ATOM | 1807 | CD1 | LEU | A | 229 | 86.613 | 31.384 | −6.209 | 1.00 75.47 | C |
| ATOM | 1808 | CD2 | LEU | A | 229 | 86.015 | 30.802 | −3.857 | 1.00 76.28 | C |
| ATOM | 1809 | C | LEU | A | 229 | 88.607 | 34.376 | −3.803 | 1.00 76.75 | C |
| ATOM | 1810 | O | LEU | A | 229 | 87.518 | 34.435 | −3.232 | 1.00 78.50 | O |
| ATOM | 1811 | N | GLN | A | 230 | 89.568 | 35.277 | −3.607 | 1.00 78.20 | N |
| ATOM | 1812 | CA | GLN | A | 230 | 89.400 | 36.389 | −2.668 | 1.00 81.19 | C |
| ATOM | 1813 | CB | GLN | A | 230 | 90.621 | 36.510 | −1.748 | 1.00 80.60 | C |
| ATOM | 1814 | CG | GLN | A | 230 | 90.956 | 35.236 | −0.995 | 1.00 80.41 | C |
| ATOM | 1815 | CD | GLN | A | 230 | 89.756 | 34.657 | −0.267 | 1.00 80.81 | C |
| ATOM | 1816 | OE1 | GLN | A | 230 | 89.005 | 35.378 | 0.385 | 1.00 81.10 | O |
| ATOM | 1817 | NE2 | GLN | A | 230 | 89.567 | 33.350 | −0.383 | 1.00 80.45 | N |
| ATOM | 1818 | C | GLN | A | 230 | 89.173 | 37.707 | −3.393 | 1.00 83.69 | C |
| ATOM | 1819 | O | GLN | A | 230 | 89.290 | 37.785 | −4.615 | 1.00 85.42 | O |
| ATOM | 1820 | N | LYS | A | 231 | 88.852 | 38.741 | −2.625 | 1.00 86.49 | N |
| ATOM | 1821 | CA | LYS | A | 231 | 88.640 | 40.070 | −3.173 | 1.00 87.59 | C |
| ATOM | 1822 | CB | LYS | A | 231 | 87.760 | 40.892 | −2.231 | 1.00 91.33 | C |
| ATOM | 1823 | CG | LYS | A | 231 | 87.261 | 42.196 | −2.825 | 1.00 95.93 | C |
| ATOM | 1824 | CD | LYS | A | 231 | 86.089 | 42.745 | −2.028 | 1.00 98.90 | C |
| ATOM | 1825 | CE | LYS | A | 231 | 85.572 | 44.044 | −2.621 | 1.00102.14 | C |
| ATOM | 1826 | NZ | LYS | A | 231 | 84.321 | 44.487 | −1.950 | 1.00104.65 | N |
| ATOM | 1827 | C | LYS | A | 231 | 89.971 | 40.778 | −3.412 | 1.00 87.28 | C |
| ATOM | 1828 | O | LYS | A | 231 | 90.073 | 41.614 | −4.307 | 1.00 88.94 | O |
| ATOM | 1829 | N | GLU | A | 232 | 90.986 | 40.440 | −2.615 | 1.00 89.06 | N |
| ATOM | 1830 | CA | GLU | A | 232 | 92.305 | 41.067 | −2.724 | 1.00 90.80 | C |
| ATOM | 1831 | CB | GLU | A | 232 | 92.448 | 42.184 | −1.683 | 1.00 94.95 | C |
| ATOM | 1832 | CG | GLU | A | 232 | 91.340 | 43.231 | −1.678 | 1.00 98.24 | C |
| ATOM | 1833 | CD | GLU | A | 232 | 91.492 | 44.250 | −0.559 | 1.00 99.79 | C |
| ATOM | 1834 | OE1 | GLU | A | 232 | 92.363 | 44.064 | 0.319 | 1.00100.02 | O |
| ATOM | 1835 | OE2 | GLU | A | 232 | 90.727 | 45.237 | −0.549 | 1.00101.71 | O |
| ATOM | 1836 | C | GLU | A | 232 | 93.449 | 40.074 | −2.508 | 1.00 88.75 | C |
| ATOM | 1837 | O | GLU | A | 232 | 93.289 | 39.063 | −1.825 | 1.00 87.80 | O |
| ATOM | 1838 | N | GLY | A | 233 | 94.604 | 40.383 | −3.095 | 1.00 87.81 | N |
| ATOM | 1839 | CA | GLY | A | 233 | 95.838 | 39.625 | −2.874 | 1.00 86.80 | C |
| ATOM | 1840 | C | GLY | A | 233 | 95.893 | 38.203 | −3.401 | 1.00 85.56 | C |
| ATOM | 1841 | O | GLY | A | 233 | 96.456 | 37.325 | −2.750 | 1.00 86.63 | O |
| ATOM | 1842 | N | ASN | A | 234 | 95.327 | 37.978 | −4.582 | 1.00 85.54 | N |
| ATOM | 1843 | CA | ASN | A | 234 | 95.341 | 36.658 | −5.210 | 1.00 85.24 | C |
| ATOM | 1844 | CB | ASN | A | 234 | 94.073 | 36.455 | −6.043 | 1.00 85.03 | C |
| ATOM | 1845 | CG | ASN | A | 234 | 92.838 | 36.264 | −5.192 | 1.00 84.34 | C |
| ATOM | 1846 | OD1 | ASN | A | 234 | 92.732 | 35.297 | −4.442 | 1.00 86.46 | O |
| ATOM | 1847 | ND2 | ASN | A | 234 | 91.886 | 37.171 | −5.322 | 1.00 85.12 | N |
| ATOM | 1848 | C | ASN | A | 234 | 96.554 | 36.467 | −6.115 | 1.00 86.89 | C |
| ATOM | 1849 | O | ASN | A | 234 | 96.942 | 37.384 | −6.843 | 1.00 87.53 | O |
| ATOM | 1850 | N | ASN | A | 235 | 97.151 | 35.278 | −6.060 | 1.00 86.38 | N |
| ATOM | 1851 | CA | ASN | A | 235 | 98.233 | 34.899 | −6.979 | 1.00 86.26 | C |
| ATOM | 1852 | CB | ASN | A | 235 | 99.465 | 34.387 | −6.221 | 1.00 86.78 | C |
| ATOM | 1853 | CG | ASN | A | 235 | 99.156 | 33.219 | −5.310 | 1.00 89.65 | C |
| ATOM | 1854 | OD1 | ASN | A | 235 | 98.019 | 32.754 | −5.232 | 1.00 91.82 | O |
| ATOM | 1855 | ND2 | ASN | A | 235 | 100.173 | 32.742 | −4.603 | 1.00 93.67 | N |
| ATOM | 1856 | C | ASN | A | 235 | 97.753 | 33.875 | −8.019 | 1.00 86.12 | C |
| ATOM | 1857 | O | ASN | A | 235 | 98.416 | 33.661 | −9.035 | 1.00 86.94 | O |
| ATOM | 1858 | N | GLU | A | 236 | 96.610 | 33.240 | −7.749 | 1.00 84.08 | N |
| ATOM | 1859 | CA | GLU | A | 236 | 95.962 | 32.338 | −8.697 | 1.00 84.15 | C |
| ATOM | 1860 | CB | GLU | A | 236 | 95.678 | 30.980 | −8.058 | 1.00 86.71 | C |
| ATOM | 1861 | CG | GLU | A | 236 | 96.901 | 30.285 | −7.479 | 1.00 87.83 | C |
| ATOM | 1862 | CD | GLU | A | 236 | 97.991 | 30.039 | −8.502 | 1.00 88.15 | C |
| ATOM | 1863 | OE1 | GLU | A | 236 | 97.673 | 29.826 | −9.691 | 1.00 90.60 | O |
| ATOM | 1864 | OE2 | GLU | A | 236 | 99.171 | 30.051 | −8.107 | 1.00 91.39 | O |
| ATOM | 1865 | C | GLU | A | 236 | 94.652 | 32.977 | −9.136 | 1.00 83.78 | C |
| ATOM | 1866 | O | GLU | A | 236 | 93.927 | 33.542 | −8.314 | 1.00 82.36 | O |
| ATOM | 1867 | N | PHE | A | 237 | 94.351 | 32.889 | −10.431 | 1.00 82.30 | N |
| ATOM | 1868 | CA | PHE | A | 237 | 93.163 | 33.530 | −10.991 | 1.00 79.56 | C |
| ATOM | 1869 | CB | PHE | A | 237 | 93.587 | 34.682 | −11.900 | 1.00 78.27 | C |
| ATOM | 1870 | CG | PHE | A | 237 | 94.560 | 35.626 | −11.260 | 1.00 77.48 | C |
| ATOM | 1871 | CD1 | PHE | A | 237 | 94.115 | 36.639 | −10.424 | 1.00 78.26 | C |
| ATOM | 1872 | CE1 | PHE | A | 237 | 95.011 | 37.512 | −9.830 | 1.00 79.61 | C |
| ATOM | 1873 | CZ | PHE | A | 237 | 96.368 | 37.378 | −10.069 | 1.00 80.36 | C |
| ATOM | 1874 | CE2 | PHE | A | 237 | 96.825 | 36.370 | −10.900 | 1.00 79.83 | C |
| ATOM | 1875 | CD2 | PHE | A | 237 | 95.922 | 35.499 | −11.489 | 1.00 78.69 | C |
| ATOM | 1876 | C | PHE | A | 237 | 92.275 | 32.558 | −11.763 | 1.00 79.29 | C |
| ATOM | 1877 | O | PHE | A | 237 | 91.539 | 32.967 | −12.664 | 1.00 81.99 | O |
| ATOM | 1878 | N | ASP | A | 238 | 92.327 | 31.279 | −11.400 | 1.00 77.44 | N |
| ATOM | 1879 | CA | ASP | A | 238 | 91.510 | 30.265 | −12.062 | 1.00 77.10 | C |
| ATOM | 1880 | CB | ASP | A | 238 | 91.932 | 28.856 | −11.639 | 1.00 78.33 | C |
| ATOM | 1881 | CG | ASP | A | 238 | 93.362 | 28.523 | −12.033 | 1.00 79.57 | C |
| ATOM | 1882 | OD1 | ASP | A | 238 | 94.279 | 29.312 | −11.712 | 1.00 79.76 | O |
| ATOM | 1883 | OD2 | ASP | A | 238 | 93.567 | 27.455 | −12.648 | 1.00 82.19 | O |
| ATOM | 1884 | C | ASP | A | 238 | 90.031 | 30.473 | −11.746 | 1.00 75.52 | C |
| ATOM | 1885 | O | ASP | A | 238 | 89.684 | 31.143 | −10.775 | 1.00 73.58 | O |
| ATOM | 1886 | N | ASP | A | 239 | 89.164 | 29.900 | −12.572 | 1.00 77.44 | N |
| ATOM | 1887 | CA | ASP | A | 239 | 87.727 | 30.036 | −12.373 | 1.00 78.86 | C |
| ATOM | 1888 | CB | ASP | A | 239 | 86.955 | 29.654 | −13.642 | 1.00 81.52 | C |
| ATOM | 1889 | CG | ASP | A | 239 | 85.477 | 30.011 | −13.560 | 1.00 82.95 | C |
| ATOM | 1890 | OD1 | ASP | A | 239 | 84.643 | 29.085 | −13.509 | 1.00 78.59 | O |
| ATOM | 1891 | OD2 | ASP | A | 239 | 85.153 | 31.218 | −13.519 | 1.00 85.56 | O |
| ATOM | 1892 | C | ASP | A | 239 | 87.260 | 29.164 | −11.216 | 1.00 77.00 | C |
| ATOM | 1893 | O | ASP | A | 239 | 87.864 | 28.137 | −10.916 | 1.00 77.36 | O |
| ATOM | 1894 | N | TYR | A | 240 | 86.175 | 29.599 | −10.584 | 1.00 77.15 | N |
| ATOM | 1895 | CA | TYR | A | 240 | 85.514 | 28.897 | −9.484 | 1.00 78.20 | C |
| ATOM | 1896 | CB | TYR | A | 240 | 84.096 | 29.465 | −9.351 | 1.00 78.56 | C |
| ATOM | 1897 | CG | TYR | A | 240 | 83.250 | 28.928 | −8.219 | 1.00 78.85 | C |
| ATOM | 1898 | CD1 | TYR | A | 240 | 82.506 | 27.762 | −8.372 | 1.00 80.37 | C |
| ATOM | 1899 | CE1 | TYR | A | 240 | 81.712 | 27.279 | −7.344 | 1.00 81.09 | C |
| ATOM | 1900 | CZ | TYR | A | 240 | 81.641 | 27.975 | −6.150 | 1.00 79.15 | C |
| ATOM | 1901 | OH | TYR | A | 240 | 80.854 | 27.504 | −5.126 | 1.00 80.96 | O |
| ATOM | 1902 | CE2 | TYR | A | 240 | 82.359 | 29.141 | −5.979 | 1.00 77.95 | C |
| ATOM | 1903 | CD2 | TYR | A | 240 | 83.152 | 29.616 | −7.011 | 1.00 77.64 | C |
| ATOM | 1904 | C | TYR | A | 240 | 85.456 | 27.376 | −9.667 | 1.00 80.18 | C |
| ATOM | 1905 | O | TYR | A | 240 | 85.729 | 26.629 | −8.727 | 1.00 81.92 | O |
| ATOM | 1906 | N | ILE | A | 241 | 85.119 | 26.926 | −10.874 | 1.00 83.74 | N |
| ATOM | 1907 | CA | ILE | A | 241 | 84.960 | 25.487 | −11.153 | 1.00 86.17 | C |
| ATOM | 1908 | CB | ILE | A | 241 | 83.893 | 25.231 | −12.245 | 1.00 88.43 | C |
| ATOM | 1909 | CG1 | ILE | A | 241 | 84.370 | 25.716 | −13.623 | 1.00 93.04 | C |
| ATOM | 1910 | CD1 | ILE | A | 241 | 83.371 | 25.475 | −14.738 | 1.00 94.29 | C |
| ATOM | 1911 | CG2 | ILE | A | 241 | 82.582 | 25.897 | −11.854 | 1.00 88.01 | C |
| ATOM | 1912 | C | ILE | A | 241 | 86.254 | 24.744 | −11.523 | 1.00 83.81 | C |
| ATOM | 1913 | O | ILE | A | 241 | 86.244 | 23.519 | −11.653 | 1.00 82.49 | O |
| ATOM | 1914 | N | ALA | A | 242 | 87.356 | 25.474 | −11.682 | 1.00 81.78 | N |
| ATOM | 1915 | CA | ALA | A | 242 | 88.653 | 24.872 | −12.006 | 1.00 82.72 | C |
| ATOM | 1916 | CB | ALA | A | 242 | 89.058 | 25.243 | −13.426 | 1.00 83.77 | C |
| ATOM | 1917 | C | ALA | A | 242 | 89.720 | 25.330 | −11.007 | 1.00 81.87 | C |
| ATOM | 1918 | O | ALA | A | 242 | 90.746 | 25.886 | −11.403 | 1.00 86.24 | O |
| ATOM | 1919 | N | PRO | A | 243 | 89.491 | 25.078 | −9.706 | 1.00 78.01 | N |
| ATOM | 1920 | CA | PRO | A | 243 | 90.380 | 25.570 | −8.659 | 1.00 76.24 | C |
| ATOM | 1921 | CB | PRO | A | 243 | 89.671 | 25.135 | −7.374 | 1.00 76.42 | C |
| ATOM | 1922 | CG | PRO | A | 243 | 88.922 | 23.922 | −7.762 | 1.00 76.32 | C |
| ATOM | 1923 | CD | PRO | A | 243 | 88.454 | 24.189 | −9.159 | 1.00 77.94 | C |
| ATOM | 1924 | C | PRO | A | 243 | 91.775 | 24.963 | −8.688 | 1.00 76.10 | C |
| ATOM | 1925 | O | PRO | A | 243 | 91.943 | 23.840 | −9.163 | 1.00 79.70 | O |
| ATOM | 1926 | N | PRO | A | 244 | 92.774 | 25.704 | −8.180 | 1.00 75.98 | N |
| ATOM | 1927 | CA | PRO | A | 244 | 94.118 | 25.170 | −8.030 | 1.00 76.31 | C |
| ATOM | 1928 | CB | PRO | A | 244 | 94.962 | 26.420 | −7.789 | 1.00 76.74 | C |
| ATOM | 1929 | CG | PRO | A | 244 | 94.036 | 27.354 | −7.101 | 1.00 78.02 | C |
| ATOM | 1930 | CD | PRO | A | 244 | 92.691 | 27.103 | −7.721 | 1.00 78.51 | C |
| ATOM | 1931 | C | PRO | A | 244 | 94.177 | 24.244 | −6.818 | 1.00 76.54 | C |
| ATOM | 1932 | O | PRO | A | 244 | 93.361 | 24.386 | −5.906 | 1.00 77.43 | O |
| ATOM | 1933 | N | PHE | A | 245 | 95.121 | 23.306 | −6.813 | 1.00 74.44 | N |
| ATOM | 1934 | CA | PHE | A | 245 | 95.264 | 22.362 | −5.706 | 1.00 73.61 | C |
| ATOM | 1935 | CB | PHE | A | 245 | 94.775 | 20.975 | −6.115 | 1.00 74.52 | C |
| ATOM | 1936 | CG | PHE | A | 245 | 93.368 | 20.944 | −6.629 | 1.00 73.98 | C |
| ATOM | 1937 | CD1 | PHE | A | 245 | 92.323 | 21.415 | −5.855 | 1.00 74.62 | C |
| ATOM | 1938 | CE1 | PHE | A | 245 | 91.024 | 21.367 | −6.321 | 1.00 75.77 | C |
| ATOM | 1939 | CZ | PHE | A | 245 | 90.755 | 20.825 | −7.565 | 1.00 75.22 | C |
| ATOM | 1940 | CE2 | PHE | A | 245 | 91.788 | 20.338 | −8.340 | 1.00 74.75 | C |
| ATOM | 1941 | CD2 | PHE | A | 245 | 93.084 | 20.393 | −7.869 | 1.00 74.29 | C |
| ATOM | 1942 | C | PHE | A | 245 | 96.709 | 22.221 | −5.263 | 1.00 72.53 | C |
| ATOM | 1943 | O | PHE | A | 245 | 97.627 | 22.606 | −5.982 | 1.00 73.25 | O |
| ATOM | 1944 | N | TYR | A | 246 | 96.897 | 21.653 | −4.074 | 1.00 72.91 | N |
| ATOM | 1945 | CA | TYR | A | 246 | 98.229 | 21.308 | −3.579 | 1.00 72.22 | C |
| ATOM | 1946 | CB | TYR | A | 246 | 98.327 | 21.497 | −2.059 | 1.00 68.60 | C |
| ATOM | 1947 | CG | TYR | A | 246 | 98.162 | 22.946 | −1.635 | 1.00 64.47 | C |
| ATOM | 1948 | CD1 | TYR | A | 246 | 99.206 | 23.855 | −1.774 | 1.00 62.58 | C |
| ATOM | 1949 | CE1 | TYR | A | 246 | 99.059 | 25.179 | −1.400 | 1.00 61.70 | C |
| ATOM | 1950 | CZ | TYR | A | 246 | 97.859 | 25.611 | −0.878 | 1.00 63.67 | C |
| ATOM | 1951 | OH | TYR | A | 246 | 97.704 | 26.932 | −0.505 | 1.00 65.65 | O |
| ATOM | 1952 | CE2 | TYR | A | 246 | 96.807 | 24.728 | −0.731 | 1.00 64.18 | C |
| ATOM | 1953 | CD2 | TYR | A | 246 | 96.963 | 23.406 | −1.110 | 1.00 63.71 | C |
| ATOM | 1954 | C | TYR | A | 246 | 98.484 | 19.868 | −4.014 | 1.00 75.37 | C |
| ATOM | 1955 | O | TYR | A | 246 | 98.487 | 18.939 | −3.204 | 1.00 74.50 | O |
| ATOM | 1956 | N | ASN | A | 247 | 98.678 | 19.720 | −5.325 | 1.00 79.83 | N |
| ATOM | 1957 | CA | ASN | A | 247 | 98.830 | 18.434 | −6.003 | 1.00 80.51 | C |
| ATOM | 1958 | CB | ASN | A | 247 | 97.544 | 18.107 | −6.774 | 1.00 80.27 | C |
| ATOM | 1959 | CG | ASN | A | 247 | 96.464 | 17.522 | −5.903 | 1.00 83.93 | C |
| ATOM | 1960 | OD1 | ASN | A | 247 | 96.733 | 16.963 | −4.840 | 1.00 93.05 | O |
| ATOM | 1961 | ND2 | ASN | A | 247 | 95.225 | 17.621 | −6.366 | 1.00 85.79 | N |
| ATOM | 1962 | C | ASN | A | 247 | 99.952 | 18.480 | −7.020 | 1.00 82.01 | C |
| ATOM | 1963 | O | ASN | A | 247 | 100.646 | 19.491 | −7.160 | 1.00 78.89 | O |
| ATOM | 1964 | N | ASP | A | 248 | 100.112 | 17.371 | −7.739 | 1.00 84.71 | N |
| ATOM | 1965 | CA | ASP | A | 248 | 101.017 | 17.314 | −8.880 | 1.00 84.53 | C |
| ATOM | 1966 | CB | ASP | A | 248 | 100.996 | 15.918 | −9.516 | 1.00 87.09 | C |
| ATOM | 1967 | CG | ASP | A | 248 | 101.481 | 14.828 | −8.570 | 1.00 88.71 | C |
| ATOM | 1968 | OD1 | ASP | A | 248 | 102.396 | 15.092 | −7.764 | 1.00 90.98 | O |
| ATOM | 1969 | OD2 | ASP | A | 248 | 100.952 | 13.698 | −8.642 | 1.00 92.33 | O |
| ATOM | 1970 | C | ASP | A | 248 | 100.591 | 18.374 | −9.902 | 1.00 81.24 | C |
| ATOM | 1971 | O | ASP | A | 248 | 101.433 | 18.957 | −10.579 | 1.00 81.25 | O |
| ATOM | 1972 | N | LEU | A | 249 | 99.286 | 18.641 | −9.978 | 1.00 77.42 | N |
| ATOM | 1973 | CA | LEU | A | 249 | 98.732 | 19.660 | −10.873 | 1.00 75.67 | C |
| ATOM | 1974 | CB | LEU | A | 249 | 97.238 | 19.847 | −10.598 | 1.00 75.24 | C |
| ATOM | 1975 | CG | LEU | A | 249 | 96.473 | 20.856 | −11.463 | 1.00 74.40 | C |
| ATOM | 1976 | CD1 | LEU | A | 249 | 96.534 | 20.491 | −12.938 | 1.00 74.67 | C |
| ATOM | 1977 | CD2 | LEU | A | 249 | 95.028 | 20.947 | −11.005 | 1.00 74.24 | C |
| ATOM | 1978 | C | LEU | A | 249 | 99.442 | 21.014 | −10.787 | 1.00 76.45 | C |
| ATOM | 1979 | O | LEU | A | 249 | 99.513 | 21.734 | −11.782 | 1.00 77.11 | O |
| ATOM | 1980 | N | TYR | A | 250 | 99.941 | 21.366 | −9.604 | 1.00 77.44 | N |
| ATOM | 1981 | CA | TYR | A | 250 | 100.709 | 22.604 | −9.418 | 1.00 76.72 | C |
| ATOM | 1982 | CB | TYR | A | 250 | 99.971 | 23.544 | −8.460 | 1.00 76.13 | C |
| ATOM | 1983 | CG | TYR | A | 250 | 98.875 | 24.317 | −9.151 | 1.00 77.89 | C |
| ATOM | 1984 | CD1 | TYR | A | 250 | 99.041 | 25.661 | −9.470 | 1.00 78.85 | C |
| ATOM | 1985 | CE1 | TYR | A | 250 | 98.046 | 26.370 | −10.121 | 1.00 79.13 | C |
| ATOM | 1986 | CZ | TYR | A | 250 | 96.873 | 25.733 | −10.478 | 1.00 77.99 | C |
| ATOM | 1987 | OH | TYR | A | 250 | 95.886 | 26.441 | −11.125 | 1.00 78.48 | O |
| ATOM | 1988 | CE2 | TYR | A | 250 | 96.689 | 24.397 | −10.183 | 1.00 78.51 | C |
| ATOM | 1989 | CD2 | TYR | A | 250 | 97.690 | 23.696 | −9.527 | 1.00 78.94 | C |
| ATOM | 1990 | C | TYR | A | 250 | 102.139 | 22.314 | −8.949 | 1.00 77.37 | C |
| ATOM | 1991 | O | TYR | A | 250 | 102.786 | 23.154 | −8.319 | 1.00 79.16 | O |
| ATOM | 1992 | N | ASN | A | 251 | 102.627 | 21.124 | −9.302 | 1.00 75.93 | N |
| ATOM | 1993 | CA | ASN | A | 251 | 103.972 | 20.654 | −8.959 | 1.00 71.67 | C |
| ATOM | 1994 | CB | ASN | A | 251 | 105.034 | 21.535 | −9.629 | 1.00 68.52 | C |
| ATOM | 1995 | CG | ASN | A | 251 | 106.359 | 20.814 | −9.825 | 1.00 66.38 | C |
| ATOM | 1996 | OD1 | ASN | A | 251 | 106.408 | 19.585 | −9.907 | 1.00 63.22 | O |
| ATOM | 1997 | ND2 | ASN | A | 251 | 107.446 | 21.583 | −9.909 | 1.00 65.44 | N |
| ATOM | 1998 | C | ASN | A | 251 | 104.234 | 20.545 | −7.451 | 1.00 72.30 | C |
| ATOM | 1999 | O | ASN | A | 251 | 105.375 | 20.673 | −6.999 | 1.00 71.50 | O |
| ATOM | 2000 | N | PHE | A | 252 | 103.179 | 20.306 | −6.678 | 1.00 72.57 | N |
| ATOM | 2001 | CA | PHE | A | 252 | 103.325 | 20.072 | −5.247 | 1.00 73.19 | C |
| ATOM | 2002 | CB | PHE | A | 252 | 102.166 | 20.686 | −4.453 | 1.00 71.29 | C |
| ATOM | 2003 | CG | PHE | A | 252 | 102.198 | 22.192 | −4.393 | 1.00 70.12 | C |
| ATOM | 2004 | CD1 | PHE | A | 252 | 103.206 | 22.851 | −3.699 | 1.00 69.07 | C |
| ATOM | 2005 | CE1 | PHE | A | 252 | 103.239 | 24.236 | −3.636 | 1.00 67.34 | C |
| ATOM | 2006 | CZ | PHE | A | 252 | 102.253 | 24.980 | −4.258 | 1.00 66.91 | C |
| ATOM | 2007 | CE2 | PHE | A | 252 | 101.236 | 24.338 | −4.944 | 1.00 67.96 | C |
| ATOM | 2008 | CD2 | PHE | A | 252 | 101.209 | 22.952 | −5.006 | 1.00 69.30 | C |
| ATOM | 2009 | C | PHE | A | 252 | 103.407 | 18.563 | −5.028 | 1.00 75.80 | C |
| ATOM | 2010 | O | PHE | A | 252 | 102.383 | 17.886 | −4.913 | 1.00 72.23 | O |
| ATOM | 2011 | N | LYS | A | 253 | 104.634 | 18.045 | −4.987 | 1.00 81.32 | N |
| ATOM | 2012 | CA | LYS | A | 253 | 104.864 | 16.615 | −4.785 | 1.00 85.77 | C |
| ATOM | 2013 | CB | LYS | A | 253 | 106.331 | 16.241 | −5.026 | 1.00 92.98 | C |
| ATOM | 2014 | CG | LYS | A | 253 | 106.767 | 16.408 | −6.472 | 1.00 99.97 | C |
| ATOM | 2015 | CD | LYS | A | 253 | 108.226 | 16.049 | −6.675 | 1.00106.09 | C |
| ATOM | 2016 | CE | LYS | A | 253 | 109.129 | 17.247 | −6.439 | 1.00111.70 | C |
| ATOM | 2017 | NZ | LYS | A | 253 | 110.541 | 16.971 | −6.825 | 1.00115.13 | N |
| ATOM | 2018 | C | LYS | A | 253 | 104.424 | 16.191 | −3.387 | 1.00 86.29 | C |
| ATOM | 2019 | O | LYS | A | 253 | 104.542 | 16.954 | −2.421 | 1.00 88.83 | O |
| ATOM | 2020 | N | ASP | A | 254 | 103.890 | 14.979 | −3.291 | 1.00 84.69 | N |
| ATOM | 2021 | CA | ASP | A | 254 | 103.447 | 14.441 | −2.014 | 1.00 79.32 | C |
| ATOM | 2022 | CB | ASP | A | 254 | 102.777 | 13.076 | −2.216 | 1.00 79.03 | C |
| ATOM | 2023 | CG | ASP | A | 254 | 101.821 | 12.712 | −1.095 | 1.00 79.95 | C |
| ATOM | 2024 | OD1 | ASP | A | 254 | 101.391 | 13.611 | −0.345 | 1.00 79.56 | O |
| ATOM | 2025 | OD2 | ASP | A | 254 | 101.476 | 11.517 | −0.979 | 1.00 83.79 | O |
| ATOM | 2026 | C | ASP | A | 254 | 104.684 | 14.321 | −1.131 | 1.00 77.94 | C |
| ATOM | 2027 | O | ASP | A | 254 | 105.735 | 13.878 | −1.592 | 1.00 77.26 | O |
| ATOM | 2028 | N | GLY | A | 255 | 104.570 | 14.750 | 0.122 | 1.00 77.82 | N |
| ATOM | 2029 | CA | GLY | A | 255 | 105.690 | 14.703 | 1.061 | 1.00 75.19 | C |
| ATOM | 2030 | C | GLY | A | 255 | 106.428 | 16.024 | 1.162 | 1.00 73.47 | C |
| ATOM | 2031 | O | GLY | A | 255 | 105.882 | 17.078 | 0.847 | 1.00 73.15 | O |
| ATOM | 2032 | N | ALA | A | 256 | 107.681 | 15.961 | 1.599 | 1.00 73.11 | N |
| ATOM | 2033 | CA | ALA | A | 256 | 108.483 | 17.159 | 1.825 | 1.00 73.60 | C |
| ATOM | 2034 | CB | ALA | A | 256 | 109.684 | 16.829 | 2.703 | 1.00 72.94 | C |
| ATOM | 2035 | C | ALA | A | 256 | 108.948 | 17.803 | 0.522 | 1.00 74.79 | C |
| ATOM | 2036 | O | ALA | A | 256 | 109.271 | 17.110 | −0.443 | 1.00 75.91 | O |
| ATOM | 2037 | N | GLN | A | 257 | 108.985 | 19.132 | 0.514 | 1.00 74.46 | N |
| ATOM | 2038 | CA | GLN | A | 257 | 109.469 | 19.893 | −0.628 | 1.00 74.65 | C |
| ATOM | 2039 | CB | GLN | A | 257 | 108.696 | 21.213 | −0.770 | 1.00 74.41 | C |
| ATOM | 2040 | CG | GLN | A | 257 | 107.178 | 21.068 | −0.734 | 1.00 74.48 | C |
| ATOM | 2041 | CD | GLN | A | 257 | 106.627 | 20.175 | −1.841 | 1.00 75.54 | C |
| ATOM | 2042 | OE1 | GLN | A | 257 | 106.881 | 20.398 | −3.028 | 1.00 72.96 | O |
| ATOM | 2043 | NE2 | GLN | A | 257 | 105.852 | 19.169 | −1.456 | 1.00 76.19 | N |
| ATOM | 2044 | C | GLN | A | 257 | 110.958 | 20.148 | −0.401 | 1.00 75.86 | C |
| ATOM | 2045 | O | GLN | A | 257 | 111.344 | 21.135 | 0.226 | 1.00 78.46 | O |
| ATOM | 2046 | N | ASN | A | 258 | 111.786 | 19.244 | −0.918 | 1.00 77.15 | N |
| ATOM | 2047 | CA | ASN | A | 258 | 113.235 | 19.270 | −0.685 | 1.00 78.64 | C |
| ATOM | 2048 | CB | ASN | A | 258 | 113.801 | 17.857 | −0.862 | 1.00 79.34 | C |
| ATOM | 2049 | CG | ASN | A | 258 | 113.222 | 16.867 | 0.135 | 1.00 80.33 | C |
| ATOM | 2050 | OD1 | ASN | A | 258 | 112.547 | 15.911 | −0.245 | 1.00 81.10 | O |
| ATOM | 2051 | ND2 | ASN | A | 258 | 113.475 | 17.098 | 1.417 | 1.00 80.45 | N |
| ATOM | 2052 | C | ASN | A | 258 | 114.042 | 20.244 | −1.549 | 1.00 79.55 | C |
| ATOM | 2053 | O | ASN | A | 258 | 115.225 | 20.467 | −1.285 | 1.00 79.77 | O |
| ATOM | 2054 | N | ASP | A | 259 | 113.409 | 20.832 | −2.560 | 1.00 81.95 | N |
| ATOM | 2055 | CA | ASP | A | 259 | 114.106 | 21.715 | −3.508 | 1.00 84.57 | C |
| ATOM | 2056 | CB | ASP | A | 259 | 113.758 | 21.302 | −4.946 | 1.00 85.64 | C |
| ATOM | 2057 | CG | ASP | A | 259 | 112.263 | 21.364 | −5.237 | 1.00 86.82 | C |
| ATOM | 2058 | OD1 | ASP | A | 259 | 111.458 | 21.428 | −4.282 | 1.00 84.66 | O |
| ATOM | 2059 | OD2 | ASP | A | 259 | 111.893 | 21.328 | −6.427 | 1.00 87.99 | O |
| ATOM | 2060 | C | ASP | A | 259 | 113.808 | 23.204 | −3.304 | 1.00 84.43 | C |
| ATOM | 2061 | O | ASP | A | 259 | 113.875 | 23.990 | −4.254 | 1.00 89.08 | O |
| ATOM | 2062 | N | ILE | A | 260 | 113.514 | 23.590 | −2.064 | 1.00 81.70 | N |
| ATOM | 2063 | CA | ILE | A | 260 | 113.126 | 24.971 | −1.751 | 1.00 79.45 | C |
| ATOM | 2064 | CB | ILE | A | 260 | 112.008 | 25.012 | −0.679 | 1.00 78.65 | C |
| ATOM | 2065 | CG1 | ILE | A | 260 | 112.502 | 24.469 | 0.671 | 1.00 79.64 | C |
| ATOM | 2066 | CD1 | ILE | A | 260 | 111.509 | 24.642 | 1.801 | 1.00 79.25 | C |
| ATOM | 2067 | CG2 | ILE | A | 260 | 110.794 | 24.227 | −1.154 | 1.00 78.74 | C |
| ATOM | 2068 | C | ILE | A | 260 | 114.287 | 25.851 | −1.286 | 1.00 78.23 | C |
| ATOM | 2069 | O | ILE | A | 260 | 114.076 | 27.007 | −0.919 | 1.00 78.94 | O |
| ATOM | 2070 | N | PHE | A | 261 | 115.505 | 25.319 | −1.321 | 1.00 78.47 | N |
| ATOM | 2071 | CA | PHE | A | 261 | 116.668 | 26.030 | −0.788 | 1.00 81.03 | C |
| ATOM | 2072 | CB | PHE | A | 261 | 117.508 | 25.080 | 0.067 | 1.00 79.65 | C |
| ATOM | 2073 | CG | PHE | A | 261 | 116.687 | 24.280 | 1.040 | 1.00 77.23 | C |
| ATOM | 2074 | CD1 | PHE | A | 261 | 116.206 | 24.862 | 2.201 | 1.00 76.79 | C |
| ATOM | 2075 | CE1 | PHE | A | 261 | 115.435 | 24.136 | 3.089 | 1.00 75.75 | C |
| ATOM | 2076 | CZ | PHE | A | 261 | 115.130 | 22.814 | 2.818 | 1.00 75.19 | C |
| ATOM | 2077 | CE2 | PHE | A | 261 | 115.597 | 22.224 | 1.660 | 1.00 75.46 | C |
| ATOM | 2078 | CD2 | PHE | A | 261 | 116.367 | 22.957 | 0.776 | 1.00 76.57 | C |
| ATOM | 2079 | C | PHE | A | 261 | 117.507 | 26.667 | −1.884 | 1.00 83.24 | C |
| ATOM | 2080 | O | PHE | A | 261 | 117.356 | 26.345 | −3.063 | 1.00 86.36 | O |
| ATOM | 2081 | N | VAL | A | 262 | 118.393 | 27.575 | −1.481 | 1.00 86.71 | N |
| ATOM | 2082 | CA | VAL | A | 262 | 119.223 | 28.325 | −2.422 | 1.00 88.45 | C |
| ATOM | 2083 | CB | VAL | A | 262 | 120.044 | 29.424 | −1.709 | 1.00 89.79 | C |
| ATOM | 2084 | CG1 | VAL | A | 262 | 120.955 | 30.146 | −2.697 | 1.00 91.60 | C |
| ATOM | 2085 | CG2 | VAL | A | 262 | 119.120 | 30.418 | −1.016 | 1.00 90.85 | C |
| ATOM | 2086 | C | VAL | A | 262 | 120.166 | 27.404 | −3.198 | 1.00 90.80 | C |
| ATOM | 2087 | O | VAL | A | 262 | 120.316 | 27.547 | −4.413 | 1.00 95.70 | O |
| ATOM | 2088 | N | ASP | A | 263 | 120.784 | 26.457 | −2.497 | 1.00 90.45 | N |
| ATOM | 2089 | CA | ASP | A | 263 | 121.726 | 25.519 | −3.118 | 1.00 88.68 | C |
| ATOM | 2090 | CB | ASP | A | 263 | 122.763 | 25.043 | −2.087 | 1.00 89.04 | C |
| ATOM | 2091 | CG | ASP | A | 263 | 122.158 | 24.179 | −0.984 | 1.00 88.58 | C |
| ATOM | 2092 | OD1 | ASP | A | 263 | 120.943 | 24.289 | −0.721 | 1.00 89.30 | O |
| ATOM | 2093 | OD2 | ASP | A | 263 | 122.909 | 23.391 | −0.373 | 1.00 89.57 | O |
| ATOM | 2094 | C | ASP | A | 263 | 121.055 | 24.306 | −3.779 | 1.00 87.09 | C |
| ATOM | 2095 | O | ASP | A | 263 | 121.750 | 23.446 | −4.325 | 1.00 85.49 | O |
| ATOM | 2096 | N | SER | A | 264 | 119.723 | 24.235 | −3.731 | 1.00 87.15 | N |
| ATOM | 2097 | CA | SER | A | 264 | 118.981 | 23.097 | −4.287 | 1.00 87.74 | C |
| ATOM | 2098 | CB | SER | A | 264 | 117.471 | 23.291 | −4.125 | 1.00 91.73 | C |
| ATOM | 2099 | OG | SER | A | 264 | 117.102 | 23.275 | −2.756 | 1.00 94.76 | O |
| ATOM | 2100 | C | SER | A | 264 | 119.312 | 22.849 | −5.749 | 1.00 85.13 | C |
| ATOM | 2101 | O | SER | A | 264 | 119.709 | 21.745 | −6.108 | 1.00 82.83 | O |
| ATOM | 2102 | N | TYR | A | 265 | 119.149 | 23.876 | −6.579 | 1.00 86.01 | N |
| ATOM | 2103 | CA | TYR | A | 265 | 119.461 | 23.775 | −8.008 | 1.00 87.83 | C |
| ATOM | 2104 | CB | TYR | A | 265 | 118.352 | 24.418 | −8.842 | 1.00 84.66 | C |
| ATOM | 2105 | CG | TYR | A | 265 | 117.039 | 23.670 | −8.788 | 1.00 81.02 | C |
| ATOM | 2106 | CD1 | TYR | A | 265 | 116.038 | 24.033 | −7.890 | 1.00 80.85 | C |
| ATOM | 2107 | CE1 | TYR | A | 265 | 114.836 | 23.343 | −7.840 | 1.00 78.37 | C |
| ATOM | 2108 | CZ | TYR | A | 265 | 114.629 | 22.274 | −8.691 | 1.00 77.16 | C |
| ATOM | 2109 | OH | TYR | A | 265 | 113.448 | 21.578 | −8.654 | 1.00 73.76 | O |
| ATOM | 2110 | CE2 | TYR | A | 265 | 115.605 | 21.897 | −9.589 | 1.00 77.96 | C |
| ATOM | 2111 | CD2 | TYR | A | 265 | 116.801 | 22.592 | −9.633 | 1.00 79.46 | C |
| ATOM | 2112 | C | TYR | A | 265 | 120.810 | 24.444 | −8.309 | 1.00 93.04 | C |
| ATOM | 2113 | O | TYR | A | 265 | 120.899 | 25.672 | −8.296 | 1.00 95.54 | O |
| ATOM | 2114 | N | PRO | A | 266 | 121.863 | 23.643 | −8.588 | 1.00 97.30 | N |
| ATOM | 2115 | CA | PRO | A | 266 | 123.201 | 24.199 | −8.834 | 1.00 98.80 | C |
| ATOM | 2116 | CB | PRO | A | 266 | 124.093 | 22.954 | −8.953 | 1.00 99.77 | C |
| ATOM | 2117 | CG | PRO | A | 266 | 123.288 | 21.837 | −8.389 | 1.00 99.49 | C |
| ATOM | 2118 | CD | PRO | A | 266 | 121.873 | 22.178 | −8.724 | 1.00 98.32 | C |
| ATOM | 2119 | C | PRO | A | 266 | 123.274 | 25.015 | −10.113 | 1.00 99.63 | C |
| ATOM | 2120 | O | PRO | A | 266 | 123.742 | 26.152 | −10.089 | 1.00100.28 | O |
| ATOM | 2121 | N | ASP | A | 267 | 122.797 | 24.431 | −11.210 | 1.00102.03 | N |
| ATOM | 2122 | CA | ASP | A | 267 | 122.783 | 25.090 | −12.511 | 1.00104.39 | C |
| ATOM | 2123 | CB | ASP | A | 267 | 122.805 | 24.049 | −13.637 | 1.00107.27 | C |
| ATOM | 2124 | CG | ASP | A | 267 | 124.012 | 23.135 | −13.566 | 1.00110.11 | C |
| ATOM | 2125 | OD1 | ASP | A | 267 | 125.147 | 23.647 | −13.457 | 1.00111.81 | O |
| ATOM | 2126 | OD2 | ASP | A | 267 | 123.821 | 21.904 | −13.637 | 1.00110.35 | O |
| ATOM | 2127 | C | ASP | A | 267 | 121.564 | 26.002 | −12.678 | 1.00103.12 | C |
| ATOM | 2128 | O | ASP | A | 267 | 121.354 | 26.534 | −13.764 | 1.00102.02 | O |
| ATOM | 2129 | N | GLY | A | 268 | 120.760 | 26.172 | −11.625 | 1.00101.76 | N |
| ATOM | 2130 | CA | GLY | A | 268 | 119.619 | 27.079 | −11.662 | 1.00100.79 | C |
| ATOM | 2131 | C | GLY | A | 268 | 118.421 | 26.487 | −12.372 | 1.00100.45 | C |
| ATOM | 2132 | O | GLY | A | 268 | 118.362 | 25.281 | −12.615 | 1.00101.51 | O |
| ATOM | 2133 | N | ILE | A | 269 | 117.471 | 27.354 | −12.712 | 1.00 99.07 | N |
| ATOM | 2134 | CA | ILE | A | 269 | 116.263 | 26.968 | −13.431 | 1.00 97.44 | C |
| ATOM | 2135 | CB | ILE | A | 269 | 114.996 | 27.165 | −12.561 | 1.00 97.79 | C |
| ATOM | 2136 | CG1 | ILE | A | 269 | 115.128 | 26.365 | −11.255 | 1.00 96.99 | C |
| ATOM | 2137 | CD1 | ILE | A | 269 | 115.244 | 27.208 | −10.004 | 1.00 97.80 | C |
| ATOM | 2138 | CG2 | ILE | A | 269 | 113.731 | 26.752 | −13.309 | 1.00 99.53 | C |
| ATOM | 2139 | C | ILE | A | 269 | 116.176 | 27.798 | −14.713 | 1.00 97.53 | C |
| ATOM | 2140 | O | ILE | A | 269 | 115.593 | 28.878 | −14.708 | 1.00 98.28 | O |
| ATOM | 2141 | N | PRO | A | 270 | 116.793 | 27.312 | −15.808 | 1.00 98.11 | N |
| ATOM | 2142 | CA | PRO | A | 270 | 116.708 | 27.974 | −17.115 | 1.00 98.64 | C |
| ATOM | 2143 | CB | PRO | A | 270 | 117.383 | 26.973 | −18.050 | 1.00 99.56 | C |
| ATOM | 2144 | CG | PRO | A | 270 | 118.402 | 26.311 | −17.193 | 1.00100.10 | C |
| ATOM | 2145 | CD | PRO | A | 270 | 117.822 | 26.255 | −15.806 | 1.00 99.54 | C |
| ATOM | 2146 | C | PRO | A | 270 | 115.277 | 28.264 | −17.590 | 1.00 97.88 | C |
| ATOM | 2147 | O | PRO | A | 270 | 115.063 | 29.227 | −18.329 | 1.00 99.51 | O |
| ATOM | 2148 | N | LEU | A | 271 | 114.317 | 27.437 | −17.170 | 1.00 95.54 | N |
| ATOM | 2149 | CA | LEU | A | 271 | 112.901 | 27.631 | −17.509 | 1.00 95.53 | C |
| ATOM | 2150 | CB | LEU | A | 271 | 112.035 | 26.515 | −16.919 | 1.00 93.02 | C |
| ATOM | 2151 | CG | LEU | A | 271 | 111.789 | 25.274 | −17.775 | 1.00 91.39 | C |
| ATOM | 2152 | CD1 | LEU | A | 271 | 113.066 | 24.535 | −18.101 | 1.00 89.61 | C |
| ATOM | 2153 | CD2 | LEU | A | 271 | 110.834 | 24.345 | −17.046 | 1.00 93.51 | C |
| ATOM | 2154 | C | LEU | A | 271 | 112.338 | 28.975 | −17.055 | 1.00100.00 | C |
| ATOM | 2155 | O | LEU | A | 271 | 111.328 | 29.430 | −17.586 | 1.00103.24 | O |
| ATOM | 2156 | N | GLU | A | 272 | 112.974 | 29.598 | −16.068 | 1.00105.60 | N |
| ATOM | 2157 | CA | GLU | A | 272 | 112.547 | 30.912 | −15.595 | 1.00111.95 | C |
| ATOM | 2158 | CB | GLU | A | 272 | 113.237 | 31.255 | −14.272 | 1.00115.04 | C |
| ATOM | 2159 | CG | GLU | A | 272 | 112.796 | 30.355 | −13.123 | 1.00115.05 | C |
| ATOM | 2160 | CD | GLU | A | 272 | 113.542 | 30.617 | −11.825 | 1.00116.07 | C |
| ATOM | 2161 | OE1 | GLU | A | 272 | 114.662 | 31.175 | −11.865 | 1.00115.23 | O |
| ATOM | 2162 | OE2 | GLU | A | 272 | 113.005 | 30.252 | −10.757 | 1.00113.02 | O |
| ATOM | 2163 | C | GLU | A | 272 | 112.778 | 32.014 | −16.640 | 1.00114.41 | C |
| ATOM | 2164 | O | GLU | A | 272 | 112.257 | 33.118 | −16.497 | 1.00115.45 | O |
| ATOM | 2165 | N | GLN | A | 273 | 113.557 | 31.715 | −17.681 | 1.00118.50 | N |
| ATOM | 2166 | CA | GLN | A | 273 | 113.747 | 32.640 | −18.800 | 1.00125.42 | C |
| ATOM | 2167 | CB | GLN | A | 273 | 114.790 | 32.092 | −19.781 | 1.00125.39 | C |
| ATOM | 2168 | CG | GLN | A | 273 | 115.017 | 32.962 | −21.010 | 1.00125.94 | C |
| ATOM | 2169 | CD | GLN | A | 273 | 116.022 | 32.366 | −21.977 | 1.00127.86 | C |
| ATOM | 2170 | OE1 | GLN | A | 273 | 115.685 | 32.049 | −23.118 | 1.00127.00 | O |
| ATOM | 2171 | NE2 | GLN | A | 273 | 117.259 | 32.198 | −21.522 | 1.00129.88 | N |
| ATOM | 2172 | C | GLN | A | 273 | 112.422 | 32.879 | −19.529 | 1.00132.03 | C |
| ATOM | 2173 | O | GLN | A | 273 | 112.161 | 33.984 | −20.010 | 1.00133.87 | O |
| ATOM | 2174 | N | LYS | A | 274 | 111.592 | 31.840 | −19.601 | 1.00138.59 | N |
| ATOM | 2175 | CA | LYS | A | 274 | 110.282 | 31.923 | −20.253 | 1.00141.98 | C |
| ATOM | 2176 | CB | LYS | A | 274 | 109.711 | 30.519 | −20.480 | 1.00144.49 | C |
| ATOM | 2177 | CG | LYS | A | 274 | 110.568 | 29.656 | −21.397 | 1.00147.46 | C |
| ATOM | 2178 | CD | LYS | A | 274 | 110.051 | 28.230 | −21.495 | 1.00149.98 | C |
| ATOM | 2179 | CE | LYS | A | 274 | 110.973 | 27.373 | −22.348 | 1.00151.33 | C |
| ATOM | 2180 | NZ | LYS | A | 274 | 110.500 | 25.966 | −22.455 | 1.00152.26 | N |
| ATOM | 2181 | C | LYS | A | 274 | 109.295 | 32.783 | −19.456 | 1.00143.02 | C |
| ATOM | 2182 | O | LYS | A | 274 | 108.337 | 33.311 | −20.018 | 1.00141.96 | O |
| ATOM | 2183 | N | LEU | A | 275 | 109.533 | 32.916 | −18.152 | 1.00149.30 | N |
| ATOM | 2184 | CA | LEU | A | 275 | 108.716 | 33.777 | −17.291 | 1.00154.74 | C |
| ATOM | 2185 | CB | LEU | A | 275 | 108.867 | 33.374 | −15.819 | 1.00158.31 | C |
| ATOM | 2186 | CG | LEU | A | 275 | 108.535 | 31.925 | −15.445 | 1.00162.45 | C |
| ATOM | 2187 | CD1 | LEU | A | 275 | 108.845 | 31.673 | −13.976 | 1.00161.31 | C |
| ATOM | 2188 | CD2 | LEU | A | 275 | 107.082 | 31.590 | −15.755 | 1.00162.69 | C |
| ATOM | 2189 | C | LEU | A | 275 | 109.105 | 35.249 | −17.455 | 1.00154.01 | C |
| ATOM | 2190 | O | LEU | A | 275 | 108.273 | 36.138 | −17.273 | 1.00153.38 | O |
| ATOM | 2191 | N | ILE | A | 276 | 110.371 | 35.496 | −17.792 | 1.00155.71 | N |
| ATOM | 2192 | CA | ILE | A | 276 | 110.884 | 36.858 | −17.988 | 1.00155.92 | C |
| ATOM | 2193 | CB | ILE | A | 276 | 112.436 | 36.874 | −18.018 | 1.00156.41 | C |
| ATOM | 2194 | CG1 | ILE | A | 276 | 112.996 | 36.460 | −16.651 | 1.00155.75 | C |
| ATOM | 2195 | CD1 | ILE | A | 276 | 114.482 | 36.168 | −16.645 | 1.00153.90 | C |
| ATOM | 2196 | CG2 | ILE | A | 276 | 112.967 | 38.256 | −18.385 | 1.00155.85 | C |
| ATOM | 2197 | C | ILE | A | 276 | 110.315 | 37.506 | −19.259 | 1.00154.59 | C |
| ATOM | 2198 | O | ILE | A | 276 | 110.170 | 38.728 | −19.320 | 1.00152.55 | O |
| ATOM | 2199 | N | SER | A | 277 | 109.987 | 36.690 | −20.261 | 1.00154.98 | N |
| ATOM | 2200 | CA | SER | A | 277 | 109.430 | 37.192 | −21.524 | 1.00153.95 | C |
| ATOM | 2201 | CB | SER | A | 277 | 109.402 | 36.082 | −22.582 | 1.00150.69 | C |
| ATOM | 2202 | OG | SER | A | 277 | 108.569 | 35.007 | −22.185 | 1.00147.26 | O |
| ATOM | 2203 | C | SER | A | 277 | 108.027 | 37.791 | −21.363 | 1.00156.19 | C |
| ATOM | 2204 | O | SER | A | 277 | 107.674 | 38.743 | −22.061 | 1.00157.19 | O |
| ATOM | 2205 | N | GLU | A | 278 | 107.239 | 37.231 | −20.446 | 1.00156.49 | N |
| ATOM | 2206 | CA | GLU | A | 278 | 105.871 | 37.700 | −20.194 | 1.00152.92 | C |
| ATOM | 2207 | CB | GLU | A | 278 | 105.094 | 36.667 | −19.369 | 1.00151.29 | C |
| ATOM | 2208 | CG | GLU | A | 278 | 104.885 | 35.335 | −20.078 | 1.00150.66 | C |
| ATOM | 2209 | CD | GLU | A | 278 | 104.176 | 34.299 | −19.219 | 1.00149.23 | C |
| ATOM | 2210 | OE1 | GLU | A | 278 | 104.099 | 34.478 | −17.984 | 1.00145.23 | O |
| ATOM | 2211 | OE2 | GLU | A | 278 | 103.700 | 33.292 | −19.784 | 1.00148.65 | O |
| ATOM | 2212 | C | GLU | A | 278 | 105.854 | 39.051 | −19.480 | 1.00149.61 | C |
| ATOM | 2213 | O | GLU | A | 278 | 106.758 | 39.374 | −18.709 | 1.00145.55 | O |
| HETATM | 2214 | C1 | NAG | A | 300 | 92.231 | 29.653 | 25.803 | 1.00 88.06 | C |
| HETATM | 2215 | C2 | NAG | A | 300 | 93.546 | 29.976 | 26.521 | 1.00 88.70 | C |
| HETATM | 2216 | N2 | NAG | A | 300 | 94.628 | 30.106 | 25.559 | 1.00 88.07 | N |
| HETATM | 2217 | C7 | NAG | A | 300 | 94.728 | 31.148 | 24.730 | 1.00 88.36 | C |
| HETATM | 2218 | O7 | NAG | A | 300 | 93.922 | 32.075 | 24.698 | 1.00 86.86 | O |
| HETATM | 2219 | C8 | NAG | A | 300 | 95.899 | 31.162 | 23.792 | 1.00 89.47 | C |
| HETATM | 2220 | C3 | NAG | A | 300 | 93.936 | 28.982 | 27.618 | 1.00 93.40 | C |
| HETATM | 2221 | O3 | NAG | A | 300 | 94.906 | 29.554 | 28.468 | 1.00 91.90 | O |
| HETATM | 2222 | C4 | NAG | A | 300 | 92.725 | 28.576 | 28.442 | 1.00 97.32 | C |
| HETATM | 2223 | O4 | NAG | A | 300 | 93.069 | 27.586 | 29.395 | 1.00107.70 | O |
| HETATM | 2224 | C5 | NAG | A | 300 | 91.667 | 28.059 | 27.480 | 1.00 94.56 | C |
| HETATM | 2225 | C6 | NAG | A | 300 | 90.444 | 27.489 | 28.189 | 1.00 95.47 | C |
| HETATM | 2226 | O6 | NAG | A | 300 | 89.905 | 28.462 | 29.053 | 1.00 98.19 | O |
| HETATM | 2227 | O5 | NAG | A | 300 | 91.261 | 29.151 | 26.700 | 1.00 88.89 | O |
| HETATM | 2228 | C1 | NAG | A | 301 | 93.078 | 28.120 | 30.733 | 1.00119.47 | C |
| HETATM | 2229 | C2 | NAG | A | 301 | 92.849 | 26.984 | 31.739 | 1.00125.81 | C |
| HETATM | 2230 | N2 | NAG | A | 301 | 91.499 | 26.413 | 31.646 | 1.00126.51 | N |
| HETATM | 2231 | C7 | NAG | A | 301 | 90.352 | 27.055 | 31.942 | 1.00121.91 | C |
| HETATM | 2232 | O7 | NAG | A | 301 | 90.283 | 28.222 | 32.329 | 1.00120.28 | O |
| HETATM | 2233 | C8 | NAG | A | 301 | 89.070 | 26.285 | 31.782 | 1.00117.84 | C |
| HETATM | 2234 | C3 | NAG | A | 301 | 93.193 | 27.361 | 33.192 | 1.00131.36 | C |
| HETATM | 2235 | O3 | NAG | A | 301 | 93.482 | 26.189 | 33.923 | 1.00130.30 | O |
| HETATM | 2236 | C4 | NAG | A | 301 | 94.367 | 28.330 | 33.322 | 1.00133.81 | C |
| HETATM | 2237 | O4 | NAG | A | 301 | 94.322 | 28.954 | 34.594 | 1.00141.80 | O |
| HETATM | 2238 | C5 | NAG | A | 301 | 94.319 | 29.408 | 32.244 | 1.00130.44 | C |
| HETATM | 2239 | C6 | NAG | A | 301 | 95.511 | 30.359 | 32.319 | 1.00130.22 | C |
| HETATM | 2240 | O6 | NAG | A | 301 | 96.715 | 29.646 | 32.140 | 1.00133.23 | O |
| HETATM | 2241 | O5 | NAG | A | 301 | 94.303 | 28.769 | 30.987 | 1.00122.75 | O |
| HETATM | 2242 | C1 | BMA | A | 302 | 95.375 | 28.514 | 35.477 | 1.00149.86 | C |
| HETATM | 2243 | O5 | BMA | A | 302 | 94.924 | 27.535 | 36.414 | 1.00151.87 | O |
| HETATM | 2244 | C5 | BMA | A | 302 | 95.978 | 27.001 | 37.222 | 1.00152.13 | C |
| HETATM | 2245 | C6 | BMA | A | 302 | 95.407 | 25.922 | 38.135 | 1.00149.41 | C |
| HETATM | 2246 | O6 | BMA | A | 302 | 96.469 | 25.123 | 38.669 | 1.00146.07 | O |
| HETATM | 2247 | C4 | BMA | A | 302 | 96.644 | 28.114 | 38.030 | 1.00154.34 | C |
| HETATM | 2248 | O4 | BMA | A | 302 | 97.780 | 27.590 | 38.726 | 1.00158.54 | O |
| HETATM | 2249 | C3 | BMA | A | 302 | 97.086 | 29.262 | 37.127 | 1.00152.99 | C |
| HETATM | 2250 | O3 | BMA | A | 302 | 97.557 | 30.359 | 37.919 | 1.00152.44 | O |
| HETATM | 2251 | C2 | BMA | A | 302 | 95.929 | 29.708 | 36.241 | 1.00151.92 | C |
| HETATM | 2252 | O2 | BMA | A | 302 | 94.893 | 30.288 | 37.041 | 1.00152.03 | O |
| HETATM | 2253 | C1 | NAG | A | 310 | 94.583 | 20.869 | 27.777 | 1.00109.65 | C |
| HETATM | 2254 | C2 | NAG | A | 310 | 95.465 | 21.038 | 29.004 | 1.00117.28 | C |
| HETATM | 2255 | N2 | NAG | A | 310 | 94.903 | 22.024 | 29.913 | 1.00119.62 | N |
| HETATM | 2256 | C7 | NAG | A | 310 | 94.647 | 21.772 | 31.201 | 1.00120.08 | C |
| HETATM | 2257 | O7 | NAG | A | 310 | 94.862 | 20.689 | 31.749 | 1.00116.82 | O |
| HETATM | 2258 | C8 | NAG | A | 310 | 94.062 | 22.903 | 31.995 | 1.00120.22 | C |
| HETATM | 2259 | C3 | NAG | A | 310 | 96.852 | 21.445 | 28.534 | 1.00119.67 | C |
| HETATM | 2260 | O3 | NAG | A | 310 | 97.726 | 21.550 | 29.634 | 1.00119.10 | O |
| HETATM | 2261 | C4 | NAG | A | 310 | 97.370 | 20.405 | 27.550 | 1.00122.93 | C |
| HETATM | 2262 | O4 | NAG | A | 310 | 98.524 | 20.918 | 26.929 | 1.00135.19 | O |
| HETATM | 2263 | C5 | NAG | A | 310 | 96.349 | 20.102 | 26.453 | 1.00118.12 | C |
| HETATM | 2264 | C6 | NAG | A | 310 | 96.764 | 18.929 | 25.573 | 1.00118.70 | C |
| HETATM | 2265 | O6 | NAG | A | 310 | 96.949 | 17.778 | 26.368 | 1.00116.86 | O |
| HETATM | 2266 | O5 | NAG | A | 310 | 95.092 | 19.805 | 27.013 | 1.00111.69 | O |
| HETATM | 2267 | C1 | NAG | A | 311 | 99.746 | 20.381 | 27.460 | 1.00146.81 | C |
| HETATM | 2268 | C2 | NAG | A | 311 | 100.639 | 20.109 | 26.250 | 1.00148.11 | C |
| HETATM | 2269 | N2 | NAG | A | 311 | 100.161 | 18.923 | 25.555 | 1.00144.86 | N |
| HETATM | 2270 | C7 | NAG | A | 311 | 100.351 | 18.733 | 24.248 | 1.00143.26 | C |
| HETATM | 2271 | O7 | NAG | A | 311 | 100.947 | 19.539 | 23.536 | 1.00143.90 | O |
| HETATM | 2272 | C8 | NAG | A | 311 | 99.804 | 17.470 | 23.646 | 1.00142.28 | C |
| HETATM | 2273 | C3 | NAG | A | 311 | 102.113 | 19.945 | 26.600 | 1.00155.81 | C |
| HETATM | 2274 | O3 | NAG | A | 311 | 102.863 | 20.077 | 25.415 | 1.00155.79 | O |
| HETATM | 2275 | C4 | NAG | A | 311 | 102.529 | 20.992 | 27.627 | 1.00163.01 | C |
| HETATM | 2276 | O4 | NAG | A | 311 | 103.872 | 20.824 | 28.030 | 1.00170.20 | O |
| HETATM | 2277 | C5 | NAG | A | 311 | 101.598 | 20.888 | 28.828 | 1.00162.22 | C |
| HETATM | 2278 | C6 | NAG | A | 311 | 102.043 | 21.767 | 29.996 | 1.00160.93 | C |
| HETATM | 2279 | O6 | NAG | A | 311 | 101.095 | 21.690 | 31.037 | 1.00156.34 | O |
| HETATM | 2280 | O5 | NAG | A | 311 | 100.320 | 21.283 | 28.383 | 1.00155.90 | O |
| HETATM | 2281 | C1 | BMA | A | 312 | 104.801 | 21.457 | 27.128 | 1.00173.77 | C |
| HETATM | 2282 | O5 | BMA | A | 312 | 105.738 | 20.504 | 26.637 | 1.00175.56 | O |
| HETATM | 2283 | C5 | BMA | A | 312 | 106.642 | 21.022 | 25.661 | 1.00174.38 | C |
| HETATM | 2284 | C6 | BMA | A | 312 | 107.457 | 19.854 | 25.107 | 1.00173.09 | C |
| HETATM | 2285 | O6 | BMA | A | 312 | 106.897 | 19.386 | 23.867 | 1.00173.14 | O |
| HETATM | 2286 | C4 | BMA | A | 312 | 107.488 | 22.118 | 26.308 | 1.00174.90 | C |
| HETATM | 2287 | O4 | BMA | A | 312 | 108.372 | 22.697 | 25.341 | 1.00170.26 | O |
| HETATM | 2288 | C3 | BMA | A | 312 | 106.560 | 23.196 | 26.872 | 1.00176.32 | C |
| HETATM | 2289 | O3 | BMA | A | 312 | 107.308 | 24.319 | 27.392 | 1.00179.24 | O |
| HETATM | 2290 | C2 | BMA | A | 312 | 105.549 | 22.571 | 27.845 | 1.00175.80 | C |
| HETATM | 2291 | O2 | BMA | A | 312 | 106.163 | 21.983 | 28.997 | 1.00179.19 | O |
| HETATM | 2292 | C1 | MAN | A | 313 | 105.668 | 18.645 | 24.052 | 1.00173.82 | C |
| HETATM | 2293 | C2 | MAN | A | 313 | 105.951 | 17.157 | 24.240 | 1.00173.85 | C |
| HETATM | 2294 | O2 | MAN | A | 313 | 104.749 | 16.562 | 24.671 | 1.00170.58 | O |
| HETATM | 2295 | C3 | MAN | A | 313 | 106.456 | 16.548 | 22.927 | 1.00175.11 | C |
| HETATM | 2296 | O3 | MAN | A | 313 | 106.892 | 15.191 | 23.021 | 1.00179.56 | O |
| HETATM | 2297 | C4 | MAN | A | 313 | 105.468 | 16.852 | 21.794 | 1.00173.27 | C |
| HETATM | 2298 | O4 | MAN | A | 313 | 106.007 | 16.404 | 20.573 | 1.00171.08 | O |
| HETATM | 2299 | C5 | MAN | A | 313 | 105.196 | 18.355 | 21.719 | 1.00173.04 | C |
| HETATM | 2300 | C6 | MAN | A | 313 | 104.164 | 18.707 | 20.645 | 1.00169.96 | C |
| HETATM | 2301 | O6 | MAN | A | 313 | 102.944 | 18.034 | 20.866 | 1.00163.68 | O |
| HETATM | 2302 | O5 | MAN | A | 313 | 104.756 | 18.830 | 22.980 | 1.00175.64 | O |
| HETATM | 2303 | C1 | MAN | A | 314 | 105.922 | 14.184 | 23.394 | 1.00178.69 | C |
| HETATM | 2304 | C2 | MAN | A | 314 | 106.171 | 13.732 | 24.832 | 1.00175.17 | C |
| HETATM | 2305 | O2 | MAN | A | 314 | 105.094 | 12.927 | 25.259 | 1.00167.65 | O |
| HETATM | 2306 | C3 | MAN | A | 314 | 107.477 | 12.949 | 24.950 | 1.00175.89 | C |
| HETATM | 2307 | O3 | MAN | A | 314 | 107.610 | 12.413 | 26.248 | 1.00175.01 | O |
| HETATM | 2308 | C4 | MAN | A | 314 | 107.523 | 11.824 | 23.924 | 1.00177.70 | C |
| HETATM | 2309 | O4 | MAN | A | 314 | 108.804 | 11.232 | 23.946 | 1.00175.31 | O |
| HETATM | 2310 | C5 | MAN | A | 314 | 107.221 | 12.378 | 22.532 | 1.00179.10 | C |
| HETATM | 2311 | C6 | MAN | A | 314 | 107.184 | 11.279 | 21.474 | 1.00177.74 | C |
| HETATM | 2312 | O6 | MAN | A | 314 | 106.145 | 10.367 | 21.754 | 1.00177.31 | O |
| HETATM | 2313 | O5 | MAN | A | 314 | 105.974 | 13.053 | 22.545 | 1.00180.90 | O |
| HETATM | 2314 | C1 | MAN | A | 315 | 107.996 | 24.177 | 28.656 | 1.00178.04 | C |
| HETATM | 2315 | C2 | MAN | A | 315 | 107.861 | 25.503 | 29.400 | 1.00176.71 | C |
| HETATM | 2316 | O2 | MAN | A | 315 | 108.273 | 25.337 | 30.738 | 1.00176.35 | O |
| HETATM | 2317 | C3 | MAN | A | 315 | 108.691 | 26.587 | 28.720 | 1.00174.62 | C |
| HETATM | 2318 | O3 | MAN | A | 315 | 108.650 | 27.779 | 29.472 | 1.00174.02 | O |
| HETATM | 2319 | C4 | MAN | A | 315 | 110.134 | 26.123 | 28.555 | 1.00174.24 | C |
| HETATM | 2320 | O4 | MAN | A | 315 | 110.840 | 27.087 | 27.806 | 1.00169.00 | O |
| HETATM | 2321 | C5 | MAN | A | 315 | 110.186 | 24.763 | 27.856 | 1.00175.46 | C |
| HETATM | 2322 | C6 | MAN | A | 315 | 111.599 | 24.182 | 27.804 | 1.00172.54 | C |
| HETATM | 2323 | O6 | MAN | A | 315 | 112.577 | 25.196 | 27.752 | 1.00170.89 | O |
| HETATM | 2324 | O5 | MAN | A | 315 | 109.361 | 23.825 | 28.526 | 1.00178.20 | O |
| HETATM | 2325 | C1 | NAG | A | 320 | 99.704 | 11.932 | 5.032 | 1.00 78.00 | C |
| HETATM | 2326 | C2 | NAG | A | 320 | 101.063 | 11.242 | 5.063 | 1.00 79.63 | C |
| HETATM | 2327 | N2 | NAG | A | 320 | 101.690 | 11.279 | 3.754 | 1.00 81.27 | N |
| HETATM | 2328 | C7 | NAG | A | 320 | 102.732 | 12.057 | 3.458 | 1.00 83.86 | C |
| HETATM | 2329 | O7 | NAG | A | 320 | 103.272 | 12.831 | 4.251 | 1.00 85.89 | O |
| HETATM | 2330 | C8 | NAG | A | 320 | 103.254 | 11.957 | 2.055 | 1.00 84.99 | C |
| HETATM | 2331 | C3 | NAG | A | 320 | 100.933 | 9.788 | 5.507 | 1.00 79.53 | C |
| HETATM | 2332 | O3 | NAG | A | 320 | 102.213 | 9.258 | 5.761 | 1.00 78.00 | O |
| HETATM | 2333 | C4 | NAG | A | 320 | 100.100 | 9.685 | 6.772 | 1.00 80.67 | C |
| HETATM | 2334 | O4 | NAG | A | 320 | 99.818 | 8.327 | 7.056 | 1.00 85.62 | O |
| HETATM | 2335 | C5 | NAG | A | 320 | 98.796 | 10.457 | 6.615 | 1.00 78.92 | C |
| HETATM | 2336 | C6 | NAG | A | 320 | 97.984 | 10.436 | 7.902 | 1.00 81.04 | C |
| HETATM | 2337 | O6 | NAG | A | 320 | 98.777 | 10.903 | 8.973 | 1.00 82.77 | O |
| HETATM | 2338 | O5 | NAG | A | 320 | 99.095 | 11.795 | 6.297 | 1.00 77.52 | O |
| HETATM | 2339 | C1 | NAG | A | 321 | 100.443 | 7.908 | 8.284 | 1.00 90.18 | C |
| HETATM | 2340 | C2 | NAG | A | 321 | 99.708 | 6.704 | 8.850 | 1.00 90.56 | C |
| HETATM | 2341 | N2 | NAG | A | 321 | 98.319 | 7.015 | 9.118 | 1.00 86.90 | N |
| HETATM | 2342 | C7 | NAG | A | 321 | 97.320 | 6.575 | 8.360 | 1.00 86.04 | C |
| HETATM | 2343 | O7 | NAG | A | 321 | 97.469 | 5.880 | 7.356 | 1.00 86.02 | O |
| HETATM | 2344 | C8 | NAG | A | 321 | 95.949 | 6.988 | 8.795 | 1.00 86.14 | C |
| HETATM | 2345 | C3 | NAG | A | 321 | 100.336 | 6.267 | 10.162 | 1.00 95.00 | C |
| HETATM | 2346 | O3 | NAG | A | 321 | 99.774 | 5.031 | 10.540 | 1.00 94.11 | O |
| HETATM | 2347 | C4 | NAG | A | 321 | 101.852 | 6.146 | 10.078 | 1.00 99.90 | C |
| HETATM | 2348 | O4 | NAG | A | 321 | 102.343 | 6.206 | 11.401 | 1.00111.46 | O |
| HETATM | 2349 | C5 | NAG | A | 321 | 102.493 | 7.282 | 9.281 | 1.00 97.44 | C |
| HETATM | 2350 | C6 | NAG | A | 321 | 103.947 | 6.969 | 8.938 | 1.00 97.10 | C |
| HETATM | 2351 | O6 | NAG | A | 321 | 103.999 | 6.031 | 7.886 | 1.00 97.63 | O |
| HETATM | 2352 | O5 | NAG | A | 321 | 101.790 | 7.546 | 8.083 | 1.00 94.30 | O |
| HETATM | 2353 | C1 | BMA | A | 322 | 102.661 | 4.906 | 11.925 | 1.00122.99 | C |
| HETATM | 2354 | O5 | BMA | A | 322 | 101.525 | 4.109 | 12.255 | 1.00127.83 | O |
| HETATM | 2355 | C5 | BMA | A | 322 | 101.884 | 2.771 | 12.618 | 1.00133.51 | C |
| HETATM | 2356 | C6 | BMA | A | 322 | 100.615 | 1.981 | 12.924 | 1.00137.04 | C |
| HETATM | 2357 | O6 | BMA | A | 322 | 99.842 | 2.620 | 13.953 | 1.00142.80 | O |
| HETATM | 2358 | C4 | BMA | A | 322 | 102.860 | 2.751 | 13.801 | 1.00134.12 | C |
| HETATM | 2359 | O4 | BMA | A | 322 | 103.375 | 1.426 | 13.966 | 1.00134.46 | O |
| HETATM | 2360 | C3 | BMA | A | 322 | 104.027 | 3.719 | 13.595 | 1.00134.38 | C |
| HETATM | 2361 | O3 | BMA | A | 322 | 104.824 | 3.820 | 14.788 | 1.00140.14 | O |
| HETATM | 2362 | C2 | BMA | A | 322 | 103.480 | 5.082 | 13.190 | 1.00128.00 | C |
| HETATM | 2363 | O2 | BMA | A | 322 | 102.656 | 5.617 | 14.234 | 1.00123.99 | O |
| HETATM | 2364 | C1 | MAN | A | 323 | 106.165 | 4.289 | 14.504 | 1.00144.86 | C |
| HETATM | 2365 | C2 | MAN | A | 323 | 107.113 | 3.102 | 14.303 | 1.00145.83 | C |
| HETATM | 2366 | O2 | MAN | A | 323 | 108.221 | 3.519 | 13.532 | 1.00142.79 | O |
| HETATM | 2367 | C3 | MAN | A | 323 | 107.599 | 2.476 | 15.614 | 1.00148.36 | C |
| HETATM | 2368 | O3 | MAN | A | 323 | 108.743 | 1.684 | 15.376 | 1.00147.23 | O |
| HETATM | 2369 | C4 | MAN | A | 323 | 107.926 | 3.522 | 16.674 | 1.00148.74 | C |
| HETATM | 2370 | O4 | MAN | A | 323 | 108.112 | 2.888 | 17.920 | 1.00149.10 | O |
| HETATM | 2371 | C5 | MAN | A | 323 | 106.804 | 4.549 | 16.777 | 1.00147.56 | C |
| HETATM | 2372 | C6 | MAN | A | 323 | 107.115 | 5.600 | 17.840 | 1.00145.14 | C |
| HETATM | 2373 | O6 | MAN | A | 323 | 106.520 | 6.836 | 17.507 | 1.00142.88 | O |
| HETATM | 2374 | O5 | MAN | A | 323 | 106.646 | 5.160 | 15.511 | 1.00147.26 | O |
| HETATM | 2375 | C1 | MAN | A | 324 | 98.715 | 1.788 | 14.313 | 1.00148.42 | C |
| HETATM | 2376 | C2 | MAN | A | 324 | 97.413 | 2.512 | 13.959 | 1.00148.60 | C |
| HETATM | 2377 | O2 | MAN | A | 324 | 96.396 | 1.554 | 13.757 | 1.00150.22 | O |
| HETATM | 2378 | C3 | MAN | A | 324 | 96.973 | 3.530 | 15.015 | 1.00149.37 | C |
| HETATM | 2379 | O3 | MAN | A | 324 | 95.624 | 3.891 | 14.809 | 1.00147.64 | O |
| HETATM | 2380 | C4 | MAN | A | 324 | 97.141 | 2.995 | 16.432 | 1.00149.92 | C |
| HETATM | 2381 | O4 | MAN | A | 324 | 96.913 | 4.039 | 17.352 | 1.00148.37 | O |
| HETATM | 2382 | C5 | MAN | A | 324 | 98.542 | 2.423 | 16.616 | 1.00150.46 | C |
| HETATM | 2383 | C6 | MAN | A | 324 | 98.735 | 1.846 | 18.015 | 1.00148.18 | C |
| HETATM | 2384 | O6 | MAN | A | 324 | 98.707 | 2.883 | 18.969 | 1.00148.30 | O |
| HETATM | 2385 | O5 | MAN | A | 324 | 98.741 | 1.389 | 15.673 | 1.00152.09 | O |
| HETATM | 2386 | C1 | NAG | A | 330 | 113.415 | 26.632 | 18.299 | 1.00110.44 | C |
| HETATM | 2387 | C2 | NAG | A | 330 | 113.451 | 27.224 | 19.699 | 1.00114.33 | C |
| HETATM | 2388 | N2 | NAG | A | 330 | 112.698 | 28.467 | 19.720 | 1.00110.83 | N |
| HETATM | 2389 | C7 | NAG | A | 330 | 111.815 | 28.817 | 20.662 | 1.00109.92 | C |
| HETATM | 2390 | O7 | NAG | A | 330 | 111.216 | 29.888 | 20.603 | 1.00108.90 | O |
| HETATM | 2391 | C8 | NAG | A | 330 | 111.543 | 27.895 | 21.819 | 1.00109.21 | C |
| HETATM | 2392 | C3 | NAG | A | 330 | 114.898 | 27.468 | 20.090 | 1.00118.83 | C |
| HETATM | 2393 | O3 | NAG | A | 330 | 114.967 | 27.874 | 21.436 | 1.00118.84 | O |
| HETATM | 2394 | C4 | NAG | A | 330 | 115.703 | 26.186 | 19.892 | 1.00123.15 | C |
| HETATM | 2395 | O4 | NAG | A | 330 | 117.068 | 26.467 | 20.115 | 1.00134.68 | O |
| HETATM | 2396 | C5 | NAG | A | 330 | 115.502 | 25.610 | 18.490 | 1.00117.64 | C |
| HETATM | 2397 | C6 | NAG | A | 330 | 116.192 | 24.267 | 18.296 | 1.00115.10 | C |
| HETATM | 2398 | O6 | NAG | A | 330 | 115.595 | 23.304 | 19.137 | 1.00110.28 | O |
| HETATM | 2399 | O5 | NAG | A | 330 | 114.125 | 25.419 | 18.271 | 1.00113.09 | O |
| HETATM | 2400 | C1 | NAG | A | 331 | 117.623 | 25.577 | 21.098 | 1.00141.38 | C |
| HETATM | 2401 | C2 | NAG | A | 331 | 119.097 | 25.938 | 21.284 | 1.00142.10 | C |
| HETATM | 2402 | N2 | NAG | A | 331 | 119.820 | 25.923 | 20.014 | 1.00137.18 | N |
| HETATM | 2403 | C7 | NAG | A | 331 | 120.034 | 24.827 | 19.278 | 1.00132.94 | C |
| HETATM | 2404 | O7 | NAG | A | 331 | 119.651 | 23.703 | 19.593 | 1.00130.56 | O |
| HETATM | 2405 | C8 | NAG | A | 331 | 120.795 | 25.006 | 17.995 | 1.00132.22 | C |
| HETATM | 2406 | C3 | NAG | A | 331 | 119.740 | 25.076 | 22.372 | 1.00146.26 | C |
| HETATM | 2407 | O3 | NAG | A | 331 | 121.036 | 25.546 | 22.661 | 1.00145.00 | O |
| HETATM | 2408 | C4 | NAG | A | 331 | 118.870 | 25.127 | 23.623 | 1.00152.12 | C |
| HETATM | 2409 | O4 | NAG | A | 331 | 119.398 | 24.297 | 24.638 | 1.00162.28 | O |
| HETATM | 2410 | C5 | NAG | A | 331 | 117.443 | 24.712 | 23.273 | 1.00149.81 | C |
| HETATM | 2411 | C6 | NAG | A | 331 | 116.516 | 24.666 | 24.486 | 1.00149.59 | C |
| HETATM | 2412 | O6 | NAG | A | 331 | 116.467 | 25.929 | 25.110 | 1.00150.33 | O |
| HETATM | 2413 | O5 | NAG | A | 331 | 116.951 | 25.649 | 22.340 | 1.00144.92 | O |
| HETATM | 2414 | C1 | BMA | A | 332 | 119.957 | 25.093 | 25.701 | 1.00168.28 | C |
| HETATM | 2415 | O5 | BMA | A | 332 | 121.230 | 25.636 | 25.363 | 1.00170.32 | O |
| HETATM | 2416 | C5 | BMA | A | 332 | 121.709 | 26.581 | 26.321 | 1.00168.05 | C |
| HETATM | 2417 | C6 | BMA | A | 332 | 123.071 | 27.095 | 25.858 | 1.00163.68 | C |
| HETATM | 2418 | O6 | BMA | A | 332 | 122.918 | 28.350 | 25.187 | 1.00159.66 | O |
| HETATM | 2419 | C4 | BMA | A | 332 | 121.806 | 25.969 | 27.721 | 1.00171.45 | C |
| HETATM | 2420 | O4 | BMA | A | 332 | 121.940 | 27.054 | 28.648 | 1.00169.83 | O |
| HETATM | 2421 | C3 | BMA | A | 332 | 120.602 | 25.100 | 28.112 | 1.00173.51 | C |
| HETATM | 2422 | O3 | BMA | A | 332 | 120.940 | 24.206 | 29.186 | 1.00179.72 | O |
| HETATM | 2423 | C2 | BMA | A | 332 | 120.106 | 24.243 | 26.951 | 1.00170.13 | C |
| HETATM | 2424 | O2 | BMA | A | 332 | 121.018 | 23.165 | 26.702 | 1.00165.46 | O |
| HETATM | 2425 | C1 | MAN | A | 333 | 120.952 | 24.832 | 30.488 | 1.00181.80 | C |
| HETATM | 2426 | C2 | MAN | A | 333 | 122.387 | 24.853 | 31.001 | 1.00181.64 | C |
| HETATM | 2427 | O2 | MAN | A | 333 | 122.457 | 25.624 | 32.180 | 1.00179.83 | O |
| HETATM | 2428 | C3 | MAN | A | 333 | 122.877 | 23.433 | 31.267 | 1.00180.97 | C |
| HETATM | 2429 | O3 | MAN | A | 333 | 124.163 | 23.465 | 31.847 | 1.00178.86 | O |
| HETATM | 2430 | C4 | MAN | A | 333 | 121.903 | 22.692 | 32.178 | 1.00181.14 | C |
| HETATM | 2431 | O4 | MAN | A | 333 | 122.278 | 21.333 | 32.241 | 1.00179.06 | O |
| HETATM | 2432 | C5 | MAN | A | 333 | 120.464 | 22.813 | 31.673 | 1.00179.97 | C |
| HETATM | 2433 | C6 | MAN | A | 333 | 119.472 | 22.252 | 32.688 | 1.00175.98 | C |
| HETATM | 2434 | O6 | MAN | A | 333 | 118.313 | 21.812 | 32.017 | 1.00171.57 | O |
| HETATM | 2435 | O5 | MAN | A | 333 | 120.127 | 24.167 | 31.426 | 1.00181.86 | O |
| HETATM | 2436 | C1 | NAG | A | 340 | 100.888 | 27.243 | −17.586 | 1.00 88.88 | C |
| HETATM | 2437 | C2 | NAG | A | 340 | 99.518 | 26.573 | −17.707 | 1.00 90.13 | C |
| HETATM | 2438 | N2 | NAG | A | 340 | 99.446 | 25.405 | −18.592 | 1.00 92.46 | N |
| HETATM | 2439 | C7 | NAG | A | 340 | 99.882 | 25.283 | −19.848 | 1.00 92.58 | C |
| HETATM | 2440 | O7 | NAG | A | 340 | 99.757 | 24.207 | −20.432 | 1.00 91.08 | O |
| HETATM | 2441 | C8 | NAG | A | 340 | 100.517 | 26.438 | −20.572 | 1.00 94.41 | C |
| HETATM | 2442 | C3 | NAG | A | 340 | 98.440 | 27.624 | −17.910 | 1.00 89.36 | C |
| HETATM | 2443 | O3 | NAG | A | 340 | 97.185 | 26.988 | −17.974 | 1.00 89.82 | O |
| HETATM | 2444 | C4 | NAG | A | 340 | 98.497 | 28.556 | −16.702 | 1.00 89.59 | C |
| HETATM | 2445 | O4 | NAG | A | 340 | 97.691 | 29.694 | −16.929 | 1.00 94.05 | O |
| HETATM | 2446 | C5 | NAG | A | 340 | 99.924 | 29.020 | −16.373 | 1.00 88.48 | C |
| HETATM | 2447 | C6 | NAG | A | 340 | 99.977 | 29.712 | −15.011 | 1.00 87.61 | C |
| HETATM | 2448 | O6 | NAG | A | 340 | 99.455 | 28.875 | −14.001 | 1.00 84.45 | O |
| HETATM | 2449 | O5 | NAG | A | 340 | 100.874 | 27.967 | −16.379 | 1.00 88.47 | O |
| HETATM | 2450 | C1 | NAG | A | 341 | 96.435 | 29.594 | −16.232 | 1.00 96.35 | C |
| HETATM | 2451 | C2 | NAG | A | 341 | 95.794 | 30.976 | −16.163 | 1.00 96.82 | C |
| HETATM | 2452 | N2 | NAG | A | 341 | 96.637 | 31.906 | −15.427 | 1.00 95.78 | N |
| HETATM | 2453 | C7 | NAG | A | 341 | 97.606 | 32.617 | −16.010 | 1.00 95.58 | C |
| HETATM | 2454 | O7 | NAG | A | 341 | 97.874 | 32.543 | −17.210 | 1.00 95.58 | O |
| HETATM | 2455 | C8 | NAG | A | 341 | 98.401 | 33.540 | −15.131 | 1.00 95.35 | C |
| HETATM | 2456 | C3 | NAG | A | 341 | 94.416 | 30.892 | −15.520 | 1.00 99.78 | C |
| HETATM | 2457 | O3 | NAG | A | 341 | 93.789 | 32.152 | −15.588 | 1.00 99.67 | O |
| HETATM | 2458 | C4 | NAG | A | 341 | 93.591 | 29.840 | −16.252 | 1.00104.00 | C |
| HETATM | 2459 | O4 | NAG | A | 341 | 92.301 | 29.715 | −15.684 | 1.00111.33 | O |
| HETATM | 2460 | C5 | NAG | A | 341 | 94.327 | 28.504 | −16.243 | 1.00102.77 | C |
| HETATM | 2461 | C6 | NAG | A | 341 | 93.552 | 27.414 | −16.979 | 1.00104.49 | C |
| HETATM | 2462 | O6 | NAG | A | 341 | 93.598 | 27.650 | −18.370 | 1.00105.18 | O |
| HETATM | 2463 | O5 | NAG | A | 341 | 95.577 | 28.676 | −16.876 | 1.00 98.21 | O |
| HETATM | 2464 | C1 | BMA | A | 342 | 91.328 | 30.498 | −16.408 | 1.00119.64 | C |
| HETATM | 2465 | O5 | BMA | A | 342 | 91.085 | 31.765 | −15.791 | 1.00120.34 | O |
| HETATM | 2466 | C5 | BMA | A | 342 | 90.208 | 32.602 | −16.547 | 1.00121.32 | C |
| HETATM | 2467 | C6 | BMA | A | 342 | 90.160 | 33.985 | −15.906 | 1.00121.40 | C |
| HETATM | 2468 | O6 | BMA | A | 342 | 91.453 | 34.603 | −16.035 | 1.00124.56 | O |
| HETATM | 2469 | C4 | BMA | A | 342 | 88.838 | 31.936 | −16.610 | 1.00123.92 | C |
| HETATM | 2470 | O4 | BMA | A | 342 | 87.945 | 32.698 | −17.428 | 1.00121.93 | O |
| HETATM | 2471 | C3 | BMA | A | 342 | 88.953 | 30.537 | −17.203 | 1.00128.89 | C |
| HETATM | 2472 | O3 | BMA | A | 342 | 87.672 | 29.893 | −17.113 | 1.00140.69 | O |
| HETATM | 2473 | C2 | BMA | A | 342 | 90.035 | 29.699 | −16.517 | 1.00124.67 | C |
| HETATM | 2474 | O2 | BMA | A | 342 | 89.623 | 29.265 | −15.215 | 1.00123.88 | O |
| HETATM | 2475 | C1 | MAN | A | 343 | 87.247 | 29.408 | −18.403 | 1.00151.30 | C |
| HETATM | 2476 | C2 | MAN | A | 343 | 85.721 | 29.263 | −18.399 | 1.00154.03 | C |
| HETATM | 2477 | O2 | MAN | A | 343 | 85.137 | 28.979 | −19.667 | 1.00161.06 | O |
| HETATM | 2478 | C3 | MAN | A | 343 | 85.324 | 28.143 | −17.439 | 1.00150.64 | C |
| HETATM | 2479 | O3 | MAN | A | 343 | 83.935 | 27.917 | −17.508 | 1.00147.95 | O |
| HETATM | 2480 | C4 | MAN | A | 343 | 86.065 | 26.864 | −17.811 | 1.00151.44 | C |
| HETATM | 2481 | O4 | MAN | A | 343 | 85.807 | 25.852 | −16.867 | 1.00146.63 | O |
| HETATM | 2482 | C5 | MAN | A | 343 | 87.571 | 27.112 | −17.856 | 1.00153.70 | C |
| HETATM | 2483 | C6 | MAN | A | 343 | 88.343 | 25.860 | −18.278 | 1.00155.05 | C |
| HETATM | 2484 | O6 | MAN | A | 343 | 87.459 | 24.892 | −18.810 | 1.00158.77 | O |
| HETATM | 2485 | O5 | MAN | A | 343 | 87.851 | 28.175 | −18.751 | 1.00154.50 | O |
| HETATM | 2486 | C1 | MAN | A | 345 | 85.695 | 29.622 | −20.841 | 1.00166.37 | C |
| HETATM | 2487 | C2 | MAN | A | 345 | 85.634 | 31.158 | −20.781 | 1.00167.59 | C |
| HETATM | 2488 | O2 | MAN | A | 345 | 86.580 | 31.692 | −21.683 | 1.00167.20 | O |
| HETATM | 2489 | C3 | MAN | A | 345 | 84.245 | 31.731 | −21.075 | 1.00166.74 | C |
| HETATM | 2490 | O3 | MAN | A | 345 | 84.322 | 33.124 | −21.288 | 1.00162.83 | O |
| HETATM | 2491 | C4 | MAN | A | 345 | 83.617 | 31.055 | −22.288 | 1.00167.34 | C |
| HETATM | 2492 | O4 | MAN | A | 345 | 82.280 | 31.482 | −22.423 | 1.00162.36 | O |
| HETATM | 2493 | C5 | MAN | A | 345 | 83.684 | 29.542 | −22.101 | 1.00168.99 | C |
| HETATM | 2494 | C6 | MAN | A | 345 | 83.040 | 28.783 | −23.255 | 1.00168.07 | C |
| HETATM | 2495 | O6 | MAN | A | 345 | 83.134 | 27.399 | −22.995 | 1.00164.56 | O |
| HETATM | 2496 | O5 | MAN | A | 345 | 85.042 | 29.160 | −22.008 | 1.00168.63 | O |
| HETATM | 2497 | C1 | MAN | A | 344 | 91.467 | 35.742 | −16.924 | 1.00128.05 | C |
| HETATM | 2498 | C2 | MAN | A | 344 | 92.582 | 35.574 | −17.955 | 1.00130.27 | C |
| HETATM | 2499 | O2 | MAN | A | 344 | 92.454 | 36.565 | −18.952 | 1.00128.74 | O |
| HETATM | 2500 | C3 | MAN | A | 344 | 93.959 | 35.658 | −17.296 | 1.00130.86 | C |
| HETATM | 2501 | O3 | MAN | A | 344 | 94.974 | 35.677 | −18.278 | 1.00129.03 | O |
| HETATM | 2502 | C4 | MAN | A | 344 | 94.078 | 36.901 | −16.419 | 1.00129.88 | C |
| HETATM | 2503 | O4 | MAN | A | 344 | 95.251 | 36.794 | −15.641 | 1.00125.41 | O |
| HETATM | 2504 | C5 | MAN | A | 344 | 92.861 | 37.074 | −15.505 | 1.00129.52 | C |
| HETATM | 2505 | C6 | MAN | A | 344 | 92.875 | 38.441 | −14.825 | 1.00129.77 | C |
| HETATM | 2506 | O6 | MAN | A | 344 | 93.749 | 38.437 | −13.718 | 1.00128.61 | O |
| HETATM | 2507 | O5 | MAN | A | 344 | 91.648 | 36.961 | −16.230 | 1.00127.47 | O |
| HETATM | 2508 | C1 | MAN | A | 346 | 88.141 | 23.896 | −19.600 | 1.00161.77 | C |
| HETATM | 2509 | C2 | MAN | A | 346 | 88.219 | 24.347 | −21.060 | 1.00161.25 | C |
| HETATM | 2510 | O2 | MAN | A | 346 | 89.132 | 23.525 | −21.752 | 1.00159.91 | O |
| HETATM | 2511 | C3 | MAN | A | 346 | 86.856 | 24.287 | −21.743 | 1.00161.31 | C |
| HETATM | 2512 | O3 | MAN | A | 346 | 86.990 | 24.544 | −23.123 | 1.00161.03 | O |
| HETATM | 2513 | C4 | MAN | A | 346 | 86.221 | 22.919 | −21.532 | 1.00159.90 | C |
| HETATM | 2514 | O4 | MAN | A | 346 | 84.913 | 22.924 | −22.053 | 1.00159.25 | O |
| HETATM | 2515 | C5 | MAN | A | 346 | 86.185 | 22.604 | −20.039 | 1.00159.17 | C |
| HETATM | 2516 | C6 | MAN | A | 346 | 85.541 | 21.251 | −19.747 | 1.00155.90 | C |
| HETATM | 2517 | O6 | MAN | A | 346 | 86.162 | 20.229 | −20.493 | 1.00152.47 | O |
| HETATM | 2518 | O5 | MAN | A | 346 | 87.505 | 22.631 | −19.523 | 1.00163.55 | O |

1-15. (canceled)

16. A method for preparing a cellulose-based substrate for the manufacture of cellulose fibers, which method comprises at least the following steps:
a) providing a cellulose-based substrate which is capable of forming cellulose fibers;
b) bringing said substrate in contact, in the presence of an electron donor, with at least one enzyme belonging to the family of lytic polysaccharide monooxygenases (LPMOs) under conditions that are capable of ensuring oxidative cleavage of said cellulose fibers;
wherein said enzyme is a polysaccharide oxidase which has an amino acid sequence identity of at least 20% with a reference polypeptide of SEQ ID NO. 1, 2 or 3, by means of the BLAST-P comparison method, said BLAST-P comparison method resulting in an E-value of 10 e⁻³ or less.

17. The method for preparing a cellulose-based substrate as claimed in claim 16, wherein said enzyme has an amino acid sequence identity of at least 90% with a reference polypeptide of SEQ ID NO. 1, 2 or 3.

18. The method for preparing a cellulose-based substrate as claimed in claim 16, wherein the electron donor is chosen from the group consisting of: ascorbate, gallate, catechol, reduced glutathione, lignin fragments and fungal carbohydrate dehydrogenases.

19. The method for preparing a cellulose-based substrate as claimed in claim 16, wherein the cellulose-based substrate is obtained from a group consisting of: wood, a cellulose-rich fibrous plant, beetroot, citrus plants, annual straw plants, marine animals, algae, fungi bacteria.

20. The method for preparing a cellulose-based substrate as claimed in claim 16, wherein the cellulose-based substrate is chosen from chemical paper pulps.

21. The method for preparing a cellulose-based substrate as claimed in claim 16, wherein the cellulose-based substrate is a paper pulp derived from a group consisting of: wood, annual plants, fibrous plants.

22. A method for manufacturing cellulose fibers via a defibrillation process, comprising a step of providing a cellulose-based substrate prepared according to a method as claimed in claim 16.

23. A method for defibrillating a cellulose-based substrate, which method comprises at least the following steps:
a) providing a cellulose-based substrate which is capable of forming cellulose fibers, which has been placed beforehand in contact with an electron donor and an enzyme with polysaccharide oxidase activity, under conditions suitable for ensuring oxidative cleavage of said cellulose fibers in the presence of an electron donor, wherein said enzyme with polysaccharide oxidase activity has an amino acid sequence identity of at least 20% with a reference polypeptide of SEQ ID NO. 1, 2 or 3 by means of the BLAST-P comparison method, said BLAST-P comparison method resulting in an E-value of 10 e⁻³ or less; and
b) mechanically treating said cellulose-based substrate, so as to defibrillate said substrate.

24. The method for defibrillating a cellulose-based substrate as claimed in claim 23, wherein said at least one mechanical treatment step comprises at least one mechanical treatment selected from the group consisting of:
a homogenization treatment,
a microfluidization treatment,
an abrasion treatment, and
a cryomilling treatment.

25. The method for defibrillating a cellulose-based substrate as claimed in claim 23, wherein following said mechanical treatment step, said method comprises a post-treatment step selected from the group consisting of: an acid treatment, an enzymatic treatment, an oxidation, an acetylation, a silylation, a derivatization of chemical groups borne by said cellulose fibers.

26. A method for manufacturing cellulose fibers, which method comprises at least the following steps:
a) providing a cellulose-based substrate which is capable of forming cellulose fibers;
b) bringing said cellulose-based substrate in contact, in the presence of an electron donor, with at least one enzyme belonging to the family of lytic polysaccharide monooxygenases (LPMOs) under conditions that are capable of ensuring oxidative cleavage of said cellulose fibers;
c) mechanically treating said cellulose-based substrate, so as to manufacture said cellulose fibers from said cellulose-based substrate,
wherein said enzyme is a polysaccharide oxidase which has an amino acid sequence identity of at least 20% with a reference polypeptide of SEQ ID NO. 1, 2 or 3, by means of the BLAST-P comparison method, said BLAST-P comparison method resulting in an E-value of 10 e⁻³ or less.

27. The method for manufacturing cellulose fibers as claimed in claim 26, wherein said cellulose fibers are cellulose nanofibrils.

28. Cellulose fibers obtained from a defibrillation method as claimed in claim 23 or from a method for manufacturing cellulose fibers, which method comprises at least the following steps:
a) providing a cellulose-based substrate which is capable of forming cellulose fibers;
b) bringing said cellulose-based substrate in contact, in the presence of an electron donor, with at least one enzyme belonging to the family of lytic polysaccharide monooxygenases (LPMOs) under conditions that are capable of ensuring oxidative cleavage of said cellulose fibers;
c) mechanically treating said cellulose-based substrate, so as to manufacture said cellulose fibers from said cellulose-based substrate,
wherein said enzyme is a polysaccharide oxidase which has an amino acid sequence identity of at least 20% with a reference polypeptide of SEQ ID NO. 1, 2 or 3, by means of the BLAST-P comparison method, said BLAST-P comparison method resulting in an E-value of 10 e⁻³ or less.

29. The cellulose fibers as claimed in claim 28, wherein said fibers include glucose rings, of which at least one carbon atom is oxidized in position(s) C₁ or C₄.

30. The cellulose fibers as claimed in claim 28, wherein said fibers are cellulose nanofibrils.
